(19) United States
(12) Patent Application Publication
(10) Pub. No.: US 2023/0057581 A1
(43) Pub. Date: Feb. 23, 2023

(54) HETEROCYCLIC COMPOUND, ORGANIC LIGHT-EMITTING DIODE COMPRISING SAME, AND COMPOSITION FOR ORGANIC LAYER OF ORGANIC LIGHT-EMITTING DIODE

(71) Applicant: LT MATERIALS CO., LTD., Yongin-si, Gyeonggi-do (KR)

(72) Inventors: Ji-Yoon BYUN, Yongin-si (KR); Jun-Tae MO, Yongin-si (KR); Dong-Jun KIM, Yongin-si (KR)

(73) Assignee: LT MATERIALS CO., LTD., Yongin-si, Gyeonggi-do (KR)

(21) Appl. No.: 17/782,051

(22) PCT Filed: Dec. 17, 2020
(86) PCT No.: PCT/KR2020/018555

§ 371 Date: Jun. 2, 2022

(30) Foreign Application Priority Data
Dec. 26, 2019 (KR) 10-2019-0175166

(51) Int. Cl. H01L 51/00; C07D 405/04; C07D 405/14

(52) U.S. Cl. CPC H01L 51/0061; C07D 405/04; H01L 51/006; C07D 405/14; H01L 51/0067; H01L 51/0073; H01L 51/0052; H01L 51/5012

(57) ABSTRACT

The present application provides a heterocyclic compound, and an organic light emitting device containing the heterocyclic compound in an organic material layer.

### TECHNICAL FIELD

The present specification claims priority to and the benefits of Korean Patent Application No. 10-2019-0175166, filed with the Korean Intellectual Property Office on Dec. 26, 2019, the entire contents of which are incorporated herein by reference.

The present specification relates to a heterocyclic compound, an organic light-emitting device comprising the same, and a composition for an organic material layer of an organic light-emitting device.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

(Patent Document 1) U.S. Pat. No. 4,356,429

### DISCLOSURE

### Technical Problem

The present specification is directed to providing a heterocyclic compound, an organic light emitting device including the same, and a composition for an organic material layer of an organic light emitting device.

### Technical Solution

One embodiment of the present application provides a heterocyclic compound represented by the following Chemical Formula 1.

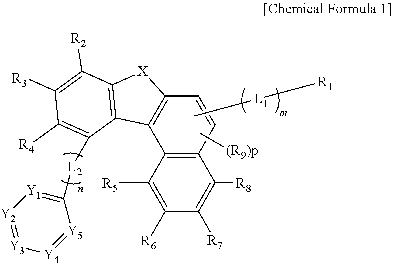

In Chemical Formula 1,

L₁ and L₂ are the same as or different from each other, and each independently a direct bond; a substituted or unsubstituted arylene group having 6 to 60 carbon atoms; or a substituted or unsubstituted heteroarylene group having 2 to 60 carbon atoms,

X is O; S; or NRa,

Y₁ to Y₅ are the same as or different from each other and each independently N or CRb, at least one or more of Y₁ to Y₅ are N, and when there are two or more CRbs, Rbs are the same as or different from each other,

R₁ is represented by the following Chemical Formula A,

R₂ to R₉ are hydrogen; deuterium; a halogen group; a cyano group; a substituted or unsubstituted alkyl group; a substituted or unsubstituted cycloalkyl group; a substituted or unsubstituted aryl group having 6 to 40 carbon atoms; or a substituted or unsubstituted heteroaryl group having 2 to 40 carbon atoms,

Ra is a substituted or unsubstituted aryl group having 6 to 40 carbon atoms,

Rb is selected from the group consisting of hydrogen; deuterium; a substituted or unsubstituted aryl group having 6 to 60 carbon atoms; and a substituted or unsubstituted heteroaryl group having 2 to 60 carbon atoms, or two or more groups adjacent to each other bond to each other to form a substituted or unsubstituted aromatic hydrocarbon ring having 6 to 60 carbon atoms or a substituted or unsubstituted heteroring having 2 to 60 carbon atoms,

m and n are each independently an integer of 0 to 3, and when m and n are each 2 or greater, substituents in the parentheses are the same as or different from each other, and

p is 0 or 1,

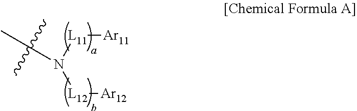

in Chemical Formula A,

L₁₁ and L₁₂ are the same as or different from each other, and each independently a direct bond; a substituted or unsubstituted arylene group having 6 to 40 carbon atoms; or a substituted or unsubstituted heteroarylene group having 2 to 40 carbon atoms,

Ar₁₁ and Ar₁₂ are the same as or different from each other, and each independently selected from the group consisting of a substituted or unsubstituted aryl group having 6 to 40 carbon atoms; and a substituted or unsubstituted heteroaryl group having 2 to 40 carbon atoms, or Ar₁₁ and Ar₁₂ bond to each other to form a substituted or unsubstituted aromatic hydrocarbon ring having 6 to 60 carbon atoms or a substituted or unsubstituted heteroring having 2 to 60 carbon atoms,

a and b are each 0 or 1, and

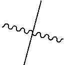

means a position bonding to L₁ of Chemical Formula 1.

In addition, one embodiment of the present application provides an organic light emitting device including a first electrode; a second electrode; and one or more organic material layers provided between the first electrode and the second electrode, wherein one or more layers of the organic material layers include the heterocyclic compound represented by Chemical Formula 1.

Lastly, one embodiment of the present application provides a composition for an organic material layer of an organic light emitting device, the composition including the heterocyclic compound represented by Chemical Formula 1 and any one of the following heterocyclic compounds 1-1 to 1-14.

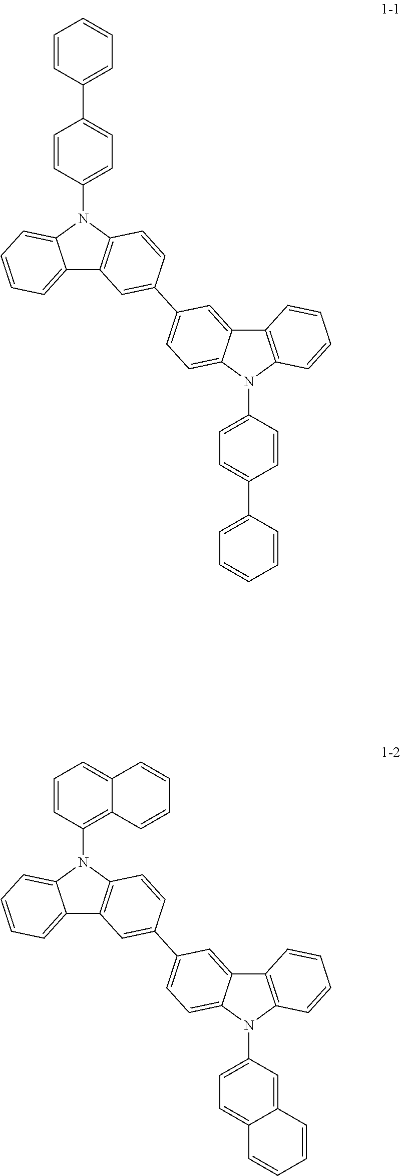 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

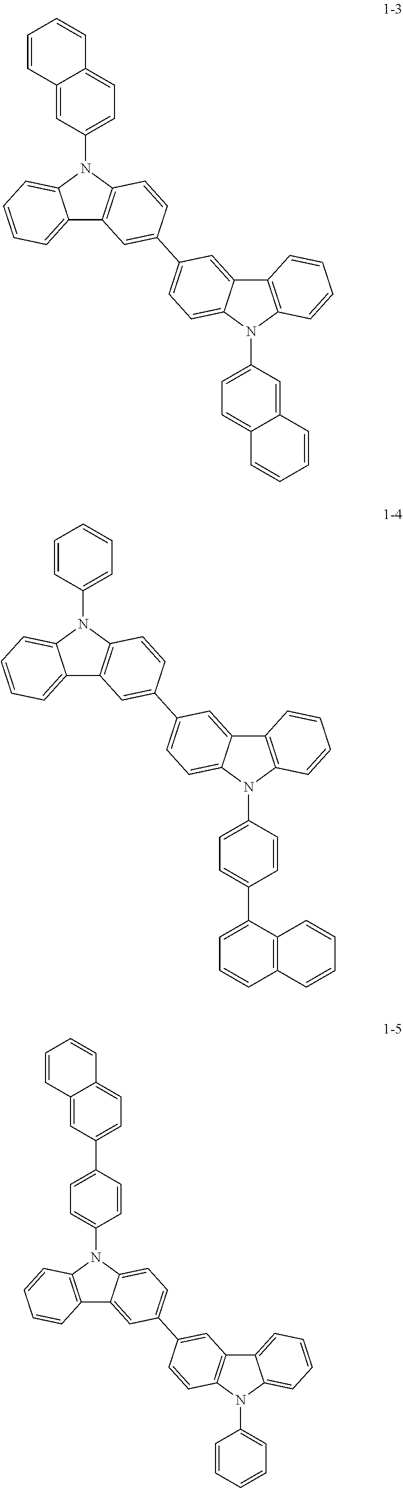 [SMILES: c1ccc2cc(-n3c4ccccc4c4cc(-c5ccc6c(c5)c5ccccc5n6-c5ccc6ccccc6c5)ccc43)ccc2c1 ; c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

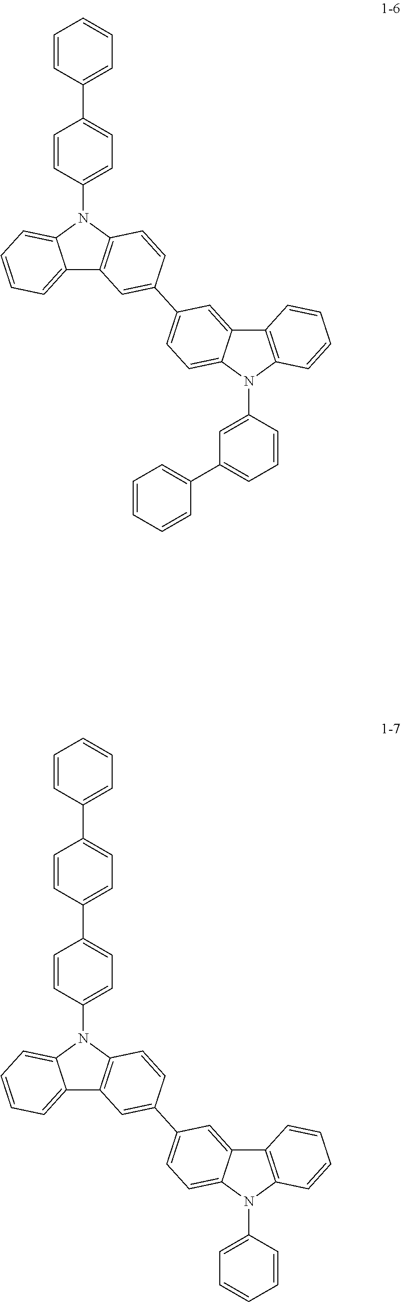 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

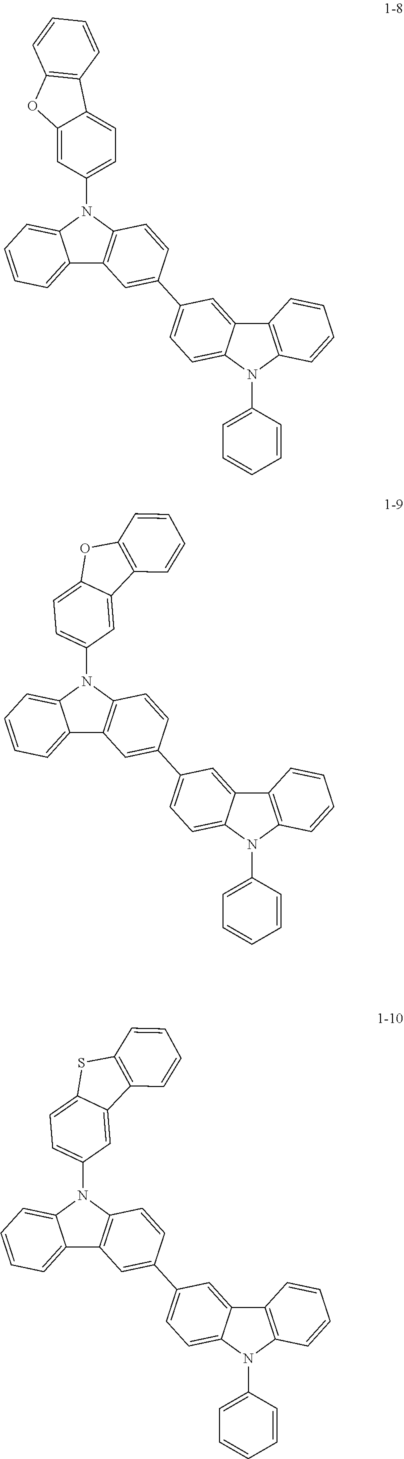 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

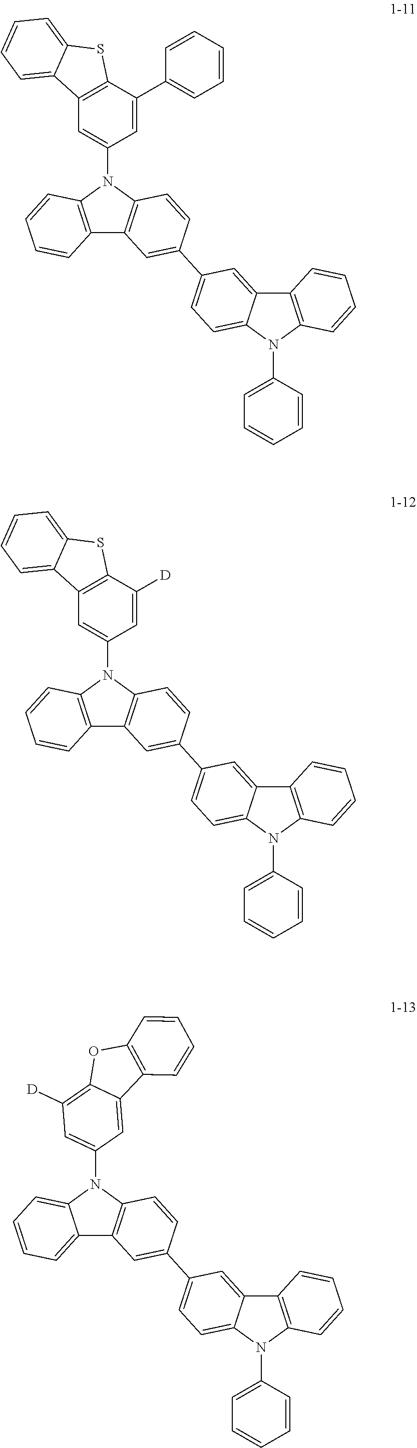 [SMILES: c1ccc(-c2cc(-n3c4ccccc4c4cc(-c5ccc6c(c5)c5ccccc5n6-c5ccccc5)ccc43)cc3c2sc2ccccc23)cc1 ; [2H]c1cc(-n2c3ccccc3c3cc(-c4ccc5c(c4)c4ccccc4n5-c4ccccc4)ccc32)cc2c1sc1ccccc12 ; [2H]c1cc(-n2c3ccccc3c3cc(-c4ccc5c(c4)c4ccccc4n5-c4ccccc4)ccc32)cc2c1oc1ccccc12]

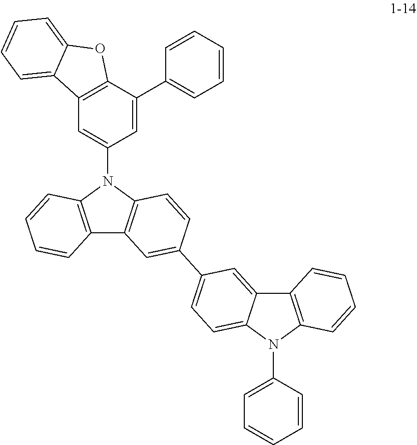 [SMILES: c1ccc(-c2cc(-n3c4ccccc4c4cc(-c5ccc6c(c5)c5ccccc5n6-c5ccccc5)ccc43)cc3c2oc2ccccc23)cc1]

### Advantageous Effects

A heterocyclic compound described in the present specification can be used as a material of an organic material layer of an organic light emitting device. In the organic light emitting device, the heterocyclic compound is capable of performing a role of a hole injection material, a hole transfer material, a light emitting material, an electron transfer material, an electron injection material or the like.

Specifically, when using the heterocyclic compound represented by Chemical Formula 1 in an organic material layer of an organic light emitting device, a driving voltage of the device can be lowered, light efficiency can be enhanced, and lifetime properties of the device can be enhanced.

### DESCRIPTION OF DRAWINGS

FIG. 1 to FIG. 3 are diagrams each illustrating a lamination structure of an organic light emitting device according to one embodiment of the present application.

### MODE FOR DISCLOSURE

Hereinafter, the present specification will be described in more detail.

In the present specification, a certain part “including (comprising)” certain constituents means capable of further including other constituents, and does not exclude other constituents unless particularly stated on the contrary.

In the present specification, the term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of a linear or branched alkyl group having 1 to 60 carbon atoms; a linear or branched alkenyl group having 2 to 60 carbon atoms; a linear or branched alkynyl group having 2 to 60 carbon atoms; a monocyclic or polycyclic cycloalkyl group having 3 to 60 carbon atoms; a monocyclic or polycyclic heterocycloalkyl group having 2 to 60 carbon atoms; a monocyclic or polycyclic aryl group having 6 to 60 carbon atoms; a monocyclic or polycyclic heteroaryl group having 2 to 60 carbon atoms; a silyl group; a phosphine oxide group; and an amine group, or being unsubstituted, or being substituted with a substituent linking two or more substituents selected from among the substituents illustrated above, or being unsubstituted.

More specifically, “substituted or unsubstituted” in the present specification means being substituted with one or more substituents selected from the group consisting of a monocyclic or polycyclic aryl group having 6 to 60 carbon atoms; or a monocyclic or polycyclic heteroaryl group having 2 to 60 carbon atoms.

In the present specification, the halogen may be fluorine, chlorine, bromine or iodine.

In the present specification, the alkyl group includes linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group and the like, but are not limited thereto.

In the present specification, the alkynyl group includes linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group includes monocyclic or polycyclic having 3 to 60 carbon atoms, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic having 2 to 60 carbon atoms, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic having 6 to 60 carbon atoms, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the phosphine oxide may include a diphenylphosphine oxide group, a dinaphthylphosphine oxide group and the like, but are not limited thereto.

In the present specification, the silyl group is a substituent including Si, having the Si atom directly linked as a radical, and is represented by —SiR104R105R106. R104 to R106 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the following spiro group may include any one of groups of the following structural formulae.

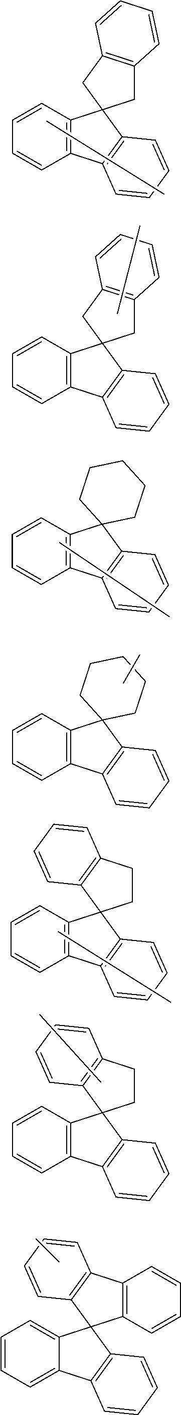

In the present specification, the heteroaryl group includes S, O, Se, N or Si as a heteroatom, includes monocyclic or polycyclic having 2 to 60 carbon atoms, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrobenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0%, a hydrogen content being 100% or substituents being all hydrogen.

In one embodiment of the present application, deuterium is one of isotopes of hydrogen, is an element having deuteron formed with one proton and one neutron as a nucleus, and may be expressed as hydrogen-2, and the elemental symbol may also be written as D or 2H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

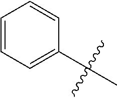 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

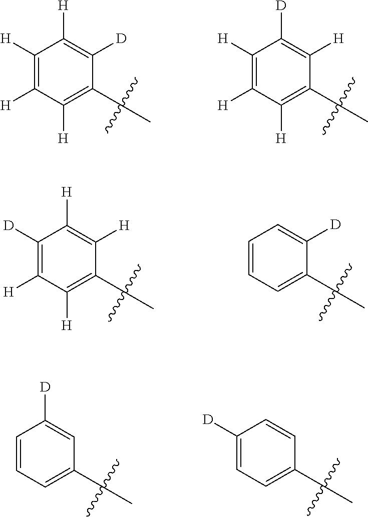 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not include a deuterium atom, that is, a phenyl group that has 5 hydrogen atoms.

One embodiment of the present application provides a heterocyclic compound represented by the following Chemical Formula 1.

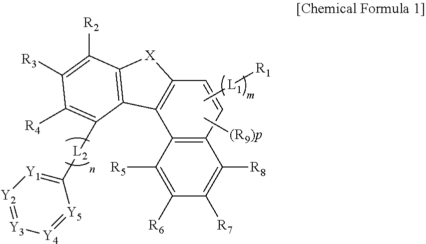

In Chemical Formula 1,

L₁ and L₂ are the same as or different from each other, and each independently a direct bond; a substituted or unsubstituted arylene group having 6 to 60 carbon atoms; or a substituted or unsubstituted heteroarylene group having 2 to 60 carbon atoms,

X is O; S; or NRa,

Y₁ to Y₅ are the same as or different from each other and each independently N or CRb, at least one or more of Y₁ to Y₅ are N, and when there are two or more CRbs, Rbs are the same as or different from each other,

R₁ is represented by the following Chemical Formula A,

R₂ to R₉ are hydrogen; deuterium; a halogen group; a cyano group; a substituted or unsubstituted alkyl group; a substituted or unsubstituted cycloalkyl group; a substituted or unsubstituted aryl group having 6 to 40 carbon atoms; or a substituted or unsubstituted heteroaryl group having 2 to 40 carbon atoms,

Ra is a substituted or unsubstituted aryl group having 6 to 40 carbon atoms,

Rb is selected from the group consisting of hydrogen; deuterium; a substituted or unsubstituted aryl group having 6 to 60 carbon atoms; and a substituted or unsubstituted heteroaryl group having 2 to 60 carbon atoms, or two or more groups adjacent to each other bond to each other to form a substituted or unsubstituted aromatic hydrocarbon ring having 6 to 60 carbon atoms or a substituted or unsubstituted heteroring having 2 to 60 carbon atoms,

m and n are each independently an integer of 0 to 3, and when m and n are each 2 or greater, substituents in the parentheses are the same as or different from each other, and

p is 0 or 1,

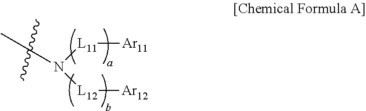

in Chemical Formula A,

L₁₁ and L₁₂ are the same as or different from each other, and each independently a direct bond; a substituted or unsubstituted arylene group having 6 to 40 carbon atoms; or a substituted or unsubstituted heteroarylene group having 2 to 40 carbon atoms,

Ar₁₁ and Ar₁₂ are the same as or different from each other, and each independently selected from the group consisting of a substituted or unsubstituted aryl group having 6 to 40 carbon atoms; and a substituted or unsubstituted heteroaryl group having 2 to 40 carbon atoms, or Ar₁₁ and Ar₁₂ bond to each other to form a substituted or unsubstituted aromatic hydrocarbon ring having 6 to 60 carbon atoms or a substituted or unsubstituted heteroring having 2 to 60 carbon atoms,

a and b are each 0 or 1, and

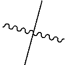

means a position bonding to L₁ of Chemical Formula 1.

The compound represented by Chemical Formula 1 has a steric placement by having both a donor having a favorable hole transfer ability and an acceptor having a favorable electron transfer ability in one molecule and fixing a substituent at a No. 11 position of the naphthobenzofuran, and spatially separates HOMO (Highest Occupied Molecular Orbital) and LUMO (Lowest Unoccupied Molecular Orbital) allowing strong charge transfer. Accordingly, high efficiency may be expected when used as an organic material in an organic light emitting device.

In one embodiment of the present application, L₁ and L₂ of Chemical Formula 1 are the same as or different from each other, and may be each independently a direct bond; a substituted or unsubstituted arylene group; or a substituted or unsubstituted heteroarylene group.

In another embodiment, L₁ and L₂ are the same as or different from each other, and may be each independently a direct bond; a substituted or unsubstituted arylene group having 6 to 60 carbon atoms; or a substituted or unsubstituted heteroarylene group having 2 to 60 carbon atoms.

In another embodiment, L₁ and L₂ are the same as or different from each other, and may be each independently a direct bond; a substituted or unsubstituted arylene group having 6 to 40 carbon atoms; or a substituted or unsubstituted heteroarylene group having 2 to 40 carbon atoms.

In another embodiment, L₁ and L₂ are the same as or different from each other, and may be each independently a direct bond; a substituted or unsubstituted arylene group having 6 to 20 carbon atoms; or a substituted or unsubstituted heteroarylene group having 2 to 20 carbon atoms.

In another embodiment, L₁ and L₂ are the same as or different from each other, and may be each independently a direct bond; a substituted or unsubstituted phenylene group; or a substituted or unsubstituted biphenylene group.

In another embodiment, L₁ and L₂ are the same as or different from each other, and may be each independently a direct bond; a phenylene group; or a biphenylene group.

In one embodiment of the present application, L₁ and L₂ may be different from each other.

In one embodiment of the present application, L₁ and L₂ may be the same as each other.

In one embodiment of the present application, L₁ may be a direct bond, and L₂ may be a substituted or unsubstituted arylene group having 6 to 60 carbon atoms; or a substituted or unsubstituted heteroarylene group having 2 to 60 carbon atoms.

In one embodiment of the present application, L₁ may be a direct bond, and L₂ may be a substituted or unsubstituted arylene group having 6 to 40 carbon atoms; or a substituted or unsubstituted heteroarylene group having 2 to 40 carbon atoms.

In one embodiment of the present application, L₁ may be a direct bond, and L₂ may be a substituted or unsubstituted arylene group having 6 to 20 carbon atoms; or a substituted or unsubstituted heteroarylene group having 2 to 20 carbon atoms.

In another embodiment, L₁ may be a direct bond; a substituted or unsubstituted phenylene group; or a substituted or unsubstituted biphenylene group.

In another embodiment, L₁ may be a direct bond; a phenylene group; or a biphenylene group.

In another embodiment, L₁ is a direct bond.

In another embodiment, L₁ is a phenylene group.

In another embodiment, L₁ is a biphenylene group.

In one embodiment of the present application, L₂ may be a direct bond, and L₁ may be a substituted or unsubstituted arylene group having 6 to 60 carbon atoms; or a substituted or unsubstituted heteroarylene group having 2 to 60 carbon atoms.

In one embodiment of the present application, L₂ may be a direct bond, and L₁ may be a substituted or unsubstituted arylene group having 6 to 40 carbon atoms; or a substituted or unsubstituted heteroarylene group having 2 to 40 carbon atoms.

In one embodiment of the present application, L₂ may be a direct bond, and L₁ may be a substituted or unsubstituted arylene group having 6 to 20 carbon atoms; or a substituted or unsubstituted heteroarylene group having 2 to 20 carbon atoms.

In one embodiment of the present application, L₂ may be a direct bond; a substituted or unsubstituted arylene group having 6 to 60 carbon atoms; or a substituted or unsubstituted heteroarylene group having 2 to 60 carbon atoms.

In another embodiment, L₂ may be a direct bond; a substituted or unsubstituted arylene group having 6 to 40 carbon atoms; or a substituted or unsubstituted heteroarylene group having 2 to 40 carbon atoms.

In another embodiment, L₂ may be a direct bond; a substituted or unsubstituted arylene group having 6 to 20 carbon atoms; or a substituted or unsubstituted heteroarylene group having 2 to 20 carbon atoms.

In another embodiment, L₂ may be a direct bond; a substituted or unsubstituted phenylene group; or a substituted or unsubstituted biphenylene group.

In another embodiment, L₂ may be a direct bond; a phenylene group; a biphenylene group; or a naphthylene group.

In another embodiment, L₂ is a direct bond.

In another embodiment, L₂ is a phenylene group.

In another embodiment, L₂ is a biphenylene group.

In one embodiment of the present application, m and n of Chemical Formula 1 are each independently an integer of 0 to 3, and when m and n are 2 or greater, substituents in the parentheses are the same as or different from each other.

In one embodiment of the present application, m is 3.

In one embodiment of the present application, m is 2.

In one embodiment of the present application, m is 1.

In one embodiment of the present application, m is 0.

In one embodiment of the present application, when m is 2 or greater, substituents in the parentheses are the same as or different from each other.

In one embodiment of the present application, n is 3.

In one embodiment of the present application, n is 2.

In one embodiment of the present application, n is 1.

In one embodiment of the present application, n is 0.

In one embodiment of the present application, when n is 2 or greater, substituents in the parentheses are the same as or different from each other.

In one embodiment of the present application, X of Chemical Formula 1 may be O; S; or NRa.

In one embodiment of the present application, X is O; or NRa.

In one embodiment of the present application, X is O.

In one embodiment of the present application, X may be NRa.

In one embodiment of the present application, Y₁ to Y₅ of Chemical Formula 1 are the same as or different from each other and each independently N or CRb, at least one or more of Y₁ to Y₅ are N, and when there are two or more CRbs, Rbs may be the same as or different from each other.

In one embodiment of the present application, there are one or more and three or less Ns in Y₁ to Y₅, and the rest are CRb, and when there are two or more CRbs, Rbs may be the same as or different from each other.

In one embodiment of the present application, Ra may be a substituted or unsubstituted aryl group having 6 to 40 carbon atoms.

In one embodiment of the present application, Ra may be a substituted or unsubstituted aryl group having 6 to 10 carbon atoms.

In one embodiment of the present application, Ra may be a substituted or unsubstituted phenyl group.

In one embodiment of the present application, Ra may be a phenyl group.

In one embodiment of the present application, Rb is selected from the group consisting of hydrogen; deuterium; a substituted or unsubstituted aryl group having 6 to 60 carbon atoms; and a substituted or unsubstituted heteroaryl group having 2 to 60 carbon atoms, or two or more groups adjacent to each other may bond to each other to form a substituted or unsubstituted aromatic hydrocarbon ring having 6 to 60 carbon atoms or a substituted or unsubstituted heteroring having 2 to 60 carbon atoms.

In one embodiment of the present application, Rb is selected from the group consisting of hydrogen; deuterium; a substituted or unsubstituted aryl group having 6 to 40 carbon atoms; and a substituted or unsubstituted heteroaryl group having 2 to 40 carbon atoms, or two or more groups adjacent to each other may bond to each other to form a substituted or unsubstituted aromatic hydrocarbon ring having 6 to 60 carbon atoms or a substituted or unsubstituted heteroring having 2 to 60 carbon atoms.

In one embodiment of the present application, Rb may be selected from the group consisting of hydrogen; deuterium; a substituted or unsubstituted aryl group having 6 to 60 carbon atoms; and a substituted or unsubstituted heteroaryl group having 2 to 60 carbon atoms.

In one embodiment of the present application, Rb may be selected from the group consisting of hydrogen; deuterium; a substituted or unsubstituted aryl group having 6 to 40 carbon atoms; and a substituted or unsubstituted heteroaryl group having 2 to 40 carbon atoms.

In one embodiment of the present application, Rb may be selected from the group consisting of hydrogen; deuterium; a substituted or unsubstituted aryl group having 6 to 20 carbon atoms; and a substituted or unsubstituted heteroaryl group having 2 to 20 carbon atoms.

In one embodiment of the present application, Rb may be hydrogen; or deuterium.

In one embodiment of the present application, Rb is hydrogen.

In one embodiment of the present application, Rb is deuterium.

In one embodiment of the present application, Rb may be selected from the group consisting of a substituted or unsubstituted aryl group having 6 to 60 carbon atoms; and a substituted or unsubstituted heteroaryl group having 2 to 60 carbon atoms.

In one embodiment of the present application, Rb may be selected from the group consisting of a substituted or unsubstituted aryl group having 6 to 40 carbon atoms; and a substituted or unsubstituted heteroaryl group having 2 to 40 carbon atoms.

In one embodiment of the present application, Rb may be selected from the group consisting of a substituted or unsubstituted aryl group having 6 to 20 carbon atoms; and a substituted or unsubstituted heteroaryl group having 2 to 20 carbon atoms.

In one embodiment of the present application, Rb may be selected from the group consisting of hydrogen; deuterium; a substituted or unsubstituted phenyl group; a substituted or unsubstituted biphenyl group; a substituted or unsubstituted naphthyl group; a substituted or unsubstituted fluorenyl group; a substituted or unsubstituted dibenzothiophene group; and a substituted or unsubstituted dibenzofuran group.

In one embodiment of the present application, Rb may be selected from the group consisting of hydrogen; deuterium; a phenyl group unsubstituted or substituted with a phenyl group or a naphthyl group; a substituted or unsubstituted biphenyl group; a substituted or unsubstituted naphthyl group; a fluorenyl group unsubstituted or substituted with an alkyl group having 2 to 10 carbon atoms; a substituted or unsubstituted dibenzothiophene group; and a substituted or unsubstituted dibenzofuran group.

In one embodiment of the present application, Rb may be selected from the group consisting of hydrogen; deuterium; a phenyl group unsubstituted or substituted with a phenyl group or a naphthyl group; a substituted or unsubstituted biphenyl group; a substituted or unsubstituted naphthyl group; a fluorenyl group unsubstituted or substituted with a methyl group; a substituted or unsubstituted dibenzothiophene group; and a substituted or unsubstituted dibenzofuran group.

In one embodiment of the present application, Chemical Formula 1 may be represented by the following Chemical Formula 1-1.

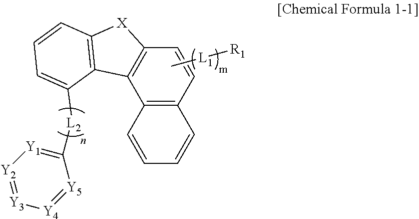

In Chemical Formula 1-1,

each substituent has the same definition as in Chemical Formula 1.

In one embodiment of the present application, Chemical Formula 1 may be represented by the following Chemical Formula 2 or 3.

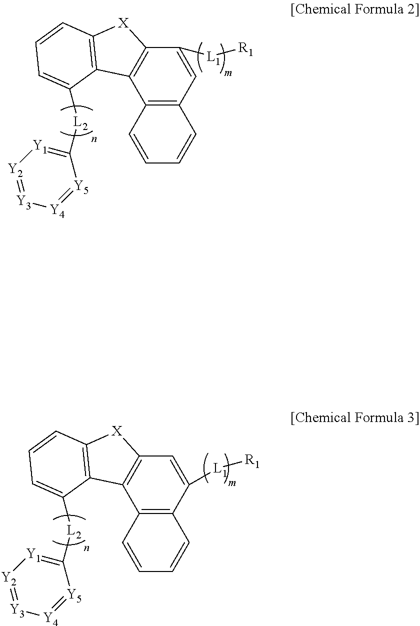

In Chemical Formulae 2 and 3,

each substituent has the same definition as in Chemical Formula 1.

In one embodiment of the present application, Chemical Formula 2 may be represented by any one of the following Chemical Formulae 2-1 to 2-6.

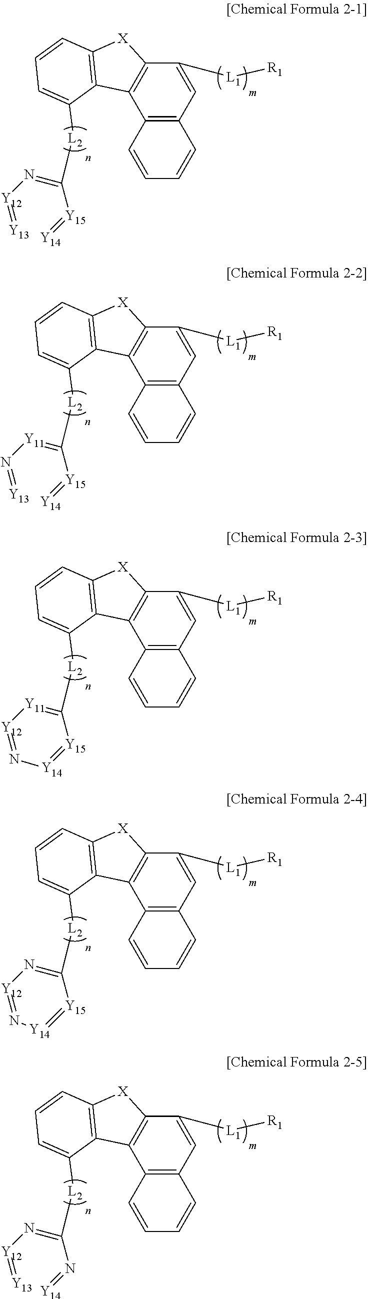

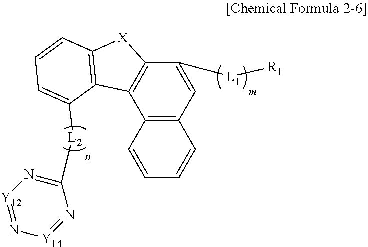

In Chemical Formulae 2-1 to 2-6,

L₁, L₂, X, R₁, m and n have the same definitions as in Chemical Formula 1, and

Y₁₁ to Y₁₅ are CRb, and Rb has the same definition as in Chemical Formula 1.

In one embodiment of the present application, Chemical Formula 3 may be represented by any one of the following Chemical Formulae 3-1 to 3-6.

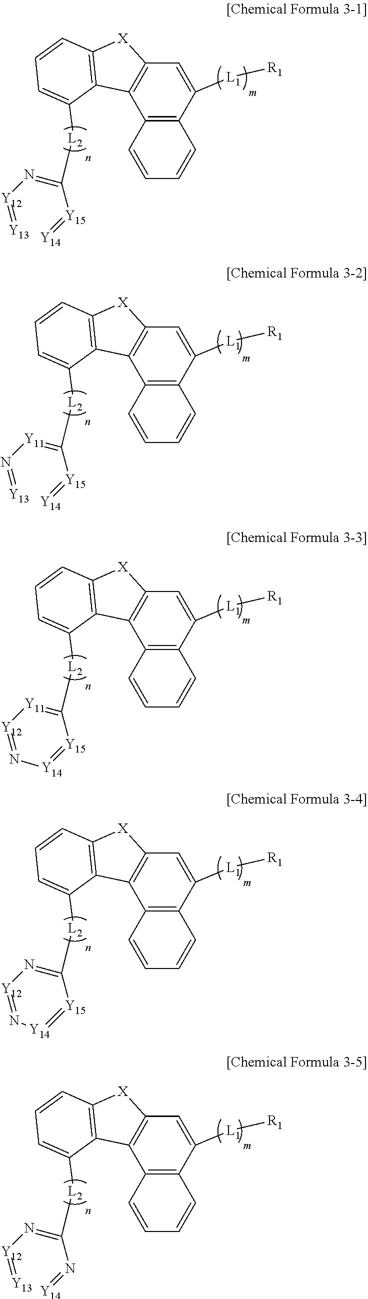

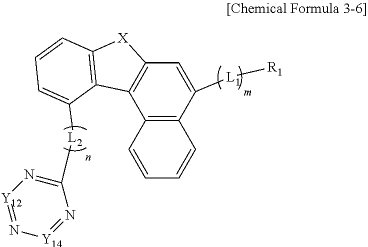

In Chemical Formulae 3-1 to 3-6,

L₁, L₂, X, R₁, m and n have the same definitions as in Chemical Formula 1, and

Y₁₁ to Y₁₅ are CRb, and Rb has the same definition as in Chemical Formula 1.

In one embodiment of the present application, R₁ may be represented by the following Chemical Formula A.

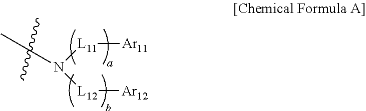

In Chemical Formula A,

L₁₁ and L₁₂ are the same as or different from each other, and each independently a direct bond; a substituted or unsubstituted arylene group having 6 to 40 carbon atoms; or a substituted or unsubstituted heteroarylene group having 2 to 40 carbon atoms,

Ar₁₁ and Ar₁₂ are the same as or different from each other, and each independently selected from the group consisting of a substituted or unsubstituted aryl group having 6 to 40 carbon atoms; and a substituted or unsubstituted heteroaryl group having 2 to 40 carbon atoms, or Ar₁₁ and Ar₁₂ bond to each other to form a substituted or unsubstituted aromatic hydrocarbon ring having 6 to 60 carbon atoms or a substituted or unsubstituted heteroring having 2 to 60 carbon atoms,

a and b are 0 or 1, and

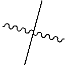

means a position bonding to L₁ of Chemical Formula 1.

In one embodiment of the present application, Chemical Formula A may be represented by any one of the following Chemical Formulae A-1 to A-5.

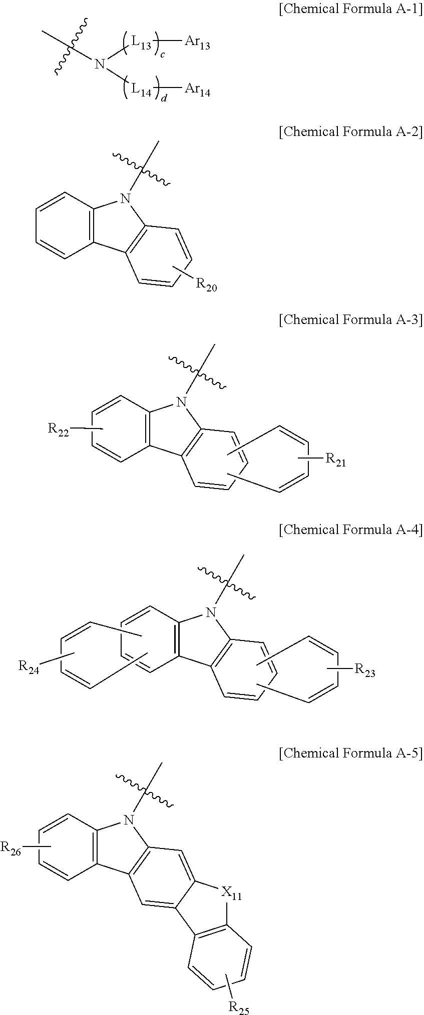

In Chemical Formulae A-1 to A-5,

L₁₃ and L₁₄ are the same as or different from each other, and each independently a direct bond; a substituted or unsubstituted arylene group having 6 to 40 carbon atoms; or a substituted or unsubstituted heteroarylene group having 2 to 40 carbon atoms,

Ar₁₃ and Ar₁₄ are the same as or different from each other, and each independently a substituted or unsubstituted aryl group having 6 to 40 carbon atoms; or a substituted or unsubstituted heteroaryl group having 2 to 40 carbon atoms,

R₂₀ to R₂₆ are each independently hydrogen; deuterium; a halogen group; a cyano group; a substituted or unsubstituted aryl group having 6 to 20 carbon atoms; or a substituted or unsubstituted heteroaryl group having 2 to 20 carbon atoms,

X₁₁ is O; S; or CRcRd, and Rc and Rd are the same as or different from each other and each independently a substituted or unsubstituted alkyl group having 1 to 10 carbon atoms,

c and d are each 0 or 1, and

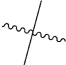

means a position bonding to L₁ of Chemical Formula 1.

In one embodiment of the present application, L₁₃ and L₁₄ of Chemical Formula A-1 are the same as or different from each other, and may be each independently a direct bond; a substituted or unsubstituted arylene group having 6 to 40 carbon atoms; or a substituted or unsubstituted heteroarylene group having 2 to 40 carbon atoms.

In one embodiment of the present application, L₁₃ and L₁₄ of Chemical Formula A-1 are the same as or different from each other, and may be each independently a direct bond; a substituted or unsubstituted arylene group having 6 to 20 carbon atoms; or a substituted or unsubstituted heteroarylene group having 2 to 20 carbon atoms.

In one embodiment of the present application, L₁₃ and L₁₄ are the same as or different from each other, and may be each independently a direct bond; or a substituted or unsubstituted arylene group having 6 to 20 carbon atoms.

In one embodiment of the present application, L₁₃ and L₁₄ are the same as or different from each other, and may be each independently a direct bond; a substituted or unsubstituted phenylene group; a substituted or unsubstituted biphenylene group; or a substituted or unsubstituted naphthylene group.

In one embodiment of the present application, L₁₃ and L₁₄ are the same as or different from each other, and may be each independently a direct bond; a phenylene group; a biphenylene group; or a naphthylene group.

In one embodiment of the present application, L₁₃ is a direct bond.

In one embodiment of the present application, L₁₃ is a phenylene group.

In one embodiment of the present application, L₁₃ is a biphenylene group.

In one embodiment of the present application, L₁₃ is a naphthylene group.

In one embodiment of the present application, L₁₄ is a direct bond.

In one embodiment of the present application, L₁₄ is a phenylene group.

In one embodiment of the present application, L₁₄ is a biphenylene group.

In one embodiment of the present application, L₁₄ is a naphthylene group.

In one embodiment of the present application, Ar₁₃ and Ar₁₄ of Chemical Formula A-1 are the same as or different from each other, and may be each independently a substituted or unsubstituted aryl group having 6 to 40 carbon atoms; or a substituted or unsubstituted heteroaryl group having 2 to 40 carbon atoms.

In one embodiment of the present application, Ar₁₃ and Ar₁₄ of Chemical Formula A-1 are the same as or different from each other, and may be each independently a substituted or unsubstituted aryl group having 6 to 20 carbon atoms; or a substituted or unsubstituted heteroaryl group having 2 to 20 carbon atoms.

In one embodiment of the present application, Ar₁₃ and Ar₁₄ are the same as or different from each other, and may be each independently a substituted or unsubstituted phenyl group; a substituted or unsubstituted biphenyl group; a substituted or unsubstituted naphthyl group; a substituted or unsubstituted fluorenyl group; a substituted or unsubstituted dibenzothiophene group; or a substituted or unsubstituted dibenzofuran group.

In one embodiment of the present application, Ar₁₃ and Ar₁₄ are the same as or different from each other, and may be each independently a substituted or unsubstituted phenyl group; a substituted or unsubstituted biphenyl group; a substituted or unsubstituted naphthyl group; a fluorenyl group unsubstituted or substituted with one or more selected from the group consisting of a phenyl group and an alkyl group having 1 to 10 carbon atoms; a substituted or unsubstituted dibenzothiophene group; or a substituted or unsubstituted dibenzofuran group.

In one embodiment of the present application, Ar₁₃ and Ar₁₄ are the same as or different from each other, and may be each independently a substituted or unsubstituted phenyl group; a substituted or unsubstituted biphenyl group; a substituted or unsubstituted naphthyl group; a fluorenyl group unsubstituted or substituted with one or more selected from the group consisting of a phenyl group and a methyl group; a substituted or unsubstituted dibenzothiophene group; or a substituted or unsubstituted dibenzofuran group.

In one embodiment of the present application, Ar₁₃ and Ar₁₄ are the same as or different from each other, and may be each independently a phenyl group; a biphenyl group; a naphthyl group; a fluorenyl group unsubstituted or substituted with one or more selected from the group consisting of a phenyl group and a methyl group; a substituted or unsubstituted dibenzothiophene group; or a substituted or unsubstituted dibenzofuran group.

In one embodiment of the present application, c and d of Chemical Formula A-1 may each be 0 or 1.

In one embodiment of the present application, c is 0.

In one embodiment of the present application, c is 1.

In one embodiment of the present application, d is 0.

In one embodiment of the present application, d is 1.

In one embodiment of the present application, R₂₀ to R₂₆ of Chemical Formulae A-1 to A-5 may be each independently hydrogen; deuterium; a halogen group; a cyano group; a substituted or unsubstituted aryl group having 6 to 20 carbon atoms; or a substituted or unsubstituted heteroaryl group having 2 to 20 carbon atoms.

In one embodiment of the present application, R₂₀ to R₂₆ may be each independently hydrogen; deuterium; or a substituted or unsubstituted aryl group having 6 to 20 carbon atoms.

In one embodiment of the present application, R₂₀ to R₂₆ may be each independently hydrogen; deuterium; or a substituted or unsubstituted phenyl group.

In one embodiment of the present application, R₂₀ to R₂₆ may be each independently hydrogen; or a phenyl group.

In one embodiment of the present application, R₂₀ may be hydrogen; or a phenyl group.

In one embodiment of the present application, R₂₀ is hydrogen.

In one embodiment of the present application, R₂₀ is a phenyl group.

In one embodiment of the present application, R₂₁ is hydrogen.

In one embodiment of the present application, R₂₂ is hydrogen.

In one embodiment of the present application, R₂₃ is hydrogen.

In one embodiment of the present application, R₂₄ is hydrogen.

In one embodiment of the present application, R₂₅ is hydrogen.

In one embodiment of the present application, R₂₆ is hydrogen.

In one embodiment of the present application, X₁₁ of Chemical Formula A-5 is O; S; or CRcRd, and Rc and Rd are the same as or different from each other and may be each independently a substituted or unsubstituted alkyl group having 1 to 10 carbon atoms.

In one embodiment of the present application, X₁₁ is O; S; or CRcRd, and Rc and Rd may all be a methyl group.

In one embodiment of the present application, X₁₁ is O.

In one embodiment of the present application, X₁₁ is S.

In one embodiment of the present application, X₁₁ is CRcRd, and Rc and Rd are all a methyl group.

In Chemical Formula 1, when the heterocyclic compound in which R₁ is Chemical Formula A-1 is used as an organic material in an organic light emitting device, the organic light emitting device has a more lowered driving voltage, and may thereby have higher efficiency. This is considered to be due to the fact that the heterocyclic compound in which R₁ is Chemical Formula A-1 has faster hole mobility.

In the heterocyclic compound provided in one embodiment of the present application, Chemical Formula 1 is represented by any one of the following compounds.

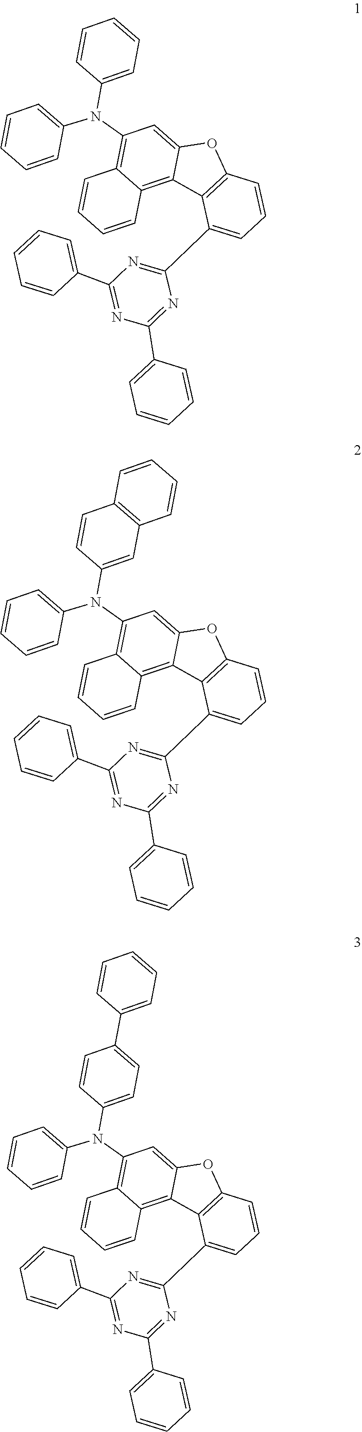

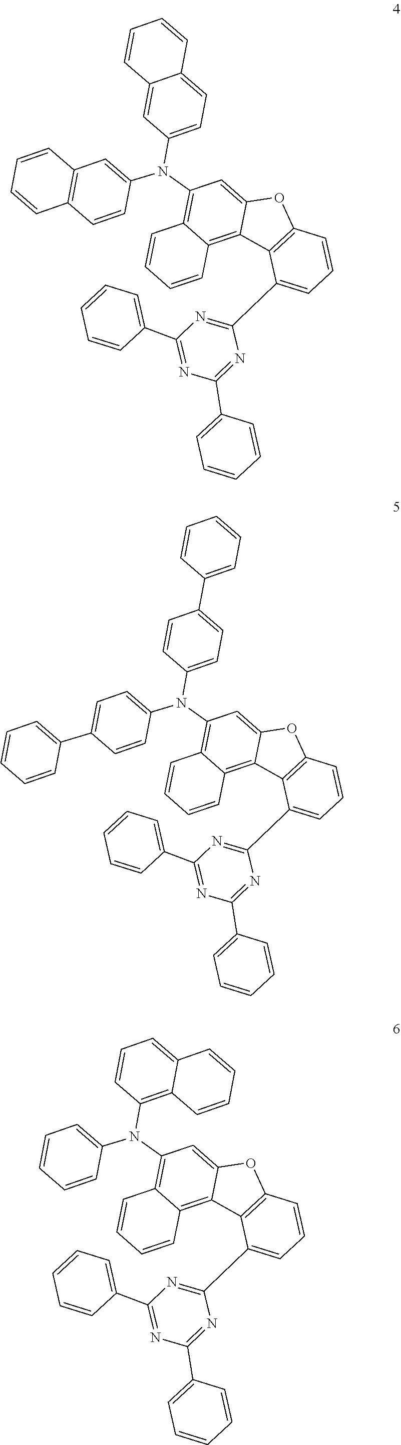

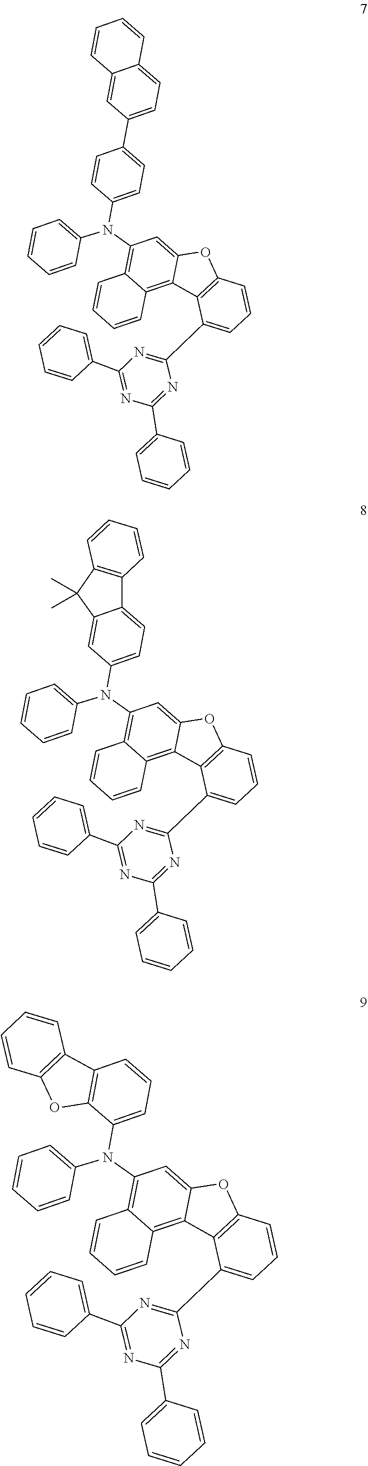 [SMILES: c1ccc(-c2nc(-c3ccccc3)nc(-c3cccc4oc5cc(N(c6ccccc6)c6ccc(-c7ccc8ccccc8c7)cc6)c6ccccc6c5c34)n2)cc1 ; CC1(C)c2ccccc2-c2ccc(N(c3ccccc3)c3cc4oc5cccc(-c6nc(-c7ccccc7)nc(-c7ccccc7)n6)c5c4c4ccccc34)cc21 ; c1ccc(-c2nc(-c3ccccc3)nc(-c3cccc4oc5cc(N(c6ccccc6)c6cccc7c6oc6ccccc67)c6ccccc6c5c34)n2)cc1]

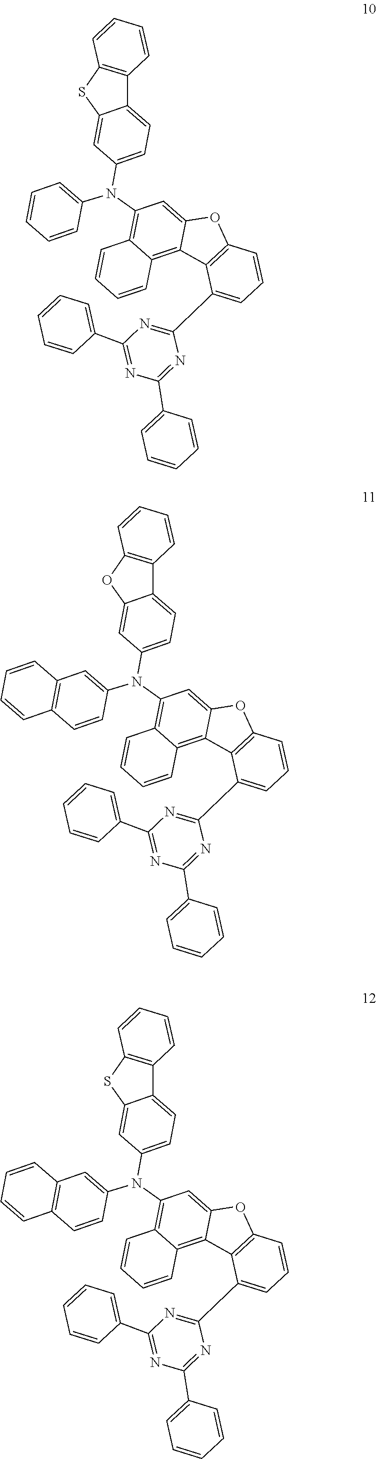 [SMILES: c1ccc(-c2nc(-c3ccccc3)nc(-c3cccc4oc5cc(N(c6ccccc6)c6ccc7c(c6)sc6ccccc67)c6ccccc6c5c34)n2)cc1 ; c1ccc(-c2nc(-c3ccccc3)nc(-c3cccc4oc5cc(N(c6ccc7ccccc7c6)c6ccc7c(c6)oc6ccccc67)c6ccccc6c5c34)n2)cc1 ; c1ccc(-c2nc(-c3ccccc3)nc(-c3cccc4oc5cc(N(c6ccc7ccccc7c6)c6ccc7c(c6)sc6ccccc67)c6ccccc6c5c34)n2)cc1]

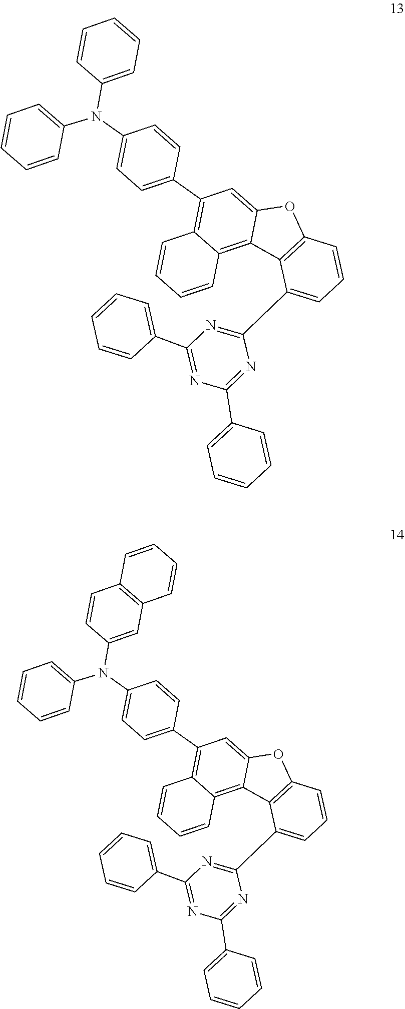 [SMILES: c1ccc(-c2nc(-c3ccccc3)nc(-c3cccc4oc5cc(-c6ccc(N(c7ccccc7)c7ccccc7)cc6)c6ccccc6c5c34)n2)cc1 ; c1ccc(-c2nc(-c3ccccc3)nc(-c3cccc4oc5cc(-c6ccc(N(c7ccccc7)c7ccc8ccccc8c7)cc6)c6ccccc6c5c34)n2)cc1]

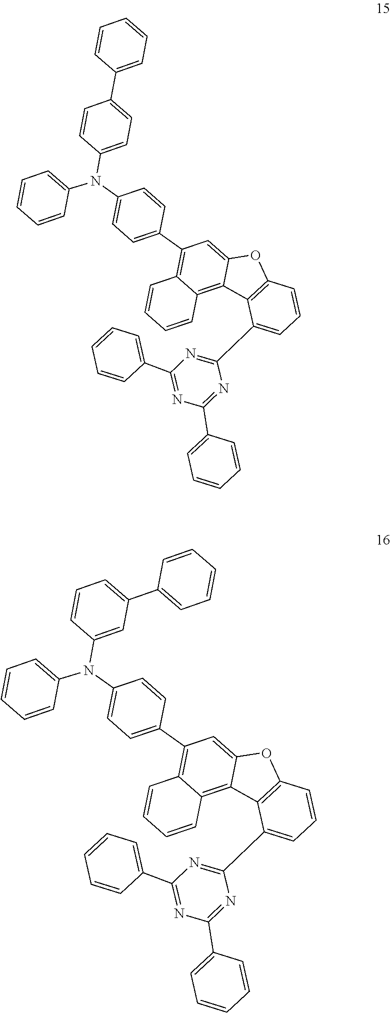 [SMILES: c1ccc(-c2ccc(N(c3ccccc3)c3ccc(-c4cc5oc6cccc(-c7nc(-c8ccccc8)nc(-c8ccccc8)n7)c6c5c5ccccc45)cc3)cc2)cc1 ; c1ccc(-c2cccc(N(c3ccccc3)c3ccc(-c4cc5oc6cccc(-c7nc(-c8ccccc8)nc(-c8ccccc8)n7)c6c5c5ccccc45)cc3)c2)cc1]

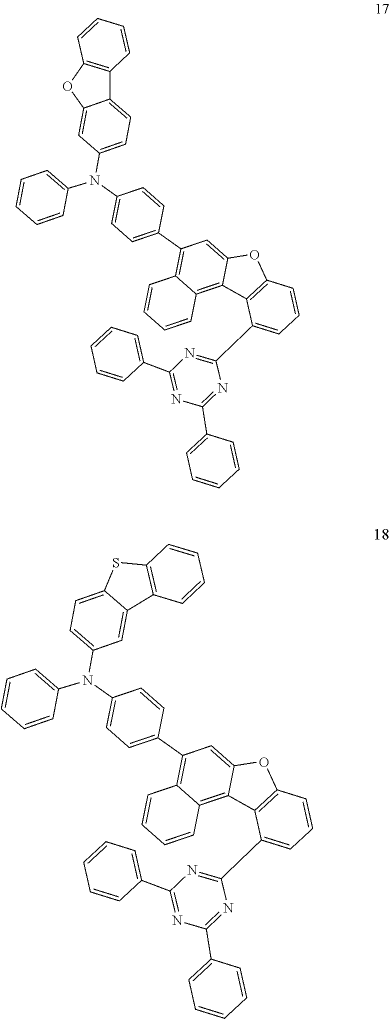 [SMILES: c1ccc(-c2nc(-c3ccccc3)nc(-c3cccc4oc5cc(-c6ccc(N(c7ccccc7)c7ccc8c(c7)oc7ccccc78)cc6)c6ccccc6c5c34)n2)cc1 ; c1ccc(-c2nc(-c3ccccc3)nc(-c3cccc4oc5cc(-c6ccc(N(c7ccccc7)c7ccc8sc9ccccc9c8c7)cc6)c6ccccc6c5c34)n2)cc1]

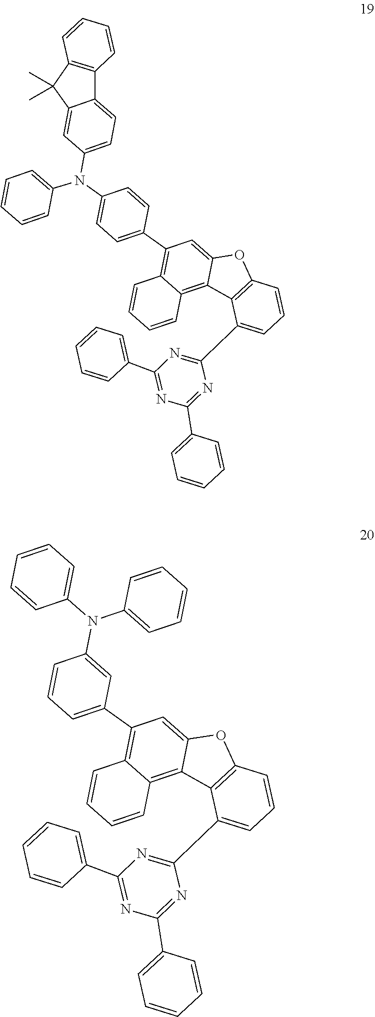 [SMILES: CC1(C)c2ccccc2-c2ccc(N(c3ccccc3)c3ccc(-c4cc5oc6cccc(-c7nc(-c8ccccc8)nc(-c8ccccc8)n7)c6c5c5ccccc45)cc3)cc21 ; c1ccc(-c2nc(-c3ccccc3)nc(-c3cccc4oc5cc(-c6cccc(N(c7ccccc7)c7ccccc7)c6)c6ccccc6c5c34)n2)cc1]

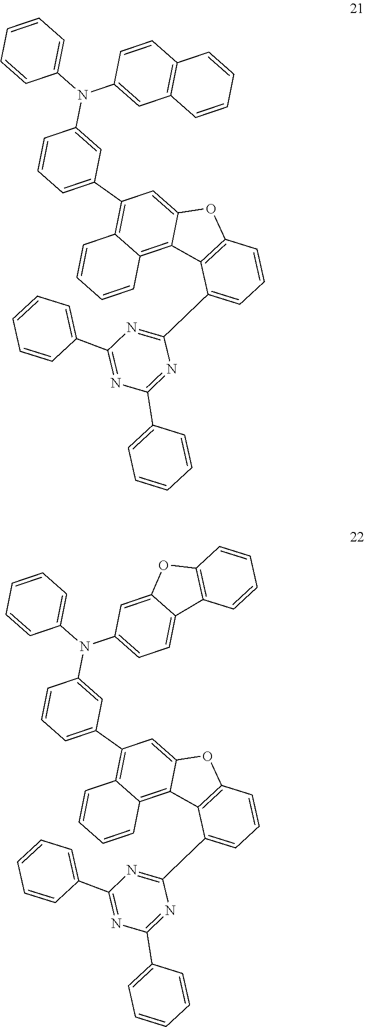 [SMILES: c1ccc(-c2nc(-c3ccccc3)nc(-c3cccc4oc5cc(-c6cccc(N(c7ccccc7)c7ccc8ccccc8c7)c6)c6ccccc6c5c34)n2)cc1 ; c1ccc(-c2nc(-c3ccccc3)nc(-c3cccc4oc5cc(-c6cccc(N(c7ccccc7)c7ccc8c(c7)oc7ccccc78)c6)c6ccccc6c5c34)n2)cc1]

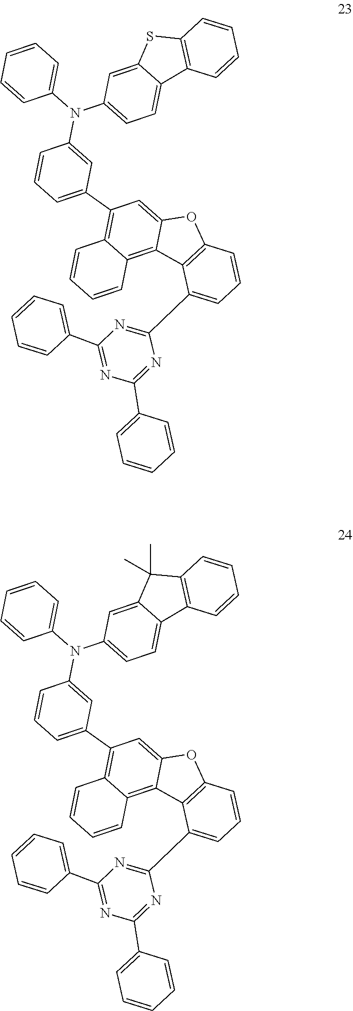 [SMILES: c1ccc(-c2nc(-c3ccccc3)nc(-c3cccc4oc5cc(-c6cccc(N(c7ccccc7)c7ccc8c(c7)sc7ccccc78)c6)c6ccccc6c5c34)n2)cc1 ; CC1(C)c2ccccc2-c2ccc(N(c3ccccc3)c3cccc(-c4cc5oc6cccc(-c7nc(-c8ccccc8)nc(-c8ccccc8)n7)c6c5c5ccccc45)c3)cc21]

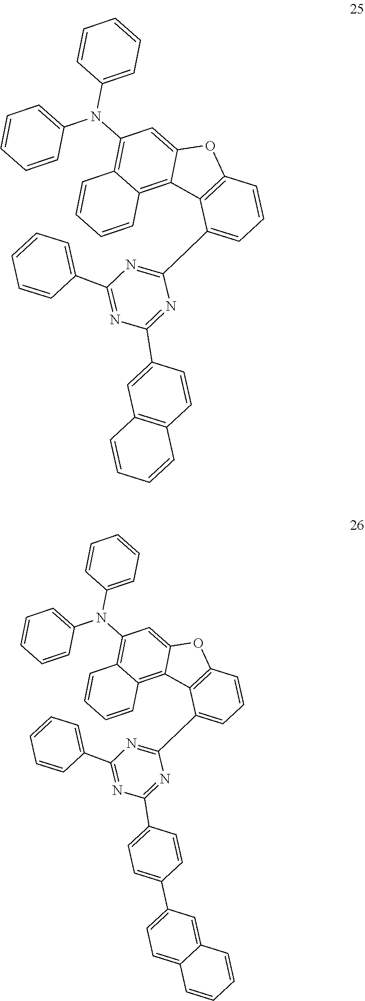 [SMILES: c1ccc(-c2nc(-c3ccc4ccccc4c3)nc(-c3cccc4oc5cc(N(c6ccccc6)c6ccccc6)c6ccccc6c5c34)n2)cc1 ; c1ccc(-c2nc(-c3ccc(-c4ccc5ccccc5c4)cc3)nc(-c3cccc4oc5cc(N(c6ccccc6)c6ccccc6)c6ccccc6c5c34)n2)cc1]

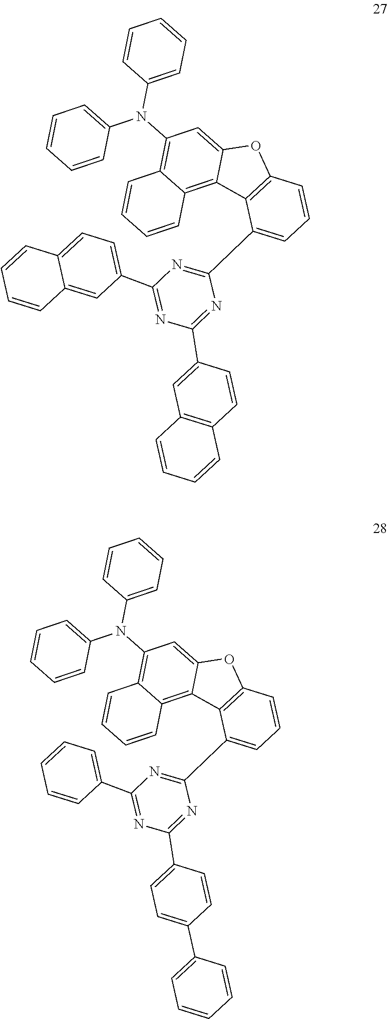 [SMILES: c1ccc(N(c2ccccc2)c2cc3oc4cccc(-c5nc(-c6ccc7ccccc7c6)nc(-c6ccc7ccccc7c6)n5)c4c3c3ccccc23)cc1 ; c1ccc(-c2ccc(-c3nc(-c4ccccc4)nc(-c4cccc5oc6cc(N(c7ccccc7)c7ccccc7)c7ccccc7c6c45)n3)cc2)cc1]

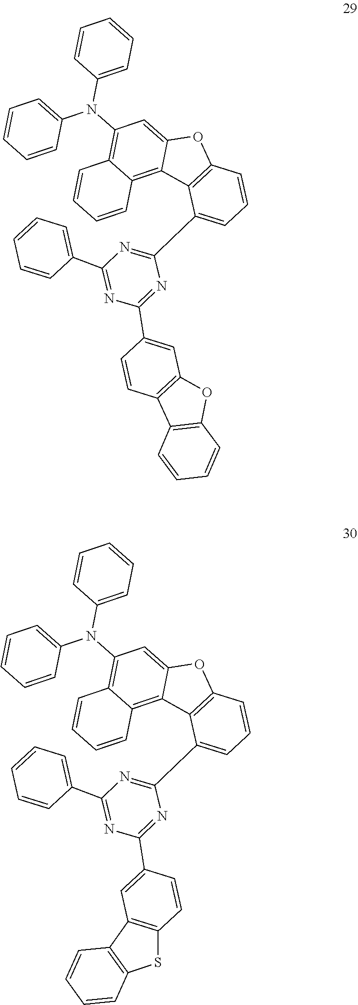 [SMILES: c1ccc(-c2nc(-c3ccc4c(c3)oc3ccccc34)nc(-c3cccc4oc5cc(N(c6ccccc6)c6ccccc6)c6ccccc6c5c34)n2)cc1 ; c1ccc(-c2nc(-c3ccc4sc5ccccc5c4c3)nc(-c3cccc4oc5cc(N(c6ccccc6)c6ccccc6)c6ccccc6c5c34)n2)cc1]

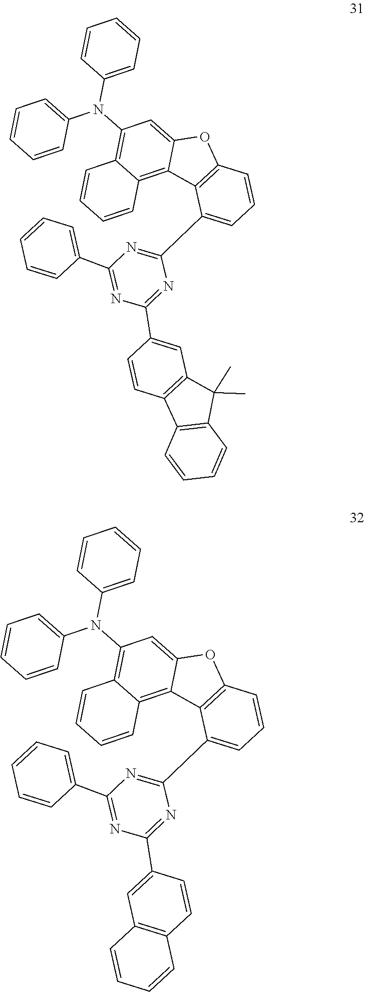 [SMILES: CC1(C)c2ccccc2-c2ccc(-c3nc(-c4ccccc4)nc(-c4cccc5oc6cc(N(c7ccccc7)c7ccccc7)c7ccccc7c6c45)n3)cc21 ; c1ccc(-c2nc(-c3ccc4ccccc4c3)nc(-c3cccc4oc5cc(N(c6ccccc6)c6ccccc6)c6ccccc6c5c34)n2)cc1]

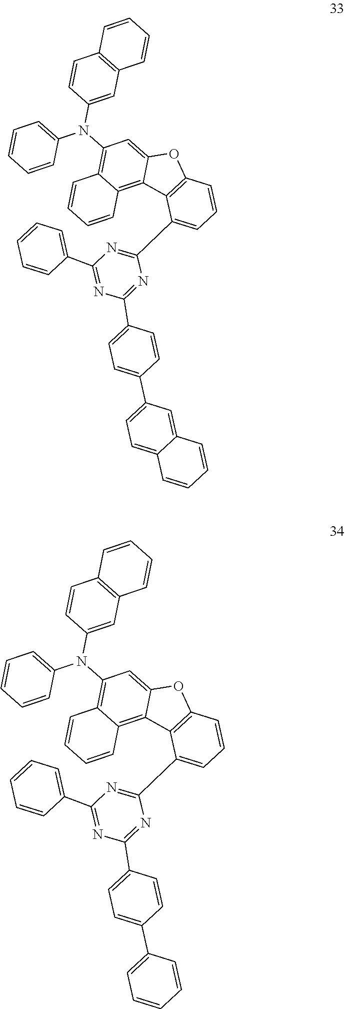 [SMILES: c1ccc(-c2nc(-c3ccc(-c4ccc5ccccc5c4)cc3)nc(-c3cccc4oc5cc(N(c6ccccc6)c6ccc7ccccc7c6)c6ccccc6c5c34)n2)cc1 ; c1ccc(-c2ccc(-c3nc(-c4ccccc4)nc(-c4cccc5oc6cc(N(c7ccccc7)c7ccc8ccccc8c7)c7ccccc7c6c45)n3)cc2)cc1]

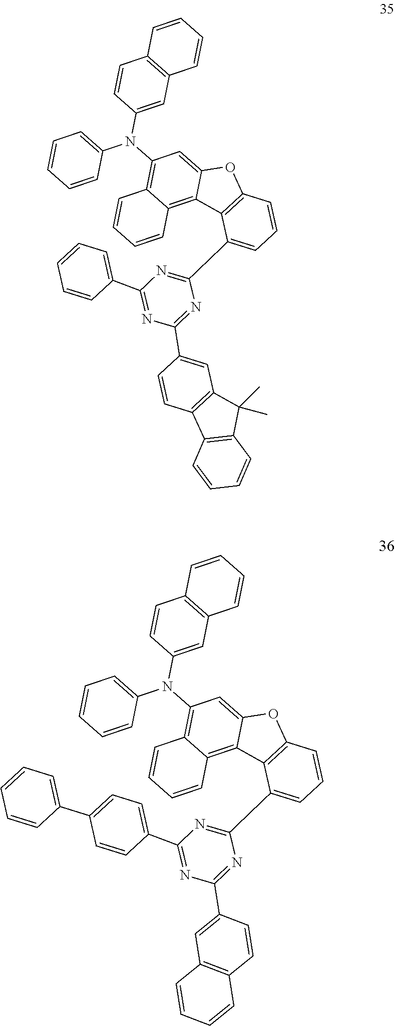 [SMILES: CC1(C)c2ccccc2-c2ccc(-c3nc(-c4ccccc4)nc(-c4cccc5oc6cc(N(c7ccccc7)c7ccc8ccccc8c7)c7ccccc7c6c45)n3)cc21 ; c1ccc(-c2ccc(-c3nc(-c4ccc5ccccc5c4)nc(-c4cccc5oc6cc(N(c7ccccc7)c7ccc8ccccc8c7)c7ccccc7c6c45)n3)cc2)cc1]

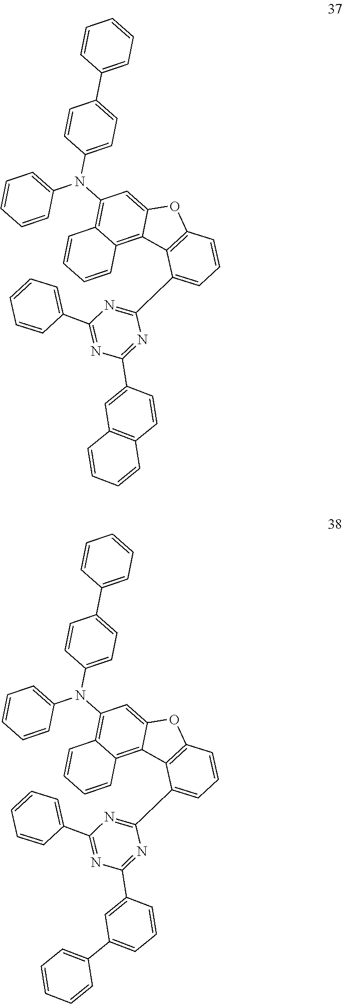 [SMILES: c1ccc(-c2ccc(N(c3ccccc3)c3cc4oc5cccc(-c6nc(-c7ccccc7)nc(-c7ccc8ccccc8c7)n6)c5c4c4ccccc34)cc2)cc1 ; c1ccc(-c2ccc(N(c3ccccc3)c3cc4oc5cccc(-c6nc(-c7ccccc7)nc(-c7cccc(-c8ccccc8)c7)n6)c5c4c4ccccc34)cc2)cc1]

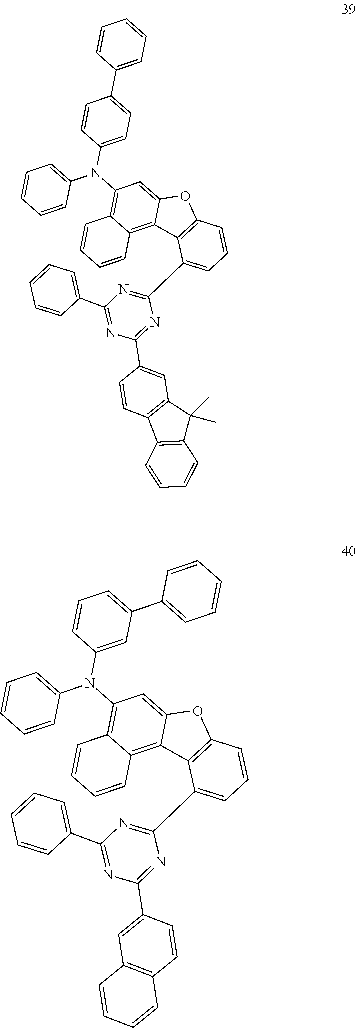 [SMILES: CC1(C)c2ccccc2-c2ccc(-c3nc(-c4ccccc4)nc(-c4cccc5oc6cc(N(c7ccccc7)c7ccc(-c8ccccc8)cc7)c7ccccc7c6c45)n3)cc21 ; c1ccc(-c2cccc(N(c3ccccc3)c3cc4oc5cccc(-c6nc(-c7ccccc7)nc(-c7ccc8ccccc8c7)n6)c5c4c4ccccc34)c2)cc1]

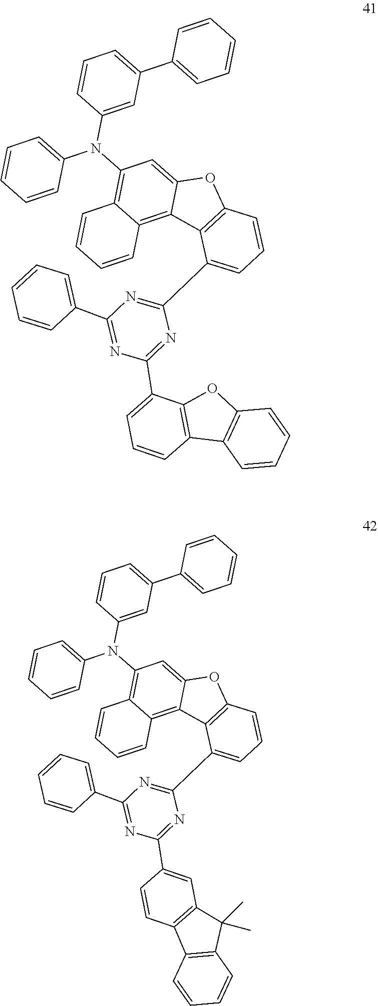 [SMILES: c1ccc(-c2cccc(N(c3ccccc3)c3cc4oc5cccc(-c6nc(-c7ccccc7)nc(-c7cccc8c7oc7ccccc78)n6)c5c4c4ccccc34)c2)cc1 ; CC1(C)c2ccccc2-c2ccc(-c3nc(-c4ccccc4)nc(-c4cccc5oc6cc(N(c7ccccc7)c7cccc(-c8ccccc8)c7)c7ccccc7c6c45)n3)cc21]

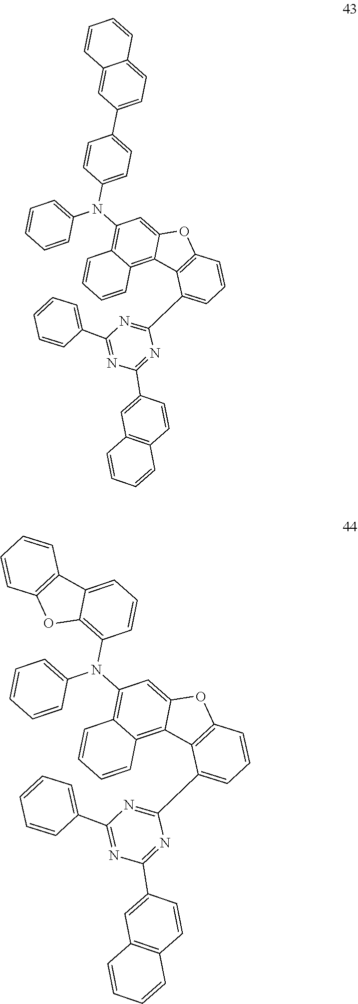 [SMILES: c1ccc(-c2nc(-c3ccc4ccccc4c3)nc(-c3cccc4oc5cc(N(c6ccccc6)c6ccc(-c7ccc8ccccc8c7)cc6)c6ccccc6c5c34)n2)cc1 ; c1ccc(-c2nc(-c3ccc4ccccc4c3)nc(-c3cccc4oc5cc(N(c6ccccc6)c6cccc7c6oc6ccccc67)c6ccccc6c5c34)n2)cc1]

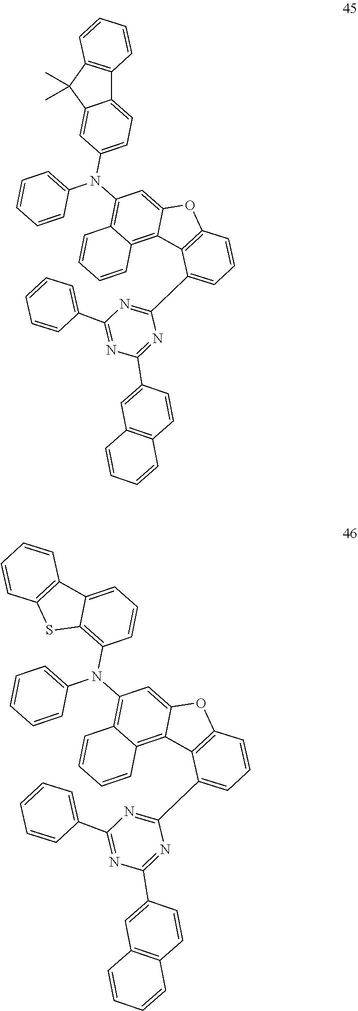 [SMILES: CC1(C)c2ccccc2-c2ccc(N(c3ccccc3)c3cc4oc5cccc(-c6nc(-c7ccccc7)nc(-c7ccc8ccccc8c7)n6)c5c4c4ccccc34)cc21 ; c1ccc(-c2nc(-c3ccc4ccccc4c3)nc(-c3cccc4oc5cc(N(c6ccccc6)c6cccc7c6sc6ccccc67)c6ccccc6c5c34)n2)cc1]

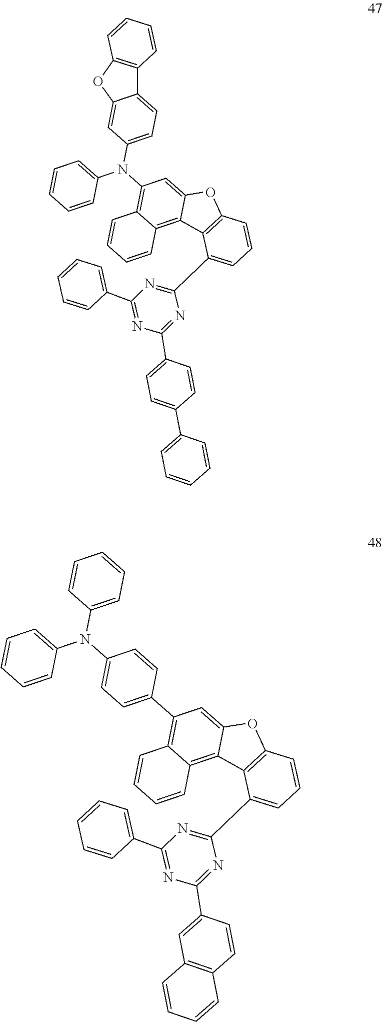 [SMILES: c1ccc(-c2ccc(-c3nc(-c4ccccc4)nc(-c4cccc5oc6cc(N(c7ccccc7)c7ccc8c(c7)oc7ccccc78)c7ccccc7c6c45)n3)cc2)cc1 ; c1ccc(-c2nc(-c3ccc4ccccc4c3)nc(-c3cccc4oc5cc(-c6ccc(N(c7ccccc7)c7ccccc7)cc6)c6ccccc6c5c34)n2)cc1]

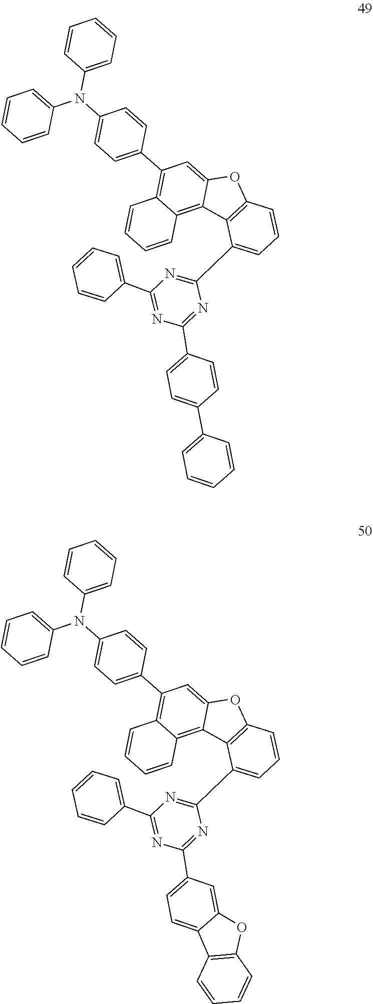 [SMILES: c1ccc(-c2ccc(-c3nc(-c4ccccc4)nc(-c4cccc5oc6cc(-c7ccc(N(c8ccccc8)c8ccccc8)cc7)c7ccccc7c6c45)n3)cc2)cc1 ; c1ccc(-c2nc(-c3ccc4c(c3)oc3ccccc34)nc(-c3cccc4oc5cc(-c6ccc(N(c7ccccc7)c7ccccc7)cc6)c6ccccc6c5c34)n2)cc1]

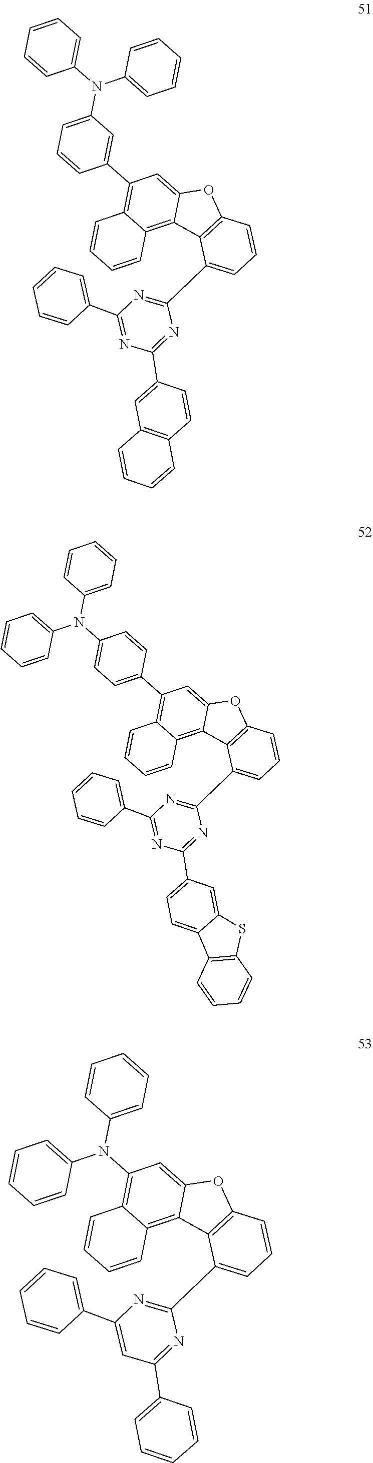 [SMILES: c1ccc(-c2nc(-c3ccc4ccccc4c3)nc(-c3cccc4oc5cc(-c6cccc(N(c7ccccc7)c7ccccc7)c6)c6ccccc6c5c34)n2)cc1 ; c1ccc(-c2nc(-c3ccc4c(c3)sc3ccccc34)nc(-c3cccc4oc5cc(-c6ccc(N(c7ccccc7)c7ccccc7)cc6)c6ccccc6c5c34)n2)cc1 ; c1ccc(-c2cc(-c3ccccc3)nc(-c3cccc4oc5cc(N(c6ccccc6)c6ccccc6)c6ccccc6c5c34)n2)cc1]

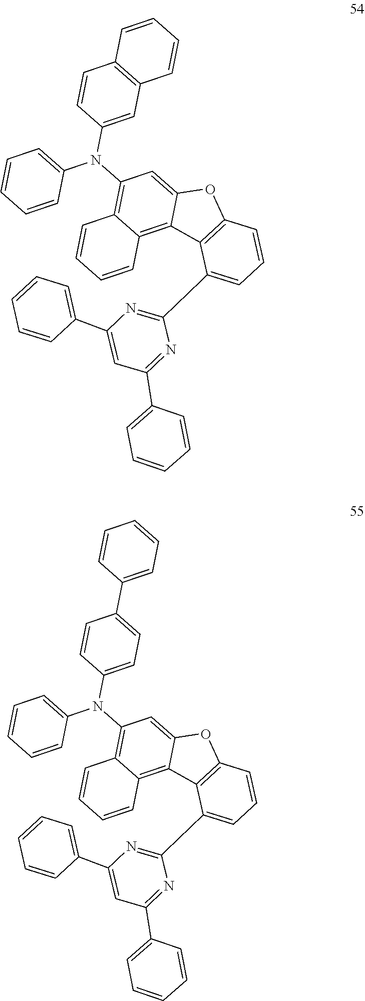 [SMILES: c1ccc(-c2cc(-c3ccccc3)nc(-c3cccc4oc5cc(N(c6ccccc6)c6ccc7ccccc7c6)c6ccccc6c5c34)n2)cc1 ; c1ccc(-c2ccc(N(c3ccccc3)c3cc4oc5cccc(-c6nc(-c7ccccc7)cc(-c7ccccc7)n6)c5c4c4ccccc34)cc2)cc1]

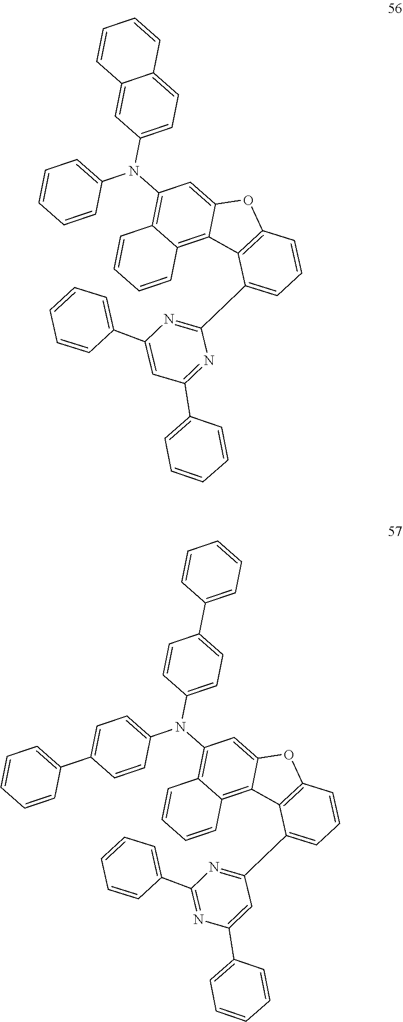 [SMILES: c1ccc(-c2cc(-c3ccccc3)nc(-c3cccc4oc5cc(N(c6ccccc6)c6ccc7ccccc7c6)c6ccccc6c5c34)n2)cc1 ; c1ccc(-c2ccc(N(c3ccc(-c4ccccc4)cc3)c3cc4oc5cccc(-c6cc(-c7ccccc7)nc(-c7ccccc7)n6)c5c4c4ccccc34)cc2)cc1]

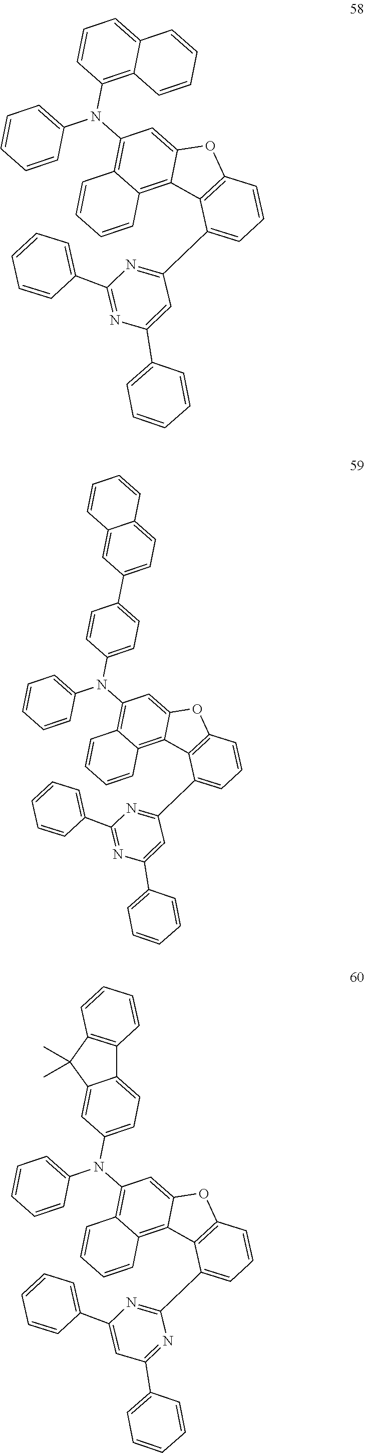 [SMILES: c1ccc(-c2cc(-c3cccc4oc5cc(N(c6ccccc6)c6cccc7ccccc67)c6ccccc6c5c34)nc(-c3ccccc3)n2)cc1 ; c1ccc(-c2cc(-c3cccc4oc5cc(N(c6ccccc6)c6ccc(-c7ccc8ccccc8c7)cc6)c6ccccc6c5c34)nc(-c3ccccc3)n2)cc1 ; CC1(C)c2ccccc2-c2ccc(N(c3ccccc3)c3cc4oc5cccc(-c6nc(-c7ccccc7)cc(-c7ccccc7)n6)c5c4c4ccccc34)cc21]

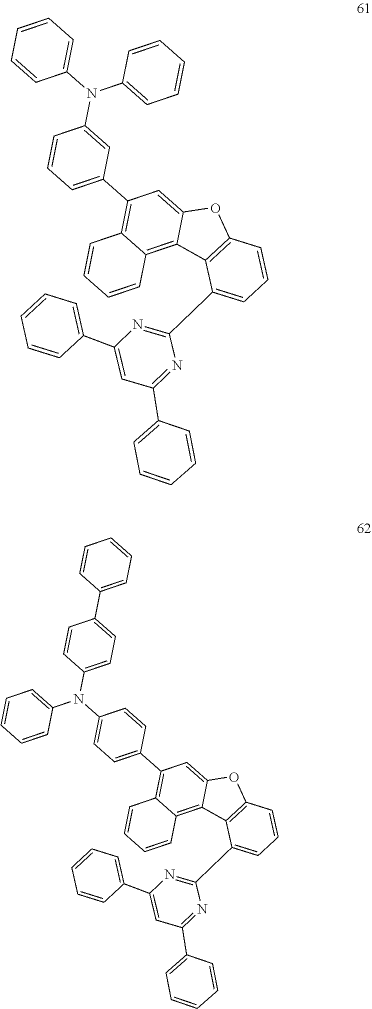 [SMILES: c1ccc(-c2cc(-c3ccccc3)nc(-c3cccc4oc5cc(-c6cccc(N(c7ccccc7)c7ccccc7)c6)c6ccccc6c5c34)n2)cc1 ; c1ccc(-c2ccc(N(c3ccccc3)c3ccc(-c4cc5oc6cccc(-c7nc(-c8ccccc8)cc(-c8ccccc8)n7)c6c5c5ccccc45)cc3)cc2)cc1]

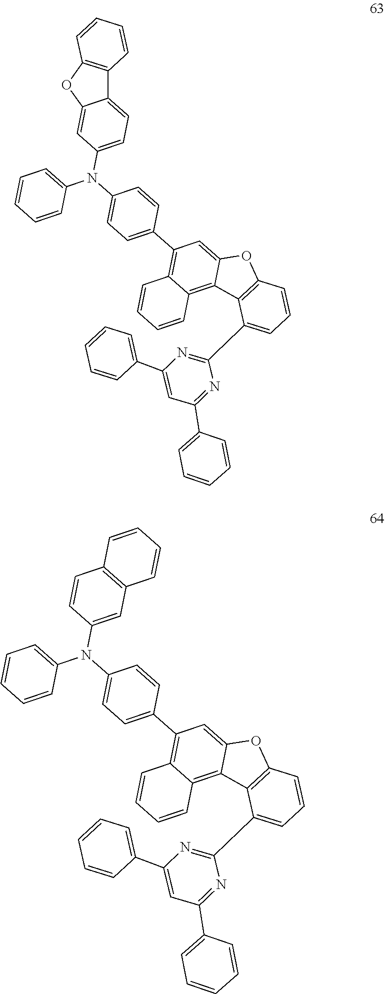 [SMILES: c1ccc(-c2cc(-c3ccccc3)nc(-c3cccc4oc5cc(-c6ccc(N(c7ccccc7)c7ccc8c(c7)oc7ccccc78)cc6)c6ccccc6c5c34)n2)cc1 ; c1ccc(-c2cc(-c3ccccc3)nc(-c3cccc4oc5cc(-c6ccc(N(c7ccccc7)c7ccc8ccccc8c7)cc6)c6ccccc6c5c34)n2)cc1]

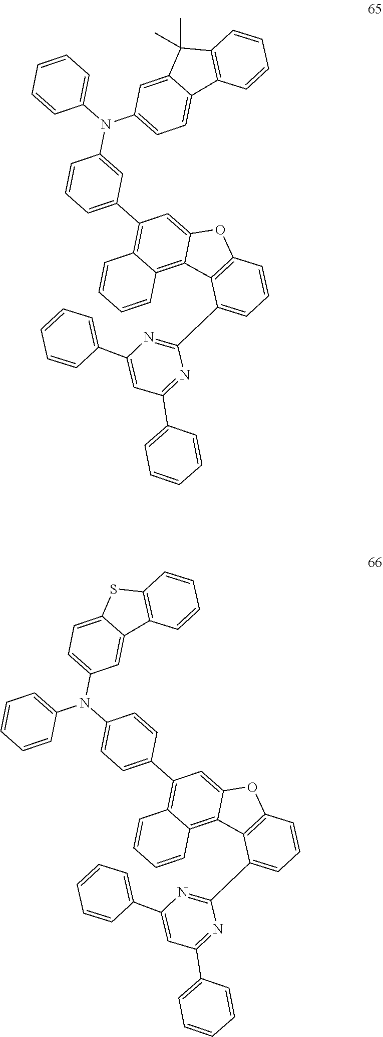 [SMILES: CC1(C)c2ccccc2-c2ccc(N(c3ccccc3)c3cccc(-c4cc5oc6cccc(-c7nc(-c8ccccc8)cc(-c8ccccc8)n7)c6c5c5ccccc45)c3)cc21 ; c1ccc(-c2cc(-c3ccccc3)nc(-c3cccc4oc5cc(-c6ccc(N(c7ccccc7)c7ccc8sc9ccccc9c8c7)cc6)c6ccccc6c5c34)n2)cc1]

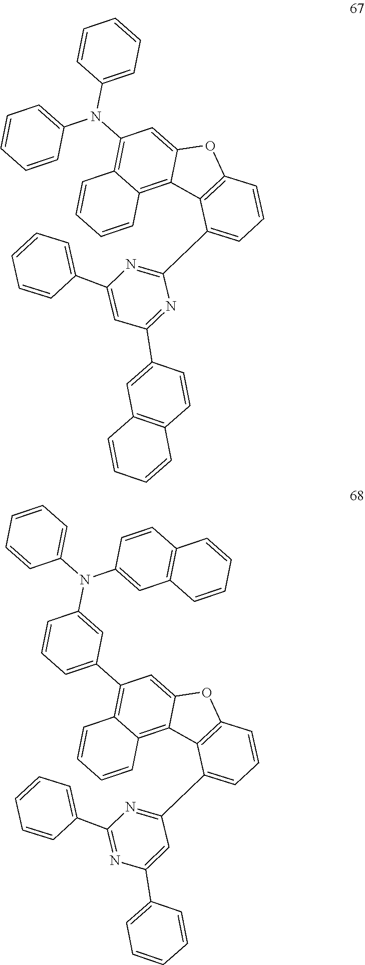 [SMILES: c1ccc(-c2cc(-c3ccc4ccccc4c3)nc(-c3cccc4oc5cc(N(c6ccccc6)c6ccccc6)c6ccccc6c5c34)n2)cc1 ; c1ccc(-c2cc(-c3cccc4oc5cc(-c6cccc(N(c7ccccc7)c7ccc8ccccc8c7)c6)c6ccccc6c5c34)nc(-c3ccccc3)n2)cc1]

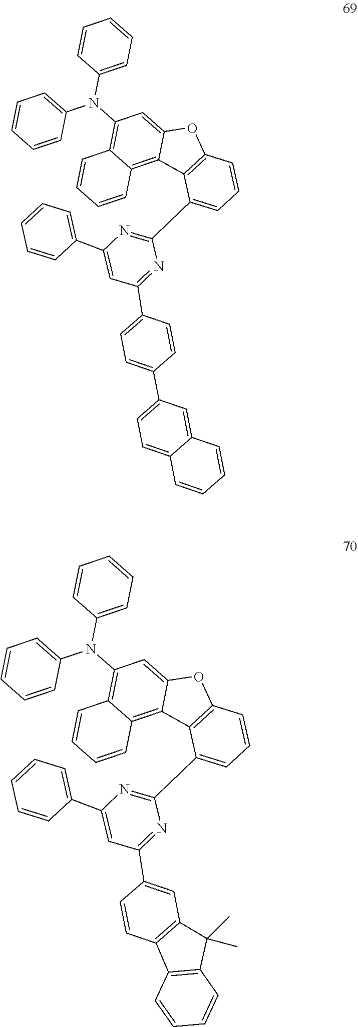 [SMILES: c1ccc(-c2cc(-c3ccc(-c4ccc5ccccc5c4)cc3)nc(-c3cccc4oc5cc(N(c6ccccc6)c6ccccc6)c6ccccc6c5c34)n2)cc1 ; CC1(C)c2ccccc2-c2ccc(-c3cc(-c4ccccc4)nc(-c4cccc5oc6cc(N(c7ccccc7)c7ccccc7)c7ccccc7c6c45)n3)cc21]

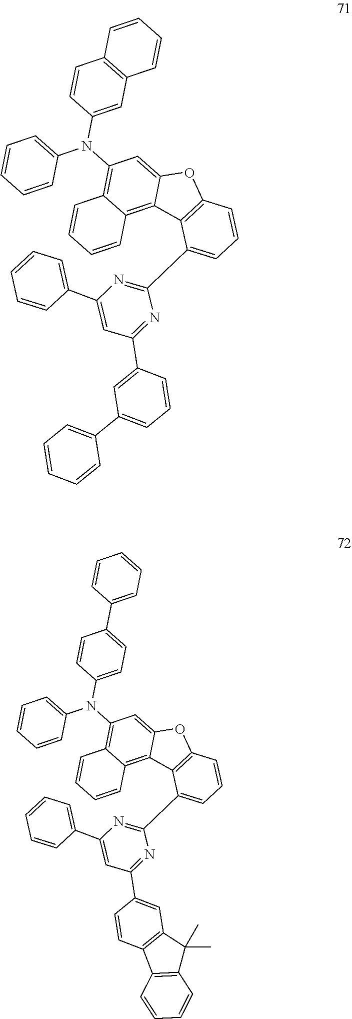 [SMILES: c1ccc(-c2cccc(-c3cc(-c4ccccc4)nc(-c4cccc5oc6cc(N(c7ccccc7)c7ccc8ccccc8c7)c7ccccc7c6c45)n3)c2)cc1 ; CC1(C)c2ccccc2-c2ccc(-c3cc(-c4ccccc4)nc(-c4cccc5oc6cc(N(c7ccccc7)c7ccc(-c8ccccc8)cc7)c7ccccc7c6c45)n3)cc21]

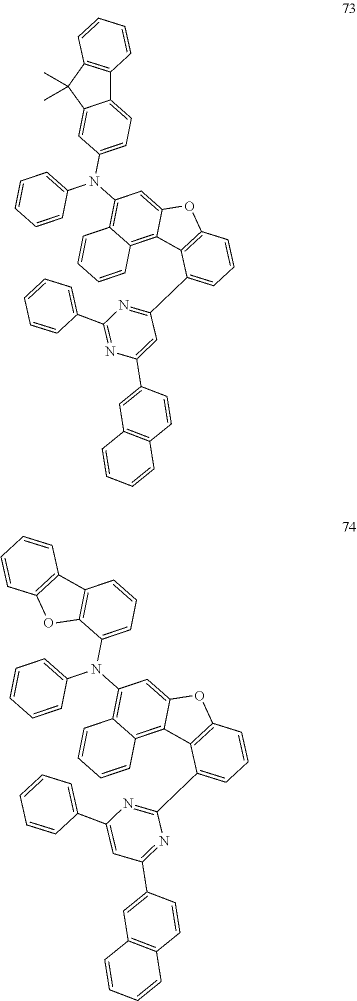 [SMILES: CC1(C)c2ccccc2-c2ccc(N(c3ccccc3)c3cc4oc5cccc(-c6cc(-c7ccc8ccccc8c7)nc(-c7ccccc7)n6)c5c4c4ccccc34)cc21 ; c1ccc(-c2cc(-c3ccc4ccccc4c3)nc(-c3cccc4oc5cc(N(c6ccccc6)c6cccc7c6oc6ccccc67)c6ccccc6c5c34)n2)cc1]

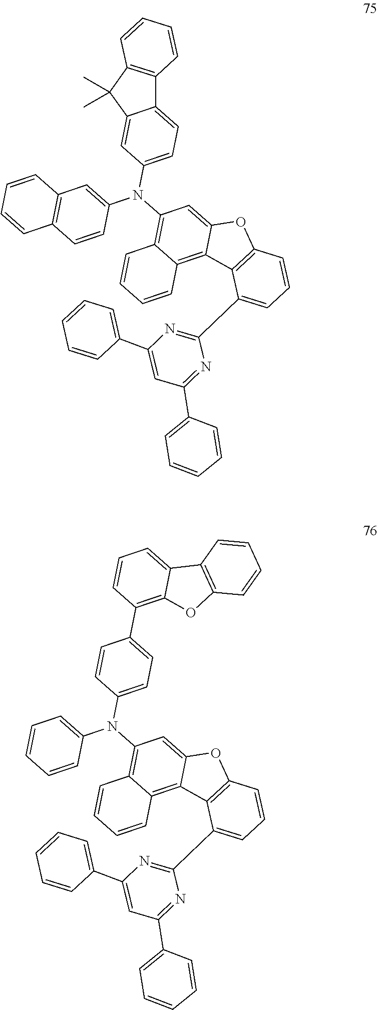 [SMILES: CC1(C)c2ccccc2-c2ccc(N(c3ccc4ccccc4c3)c3cc4oc5cccc(-c6nc(-c7ccccc7)cc(-c7ccccc7)n6)c5c4c4ccccc34)cc21 ; c1ccc(-c2cc(-c3ccccc3)nc(-c3cccc4oc5cc(N(c6ccccc6)c6ccc(-c7cccc8c7oc7ccccc78)cc6)c6ccccc6c5c34)n2)cc1]

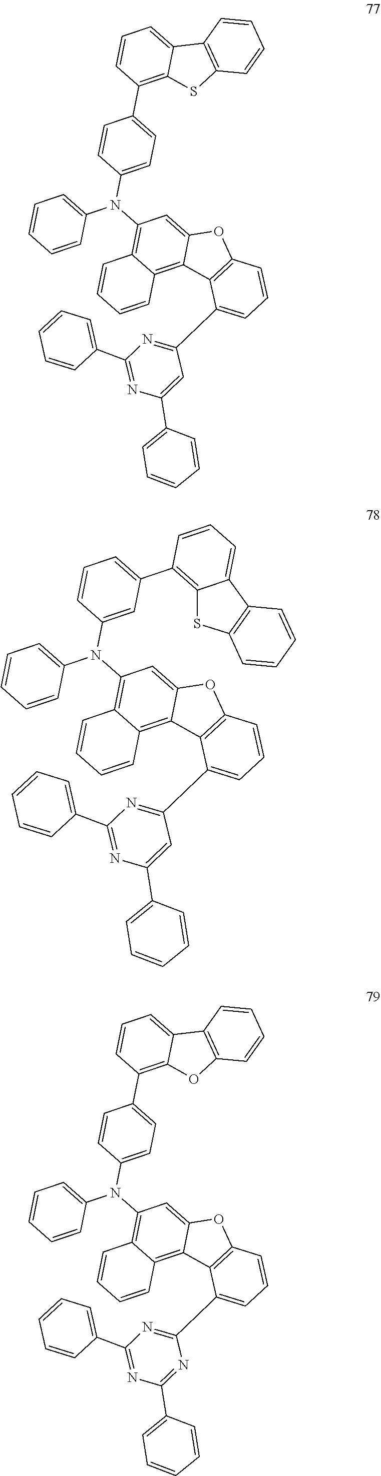 [SMILES: c1ccc(-c2cc(-c3cccc4oc5cc(N(c6ccccc6)c6ccc(-c7cccc8c7sc7ccccc78)cc6)c6ccccc6c5c34)nc(-c3ccccc3)n2)cc1 ; c1ccc(-c2cc(-c3cccc4oc5cc(N(c6ccccc6)c6cccc(-c7cccc8c7sc7ccccc78)c6)c6ccccc6c5c34)nc(-c3ccccc3)n2)cc1 ; c1ccc(-c2nc(-c3ccccc3)nc(-c3cccc4oc5cc(N(c6ccccc6)c6ccc(-c7cccc8c7oc7ccccc78)cc6)c6ccccc6c5c34)n2)cc1]

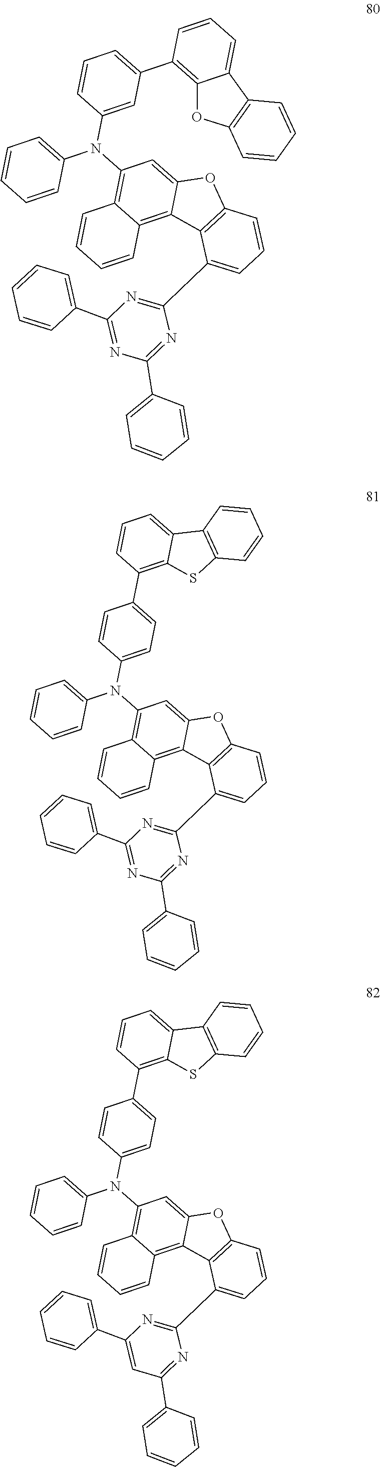 [SMILES: c1ccc(-c2nc(-c3ccccc3)nc(-c3cccc4oc5cc(N(c6ccccc6)c6cccc(-c7cccc8c7oc7ccccc78)c6)c6ccccc6c5c34)n2)cc1 ; c1ccc(-c2nc(-c3ccccc3)nc(-c3cccc4oc5cc(N(c6ccccc6)c6ccc(-c7cccc8c7sc7ccccc78)cc6)c6ccccc6c5c34)n2)cc1 ; c1ccc(-c2cc(-c3ccccc3)nc(-c3cccc4oc5cc(N(c6ccccc6)c6ccc(-c7cccc8c7sc7ccccc78)cc6)c6ccccc6c5c34)n2)cc1]

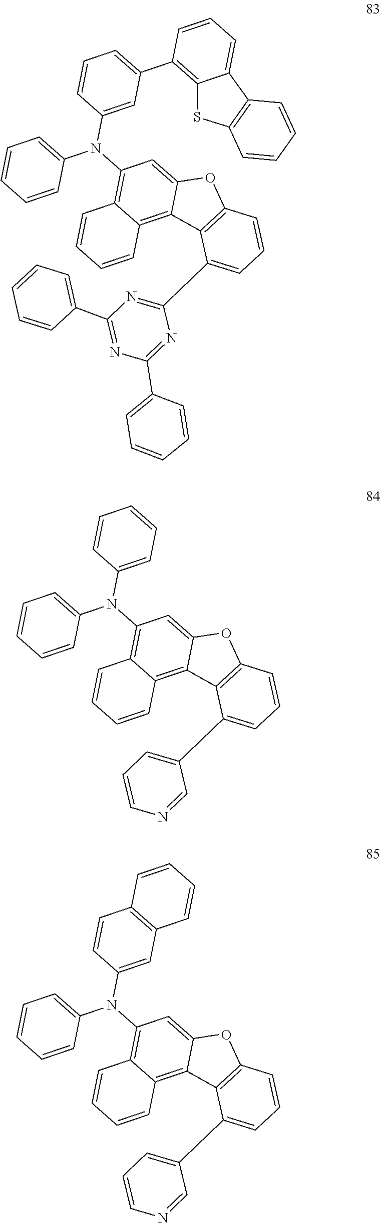 [SMILES: c1ccc(-c2nc(-c3ccccc3)nc(-c3cccc4oc5cc(N(c6ccccc6)c6cccc(-c7cccc8c7sc7ccccc78)c6)c6ccccc6c5c34)n2)cc1 ; c1ccc(N(c2ccccc2)c2cc3oc4cccc(-c5cccnc5)c4c3c3ccccc23)cc1 ; c1ccc(N(c2ccc3ccccc3c2)c2cc3oc4cccc(-c5cccnc5)c4c3c3ccccc23)cc1]

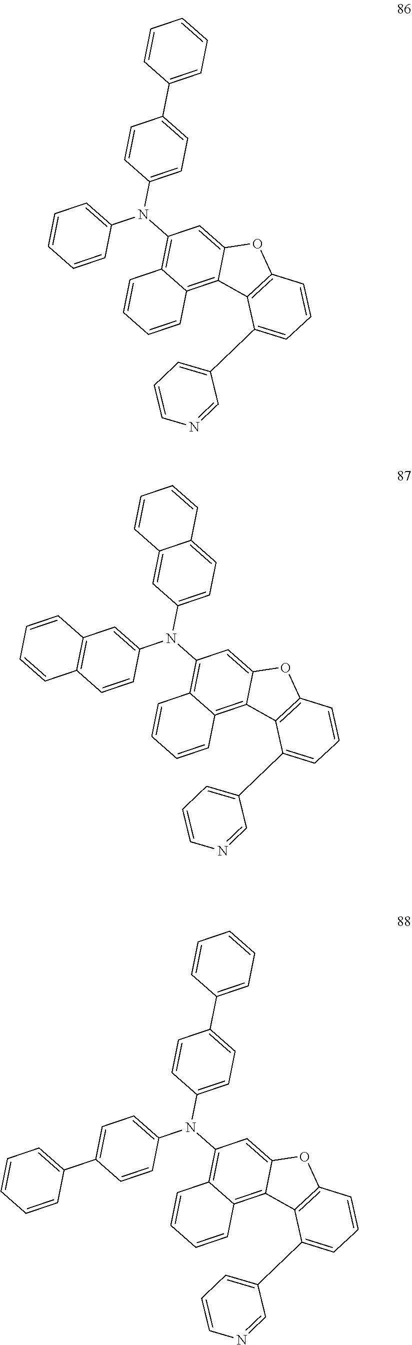 [SMILES: c1ccc(-c2ccc(N(c3ccccc3)c3cc4oc5cccc(-c6cccnc6)c5c4c4ccccc34)cc2)cc1 ; c1cncc(-c2cccc3oc4cc(N(c5ccc6ccccc6c5)c5ccc6ccccc6c5)c5ccccc5c4c23)c1 ; c1ccc(-c2ccc(N(c3ccc(-c4ccccc4)cc3)c3cc4oc5cccc(-c6cccnc6)c5c4c4ccccc34)cc2)cc1]

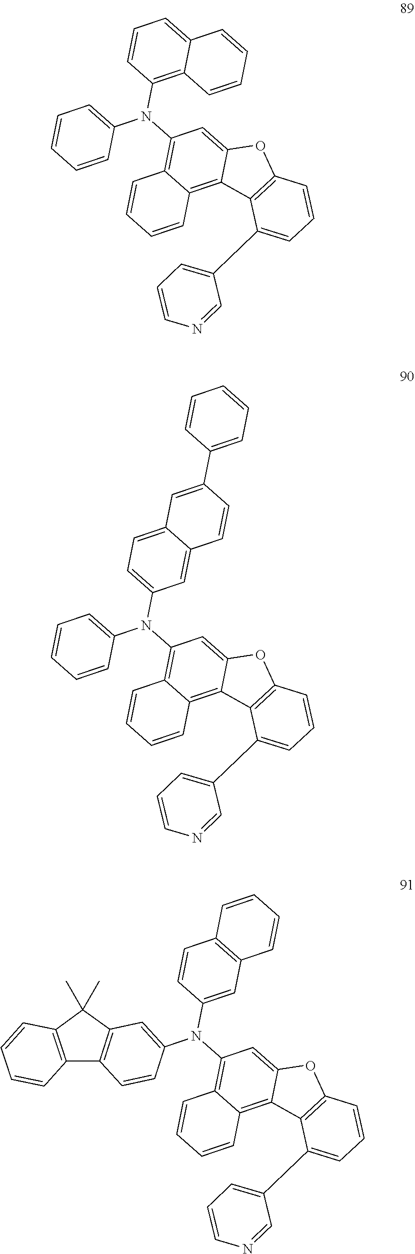 [SMILES: c1ccc(N(c2cccc3ccccc23)c2cc3oc4cccc(-c5cccnc5)c4c3c3ccccc23)cc1 ; c1ccc(-c2ccc3cc(N(c4ccccc4)c4cc5oc6cccc(-c7cccnc7)c6c5c5ccccc45)ccc3c2)cc1 ; CC1(C)c2ccccc2-c2ccc(N(c3ccc4ccccc4c3)c3cc4oc5cccc(-c6cccnc6)c5c4c4ccccc34)cc21]

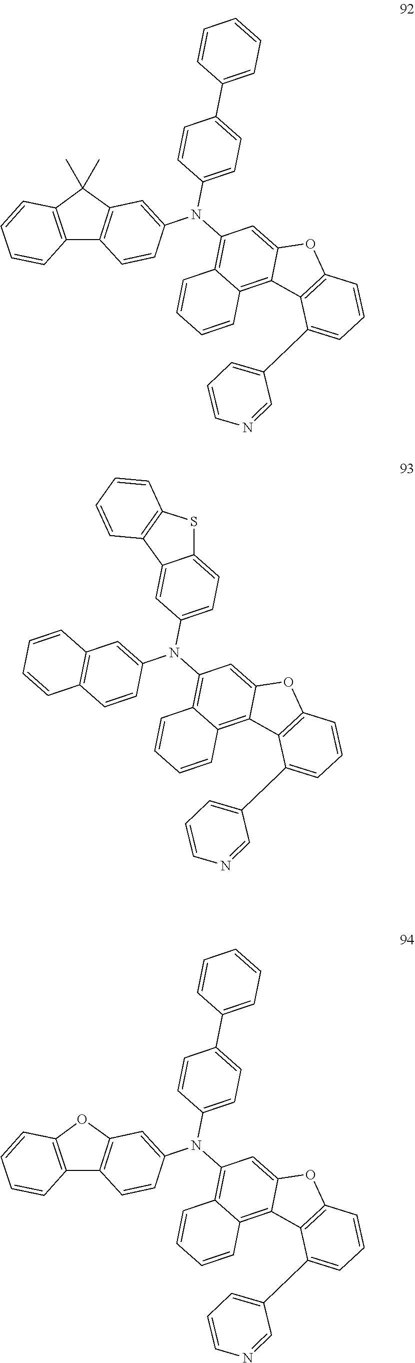 [SMILES: CC1(C)c2ccccc2-c2ccc(N(c3ccc(-c4ccccc4)cc3)c3cc4oc5cccc(-c6cccnc6)c5c4c4ccccc34)cc21 ; c1cncc(-c2cccc3oc4cc(N(c5ccc6ccccc6c5)c5ccc6sc7ccccc7c6c5)c5ccccc5c4c23)c1 ; c1ccc(-c2ccc(N(c3ccc4c(c3)oc3ccccc34)c3cc4oc5cccc(-c6cccnc6)c5c4c4ccccc34)cc2)cc1]

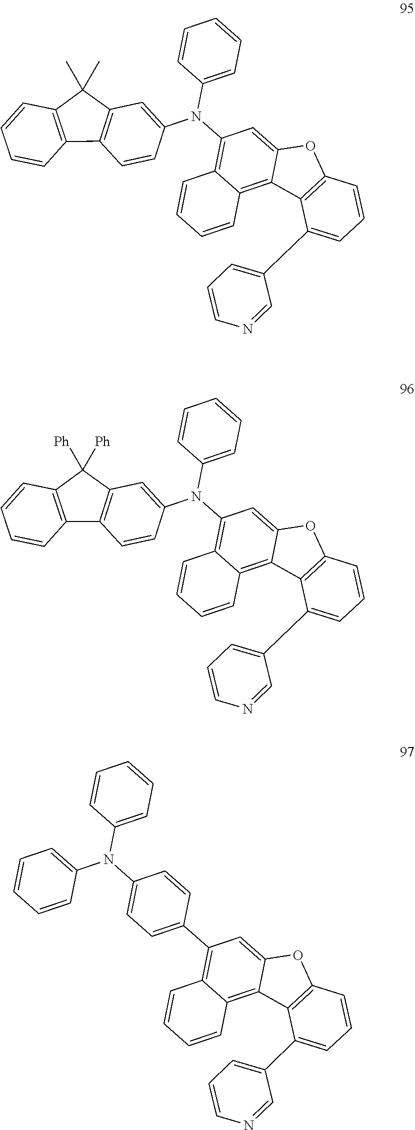 [SMILES: CC1(C)c2ccccc2-c2ccc(N(c3ccccc3)c3cc4oc5cccc(-c6cccnc6)c5c4c4ccccc34)cc21 ; c1ccc(N(c2ccc3c(c2)C(c2ccccc2)(c2ccccc2)c2ccccc2-3)c2cc3oc4cccc(-c5cccnc5)c4c3c3ccccc23)cc1 ; c1ccc(N(c2ccccc2)c2ccc(-c3cc4oc5cccc(-c6cccnc6)c5c4c4ccccc34)cc2)cc1]

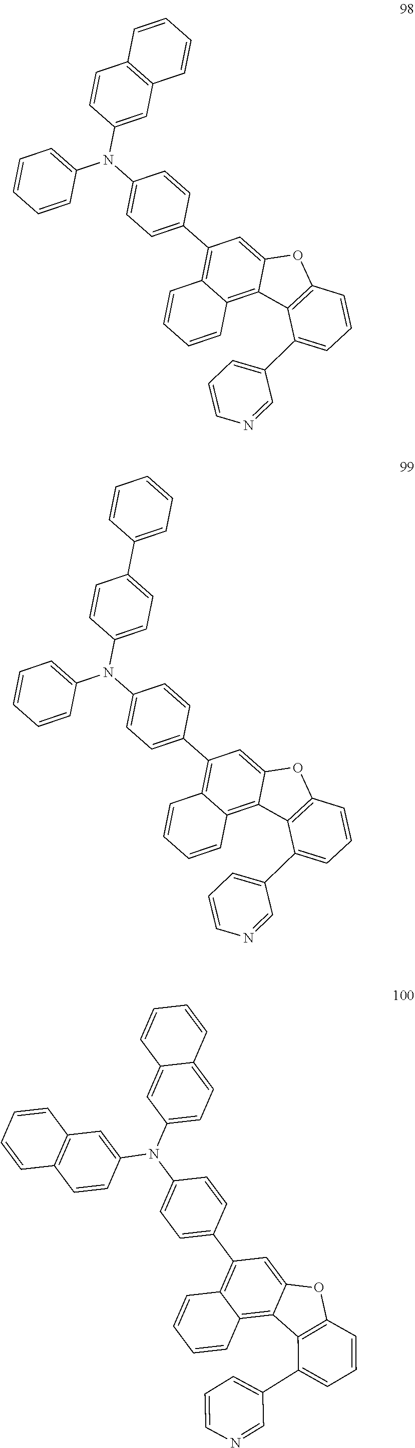 [SMILES: c1ccc(N(c2ccc(-c3cc4oc5cccc(-c6cccnc6)c5c4c4ccccc34)cc2)c2ccc3ccccc3c2)cc1 ; c1ccc(-c2ccc(N(c3ccccc3)c3ccc(-c4cc5oc6cccc(-c7cccnc7)c6c5c5ccccc45)cc3)cc2)cc1 ; c1cncc(-c2cccc3oc4cc(-c5ccc(N(c6ccc7ccccc7c6)c6ccc7ccccc7c6)cc5)c5ccccc5c4c23)c1]

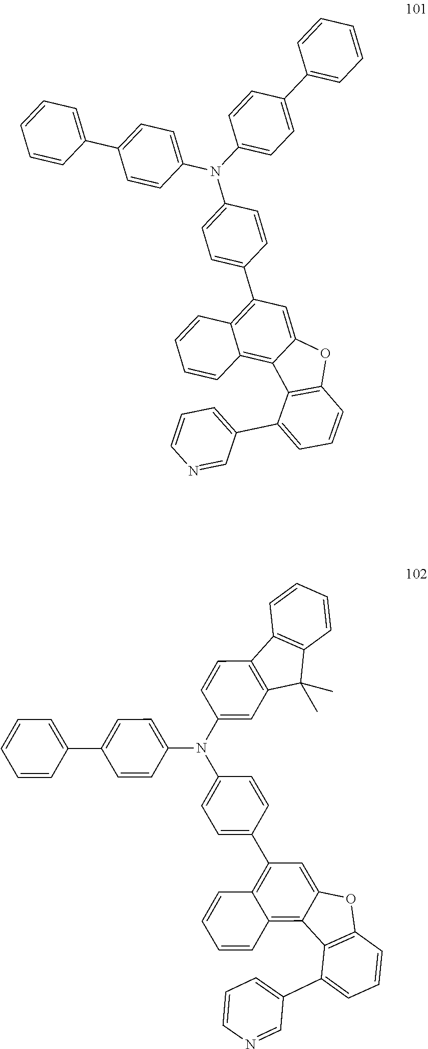 [SMILES: c1ccc(-c2ccc(N(c3ccc(-c4ccccc4)cc3)c3ccc(-c4cc5oc6cccc(-c7cccnc7)c6c5c5ccccc45)cc3)cc2)cc1 ; CC1(C)c2ccccc2-c2ccc(N(c3ccc(-c4ccccc4)cc3)c3ccc(-c4cc5oc6cccc(-c7cccnc7)c6c5c5ccccc45)cc3)cc21]

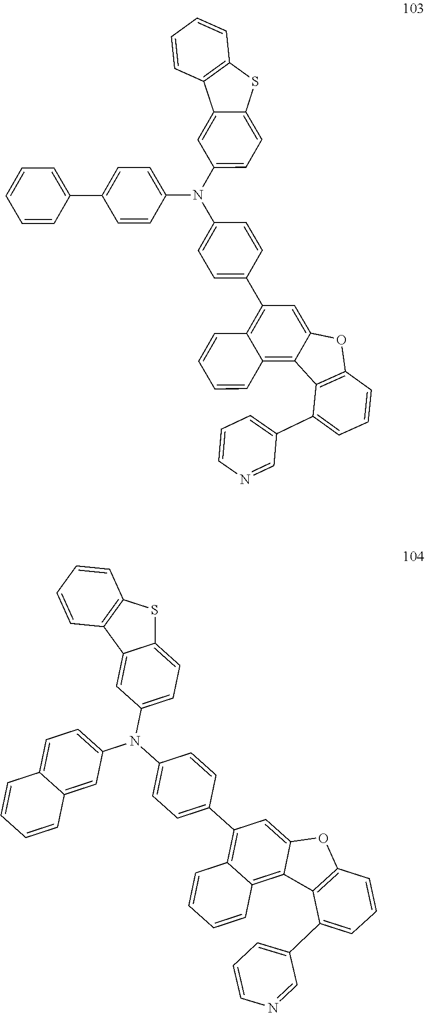 [SMILES: c1ccc(-c2ccc(N(c3ccc(-c4cc5oc6cccc(-c7cccnc7)c6c5c5ccccc45)cc3)c3ccc4sc5ccccc5c4c3)cc2)cc1 ; c1cncc(-c2cccc3oc4cc(-c5ccc(N(c6ccc7ccccc7c6)c6ccc7sc8ccccc8c7c6)cc5)c5ccccc5c4c23)c1]

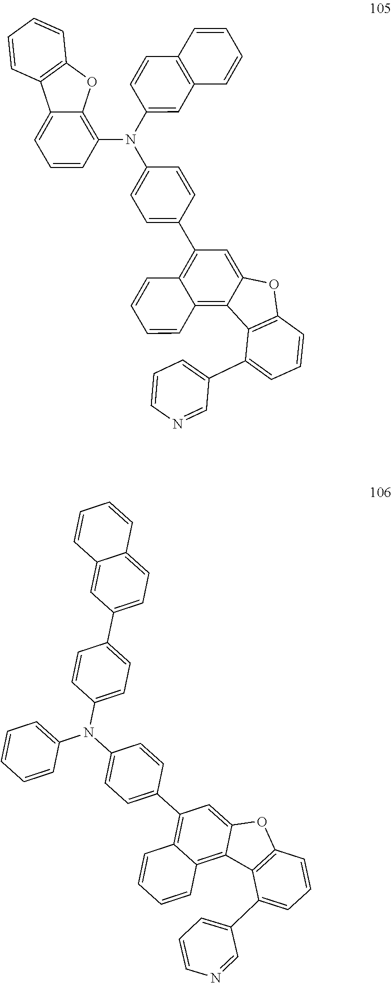 [SMILES: c1cncc(-c2cccc3oc4cc(-c5ccc(N(c6ccc7ccccc7c6)c6cccc7c6oc6ccccc67)cc5)c5ccccc5c4c23)c1 ; c1ccc(N(c2ccc(-c3ccc4ccccc4c3)cc2)c2ccc(-c3cc4oc5cccc(-c6cccnc6)c5c4c4ccccc34)cc2)cc1]

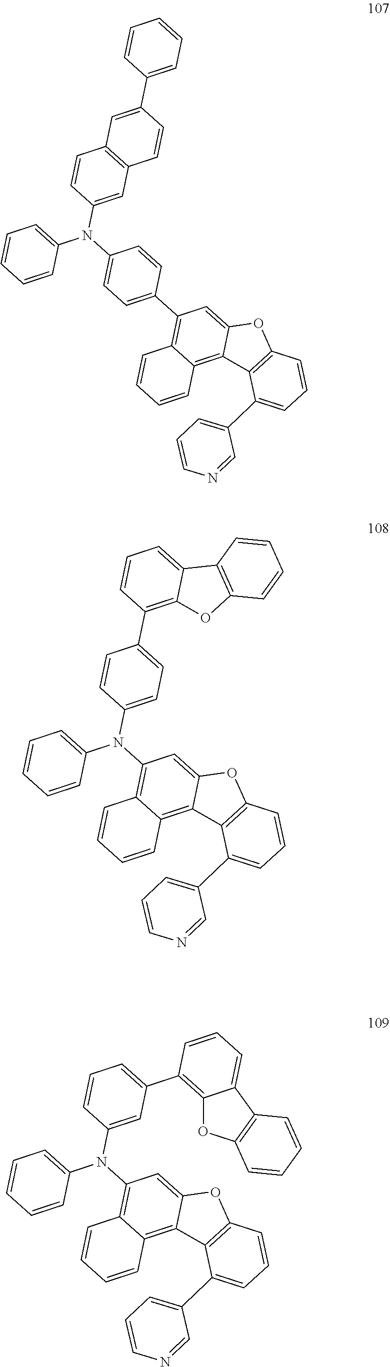 [SMILES: c1ccc(-c2ccc3cc(N(c4ccccc4)c4ccc(-c5cc6oc7cccc(-c8cccnc8)c7c6c6ccccc56)cc4)ccc3c2)cc1 ; c1ccc(N(c2ccc(-c3cccc4c3oc3ccccc34)cc2)c2cc3oc4cccc(-c5cccnc5)c4c3c3ccccc23)cc1 ; c1ccc(N(c2cccc(-c3cccc4c3oc3ccccc34)c2)c2cc3oc4cccc(-c5cccnc5)c4c3c3ccccc23)cc1]

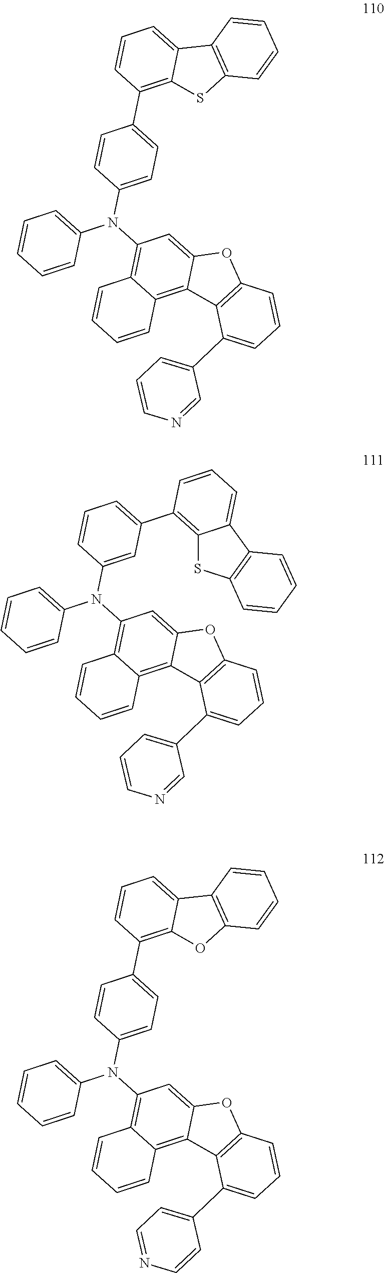 [SMILES: c1ccc(N(c2ccc(-c3cccc4c3sc3ccccc34)cc2)c2cc3oc4cccc(-c5cccnc5)c4c3c3ccccc23)cc1 ; c1ccc(N(c2cccc(-c3cccc4c3sc3ccccc34)c2)c2cc3oc4cccc(-c5cccnc5)c4c3c3ccccc23)cc1 ; c1ccc(N(c2ccc(-c3cccc4c3oc3ccccc34)cc2)c2cc3oc4cccc(-c5ccncc5)c4c3c3ccccc23)cc1]

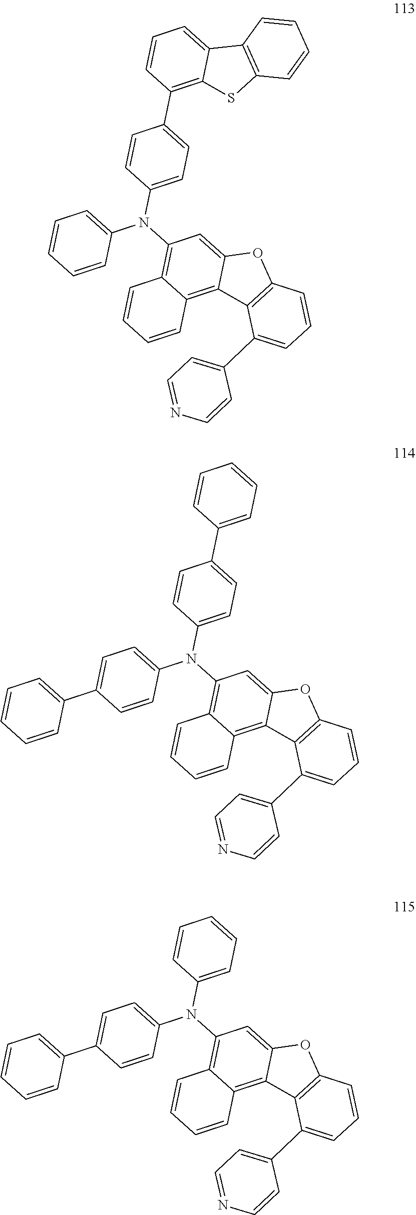 [SMILES: c1ccc(N(c2ccc(-c3cccc4c3sc3ccccc34)cc2)c2cc3oc4cccc(-c5ccncc5)c4c3c3ccccc23)cc1 ; c1ccc(-c2ccc(N(c3ccc(-c4ccccc4)cc3)c3cc4oc5cccc(-c6ccncc6)c5c4c4ccccc34)cc2)cc1 ; c1ccc(-c2ccc(N(c3ccccc3)c3cc4oc5cccc(-c6ccncc6)c5c4c4ccccc34)cc2)cc1]

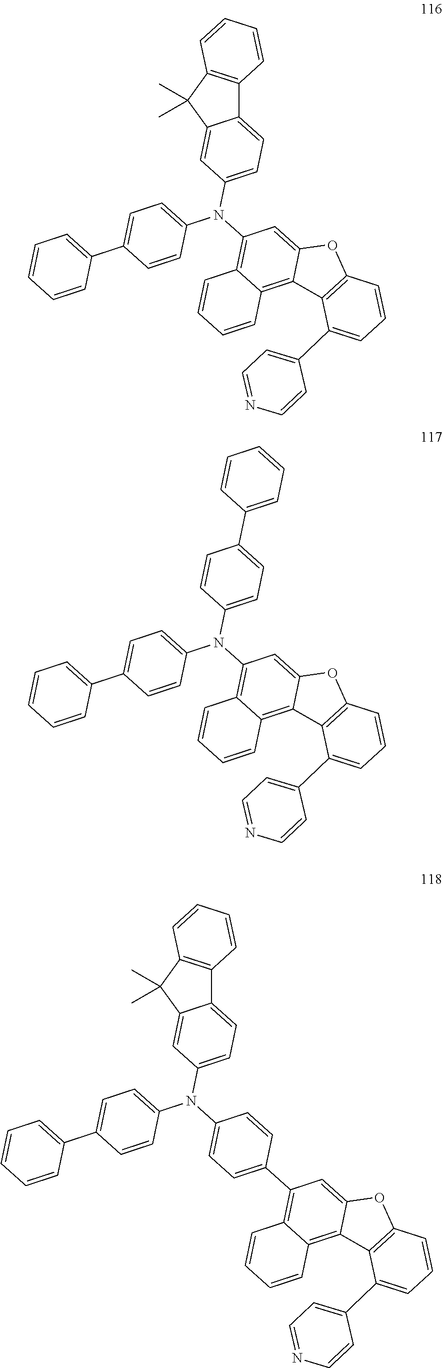

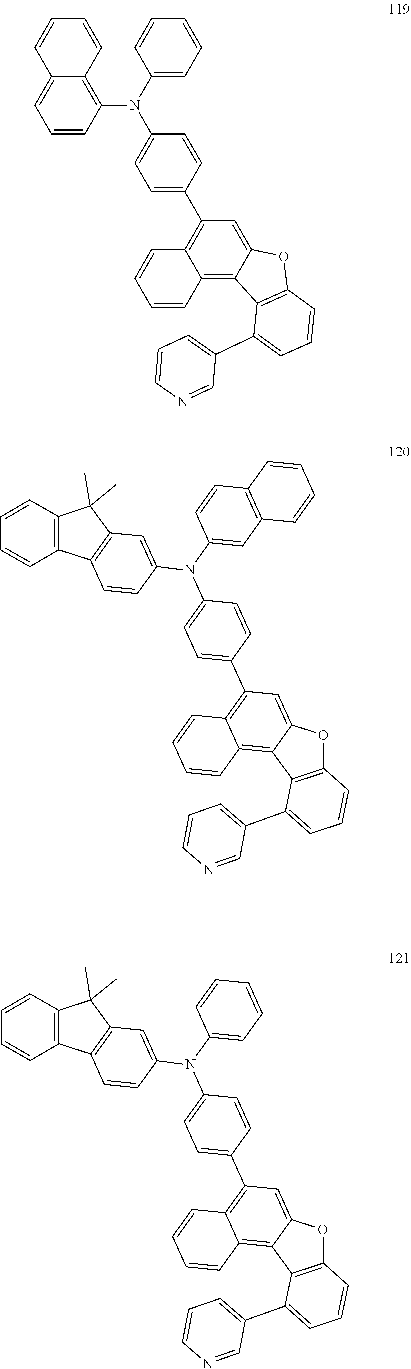 [SMILES: c1ccc(N(c2ccc(-c3cc4oc5cccc(-c6cccnc6)c5c4c4ccccc34)cc2)c2cccc3ccccc23)cc1 ; CC1(C)c2ccccc2-c2ccc(N(c3ccc(-c4cc5oc6cccc(-c7cccnc7)c6c5c5ccccc45)cc3)c3ccc4ccccc4c3)cc21 ; CC1(C)c2ccccc2-c2ccc(N(c3ccccc3)c3ccc(-c4cc5oc6cccc(-c7cccnc7)c6c5c5ccccc45)cc3)cc21]

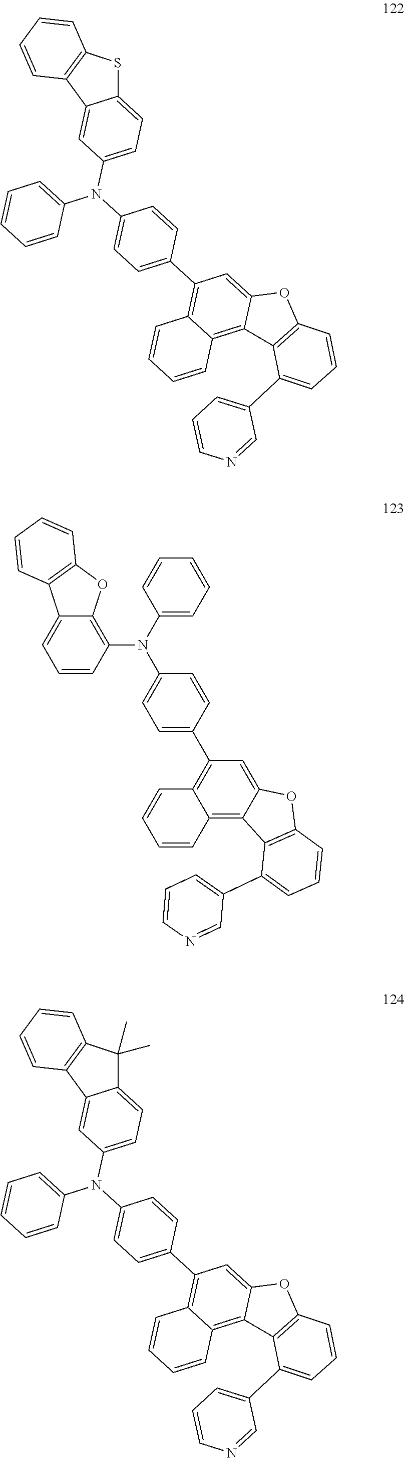 [SMILES: c1ccc(N(c2ccc(-c3cc4oc5cccc(-c6cccnc6)c5c4c4ccccc34)cc2)c2ccc3sc4ccccc4c3c2)cc1 ; c1ccc(N(c2ccc(-c3cc4oc5cccc(-c6cccnc6)c5c4c4ccccc34)cc2)c2cccc3c2oc2ccccc23)cc1 ; CC1(C)c2ccccc2-c2cc(N(c3ccccc3)c3ccc(-c4cc5oc6cccc(-c7cccnc7)c6c5c5ccccc45)cc3)ccc21]

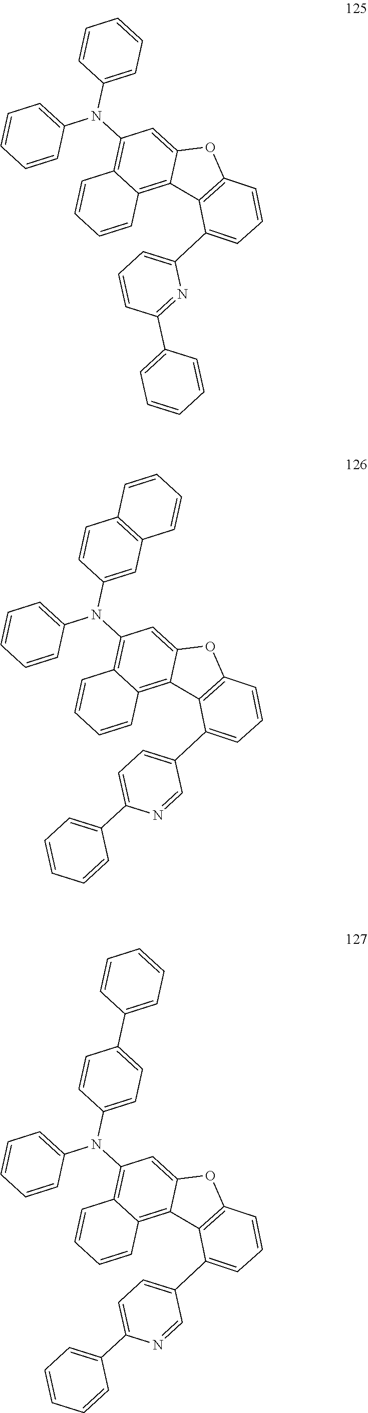 [SMILES: c1ccc(-c2cccc(-c3cccc4oc5cc(N(c6ccccc6)c6ccccc6)c6ccccc6c5c34)n2)cc1 ; c1ccc(-c2ccc(-c3cccc4oc5cc(N(c6ccccc6)c6ccc7ccccc7c6)c6ccccc6c5c34)cn2)cc1 ; c1ccc(-c2ccc(N(c3ccccc3)c3cc4oc5cccc(-c6ccc(-c7ccccc7)nc6)c5c4c4ccccc34)cc2)cc1]

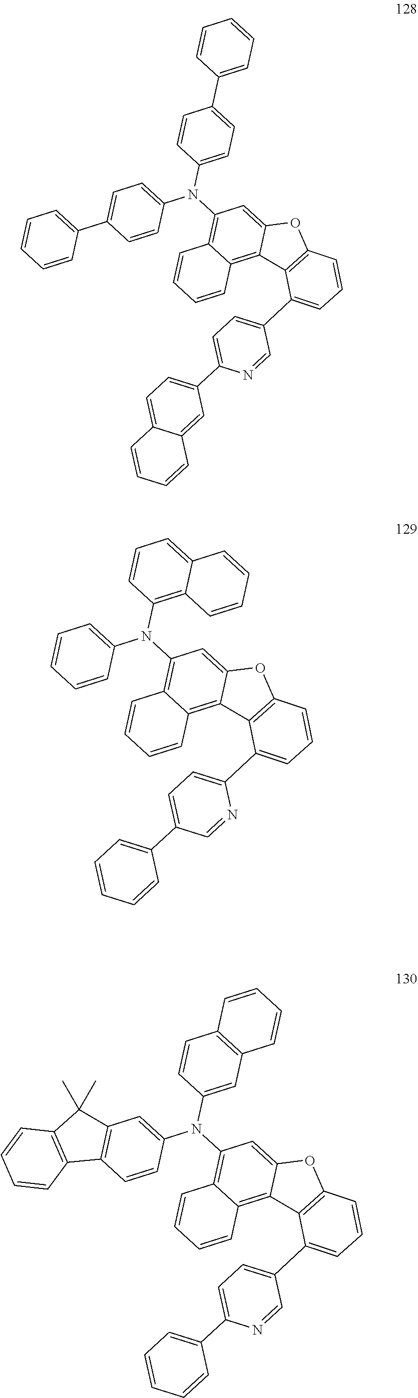 [SMILES: c1ccc(-c2ccc(N(c3ccc(-c4ccccc4)cc3)c3cc4oc5cccc(-c6ccc(-c7ccc8ccccc8c7)nc6)c5c4c4ccccc34)cc2)cc1 ; c1ccc(-c2ccc(-c3cccc4oc5cc(N(c6ccccc6)c6cccc7ccccc67)c6ccccc6c5c34)nc2)cc1 ; CC1(C)c2ccccc2-c2ccc(N(c3ccc4ccccc4c3)c3cc4oc5cccc(-c6ccc(-c7ccccc7)nc6)c5c4c4ccccc34)cc21]

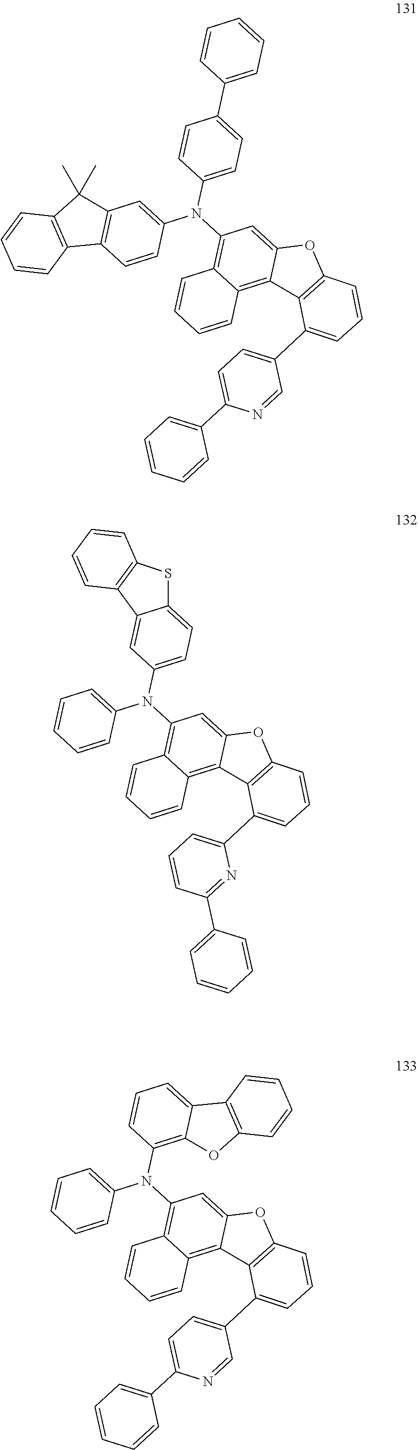 [SMILES: CC1(C)c2ccccc2-c2ccc(N(c3ccc(-c4ccccc4)cc3)c3cc4oc5cccc(-c6ccc(-c7ccccc7)nc6)c5c4c4ccccc34)cc21 ; c1ccc(-c2cccc(-c3cccc4oc5cc(N(c6ccccc6)c6ccc7sc8ccccc8c7c6)c6ccccc6c5c34)n2)cc1 ; c1ccc(-c2ccc(-c3cccc4oc5cc(N(c6ccccc6)c6cccc7c6oc6ccccc67)c6ccccc6c5c34)cn2)cc1]

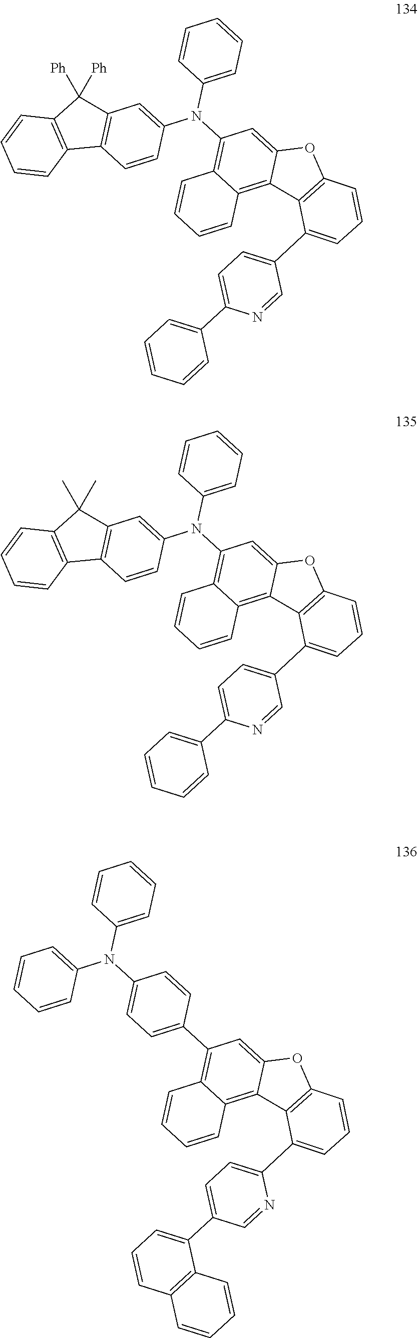 [SMILES: c1ccc(-c2ccc(-c3cccc4oc5cc(N(c6ccccc6)c6ccc7c(c6)C(c6ccccc6)(c6ccccc6)c6ccccc6-7)c6ccccc6c5c34)cn2)cc1 ; CC1(C)c2ccccc2-c2ccc(N(c3ccccc3)c3cc4oc5cccc(-c6ccc(-c7ccccc7)nc6)c5c4c4ccccc34)cc21 ; c1ccc(N(c2ccccc2)c2ccc(-c3cc4oc5cccc(-c6ccc(-c7cccc8ccccc78)cn6)c5c4c4ccccc34)cc2)cc1]

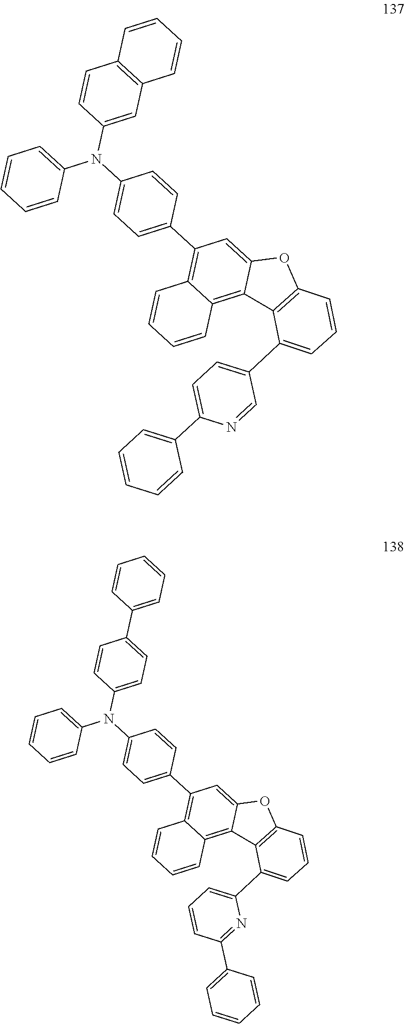 [SMILES: c1ccc(-c2ccc(-c3cccc4oc5cc(-c6ccc(N(c7ccccc7)c7ccc8ccccc8c7)cc6)c6ccccc6c5c34)cn2)cc1 ; c1ccc(-c2ccc(N(c3ccccc3)c3ccc(-c4cc5oc6cccc(-c7cccc(-c8ccccc8)n7)c6c5c5ccccc45)cc3)cc2)cc1]

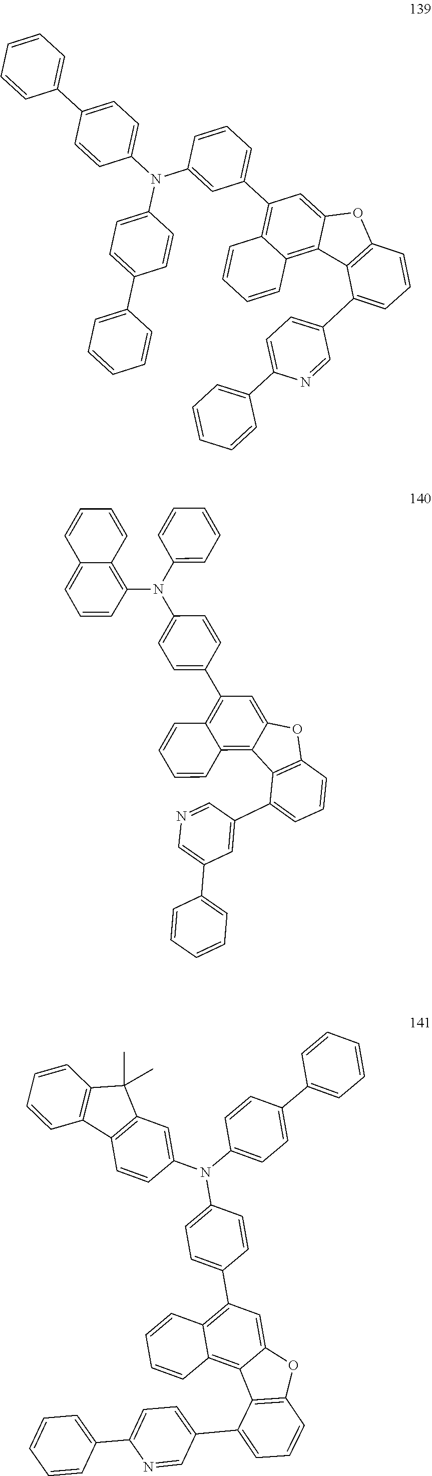 [SMILES: c1ccc(-c2ccc(N(c3ccc(-c4ccccc4)cc3)c3cccc(-c4cc5oc6cccc(-c7ccc(-c8ccccc8)nc7)c6c5c5ccccc45)c3)cc2)cc1 ; c1ccc(-c2cncc(-c3cccc4oc5cc(-c6ccc(N(c7ccccc7)c7cccc8ccccc78)cc6)c6ccccc6c5c34)c2)cc1 ; CC1(C)c2ccccc2-c2ccc(N(c3ccc(-c4ccccc4)cc3)c3ccc(-c4cc5oc6cccc(-c7ccc(-c8ccccc8)nc7)c6c5c5ccccc45)cc3)cc21]

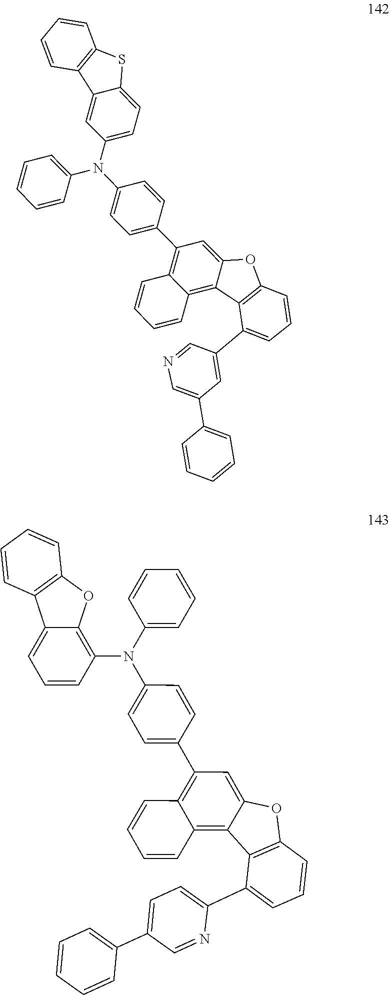 [SMILES: c1ccc(-c2cncc(-c3cccc4oc5cc(-c6ccc(N(c7ccccc7)c7ccc8sc9ccccc9c8c7)cc6)c6ccccc6c5c34)c2)cc1 ; c1ccc(-c2ccc(-c3cccc4oc5cc(-c6ccc(N(c7ccccc7)c7cccc8c7oc7ccccc78)cc6)c6ccccc6c5c34)nc2)cc1]

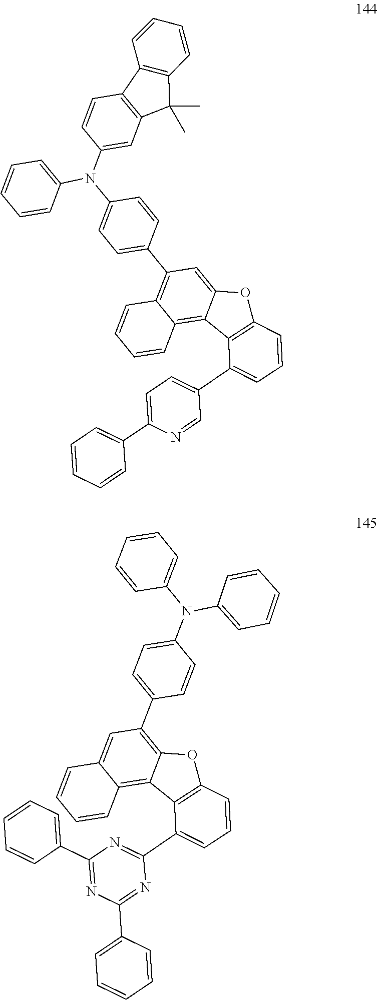 [SMILES: CC1(C)c2ccccc2-c2ccc(N(c3ccccc3)c3ccc(-c4cc5oc6cccc(-c7ccc(-c8ccccc8)nc7)c6c5c5ccccc45)cc3)cc21 ; c1ccc(-c2nc(-c3ccccc3)nc(-c3cccc4oc5c(-c6ccc(N(c7ccccc7)c7ccccc7)cc6)cc6ccccc6c5c34)n2)cc1]

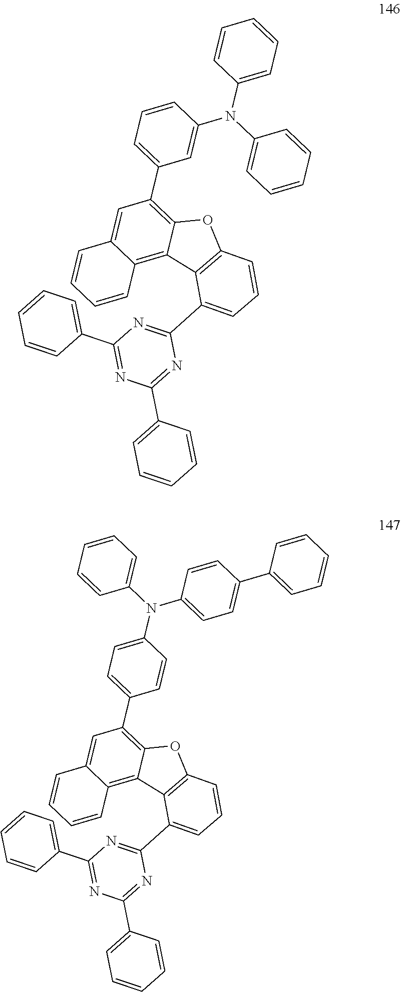 [SMILES: c1ccc(-c2nc(-c3ccccc3)nc(-c3cccc4oc5c(-c6cccc(N(c7ccccc7)c7ccccc7)c6)cc6ccccc6c5c34)n2)cc1 ; c1ccc(-c2ccc(N(c3ccccc3)c3ccc(-c4cc5ccccc5c5c4oc4cccc(-c6nc(-c7ccccc7)nc(-c7ccccc7)n6)c45)cc3)cc2)cc1]

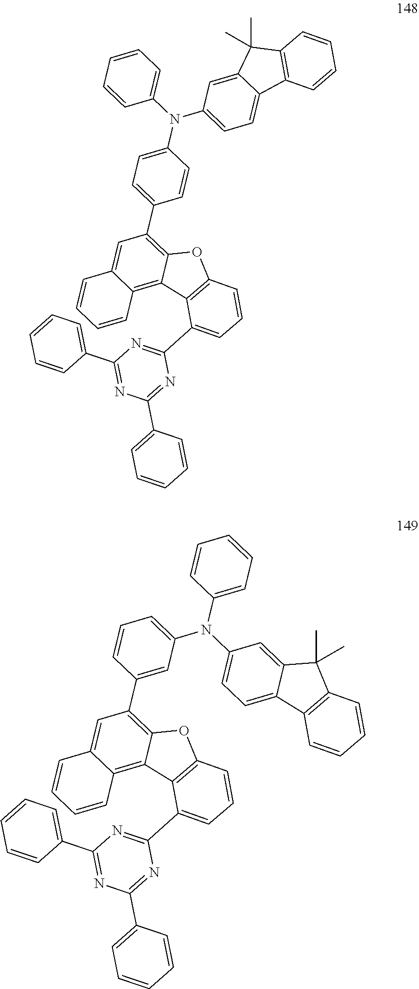 [SMILES: CC1(C)c2ccccc2-c2ccc(N(c3ccccc3)c3ccc(-c4cc5ccccc5c5c4oc4cccc(-c6nc(-c7ccccc7)nc(-c7ccccc7)n6)c45)cc3)cc21 ; CC1(C)c2ccccc2-c2ccc(N(c3ccccc3)c3cccc(-c4cc5ccccc5c5c4oc4cccc(-c6nc(-c7ccccc7)nc(-c7ccccc7)n6)c45)c3)cc21]

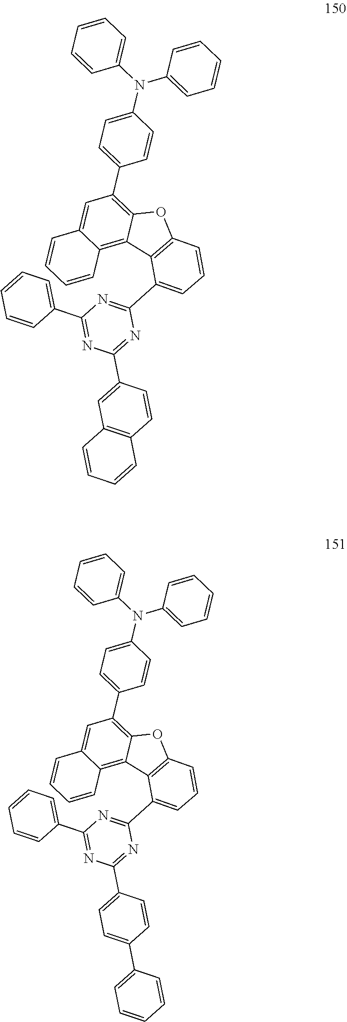 [SMILES: c1ccc(-c2nc(-c3ccc4ccccc4c3)nc(-c3cccc4oc5c(-c6ccc(N(c7ccccc7)c7ccccc7)cc6)cc6ccccc6c5c34)n2)cc1 ; c1ccc(-c2ccc(-c3nc(-c4ccccc4)nc(-c4cccc5oc6c(-c7ccc(N(c8ccccc8)c8ccccc8)cc7)cc7ccccc7c6c45)n3)cc2)cc1]

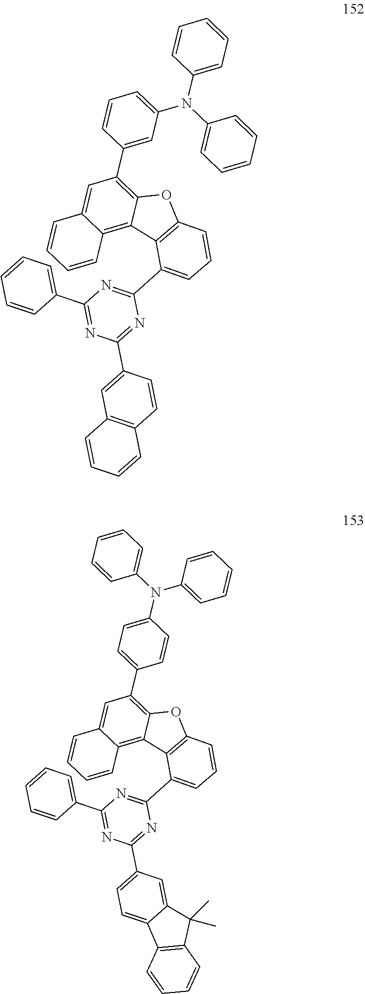 [SMILES: c1ccc(-c2nc(-c3ccc4ccccc4c3)nc(-c3cccc4oc5c(-c6cccc(N(c7ccccc7)c7ccccc7)c6)cc6ccccc6c5c34)n2)cc1 ; CC1(C)c2ccccc2-c2ccc(-c3nc(-c4ccccc4)nc(-c4cccc5oc6c(-c7ccc(N(c8ccccc8)c8ccccc8)cc7)cc7ccccc7c6c45)n3)cc21]

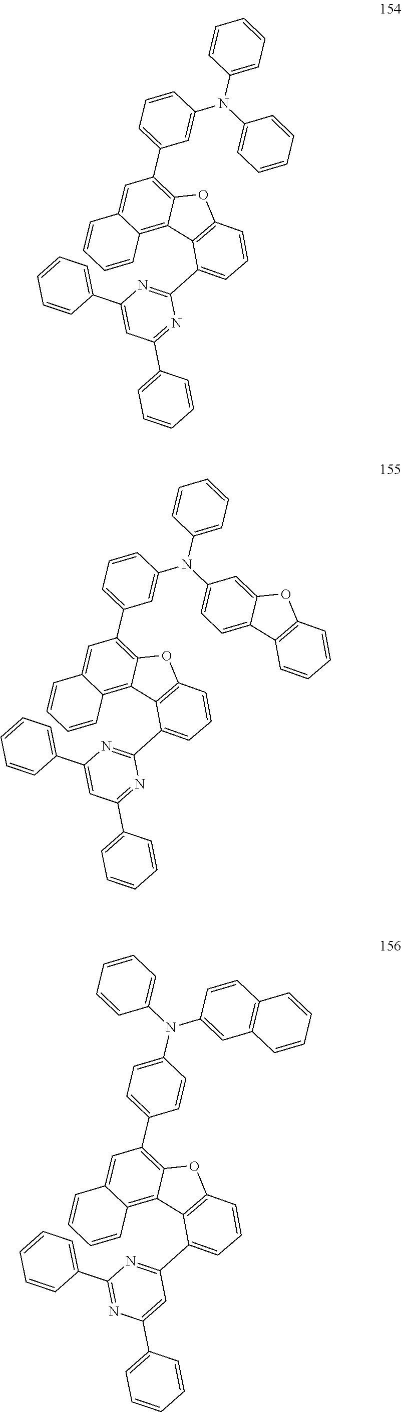 [SMILES: c1ccc(-c2cc(-c3ccccc3)nc(-c3cccc4oc5c(-c6cccc(N(c7ccccc7)c7ccccc7)c6)cc6ccccc6c5c34)n2)cc1 ; c1ccc(-c2cc(-c3ccccc3)nc(-c3cccc4oc5c(-c6cccc(N(c7ccccc7)c7ccc8c(c7)oc7ccccc78)c6)cc6ccccc6c5c34)n2)cc1 ; c1ccc(-c2cc(-c3cccc4oc5c(-c6ccc(N(c7ccccc7)c7ccc8ccccc8c7)cc6)cc6ccccc6c5c34)nc(-c3ccccc3)n2)cc1]

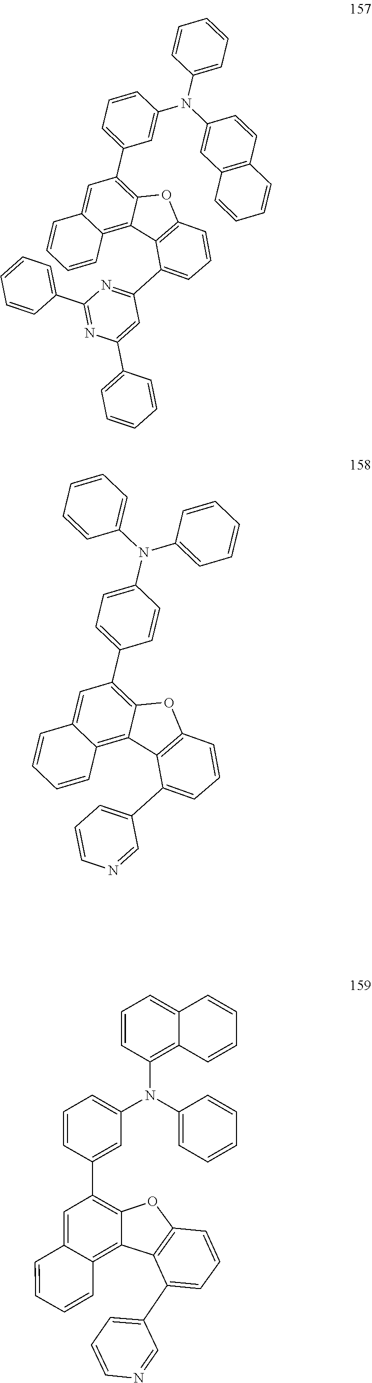 [SMILES: c1ccc(-c2cc(-c3cccc4oc5c(-c6cccc(N(c7ccccc7)c7ccc8ccccc8c7)c6)cc6ccccc6c5c34)nc(-c3ccccc3)n2)cc1 ; c1ccc(N(c2ccccc2)c2ccc(-c3cc4ccccc4c4c3oc3cccc(-c5cccnc5)c34)cc2)cc1 ; c1ccc(N(c2cccc(-c3cc4ccccc4c4c3oc3cccc(-c5cccnc5)c34)c2)c2cccc3ccccc23)cc1]

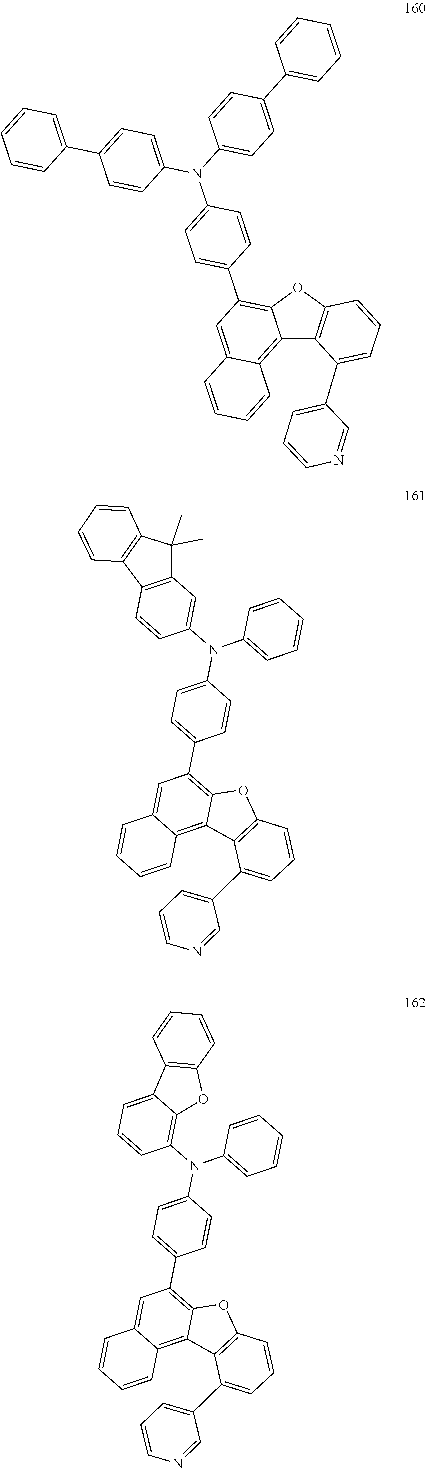 [SMILES: c1ccc(-c2ccc(N(c3ccc(-c4ccccc4)cc3)c3ccc(-c4cc5ccccc5c5c4oc4cccc(-c6cccnc6)c45)cc3)cc2)cc1 ; CC1(C)c2ccccc2-c2ccc(N(c3ccccc3)c3ccc(-c4cc5ccccc5c5c4oc4cccc(-c6cccnc6)c45)cc3)cc21 ; c1ccc(N(c2ccc(-c3cc4ccccc4c4c3oc3cccc(-c5cccnc5)c34)cc2)c2cccc3c2oc2ccccc23)cc1]

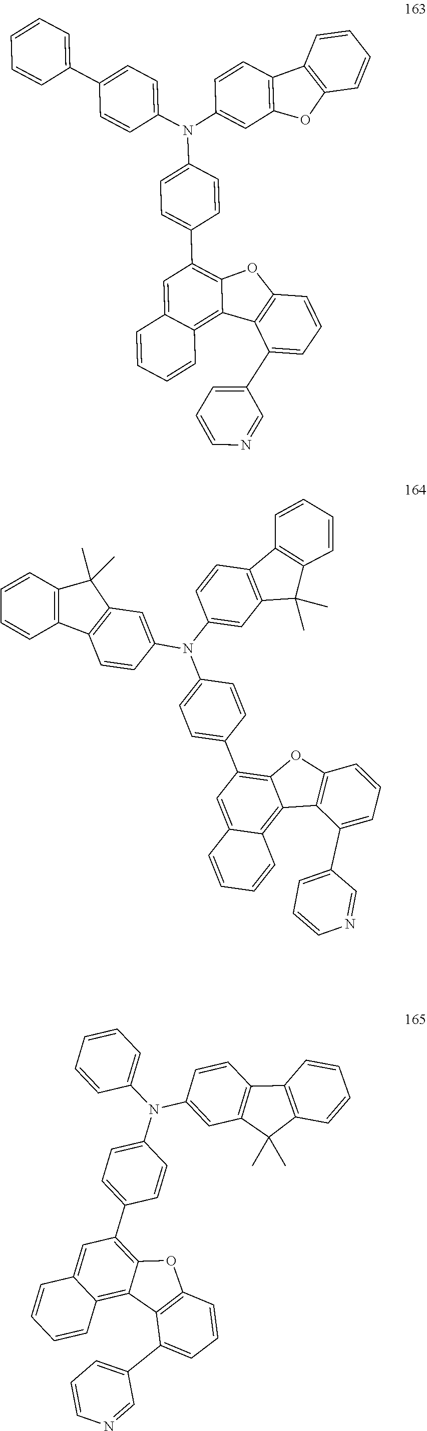 [SMILES: c1ccc(-c2ccc(N(c3ccc(-c4cc5ccccc5c5c4oc4cccc(-c6cccnc6)c45)cc3)c3ccc4c(c3)oc3ccccc34)cc2)cc1 ; CC1(C)c2ccccc2-c2ccc(N(c3ccc(-c4cc5ccccc5c5c4oc4cccc(-c6cccnc6)c45)cc3)c3ccc4c(c3)C(C)(C)c3ccccc3-4)cc21 ; CC1(C)c2ccccc2-c2ccc(N(c3ccccc3)c3ccc(-c4cc5ccccc5c5c4oc4cccc(-c6cccnc6)c45)cc3)cc21]

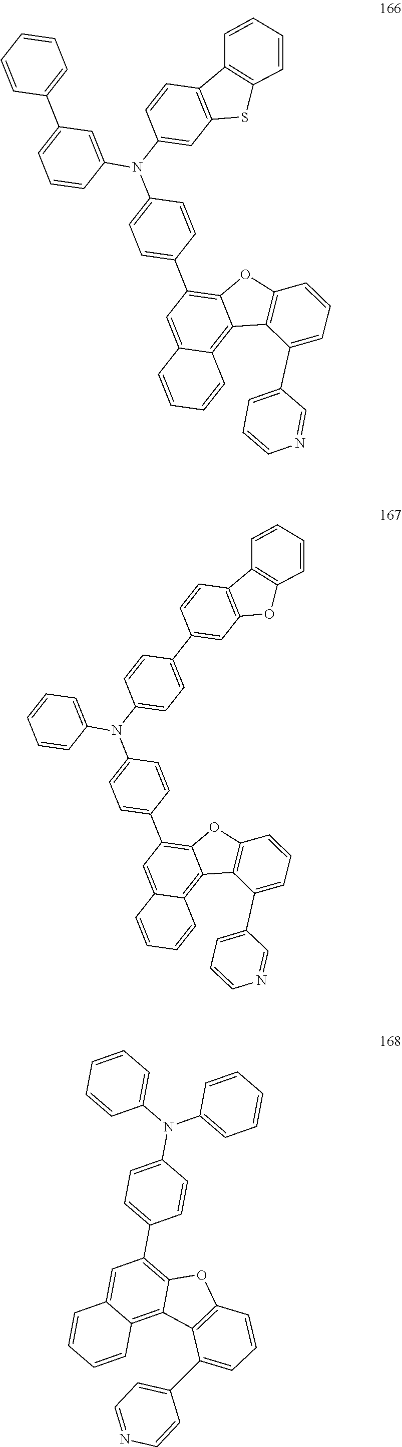 [SMILES: c1ccc(-c2cccc(N(c3ccc(-c4cc5ccccc5c5c4oc4cccc(-c6cccnc6)c45)cc3)c3ccc4c(c3)sc3ccccc34)c2)cc1 ; c1ccc(N(c2ccc(-c3ccc4c(c3)oc3ccccc34)cc2)c2ccc(-c3cc4ccccc4c4c3oc3cccc(-c5cccnc5)c34)cc2)cc1 ; c1ccc(N(c2ccccc2)c2ccc(-c3cc4ccccc4c4c3oc3cccc(-c5ccncc5)c34)cc2)cc1]

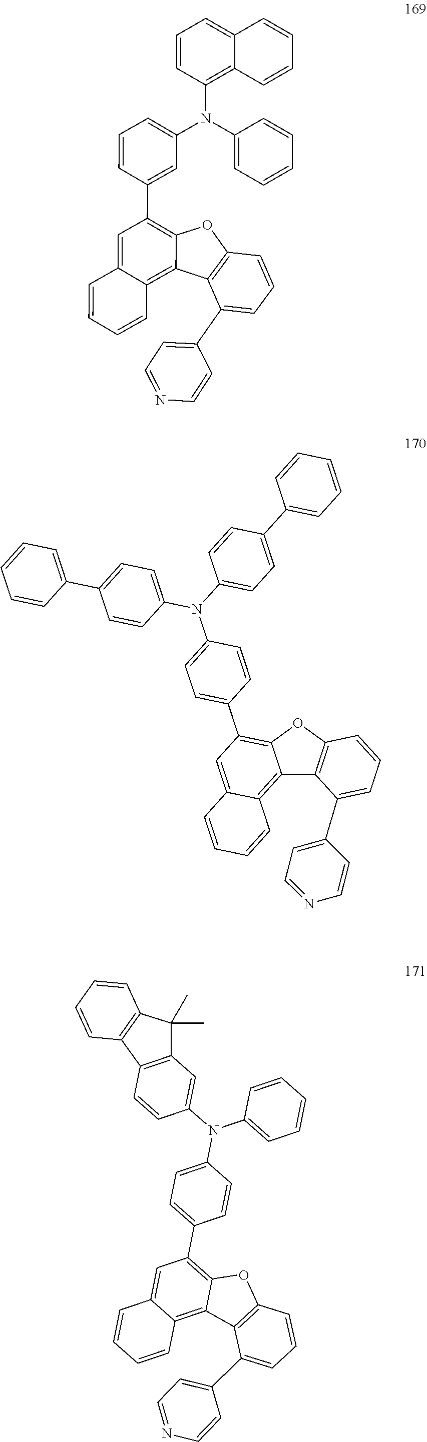 [SMILES: c1ccc(N(c2cccc(-c3cc4ccccc4c4c3oc3cccc(-c5ccncc5)c34)c2)c2cccc3ccccc23)cc1 ; c1ccc(-c2ccc(N(c3ccc(-c4ccccc4)cc3)c3ccc(-c4cc5ccccc5c5c4oc4cccc(-c6ccncc6)c45)cc3)cc2)cc1 ; CC1(C)c2ccccc2-c2ccc(N(c3ccccc3)c3ccc(-c4cc5ccccc5c5c4oc4cccc(-c6ccncc6)c45)cc3)cc21]

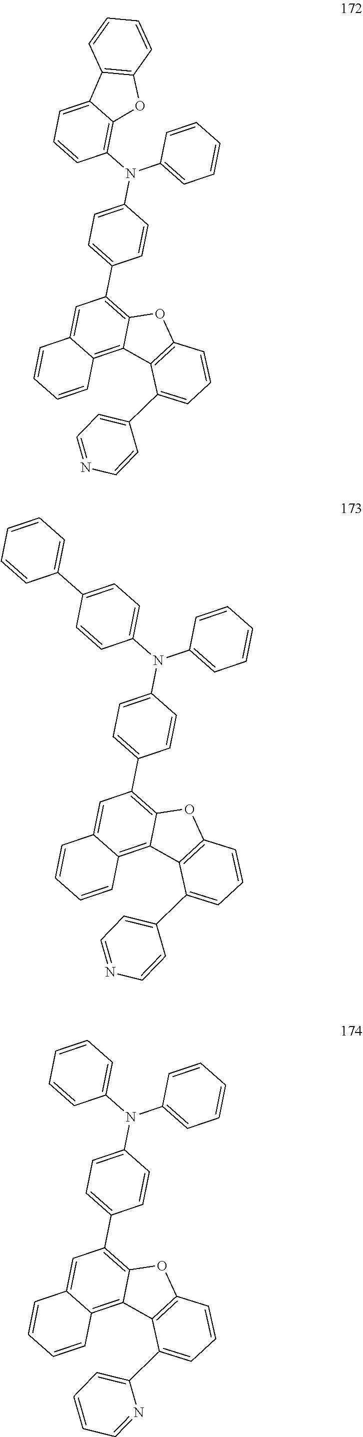 [SMILES: c1ccc(N(c2ccc(-c3cc4ccccc4c4c3oc3cccc(-c5ccncc5)c34)cc2)c2cccc3c2oc2ccccc23)cc1 ; c1ccc(-c2ccc(N(c3ccccc3)c3ccc(-c4cc5ccccc5c5c4oc4cccc(-c6ccncc6)c45)cc3)cc2)cc1 ; c1ccc(N(c2ccccc2)c2ccc(-c3cc4ccccc4c4c3oc3cccc(-c5ccccn5)c34)cc2)cc1]

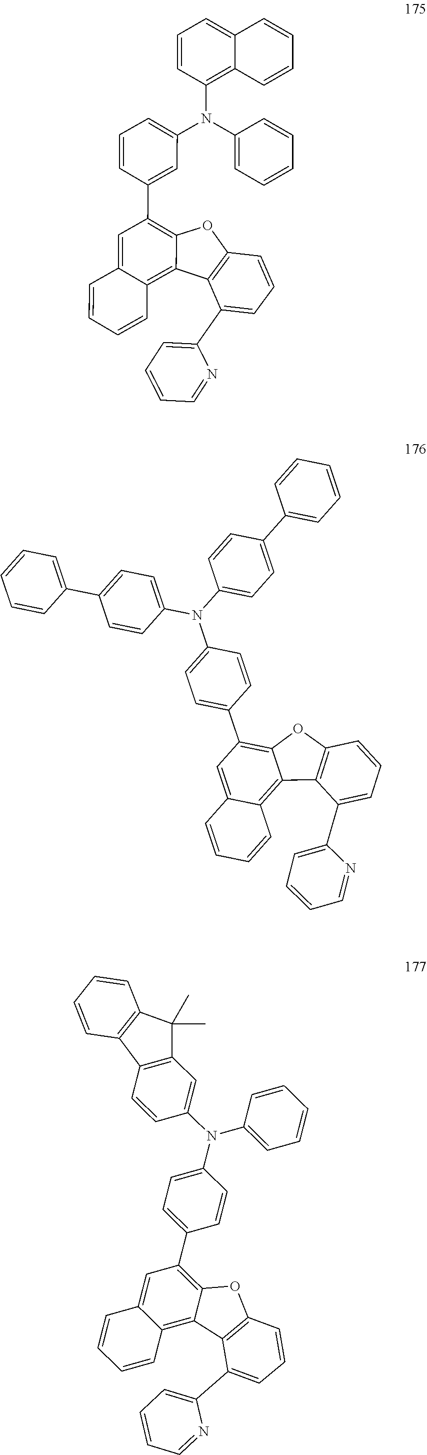 [SMILES: c1ccc(N(c2cccc(-c3cc4ccccc4c4c3oc3cccc(-c5ccccn5)c34)c2)c2cccc3ccccc23)cc1 ; c1ccc(-c2ccc(N(c3ccc(-c4ccccc4)cc3)c3ccc(-c4cc5ccccc5c5c4oc4cccc(-c6ccccn6)c45)cc3)cc2)cc1 ; CC1(C)c2ccccc2-c2ccc(N(c3ccccc3)c3ccc(-c4cc5ccccc5c5c4oc4cccc(-c6ccccn6)c45)cc3)cc21]

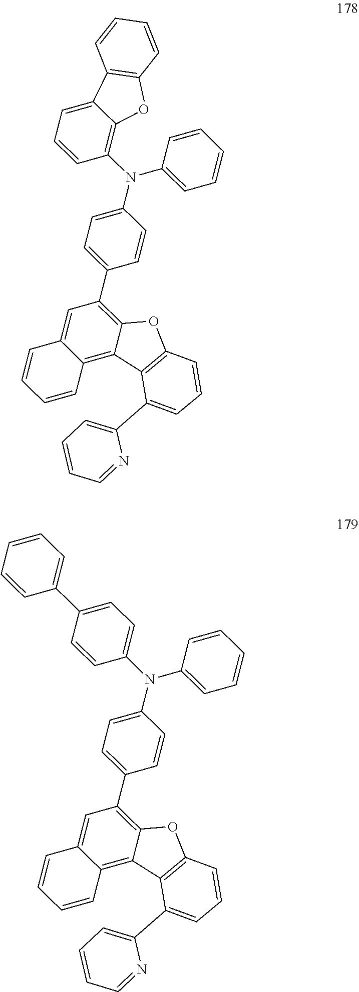 [SMILES: c1ccc(N(c2ccc(-c3cc4ccccc4c4c3oc3cccc(-c5ccccn5)c34)cc2)c2cccc3c2oc2ccccc23)cc1 ; c1ccc(-c2ccc(N(c3ccccc3)c3ccc(-c4cc5ccccc5c5c4oc4cccc(-c6ccccn6)c45)cc3)cc2)cc1]

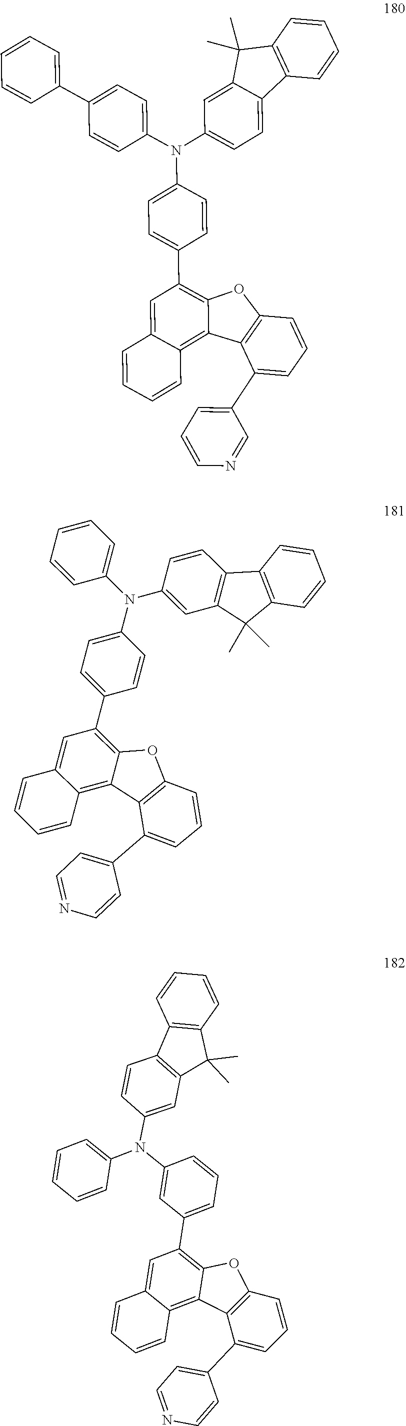 [SMILES: CC1(C)c2ccccc2-c2ccc(N(c3ccc(-c4ccccc4)cc3)c3ccc(-c4cc5ccccc5c5c4oc4cccc(-c6cccnc6)c45)cc3)cc21 ; CC1(C)c2ccccc2-c2ccc(N(c3ccccc3)c3ccc(-c4cc5ccccc5c5c4oc4cccc(-c6ccncc6)c45)cc3)cc21 ; CC1(C)c2ccccc2-c2ccc(N(c3ccccc3)c3cccc(-c4cc5ccccc5c5c4oc4cccc(-c6ccncc6)c45)c3)cc21]

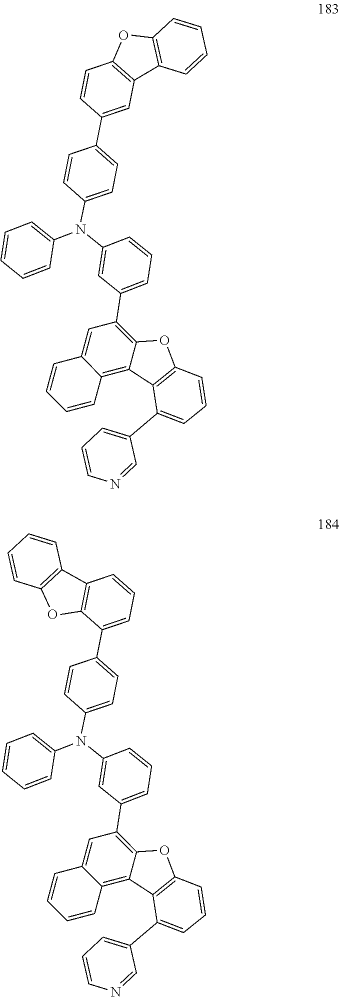 [SMILES: c1ccc(N(c2ccc(-c3ccc4oc5ccccc5c4c3)cc2)c2cccc(-c3cc4ccccc4c4c3oc3cccc(-c5cccnc5)c34)c2)cc1 ; c1ccc(N(c2ccc(-c3cccc4c3oc3ccccc34)cc2)c2cccc(-c3cc4ccccc4c4c3oc3cccc(-c5cccnc5)c34)c2)cc1]

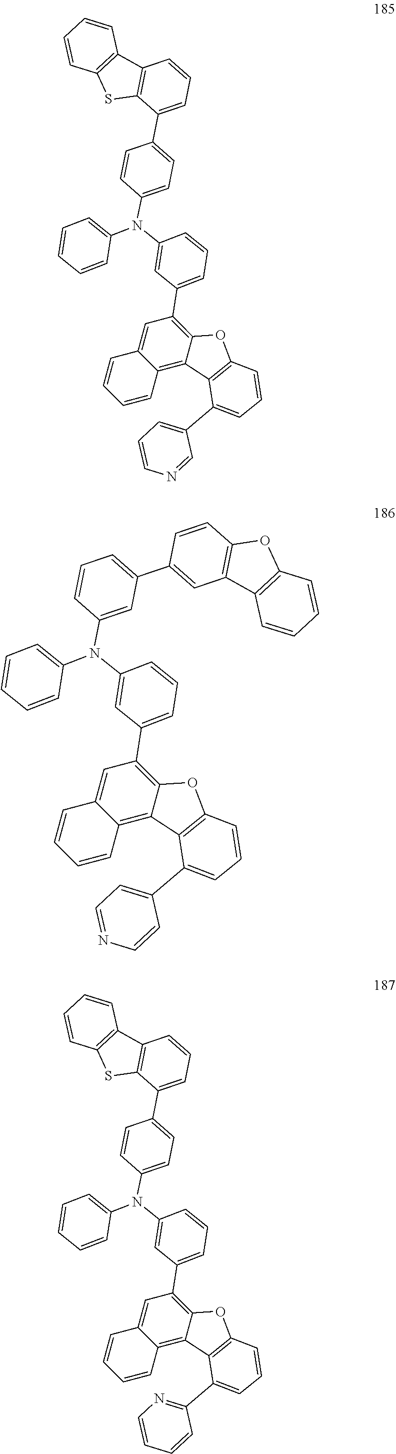 [SMILES: c1ccc(N(c2ccc(-c3cccc4c3sc3ccccc34)cc2)c2cccc(-c3cc4ccccc4c4c3oc3cccc(-c5cccnc5)c34)c2)cc1 ; c1ccc(N(c2cccc(-c3ccc4oc5ccccc5c4c3)c2)c2cccc(-c3cc4ccccc4c4c3oc3cccc(-c5ccncc5)c34)c2)cc1 ; c1ccc(N(c2ccc(-c3cccc4c3sc3ccccc34)cc2)c2cccc(-c3cc4ccccc4c4c3oc3cccc(-c5ccccn5)c34)c2)cc1]

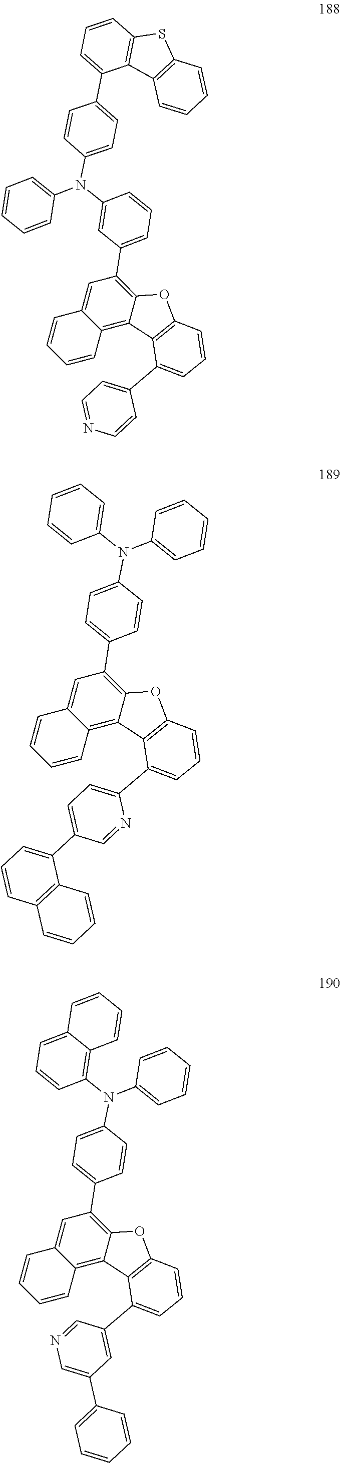 [SMILES: c1ccc(N(c2ccc(-c3cccc4sc5ccccc5c34)cc2)c2cccc(-c3cc4ccccc4c4c3oc3cccc(-c5ccncc5)c34)c2)cc1 ; c1ccc(N(c2ccccc2)c2ccc(-c3cc4ccccc4c4c3oc3cccc(-c5ccc(-c6cccc7ccccc67)cn5)c34)cc2)cc1 ; c1ccc(-c2cncc(-c3cccc4oc5c(-c6ccc(N(c7ccccc7)c7cccc8ccccc78)cc6)cc6ccccc6c5c34)c2)cc1]

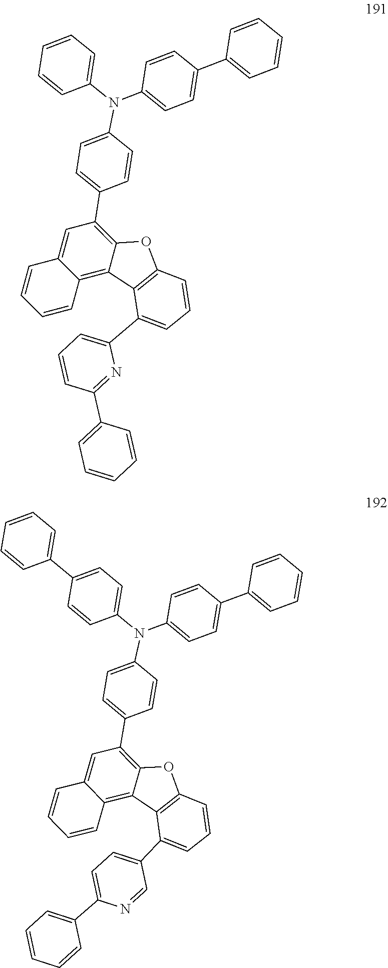 [SMILES: c1ccc(-c2ccc(N(c3ccccc3)c3ccc(-c4cc5ccccc5c5c4oc4cccc(-c6cccc(-c7ccccc7)n6)c45)cc3)cc2)cc1 ; c1ccc(-c2ccc(N(c3ccc(-c4ccccc4)cc3)c3ccc(-c4cc5ccccc5c5c4oc4cccc(-c6ccc(-c7ccccc7)nc6)c45)cc3)cc2)cc1]

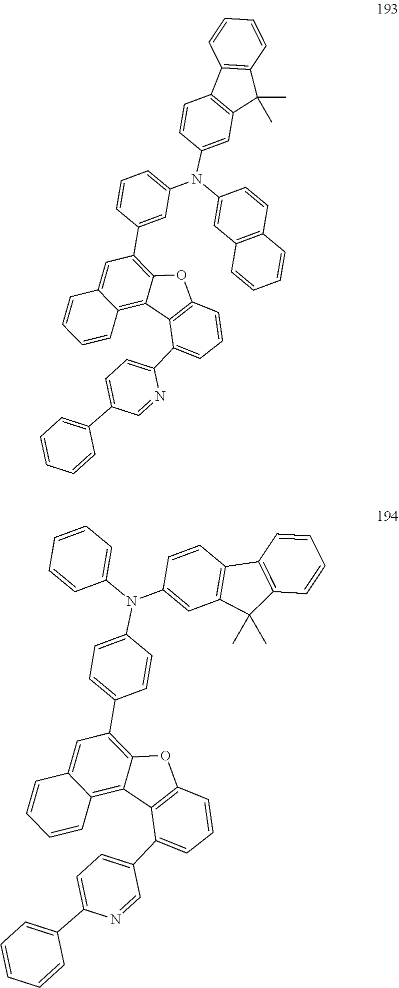 [SMILES: CC1(C)c2ccccc2-c2ccc(N(c3cccc(-c4cc5ccccc5c5c4oc4cccc(-c6ccc(-c7ccccc7)cn6)c45)c3)c3ccc4ccccc4c3)cc21 ; CC1(C)c2ccccc2-c2ccc(N(c3ccccc3)c3ccc(-c4cc5ccccc5c5c4oc4cccc(-c6ccc(-c7ccccc7)nc6)c45)cc3)cc21]

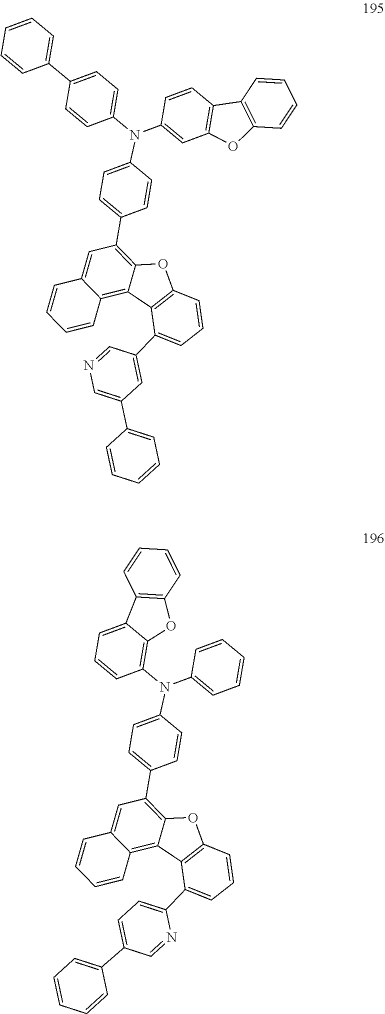 [SMILES: c1ccc(-c2ccc(N(c3ccc(-c4cc5ccccc5c5c4oc4cccc(-c6cncc(-c7ccccc7)c6)c45)cc3)c3ccc4c(c3)oc3ccccc34)cc2)cc1 ; c1ccc(-c2ccc(-c3cccc4oc5c(-c6ccc(N(c7ccccc7)c7cccc8c7oc7ccccc78)cc6)cc6ccccc6c5c34)nc2)cc1]

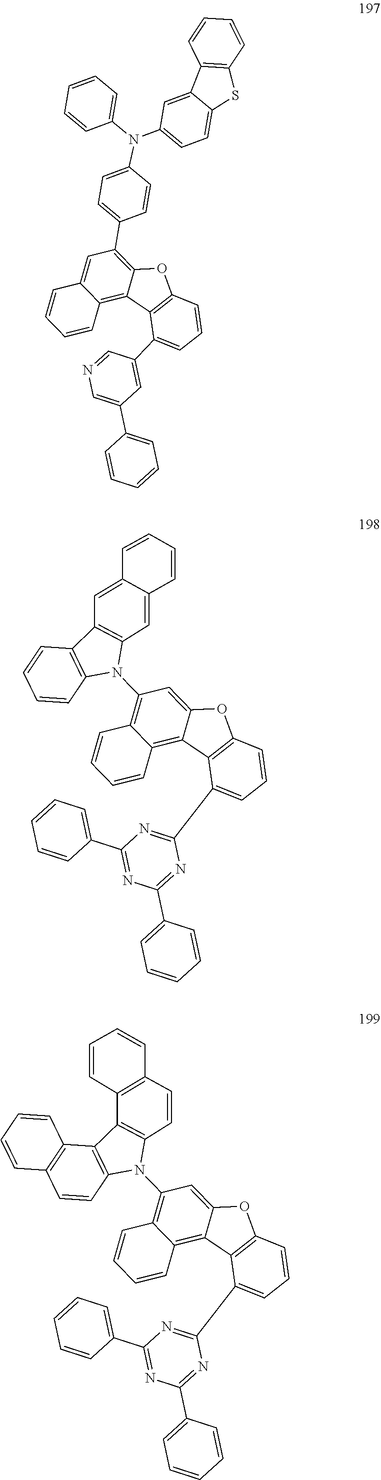 [SMILES: c1ccc(-c2cncc(-c3cccc4oc5c(-c6ccc(N(c7ccccc7)c7ccc8sc9ccccc9c8c7)cc6)cc6ccccc6c5c34)c2)cc1 ; c1ccc(-c2nc(-c3ccccc3)nc(-c3cccc4oc5cc(-n6c7ccccc7c7cc8ccccc8cc76)c6ccccc6c5c34)n2)cc1 ; c1ccc(-c2nc(-c3ccccc3)nc(-c3cccc4oc5cc(-n6c7ccc8ccccc8c7c7c8ccccc8ccc76)c6ccccc6c5c34)n2)cc1]

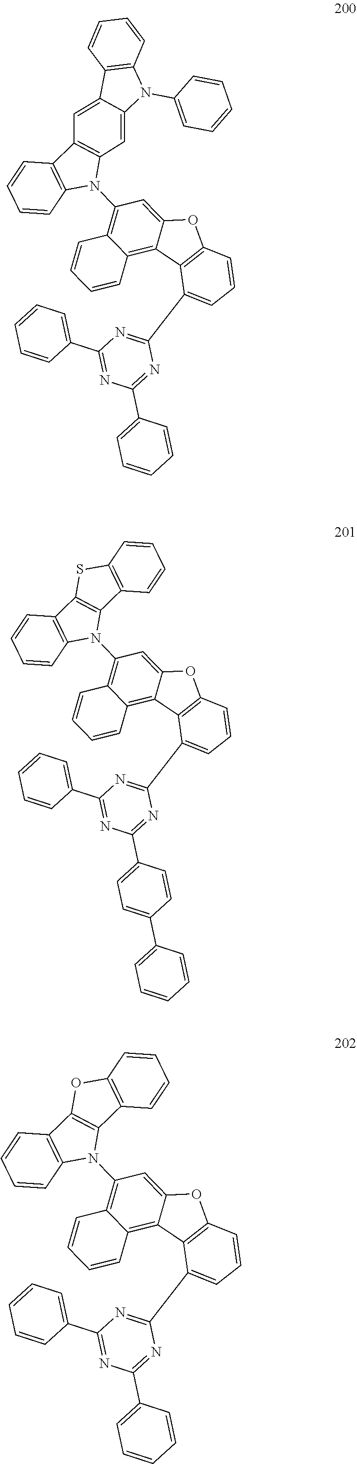 [SMILES: c1ccc(-c2nc(-c3ccccc3)nc(-c3cccc4oc5cc(-n6c7ccccc7c7cc8c9ccccc9n(-c9ccccc9)c8cc76)c6ccccc6c5c34)n2)cc1 ; c1ccc(-c2ccc(-c3nc(-c4ccccc4)nc(-c4cccc5oc6cc(-n7c8ccccc8c8sc9ccccc9c87)c7ccccc7c6c45)n3)cc2)cc1 ; c1ccc(-c2nc(-c3ccccc3)nc(-c3cccc4oc5cc(-n6c7ccccc7c7oc8ccccc8c76)c6ccccc6c5c34)n2)cc1]

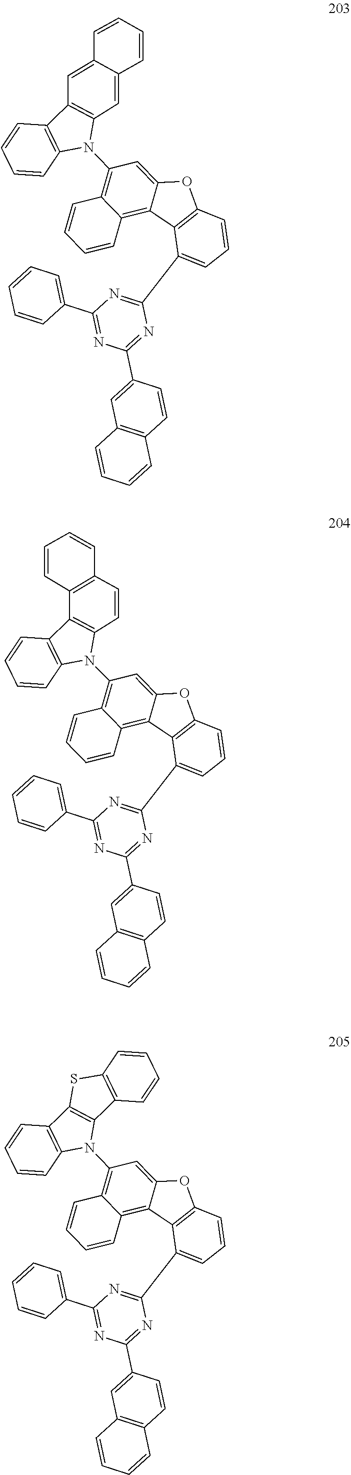 [SMILES: c1ccc(-c2nc(-c3ccc4ccccc4c3)nc(-c3cccc4oc5cc(-n6c7ccccc7c7cc8ccccc8cc76)c6ccccc6c5c34)n2)cc1 ; c1ccc(-c2nc(-c3ccc4ccccc4c3)nc(-c3cccc4oc5cc(-n6c7ccccc7c7c8ccccc8ccc76)c6ccccc6c5c34)n2)cc1 ; c1ccc(-c2nc(-c3ccc4ccccc4c3)nc(-c3cccc4oc5cc(-n6c7ccccc7c7sc8ccccc8c76)c6ccccc6c5c34)n2)cc1]

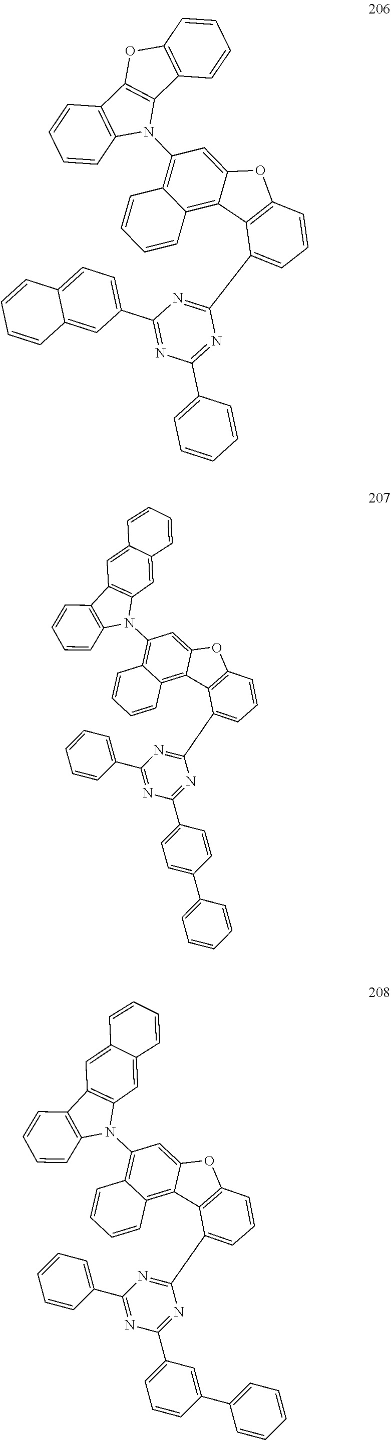 [SMILES: c1ccc(-c2nc(-c3ccc4ccccc4c3)nc(-c3cccc4oc5cc(-n6c7ccccc7c7oc8ccccc8c76)c6ccccc6c5c34)n2)cc1 ; c1ccc(-c2ccc(-c3nc(-c4ccccc4)nc(-c4cccc5oc6cc(-n7c8ccccc8c8cc9ccccc9cc87)c7ccccc7c6c45)n3)cc2)cc1 ; c1ccc(-c2cccc(-c3nc(-c4ccccc4)nc(-c4cccc5oc6cc(-n7c8ccccc8c8cc9ccccc9cc87)c7ccccc7c6c45)n3)c2)cc1]

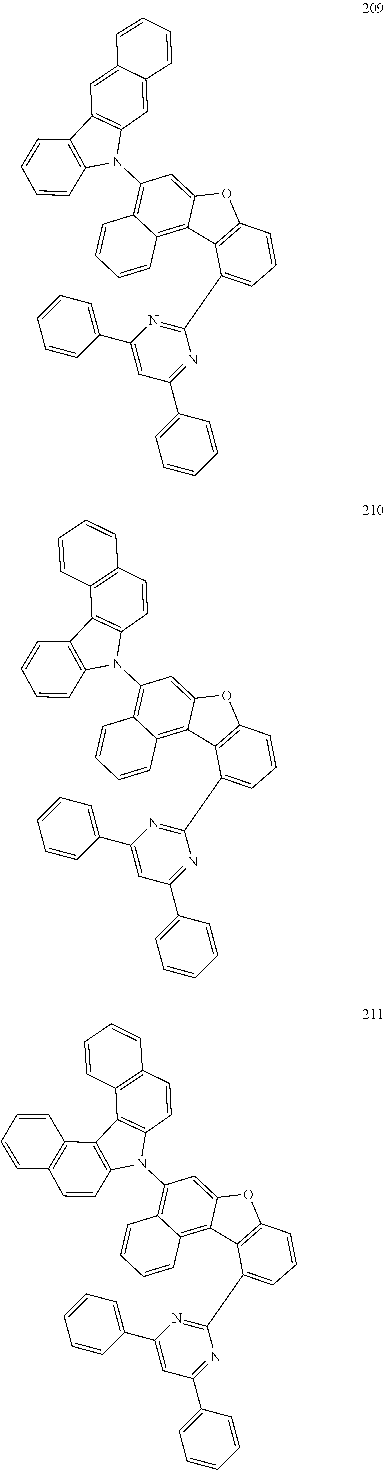 [SMILES: c1ccc(-c2cc(-c3ccccc3)nc(-c3cccc4oc5cc(-n6c7ccccc7c7cc8ccccc8cc76)c6ccccc6c5c34)n2)cc1 ; c1ccc(-c2cc(-c3ccccc3)nc(-c3cccc4oc5cc(-n6c7ccccc7c7c8ccccc8ccc76)c6ccccc6c5c34)n2)cc1 ; c1ccc(-c2cc(-c3ccccc3)nc(-c3cccc4oc5cc(-n6c7ccc8ccccc8c7c7c8ccccc8ccc76)c6ccccc6c5c34)n2)cc1]

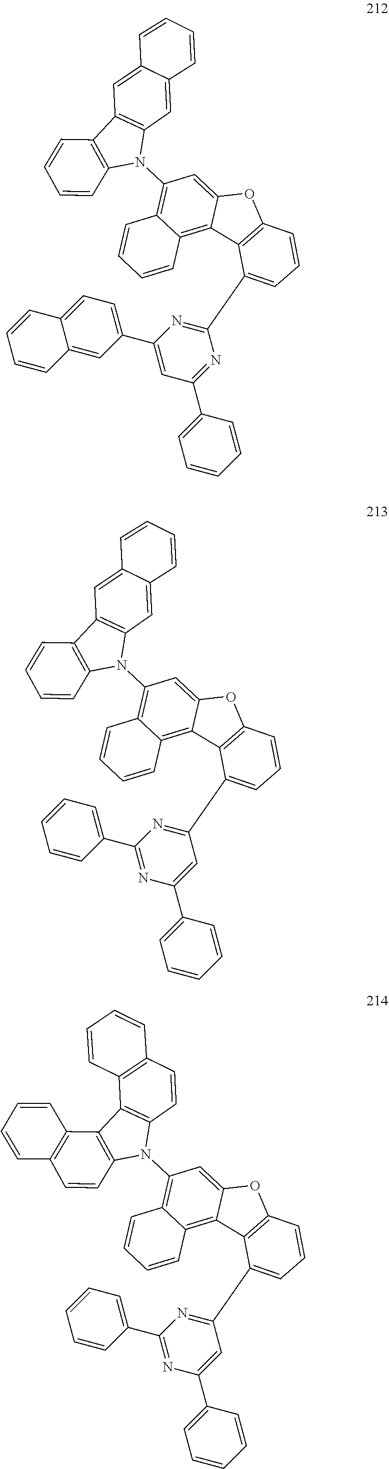 [SMILES: c1ccc(-c2cc(-c3ccc4ccccc4c3)nc(-c3cccc4oc5cc(-n6c7ccccc7c7cc8ccccc8cc76)c6ccccc6c5c34)n2)cc1 ; c1ccc(-c2cc(-c3cccc4oc5cc(-n6c7ccccc7c7cc8ccccc8cc76)c6ccccc6c5c34)nc(-c3ccccc3)n2)cc1 ; c1ccc(-c2cc(-c3cccc4oc5cc(-n6c7ccc8ccccc8c7c7c8ccccc8ccc76)c6ccccc6c5c34)nc(-c3ccccc3)n2)cc1]

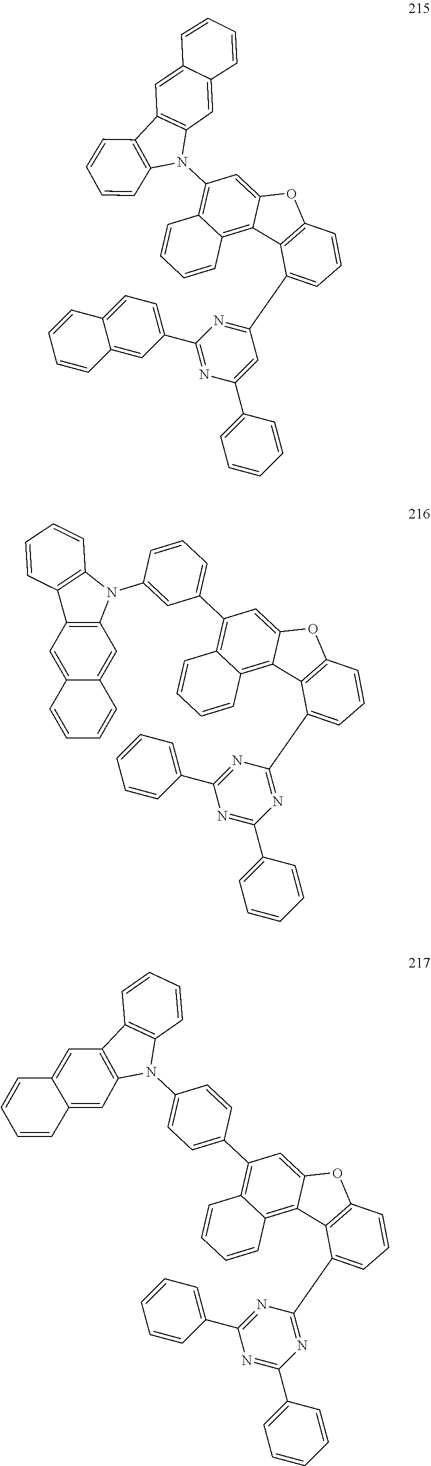 [SMILES: c1ccc(-c2cc(-c3cccc4oc5cc(-n6c7ccccc7c7cc8ccccc8cc76)c6ccccc6c5c34)nc(-c3ccc4ccccc4c3)n2)cc1 ; c1ccc(-c2nc(-c3ccccc3)nc(-c3cccc4oc5cc(-c6cccc(-n7c8ccccc8c8cc9ccccc9cc87)c6)c6ccccc6c5c34)n2)cc1 ; c1ccc(-c2nc(-c3ccccc3)nc(-c3cccc4oc5cc(-c6ccc(-n7c8ccccc8c8cc9ccccc9cc87)cc6)c6ccccc6c5c34)n2)cc1]

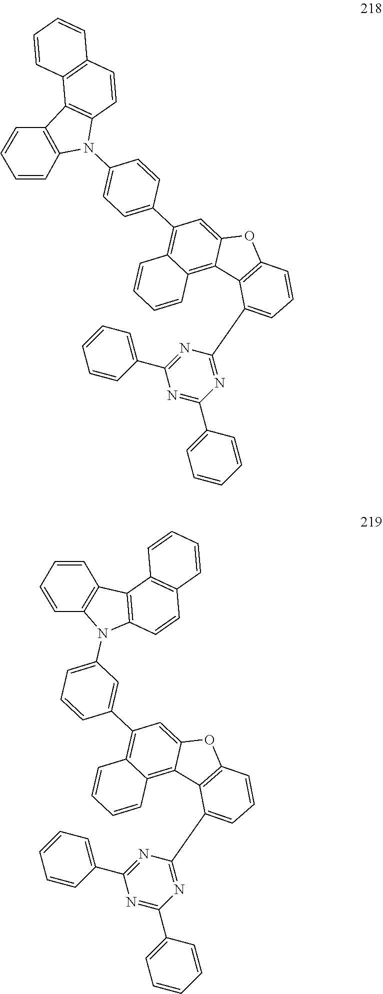 [SMILES: c1ccc(-c2nc(-c3ccccc3)nc(-c3cccc4oc5cc(-c6ccc(-n7c8ccccc8c8c9ccccc9ccc87)cc6)c6ccccc6c5c34)n2)cc1 ; c1ccc(-c2nc(-c3ccccc3)nc(-c3cccc4oc5cc(-c6cccc(-n7c8ccccc8c8c9ccccc9ccc87)c6)c6ccccc6c5c34)n2)cc1]

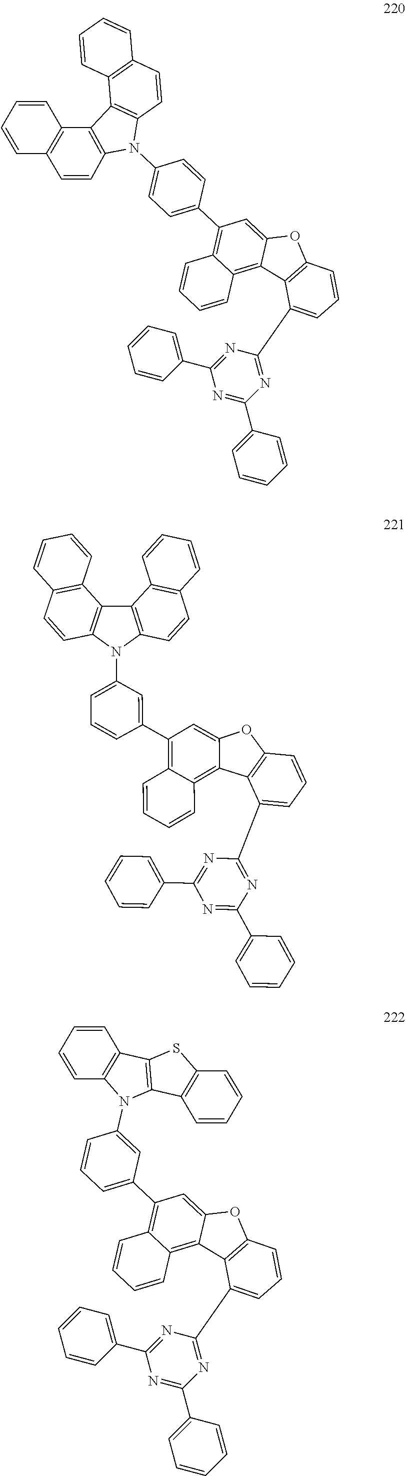 [SMILES: c1ccc(-c2nc(-c3ccccc3)nc(-c3cccc4oc5cc(-c6ccc(-n7c8ccc9ccccc9c8c8c9ccccc9ccc87)cc6)c6ccccc6c5c34)n2)cc1 ; c1ccc(-c2nc(-c3ccccc3)nc(-c3cccc4oc5cc(-c6cccc(-n7c8ccc9ccccc9c8c8c9ccccc9ccc87)c6)c6ccccc6c5c34)n2)cc1 ; c1ccc(-c2nc(-c3ccccc3)nc(-c3cccc4oc5cc(-c6cccc(-n7c8ccccc8c8sc9ccccc9c87)c6)c6ccccc6c5c34)n2)cc1]

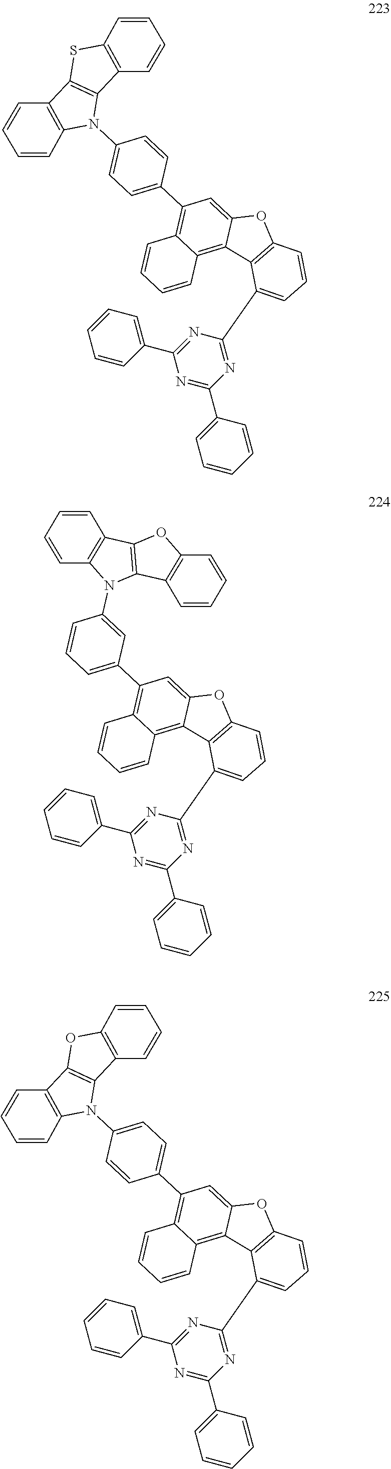 [SMILES: c1ccc(-c2nc(-c3ccccc3)nc(-c3cccc4oc5cc(-c6ccc(-n7c8ccccc8c8sc9ccccc9c87)cc6)c6ccccc6c5c34)n2)cc1 ; c1ccc(-c2nc(-c3ccccc3)nc(-c3cccc4oc5cc(-c6cccc(-n7c8ccccc8c8oc9ccccc9c87)c6)c6ccccc6c5c34)n2)cc1 ; c1ccc(-c2nc(-c3ccccc3)nc(-c3cccc4oc5cc(-c6ccc(-n7c8ccccc8c8oc9ccccc9c87)cc6)c6ccccc6c5c34)n2)cc1]

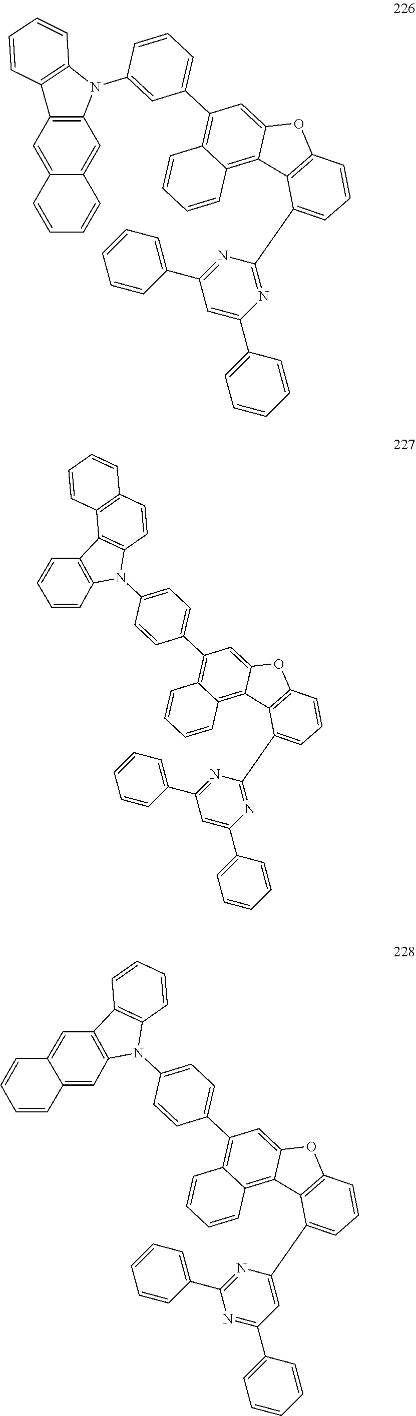 [SMILES: c1ccc(-c2cc(-c3ccccc3)nc(-c3cccc4oc5cc(-c6cccc(-n7c8ccccc8c8cc9ccccc9cc87)c6)c6ccccc6c5c34)n2)cc1 ; c1ccc(-c2cc(-c3ccccc3)nc(-c3cccc4oc5cc(-c6ccc(-n7c8ccccc8c8c9ccccc9ccc87)cc6)c6ccccc6c5c34)n2)cc1 ; c1ccc(-c2cc(-c3cccc4oc5cc(-c6ccc(-n7c8ccccc8c8cc9ccccc9cc87)cc6)c6ccccc6c5c34)nc(-c3ccccc3)n2)cc1]

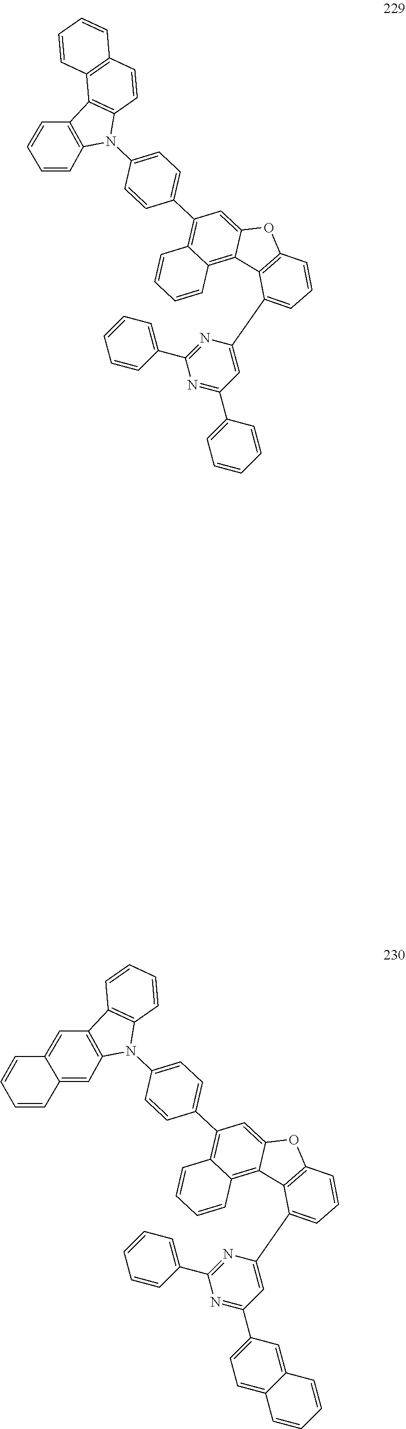 [SMILES: c1ccc(-c2cc(-c3cccc4oc5cc(-c6ccc(-n7c8ccccc8c8c9ccccc9ccc87)cc6)c6ccccc6c5c34)nc(-c3ccccc3)n2)cc1 ; c1ccc(-c2nc(-c3ccc4ccccc4c3)cc(-c3cccc4oc5cc(-c6ccc(-n7c8ccccc8c8cc9ccccc9cc87)cc6)c6ccccc6c5c34)n2)cc1]

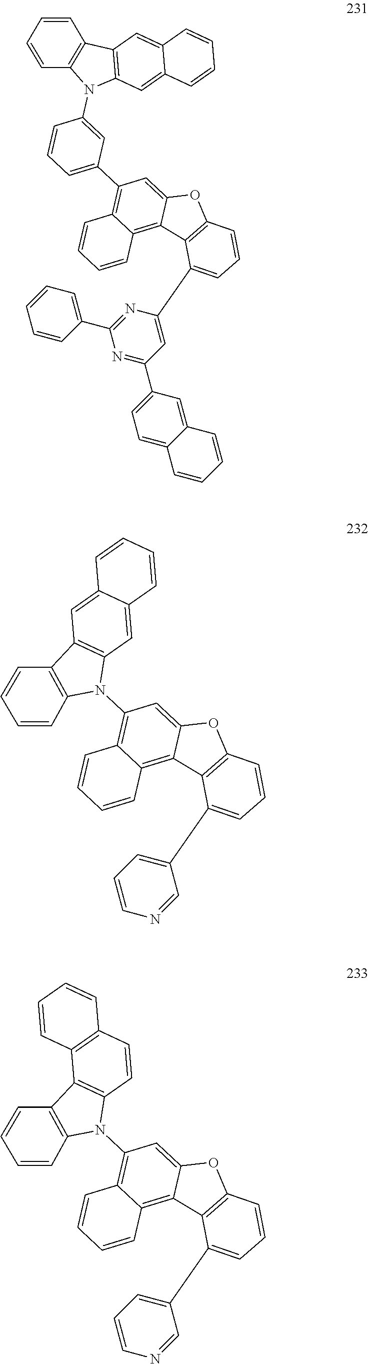 [SMILES: c1ccc(-c2nc(-c3ccc4ccccc4c3)cc(-c3cccc4oc5cc(-c6cccc(-n7c8ccccc8c8cc9ccccc9cc87)c6)c6ccccc6c5c34)n2)cc1 ; c1cncc(-c2cccc3oc4cc(-n5c6ccccc6c6cc7ccccc7cc65)c5ccccc5c4c23)c1 ; c1cncc(-c2cccc3oc4cc(-n5c6ccccc6c6c7ccccc7ccc65)c5ccccc5c4c23)c1]

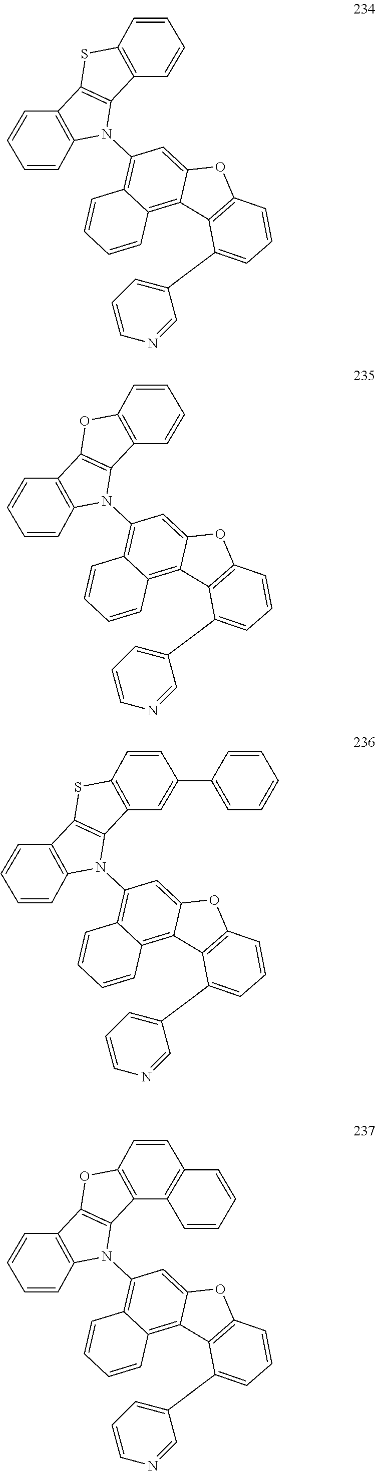

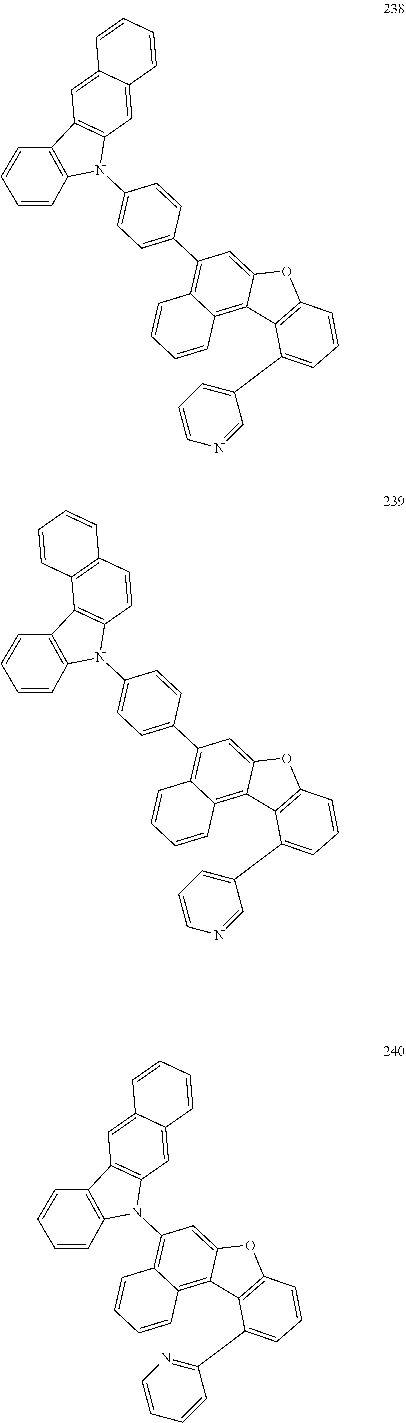 [SMILES: c1cncc(-c2cccc3oc4cc(-c5ccc(-n6c7ccccc7c7cc8ccccc8cc76)cc5)c5ccccc5c4c23)c1 ; c1cncc(-c2cccc3oc4cc(-c5ccc(-n6c7ccccc7c7c8ccccc8ccc76)cc5)c5ccccc5c4c23)c1 ; c1ccc(-c2cccc3oc4cc(-n5c6ccccc6c6cc7ccccc7cc65)c5ccccc5c4c23)nc1]

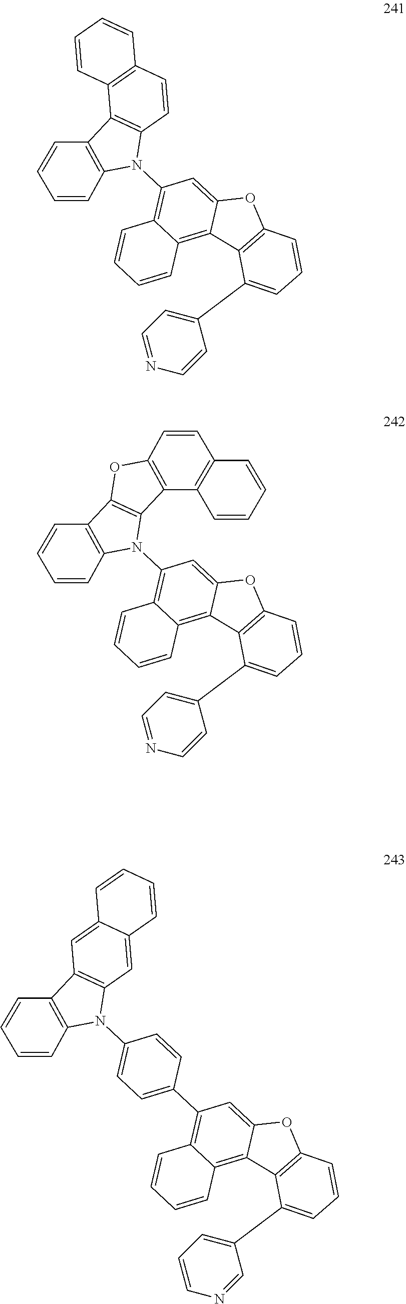 [SMILES: c1ccc2c(c1)ccc1c2c2ccccc2n1-c1cc2oc3cccc(-c4ccncc4)c3c2c2ccccc12 ; c1ccc2c(c1)ccc1oc3c4ccccc4n(-c4cc5oc6cccc(-c7ccncc7)c6c5c5ccccc45)c3c12 ; c1cncc(-c2cccc3oc4cc(-c5ccc(-n6c7ccccc7c7cc8ccccc8cc76)cc5)c5ccccc5c4c23)c1]

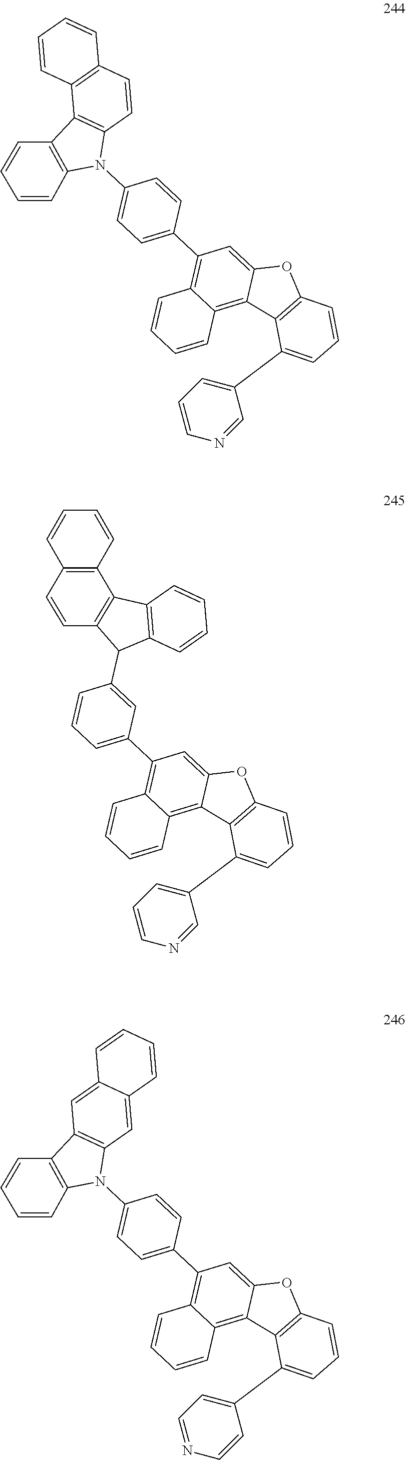 [SMILES: c1cncc(-c2cccc3oc4cc(-c5ccc(-n6c7ccccc7c7c8ccccc8ccc76)cc5)c5ccccc5c4c23)c1 ; c1cncc(-c2cccc3oc4cc(-c5cccc(C6c7ccccc7-c7c6ccc6ccccc76)c5)c5ccccc5c4c23)c1 ; c1ccc2cc3c(cc2c1)c1ccccc1n3-c1ccc(-c2cc3oc4cccc(-c5ccncc5)c4c3c3ccccc23)cc1]

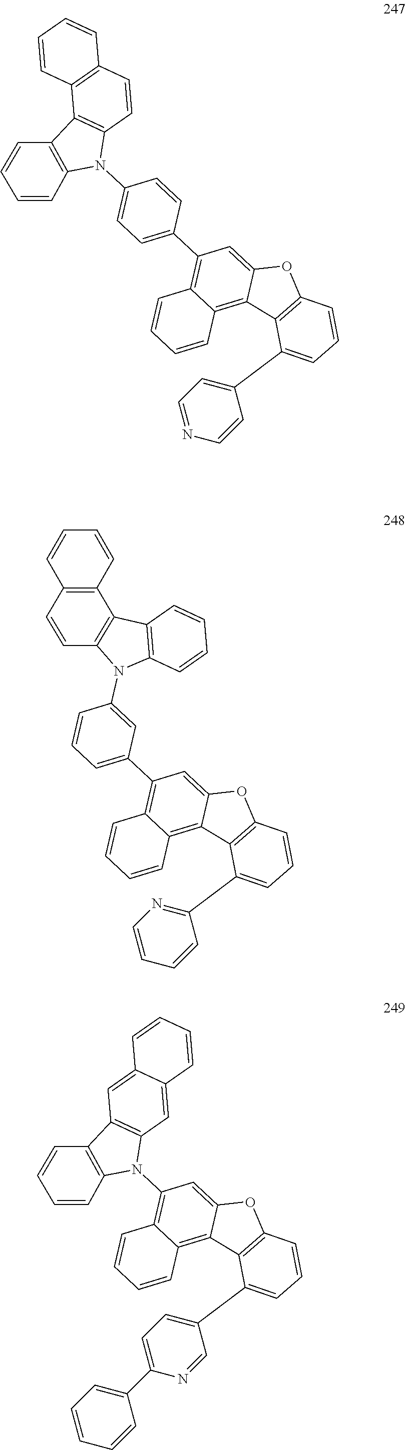 [SMILES: c1ccc2c(c1)ccc1c2c2ccccc2n1-c1ccc(-c2cc3oc4cccc(-c5ccncc5)c4c3c3ccccc23)cc1 ; c1ccc(-c2cccc3oc4cc(-c5cccc(-n6c7ccccc7c7c8ccccc8ccc76)c5)c5ccccc5c4c23)nc1 ; c1ccc(-c2ccc(-c3cccc4oc5cc(-n6c7ccccc7c7cc8ccccc8cc76)c6ccccc6c5c34)cn2)cc1]

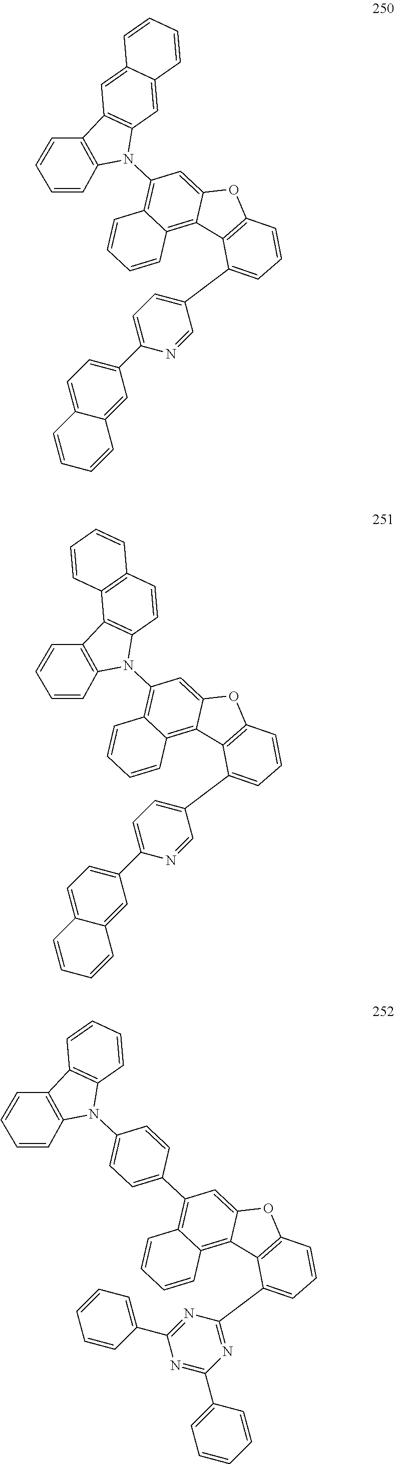

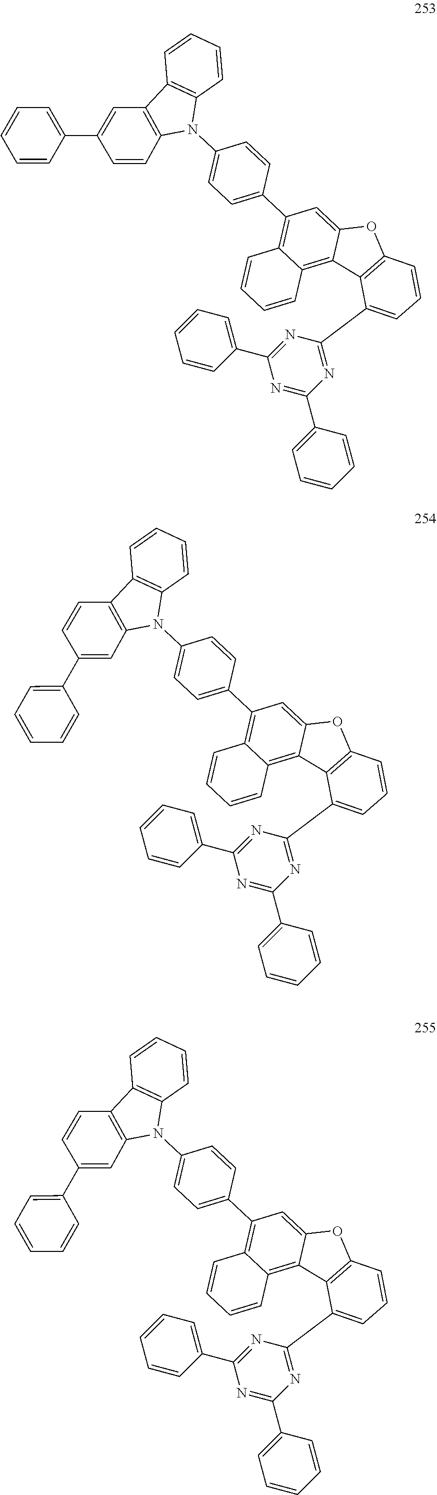 [SMILES: c1ccc(-c2ccc3c(c2)c2ccccc2n3-c2ccc(-c3cc4oc5cccc(-c6nc(-c7ccccc7)nc(-c7ccccc7)n6)c5c4c4ccccc34)cc2)cc1 ; c1ccc(-c2ccc3c4ccccc4n(-c4ccc(-c5cc6oc7cccc(-c8nc(-c9ccccc9)nc(-c9ccccc9)n8)c7c6c6ccccc56)cc4)c3c2)cc1 ; c1ccc(-c2ccc3c4ccccc4n(-c4ccc(-c5cc6oc7cccc(-c8nc(-c9ccccc9)nc(-c9ccccc9)n8)c7c6c6ccccc56)cc4)c3c2)cc1]

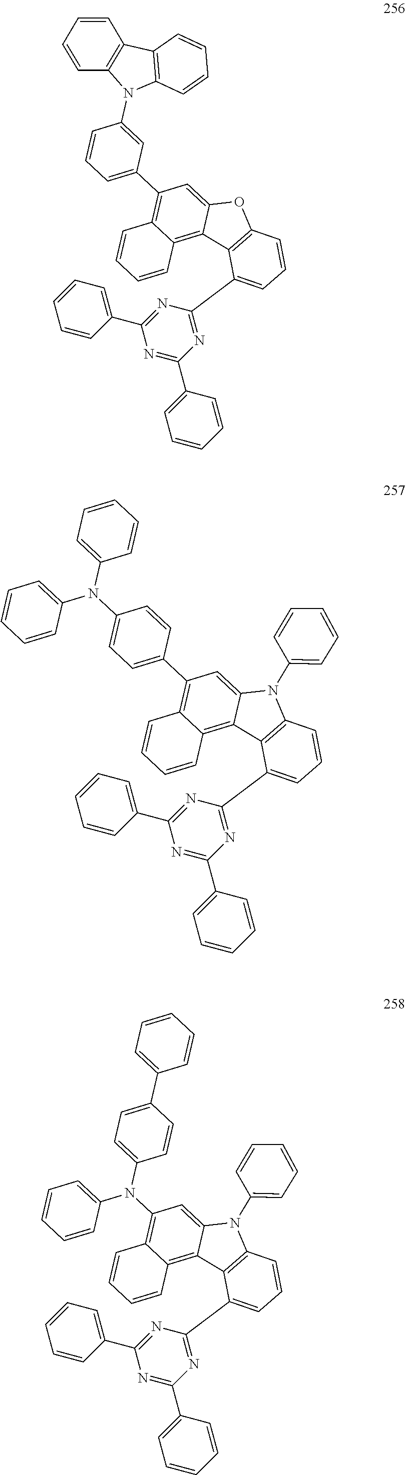 [SMILES: c1ccc(-c2nc(-c3ccccc3)nc(-c3cccc4oc5cc(-c6cccc(-n7c8ccccc8c8ccccc87)c6)c6ccccc6c5c34)n2)cc1 ; c1ccc(-c2nc(-c3ccccc3)nc(-c3cccc4c3c3c5ccccc5c(-c5ccc(N(c6ccccc6)c6ccccc6)cc5)cc3n4-c3ccccc3)n2)cc1 ; c1ccc(-c2ccc(N(c3ccccc3)c3cc4c(c5ccccc35)c3c(-c5nc(-c6ccccc6)nc(-c6ccccc6)n5)cccc3n4-c3ccccc3)cc2)cc1]

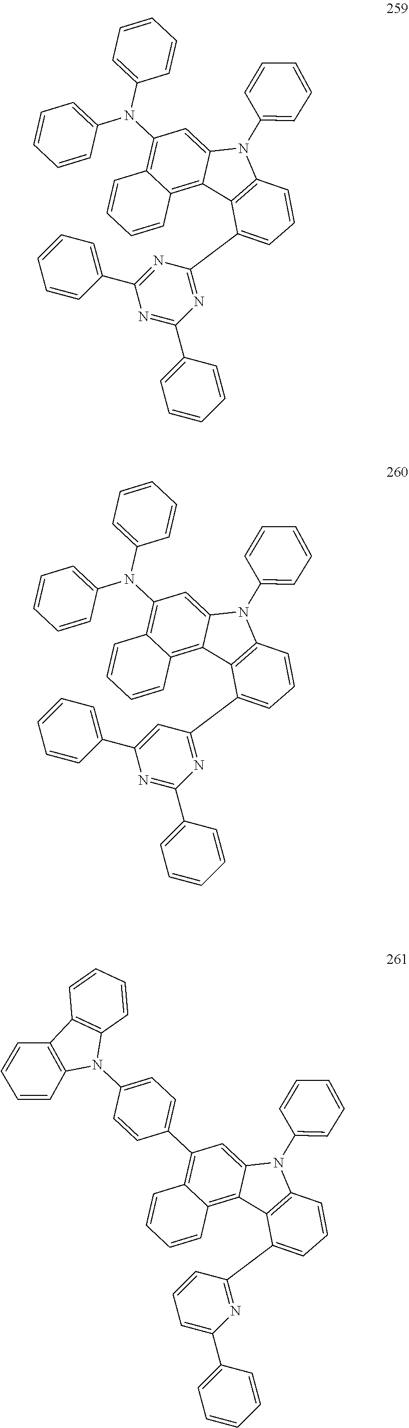 [SMILES: c1ccc(-c2nc(-c3ccccc3)nc(-c3cccc4c3c3c5ccccc5c(N(c5ccccc5)c5ccccc5)cc3n4-c3ccccc3)n2)cc1 ; c1ccc(-c2cc(-c3cccc4c3c3c5ccccc5c(N(c5ccccc5)c5ccccc5)cc3n4-c3ccccc3)nc(-c3ccccc3)n2)cc1 ; c1ccc(-c2cccc(-c3cccc4c3c3c5ccccc5c(-c5ccc(-n6c7ccccc7c7ccccc76)cc5)cc3n4-c3ccccc3)n2)cc1]

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

Meanwhile, the heterocyclic compound has a high glass transition temperature (Tg) and thereby has superior thermal stability. Such an increase in the thermal stability becomes an important factor in providing driving stability to a device.

The heterocyclic compound according to one embodiment of the present application may be prepared using a multi-step chemical reaction. Some intermediate compounds are prepared first, and from the intermediate compounds, the compound of Chemical Formula 1 may be prepared. More specifically, the heterocyclic compound according to one embodiment of the present application may be prepared based on preparation examples to describe later.

Another embodiment of the present application provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

One embodiment of the present application provides an organic light emitting device including a first electrode; a second electrode; and one or more organic material layers provided between the first electrode and the second electrode, wherein one or more layers of the organic material layers include the heterocyclic compound represented by Chemical Formula 1.

In one embodiment of the present application, the first electrode may be an anode, and the second electrode may be a cathode.

In another embodiment of the present application, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device.

In another embodiment of the present application, the organic light emitting device may be a green organic light emitting device, and the heterocyclic compound according to Chemical Formula 1 may be used as a material of the green organic light emitting device.

In another embodiment of the present application, the organic light emitting device may be a red organic light emitting device, and the heterocyclic compound according to Chemical Formula 1 may be used as a material of the red organic light emitting device.

Specific descriptions on the heterocyclic compound represented by Chemical Formula 1 are the same as the descriptions provided above.

The organic light emitting device of the present application may be manufactured using common organic light emitting device manufacturing methods and materials except that one or more of the organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present application may be formed in a single layer structure, but may be formed in a multilayer structure in which two or more organic material layers are laminated. For example, the organic light emitting device of the present disclosure may have a structure including a hole injection layer, a hole transfer layer, a hole auxiliary layer, a light emitting layer, an electron transfer layer, an electron injection layer and the like as the organic material layer. However, the structure of the organic light emitting device is not limited thereto, and may include a smaller number of organic material layers.

In the organic light emitting device of the present application, the organic material layer includes a light emitting layer, and the light emitting layer may include the heterocyclic compound. Using the heterocyclic compound in the light emitting layer spatially separates HOMO (Highest Occupied Molecular Orbital) and LUMO (Lowest Unoccupied Molecular Orbital) allowing strong charge transfer, and accordingly, superior driving, efficiency and lifetime may be obtained in the organic light emitting device.

The organic light emitting device of the present disclosure may further include one, two or more layers selected from the group consisting of a light emitting layer, a hole injection layer, a hole transfer layer, an electron injection layer, an electron transfer layer, an electron blocking layer, a hole auxiliary layer and a hole blocking layer.

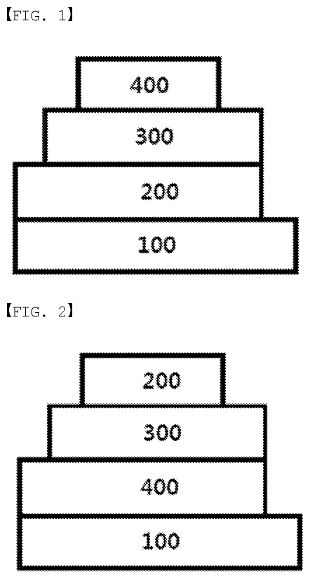

FIG. 1 to FIG. 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

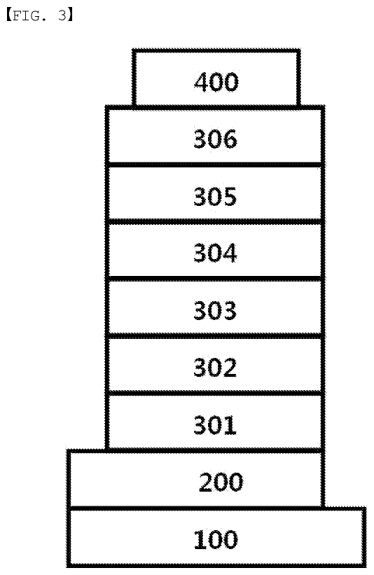

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 includes a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, layers other than the light emitting layer may not be included, and other necessary functional layers may be further added.

The organic material layer including the compound represented by Chemical Formula 1 may further include other materials as necessary.

In the organic light emitting device according to one embodiment of the present application, materials other than the compound of Chemical Formula 1 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene](PEDOT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MT DATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly (3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involving in light emission together may also be used.

When mixing light emitting material hosts, same series hosts may be mixed, or different series hosts may be mixed. For example, any two or more types of materials among n-type host materials or p-type host materials may be selected and used as a host material of a light emitting layer.

In the organic light emitting device of the present application, the organic material layer includes a light emitting layer, and the light emitting layer may include the heterocyclic compound as a host material of a light emitting material.

In the organic light emitting device of the present application, the light emitting layer may include two or more host materials, and at least one of the host materials may include the heterocyclic compound as a host material of a light emitting material.

In the organic light emitting device of the present application, the light emitting layer may use two or more host materials after pre-mixing, and at least one of the two or more host materials may include the heterocyclic compound as a host material of a light emitting material.

The pre-mixing means mixing the two or more host materials of the light emitting layer in advance in one source of supply before depositing on the organic material layer.

In the organic light emitting device of the present application, the light emitting layer may include two or more host materials, the two or more host materials each include one or more p-type host materials and n-type host materials, and at least one of the host materials may include the heterocyclic compound as a host material of a light emitting material. In this case, the organic light emitting device may have superior driving, efficiency and lifetime.

In the organic light emitting device of the present application, the light emitting layer may include the heterocyclic compound and any one of the following heterocyclic compounds 1-1 to 1-14.

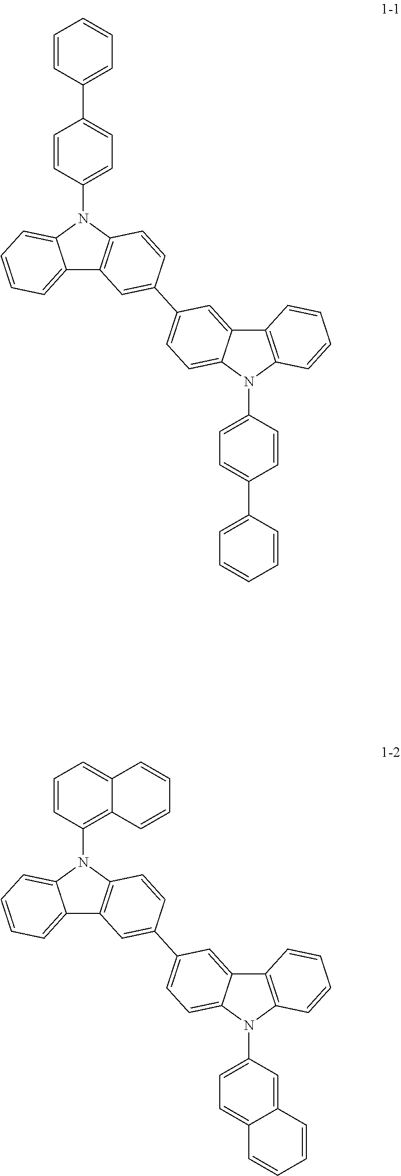 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

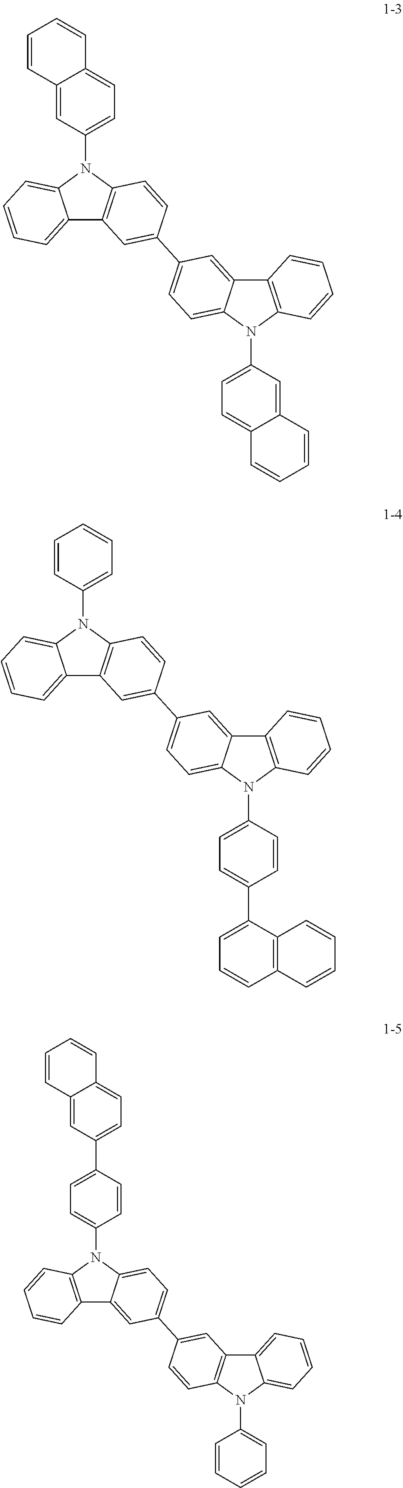 [SMILES: c1ccc2cc(-n3c4ccccc4c4cc(-c5ccc6c(c5)c5ccccc5n6-c5ccc6ccccc6c5)ccc43)ccc2c1 ; c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

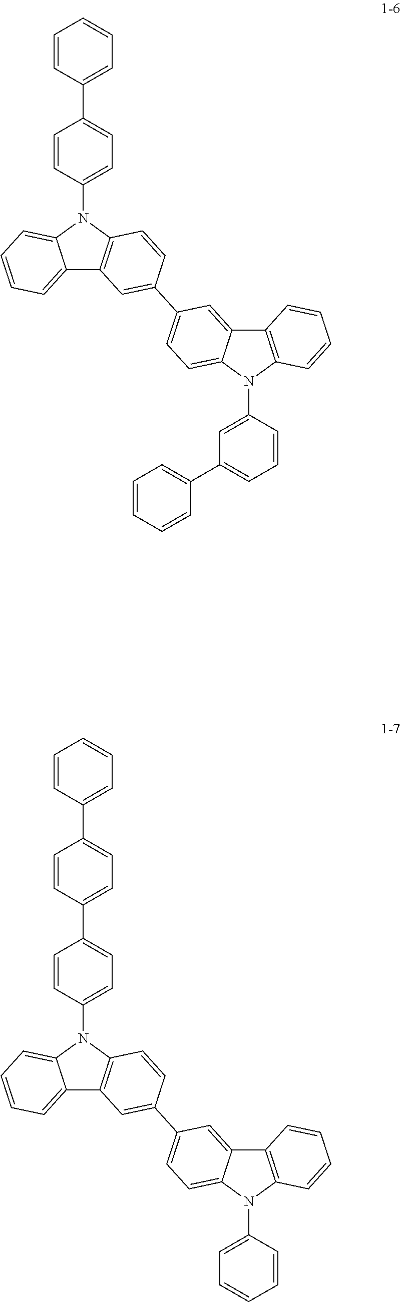 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

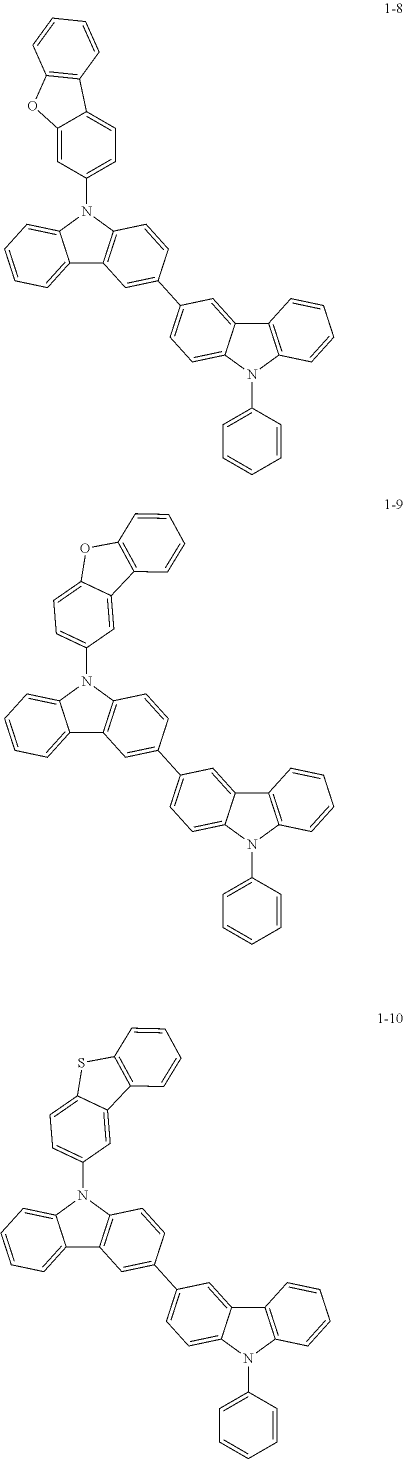 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

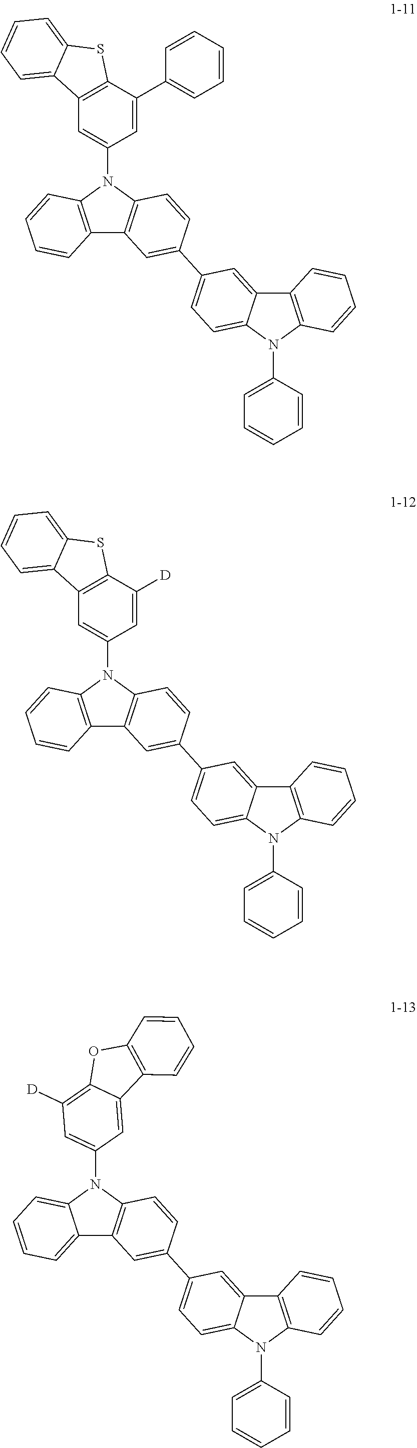 [SMILES: c1ccc(-c2cc(-n3c4ccccc4c4cc(-c5ccc6c(c5)c5ccccc5n6-c5ccccc5)ccc43)cc3c2sc2ccccc23)cc1 ; [2H]c1cc(-n2c3ccccc3c3cc(-c4ccc5c(c4)c4ccccc4n5-c4ccccc4)ccc32)cc2c1sc1ccccc12 ; [2H]c1cc(-n2c3ccccc3c3cc(-c4ccc5c(c4)c4ccccc4n5-c4ccccc4)ccc32)cc2c1oc1ccccc12]

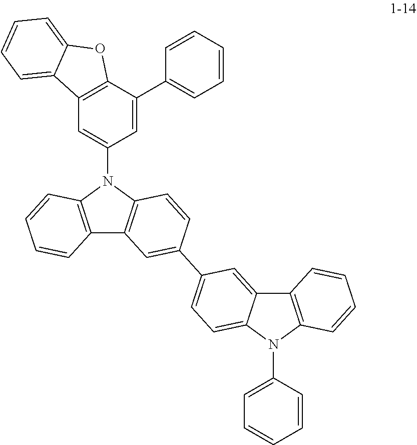 [SMILES: c1ccc(-c2cc(-n3c4ccccc4c4cc(-c5ccc6c(c5)c5ccccc5n6-c5ccccc5)ccc43)cc3c2oc2ccccc23)cc1]

In one embodiment of the present application, the heterocyclic compound represented by Chemical Formula 1 and any one of the heterocyclic compounds 1-1 to 1-14 may be used as a host material.

One embodiment of the present application provides a composition for an organic material layer of an organic light emitting device, the composition including the heterocyclic compound represented by Chemical Formula 1 and any one of the following heterocyclic compounds 1-1 to 1-14.

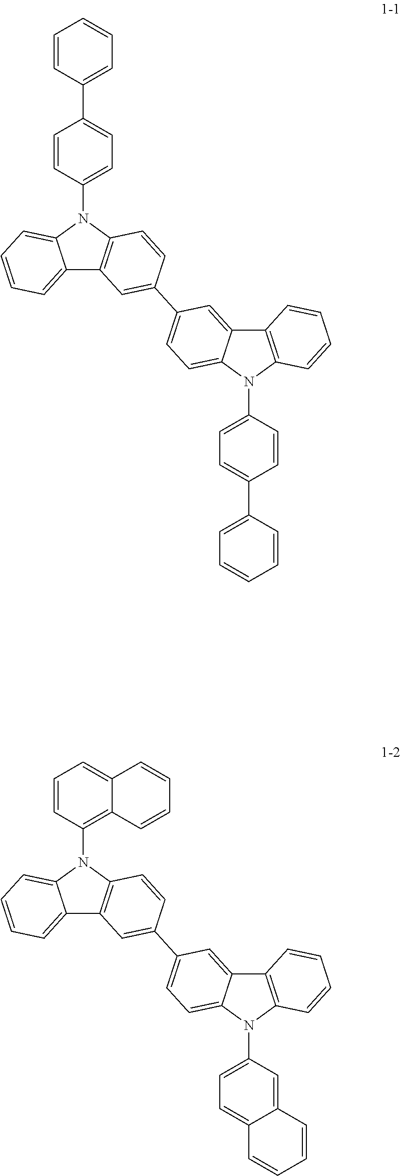 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

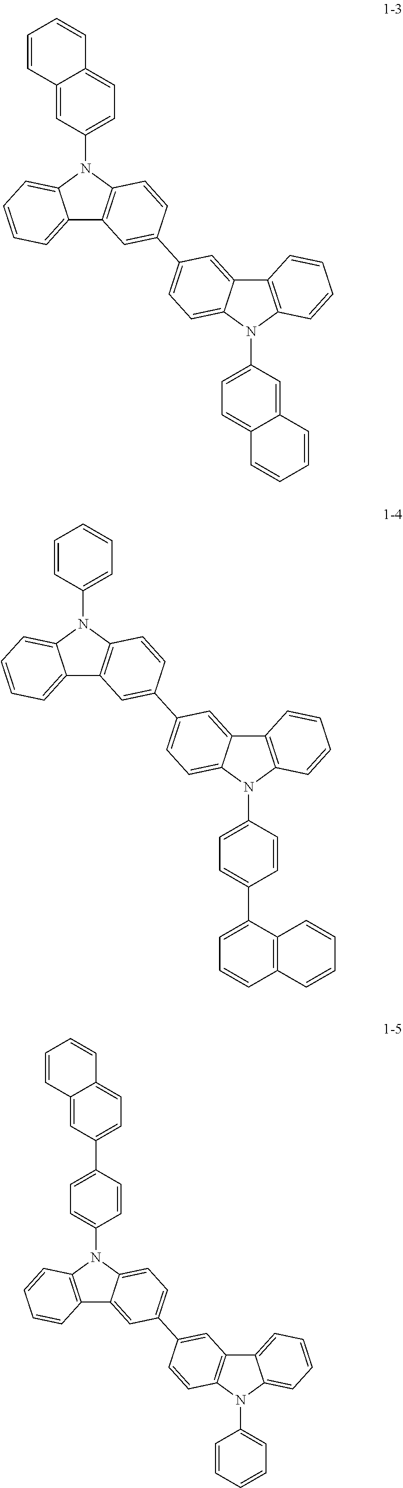 [SMILES: c1ccc2cc(-n3c4ccccc4c4cc(-c5ccc6c(c5)c5ccccc5n6-c5ccc6ccccc6c5)ccc43)ccc2c1 ; c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

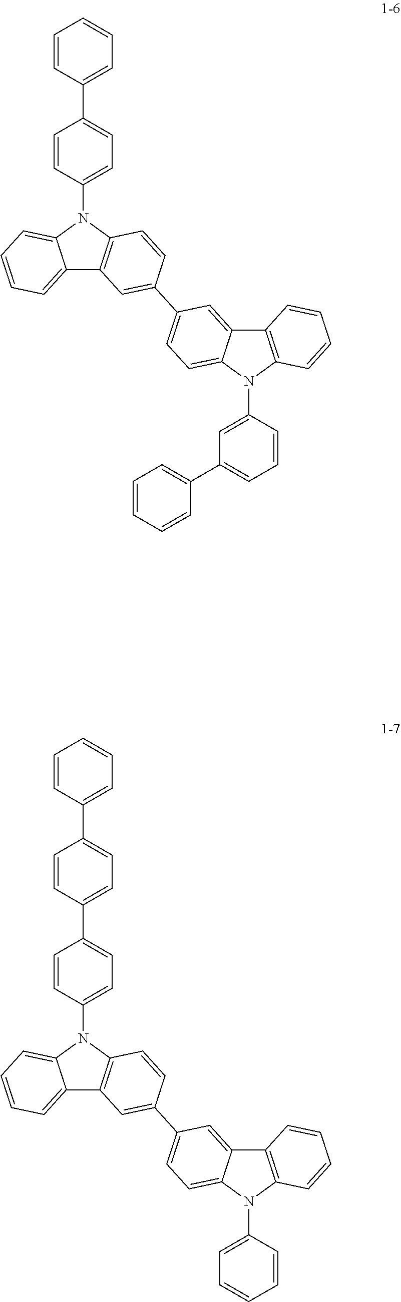 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

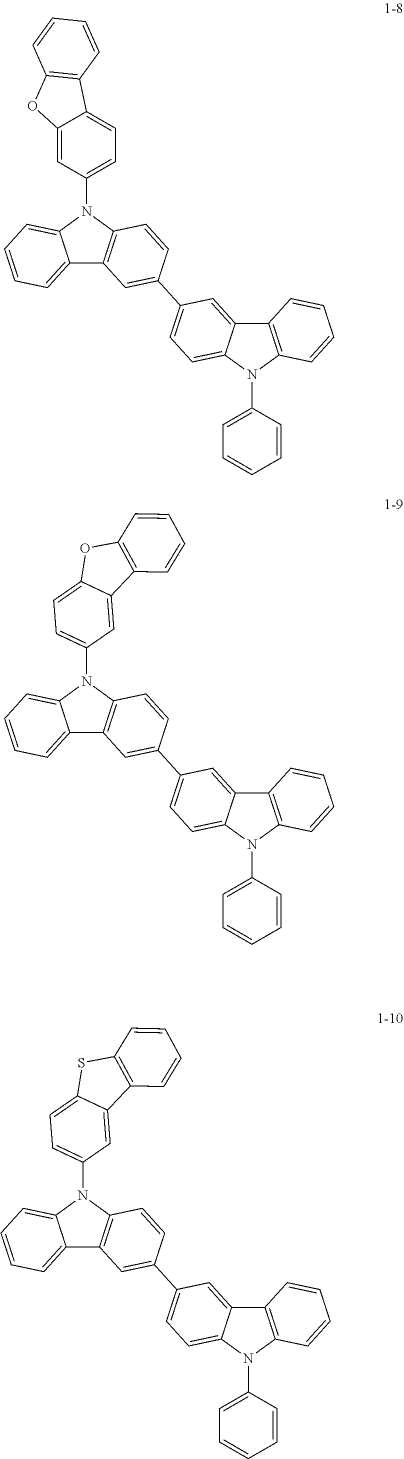 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

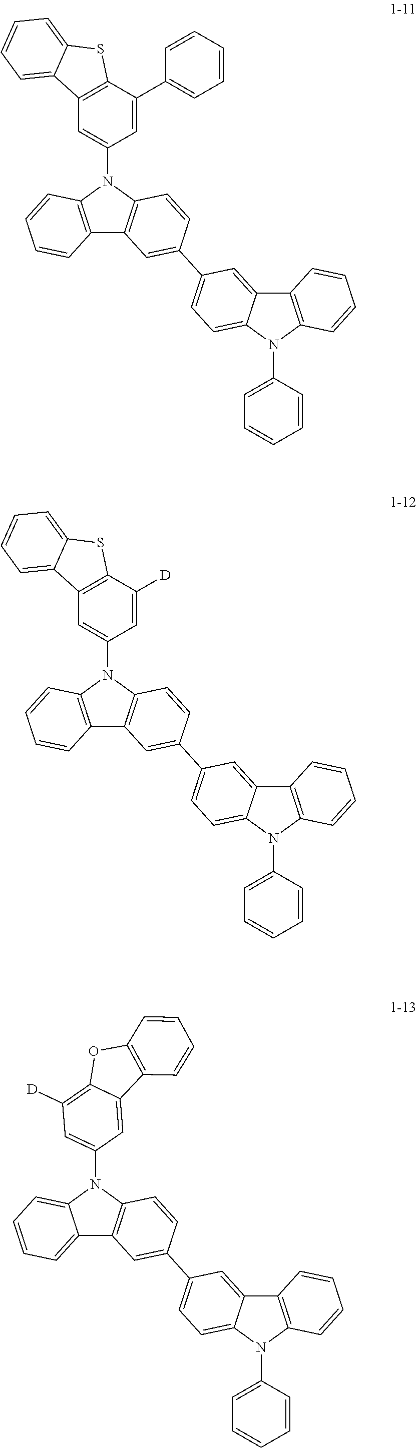 [SMILES: c1ccc(-c2cc(-n3c4ccccc4c4cc(-c5ccc6c(c5)c5ccccc5n6-c5ccccc5)ccc43)cc3c2sc2ccccc23)cc1 ; [2H]c1cc(-n2c3ccccc3c3cc(-c4ccc5c(c4)c4ccccc4n5-c4ccccc4)ccc32)cc2c1sc1ccccc12 ; [2H]c1cc(-n2c3ccccc3c3cc(-c4ccc5c(c4)c4ccccc4n5-c4ccccc4)ccc32)cc2c1oc1ccccc12]

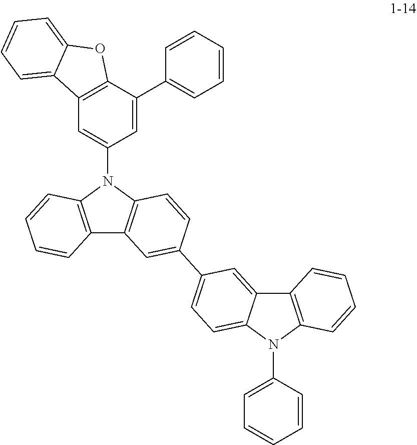 [SMILES: c1ccc(-c2cc(-n3c4ccccc4c4cc(-c5ccc6c(c5)c5ccccc5n6-c5ccccc5)ccc43)cc3c2oc2ccccc23)cc1]

In the composition, the heterocyclic compound represented by Chemical Formula 1:any one of the heterocyclic compounds 1-1 to 1-14 may have a weight ratio of 1:10 to 10:1, 1:8 to 8:1, 1:5 to 5:1 or 1:2 to 2:1, however, the weight ratio is not limited thereto.

The organic light emitting device according to one embodiment of the present application may be a top-emission type, a bottom-emission type or a dual-emission type depending on the materials used.

The heterocyclic compound according to one embodiment of the present application may also be used in an organic electronic device including an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### Preparation Example

### <Preparation Example 1> Preparation of Compound 1

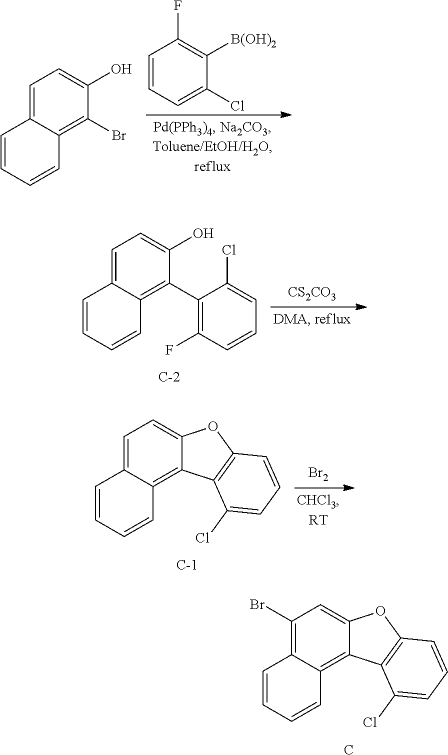

### 1) Preparation of Compound C-2

After dissolving 1-bromonaphthalen-2-ol (100 g, 448.29 mmol), (2-chloro-6-fluorophenyl)boronic acid (85.98 g, 493.12 mmol), Pd(PPh)₄ (25.9 g, 22.41 mmol) and Na₂CO₃ (95.03 g, 896.58 mmol) in toluene/ethanol/H₂O (1 L/200 mL/200 mL), the mixture was refluxed for 4 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:Hex=1:1) to obtain target Compound C-1 (48.9 g, 40%).

### 2) Preparation of Compound C-1

After dissolving Compound C-2 (49 g, 179.68 mmol) and Cs₂CO₃ (146.36 g, 449.21 mol) in dimethylacetamide (DMA) (400 mL), the mixture was refluxed for 2 hours. After the reaction was completed, salts were filtered at room temperature, and the solvent was removed using a rotary evaporator. The reaction material was purified using DCM/MeOH to obtain target Compound C-1 (27.24 g, 60%).

### 3) Preparation of Compound C

After dissolving Compound C-1 (27 g, 106.84 mmol) in chloroform (CHCl₃) (300 mL) at room temperature, Br₂ was dropped thereto, and the result was reacted. After the reaction was completed, the result was recrystallized with methanol to obtain target Compound C (26.93 g, 76%).

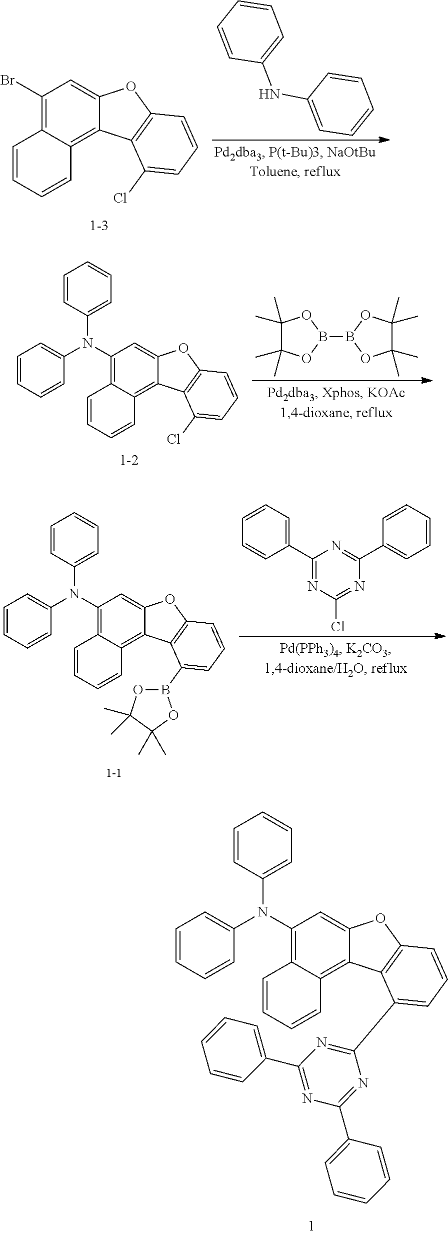

### 4) Preparation of Compound 1-2

After dissolving Compound 1-3 (Compound C) (10.0 g, 30.15 mmol), diphenylamine (5.1 g, 30.15 mmol), Pd₂(dba)₃ (1.38 g, 1.51 mmol), P(t-Bu)₃ (1.22 g, 3.02 mmol) and NaOtBu (5.67 g, 60.32 mmol) in toluene (100 mL), the mixture was refluxed for 2 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3) to obtain target Compound 1-2 (7.59 g, 60%).

### 5) Preparation of Compound 1-1

After dissolving Compound 1-2 (7.59 g, 18.08 mmol), bis(pinacolato)diboron (4,4,4′,4′,5,5,5′,5′-octamethyl-2,2′-bi-1,3,2-dioxaborolane) (5.97 g, 23.5 mmol), Pd₂(dba)₃ (0.83 g, 0.903 mmol), Xphos (0.86 g, 1.81 mmol) and KOAc (3.55 g, 36.15 mmol) in 1,4-dioxane (80 mL), the mixture was refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:2) to obtain target Compound 1-1 (4.81 g, 52%).

### 6) Preparation of Compound 1

After dissolving Compound 1-1 (4.81 g, 9.41 mmol), 2-chloro-4,6-diphenyl-1,3,5-triazine (2.52 g, 9.41 mmol), Pd(pph₃)₄ (0.54 g, 0.47 mmol) and K₂CO₃ (2.6 g, 18.81 mmol) in 1,4-dioxane/H₂O (50 mL/10 mL), the mixture was refluxed for 3 hours. After the reaction was completed, produced solids were filtered, washed with distilled water, and dried. The dried solids were boiled and dissolved in DCB, purified by silica, and the solvent was removed using a rotary evaporator. The result was recrystallized with acetone to obtain target Compound 1 (3.25 g, 56%).

Target compounds were synthesized in the same manner as in Preparation Example 1 except that Intermediate A-1 of the following Table 1 was used instead of diphenylamine, and Intermediate B-1 of the following Table 1 was used instead of 2-chloro-4,6-diphenyl-1,3,5-triazine.

TABLE 1

| Compound | Intermediate | Intermediate | | |
|---|---|---|---|---|
| No. | A-1 | B-1 | Target Compound | Yield |
| 2 | 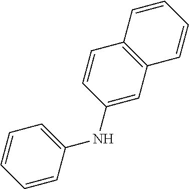 [SMILES: c1ccc(Nc2ccc3ccccc3c2)cc1] | 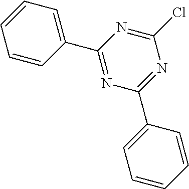 [SMILES: Clc1nc(-c2ccccc2)nc(-c2ccccc2)n1] | 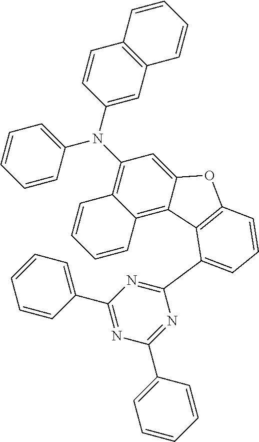 [SMILES: c1ccc(-c2nc(-c3ccccc3)nc(-c3cccc4oc5cc(N(c6ccccc6)c6ccc7ccccc7c6)c6ccccc6c5c34)n2)cc1] | 40% |
| 3 | 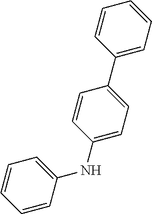 [SMILES: c1ccc(Nc2ccc(-c3ccccc3)cc2)cc1] | | 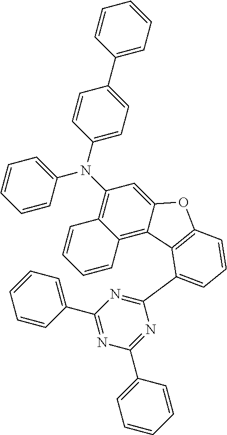 [SMILES: c1ccc(-c2ccc(N(c3ccccc3)c3cc4oc5cccc(-c6nc(-c7ccccc7)nc(-c7ccccc7)n6)c5c4c4ccccc34)cc2)cc1] | 41% |
| 5 | 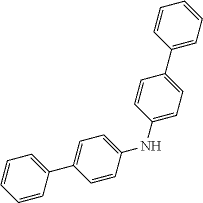 [SMILES: c1ccc(-c2ccc(Nc3ccc(-c4ccccc4)cc3)cc2)cc1] | | 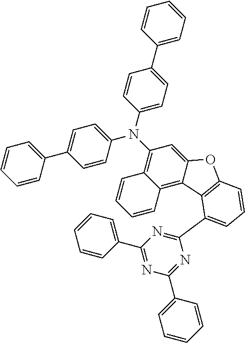 [SMILES: c1ccc(-c2ccc(N(c3ccc(-c4ccccc4)cc3)c3cc4oc5cccc(-c6nc(-c7ccccc7)nc(-c7ccccc7)n6)c5c4c4ccccc34)cc2)cc1] | 43% |
| 8 | 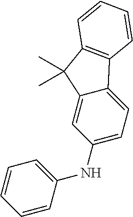 [SMILES: CC1(C)c2ccccc2-c2ccc(Nc3ccccc3)cc21] | | 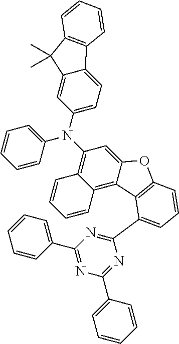 [SMILES: CC1(C)c2ccccc2-c2ccc(N(c3ccccc3)c3cc4oc5cccc(-c6nc(-c7ccccc7)nc(-c7ccccc7)n6)c5c4c4ccccc34)cc21] | 41% |
| 26 | 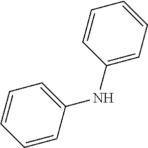 [SMILES: c1ccc(Nc2ccccc2)cc1] | 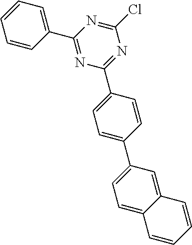 [SMILES: Clc1nc(-c2ccccc2)nc(-c2ccc(-c3ccc4ccccc4c3)cc2)n1] | 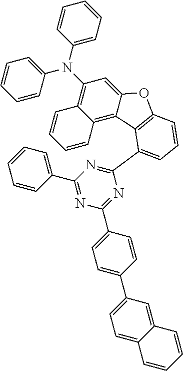 [SMILES: c1ccc(-c2nc(-c3ccc(-c4ccc5ccccc5c4)cc3)nc(-c3cccc4oc5cc(N(c6ccccc6)c6ccccc6)c6ccccc6c5c34)n2)cc1] | 40% |
| 28 | 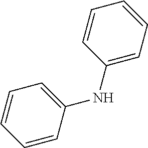 [SMILES: c1ccc(Nc2ccccc2)cc1] | 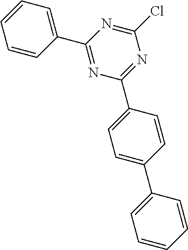 [SMILES: Clc1nc(-c2ccccc2)nc(-c2ccc(-c3ccccc3)cc2)n1] | 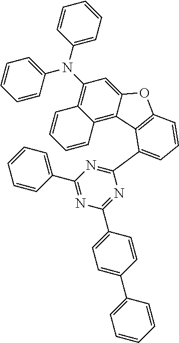 [SMILES: c1ccc(-c2ccc(-c3nc(-c4ccccc4)nc(-c4cccc5oc6cc(N(c7ccccc7)c7ccccc7)c7ccccc7c6c45)n3)cc2)cc1] | 42% |
| 32 | 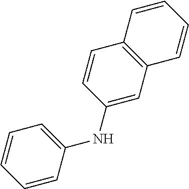 [SMILES: c1ccc(Nc2ccc3ccccc3c2)cc1] | 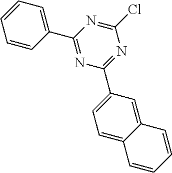 [SMILES: Clc1nc(-c2ccccc2)nc(-c2ccc3ccccc3c2)n1] | 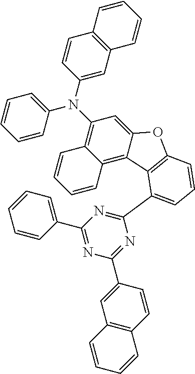 [SMILES: c1ccc(-c2nc(-c3ccc4ccccc4c3)nc(-c3cccc4oc5cc(N(c6ccccc6)c6ccc7ccccc7c6)c6ccccc6c5c34)n2)cc1] | 43% |
| 37 | 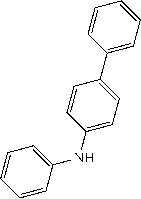 [SMILES: c1ccc(Nc2ccc(-c3ccccc3)cc2)cc1] | | 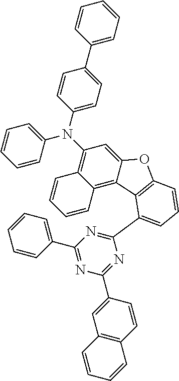 [SMILES: c1ccc(-c2ccc(N(c3ccccc3)c3cc4oc5cccc(-c6nc(-c7ccccc7)nc(-c7ccc8ccccc8c7)n6)c5c4c4ccccc34)cc2)cc1] | 52% |
| 39 | 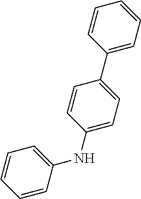 [SMILES: c1ccc(Nc2ccc(-c3ccccc3)cc2)cc1] | 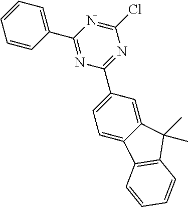 [SMILES: CC1(C)c2ccccc2-c2ccc(-c3nc(Cl)nc(-c4ccccc4)n3)cc21] | 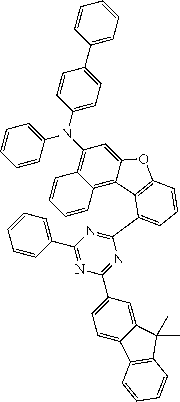 [SMILES: CC1(C)c2ccccc2-c2ccc(-c3nc(-c4ccccc4)nc(-c4cccc5oc6cc(N(c7ccccc7)c7ccc(-c8ccccc8)cc7)c7ccccc7c6c45)n3)cc21] | 48% |
| 44 | 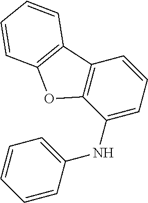 [SMILES: c1ccc(Nc2cccc3c2oc2ccccc23)cc1] | 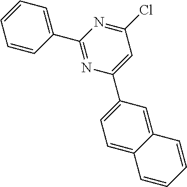 [SMILES: Clc1cc(-c2ccc3ccccc3c2)nc(-c2ccccc2)n1] | 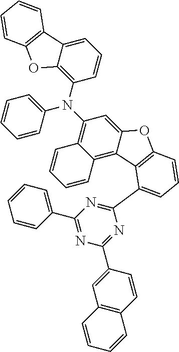 [SMILES: c1ccc(-c2nc(-c3ccc4ccccc4c3)nc(-c3cccc4oc5cc(N(c6ccccc6)c6cccc7c6oc6ccccc67)c6ccccc6c5c34)n2)cc1] | 39% |
| 57 | 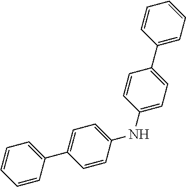 [SMILES: c1ccc(-c2ccc(Nc3ccc(-c4ccccc4)cc3)cc2)cc1] | 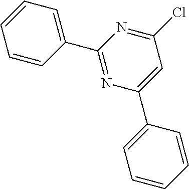 [SMILES: Clc1cc(-c2ccccc2)nc(-c2ccccc2)n1] | 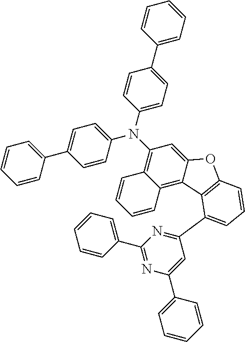 [SMILES: c1ccc(-c2ccc(N(c3ccc(-c4ccccc4)cc3)c3cc4oc5cccc(-c6cc(-c7ccccc7)nc(-c7ccccc7)n6)c5c4c4ccccc34)cc2)cc1] | 36% |
| 60 | 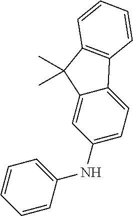 [SMILES: CC1(C)c2ccccc2-c2ccc(Nc3ccccc3)cc21] | 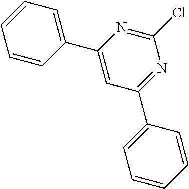 [SMILES: Clc1nc(-c2ccccc2)cc(-c2ccccc2)n1] | 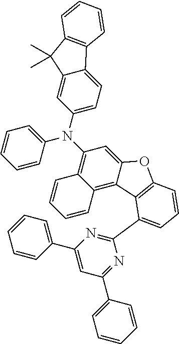 [SMILES: CC1(C)c2ccccc2-c2ccc(N(c3ccccc3)c3cc4oc5cccc(-c6nc(-c7ccccc7)cc(-c7ccccc7)n6)c5c4c4ccccc34)cc21] | 42% |
| 70 | 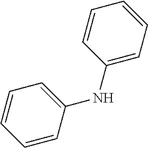 [SMILES: c1ccc(Nc2ccccc2)cc1] | 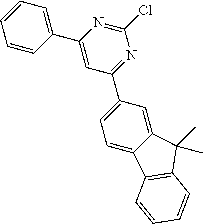 [SMILES: CC1(C)c2ccccc2-c2ccc(-c3cc(-c4ccccc4)nc(Cl)n3)cc21] | 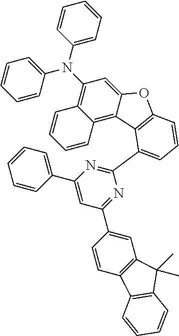 [SMILES: CC1(C)c2ccccc2-c2ccc(-c3cc(-c4ccccc4)nc(-c4cccc5oc6cc(N(c7ccccc7)c7ccccc7)c7ccccc7c6c45)n3)cc21] | 44% |
| 72 | 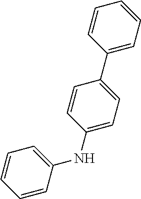 [SMILES: c1ccc(Nc2ccc(-c3ccccc3)cc2)cc1] | 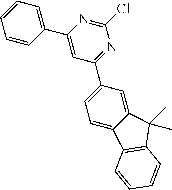 [SMILES: CC1(C)c2ccccc2-c2ccc(-c3cc(-c4ccccc4)nc(Cl)n3)cc21] | 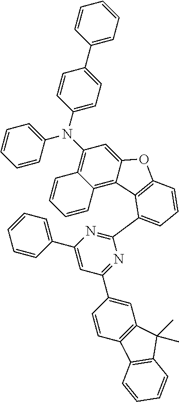 [SMILES: CC1(C)c2ccccc2-c2ccc(-c3cc(-c4ccccc4)nc(-c4cccc5oc6cc(N(c7ccccc7)c7ccc(-c8ccccc8)cc7)c7ccccc7c6c45)n3)cc21] | 35% |
| 79 | 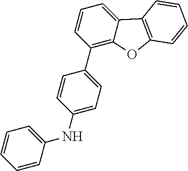 [SMILES: c1ccc(Nc2ccc(-c3cccc4c3oc3ccccc34)cc2)cc1] | 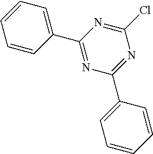 [SMILES: Clc1nc(-c2ccccc2)nc(-c2ccccc2)n1] | 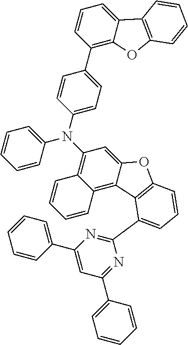 [SMILES: c1ccc(-c2cc(-c3ccccc3)nc(-c3cccc4oc5cc(N(c6ccccc6)c6ccc(-c7cccc8c7oc7ccccc78)cc6)c6ccccc6c5c34)n2)cc1] | 48% |
| 86 | 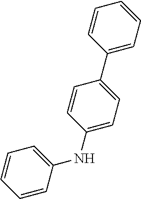 [SMILES: c1ccc(Nc2ccc(-c3ccccc3)cc2)cc1] | 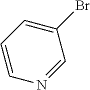 | 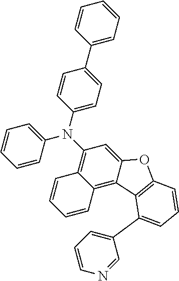 [SMILES: c1ccc(-c2ccc(N(c3ccccc3)c3cc4oc5cccc(-c6cccnc6)c5c4c4ccccc34)cc2)cc1] | 52% |
| 88 | 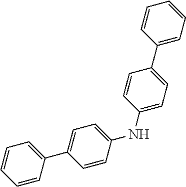 [SMILES: c1ccc(-c2ccc(Nc3ccc(-c4ccccc4)cc3)cc2)cc1] | | 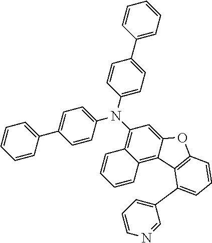 [SMILES: c1ccc(-c2ccc(N(c3ccc(-c4ccccc4)cc3)c3cc4oc5cccc(-c6cccnc6)c5c4c4ccccc34)cc2)cc1] | 51% |
| 91 | 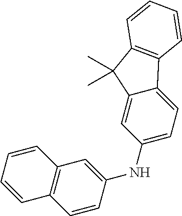 [SMILES: CC1(C)c2ccccc2-c2ccc(Nc3ccc4ccccc4c3)cc21] | | 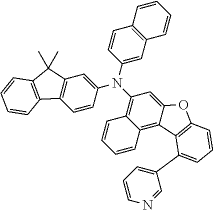 [SMILES: CC1(C)c2ccccc2-c2ccc(N(c3ccc4ccccc4c3)c3cc4oc5cccc(-c6cccnc6)c5c4c4ccccc34)cc21] | 43% |
| 92 | 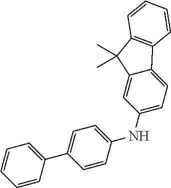 [SMILES: CC1(C)c2ccccc2-c2ccc(Nc3ccc(-c4ccccc4)cc3)cc21] | | 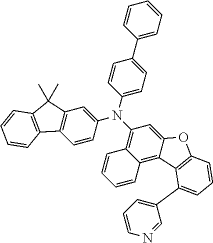 [SMILES: CC1(C)c2ccccc2-c2ccc(N(c3ccc(-c4ccccc4)cc3)c3cc4oc5cccc(-c6cccnc6)c5c4c4ccccc34)cc21] | 47% |
| 95 | 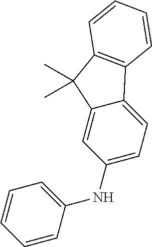 [SMILES: CC1(C)c2ccccc2-c2ccc(Nc3ccccc3)cc21] | | 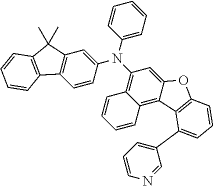 [SMILES: CC1(C)c2ccccc2-c2ccc(N(c3ccccc3)c3cc4oc5cccc(-c6cccnc6)c5c4c4ccccc34)cc21] | 46% |
| 96 | 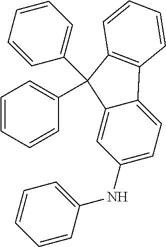 [SMILES: c1ccc(Nc2ccc3c(c2)C(c2ccccc2)(c2ccccc2)c2ccccc2-3)cc1] | | 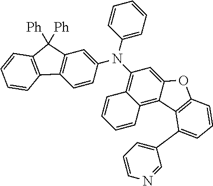 [SMILES: c1ccc(N(c2ccc3c(c2)C(c2ccccc2)(c2ccccc2)c2ccccc2-3)c2cc3oc4cccc(-c5cccnc5)c4c3c3ccccc23)cc1] | 55% |
| 114 | 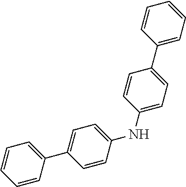 [SMILES: c1ccc(-c2ccc(Nc3ccc(-c4ccccc4)cc3)cc2)cc1] | 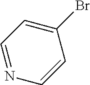 | 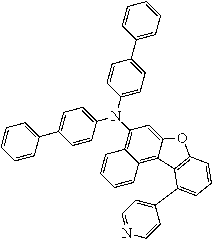 [SMILES: c1ccc(-c2ccc(N(c3ccc(-c4ccccc4)cc3)c3cc4oc5cccc(-c6ccncc6)c5c4c4ccccc34)cc2)cc1] | 45% |
| 116 | 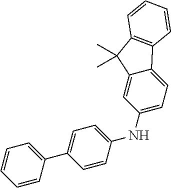 [SMILES: CC1(C)c2ccccc2-c2ccc(Nc3ccc(-c4ccccc4)cc3)cc21] | | 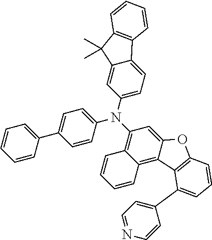 [SMILES: CC1(C)c2ccccc2-c2ccc(N(c3ccc(-c4ccccc4)cc3)c3cc4oc5cccc(-c6ccncc6)c5c4c4ccccc34)cc21] | 43% |
| 127 | 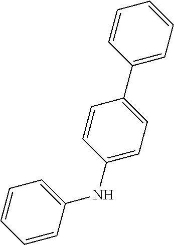 [SMILES: c1ccc(Nc2ccc(-c3ccccc3)cc2)cc1] | 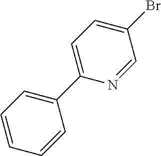 [SMILES: Brc1ccc(-c2ccccc2)nc1] | 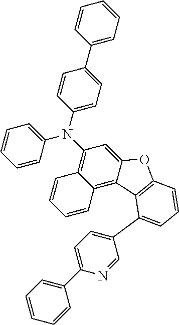 [SMILES: c1ccc(-c2ccc(N(c3ccccc3)c3cc4oc5cccc(-c6ccc(-c7ccccc7)nc6)c5c4c4ccccc34)cc2)cc1] | 49% |
| 129 | 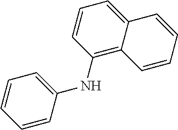 [SMILES: c1ccc(Nc2cccc3ccccc23)cc1] | | 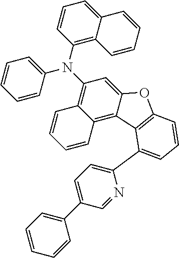 [SMILES: c1ccc(-c2ccc(-c3cccc4oc5cc(N(c6ccccc6)c6cccc7ccccc67)c6ccccc6c5c34)nc2)cc1] | 51% |
| 135 | 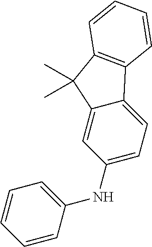 [SMILES: CC1(C)c2ccccc2-c2ccc(Nc3ccccc3)cc21] | | 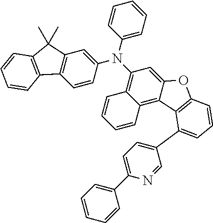 [SMILES: CC1(C)c2ccccc2-c2ccc(N(c3ccccc3)c3cc4oc5cccc(-c6ccc(-c7ccccc7)nc6)c5c4c4ccccc34)cc21] | 39% |
| 198 | 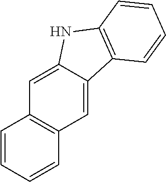 [SMILES: c1ccc2cc3c(cc2c1)[nH]c1ccccc13] | 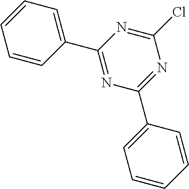 [SMILES: Clc1nc(-c2ccccc2)nc(-c2ccccc2)n1] | 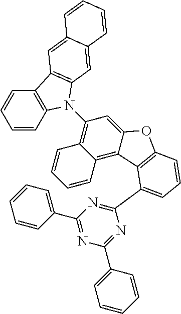 [SMILES: c1ccc(-c2nc(-c3ccccc3)nc(-c3cccc4oc5cc(-n6c7ccccc7c7cc8ccccc8cc76)c6ccccc6c5c34)n2)cc1] | 38% |
| 199 | 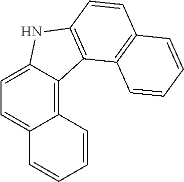 [SMILES: c1ccc2c(c1)ccc1[nH]c3ccc4ccccc4c3c12] | 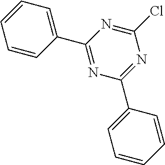 [SMILES: Clc1nc(-c2ccccc2)nc(-c2ccccc2)n1] | 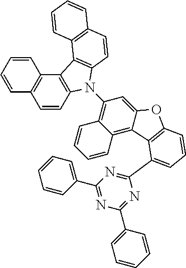 [SMILES: c1ccc(-c2nc(-c3ccccc3)nc(-c3cccc4oc5cc(-n6c7ccc8ccccc8c7c7c8ccccc8ccc76)c6ccccc6c5c34)n2)cc1] | 31% |
| 207 | 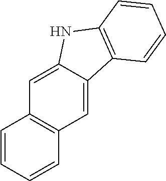 [SMILES: c1ccc2cc3c(cc2c1)[nH]c1ccccc13] | 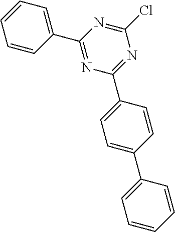 [SMILES: Clc1nc(-c2ccccc2)nc(-c2ccc(-c3ccccc3)cc2)n1] | 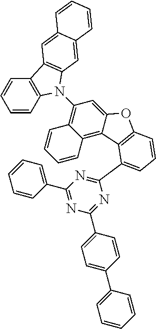 [SMILES: c1ccc(-c2ccc(-c3nc(-c4ccccc4)nc(-c4cccc5oc6cc(-n7c8ccccc8c8cc9ccccc9cc87)c7ccccc7c6c45)n3)cc2)cc1] | 40% |
| 232 | 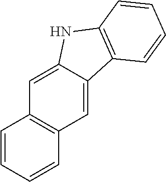 [SMILES: c1ccc2cc3c(cc2c1)[nH]c1ccccc13] | 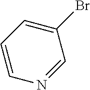 | 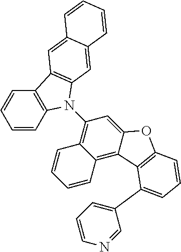 [SMILES: c1cncc(-c2cccc3oc4cc(-n5c6ccccc6c6cc7ccccc7cc65)c5ccccc5c4c23)c1] | 36% |

### <Preparation Example 2> Preparation of Compound 13

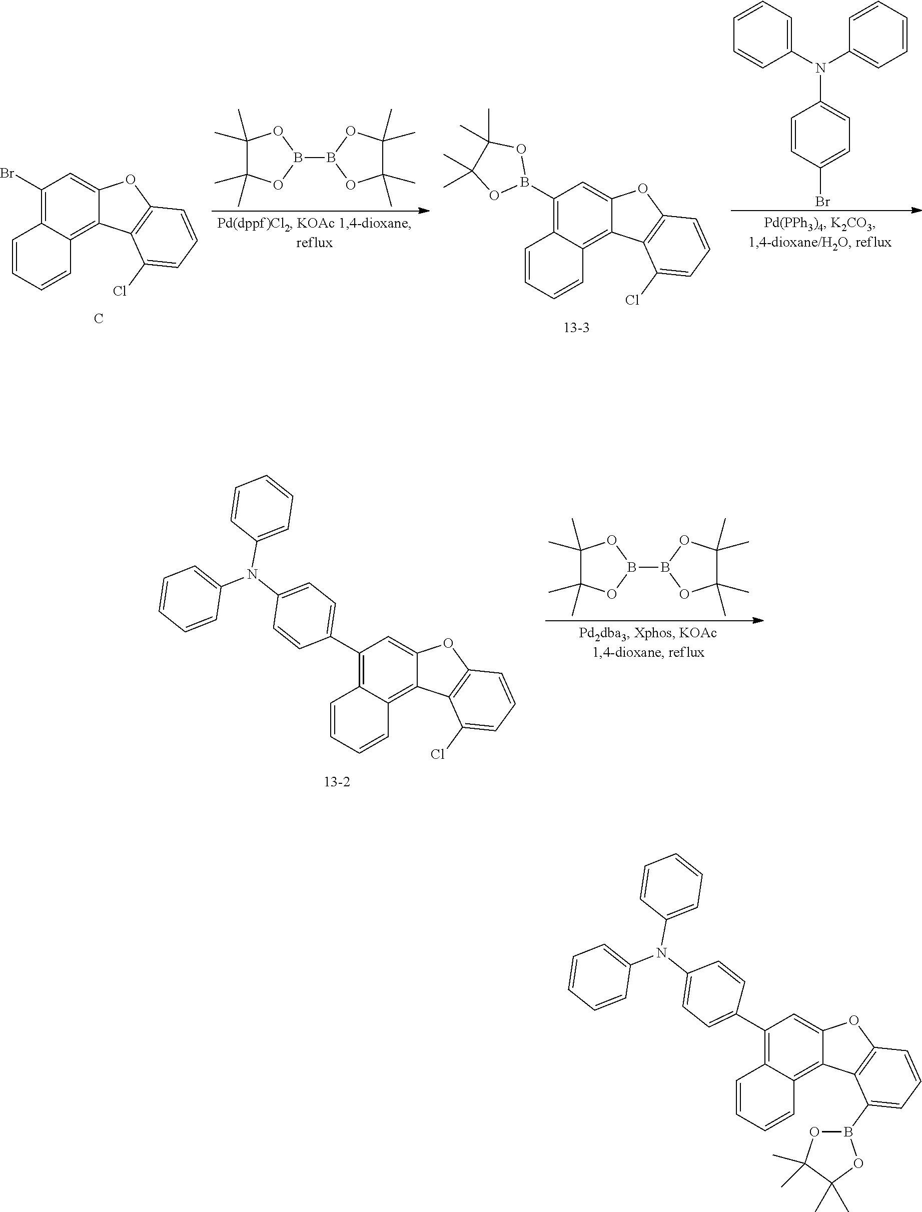

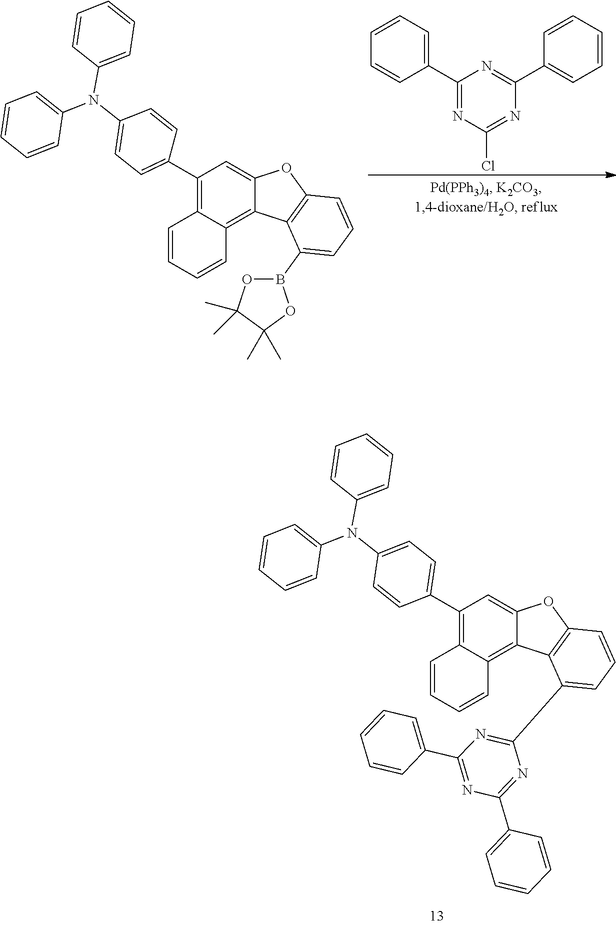 [SMILES: CC1(C)OB(c2cccc3oc4cc(-c5ccc(N(c6ccccc6)c6ccccc6)cc5)c5ccccc5c4c23)OC1(C)C ; Clc1nc(-c2ccccc2)nc(-c2ccccc2)n1 ; c1ccc(-c2nc(-c3ccccc3)nc(-c3cccc4oc5cc(-c6ccc(N(c7ccccc7)c7ccccc7)cc6)c6ccccc6c5c34)n2)cc1]

### 1) Preparation of Compound 13-3

After dissolving Compound C (10.0 g, 30.15 mmol), bis(pinacolato)diboron (9.2 g, 36.2 mmol), Pd(dppf)Cl₂ (1.1 g, 1.51 mmol) and KOAc (5.68 g, 60.32 mmol) in 1,4-dioxane (100 mL), the mixture was refluxed for 2 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:2) to obtain target Compound 13-3 (6.97 g, 61%).

### 2) Preparation of Compound 13-2

After dissolving Compound 13-3 (6.97 g, 18.41 mmol), 4-bromo-N,N-diphenylaniline (5.97 g, 18.41 mmol), Pd(pph₃)₄ (1.06 g, 9.2 mmol) and K₂CO₃ (5.09 g, 36.81 mmol) in 1,4-dioxane/H₂O (80 mL/12 mL), the mixture was refluxed for 3 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3) to obtain target Compound 13-2 (4.75 g, 52%).

### 3) Preparation of Compound 13-1

After dissolving Compound 13-2 (4.75 g, 9.58 mmol), bis(pinacolato)diboron (4,4,4′,4′,5,5,5′,5′-octamethyl-2,2′-bi-1,3,2-dioxaborolane) (3.16 g, 12.45 mmol), Pd₂(dba)₃ (0.44 g, 0.48 mmol), Xphos (0.46 g, 0.96 mmol) and KOAc (1.88 g, 19.15 mmol) in 1,4-dioxane (50 mL), the mixture was refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:2) to obtain target Compound 13-1 (4.28 g, 76%).

### 4) Preparation of Compound 13

After dissolving Compound 13-1 (4.28 g, 7.28 mmol), 2-chloro-4,6-diphenyl-1,3,5-triazine (1.95 g, 7.28 mmol), Pd(pph₃)₄ (0.42 g, 0.36 mmol) and K₂CO₃ (2.01 g, 14.57 mmol) in 1,4-dioxane/H₂O (50 mL/10 mL), the mixture was refluxed for 3 hours. After the reaction was completed, produced solids were filtered, washed with distilled water, and dried. The dried solids were boiled and dissolved in DCB, purified by silica, and the solvent was removed using a rotary evaporator. The result was recrystallized with acetone to obtain target Compound 13 (2.93 g, 58%).

Target compounds were synthesized in the same manner as in Preparation Example 2 except that Intermediate A-2 of the following Table 2 was used instead of 4-bromo-N,N-diphenylaniline, and Intermediate B-2 of the following Table 2 was used instead of 2-chloro-4,6-diphenyl-1,3,5-triazine.

TABLE 2

| Compound | Intermediate | Intermediate | | |
|---|---|---|---|---|
| No. | A-2 | B-2 | Target Compound | Yield |
| 14 | 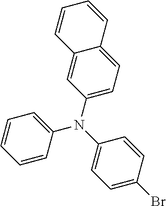 [SMILES: Brc1ccc(N(c2ccccc2)c2ccc3ccccc3c2)cc1] | 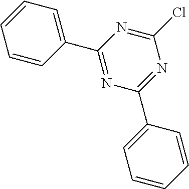 [SMILES: Clc1nc(-c2ccccc2)nc(-c2ccccc2)n1] | 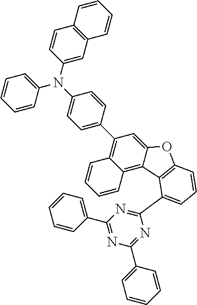 [SMILES: c1ccc(-c2nc(-c3ccccc3)nc(-c3cccc4oc5cc(-c6ccc(N(c7ccccc7)c7ccc8ccccc8c7)cc6)c6ccccc6c5c34)n2)cc1] | 56% |
| 15 | 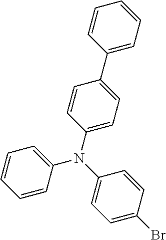 [SMILES: Brc1ccc(N(c2ccccc2)c2ccc(-c3ccccc3)cc2)cc1] | | 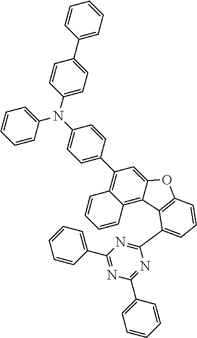 [SMILES: c1ccc(-c2ccc(N(c3ccccc3)c3ccc(-c4cc5oc6cccc(-c7nc(-c8ccccc8)nc(-c8ccccc8)n7)c6c5c5ccccc45)cc3)cc2)cc1] | 49% |
| 19 | 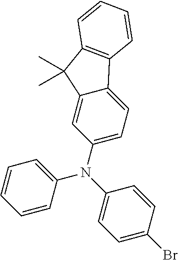 [SMILES: CC1(C)c2ccccc2-c2ccc(N(c3ccccc3)c3ccc(Br)cc3)cc21] | | 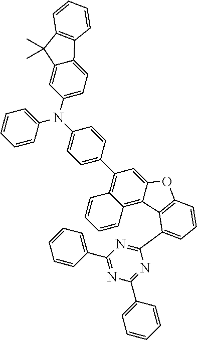 [SMILES: CC1(C)c2ccccc2-c2ccc(N(c3ccccc3)c3ccc(-c4cc5oc6cccc(-c7nc(-c8ccccc8)nc(-c8ccccc8)n7)c6c5c5ccccc45)cc3)cc21] | 49% |
| 22 | 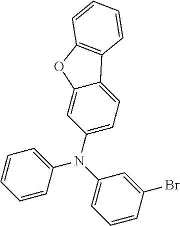 [SMILES: Brc1cccc(N(c2ccccc2)c2ccc3c(c2)oc2ccccc23)c1] | | 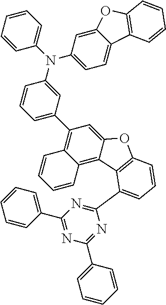 [SMILES: c1ccc(-c2nc(-c3ccccc3)nc(-c3cccc4oc5cc(-c6cccc(N(c7ccccc7)c7ccc8c(c7)oc7ccccc78)c6)c6ccccc6c5c34)n2)cc1] | 48% |
| 24 | 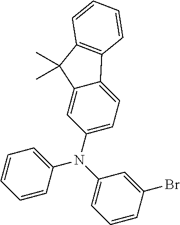 [SMILES: CC1(C)c2ccccc2-c2ccc(N(c3ccccc3)c3cccc(Br)c3)cc21] | | 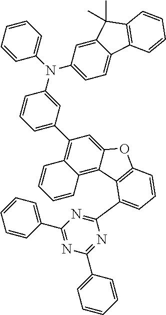 [SMILES: CC1(C)c2ccccc2-c2ccc(N(c3ccccc3)c3cccc(-c4cc5oc6cccc(-c7nc(-c8ccccc8)nc(-c8ccccc8)n7)c6c5c5ccccc45)c3)cc21] | 51% |
| 48 | 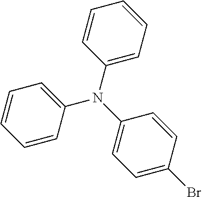 [SMILES: Brc1ccc(N(c2ccccc2)c2ccccc2)cc1] | 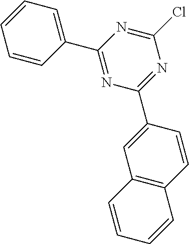 [SMILES: Clc1nc(-c2ccccc2)nc(-c2ccc3ccccc3c2)n1] | 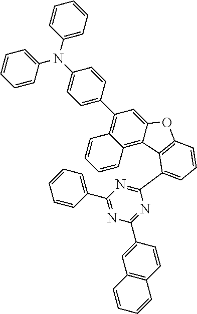 [SMILES: c1ccc(-c2nc(-c3ccc4ccccc4c3)nc(-c3cccc4oc5cc(-c6ccc(N(c7ccccc7)c7ccccc7)cc6)c6ccccc6c5c34)n2)cc1] | 57% |
| 51 | 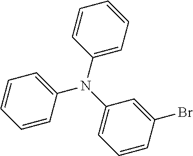 [SMILES: Brc1cccc(N(c2ccccc2)c2ccccc2)c1] | | 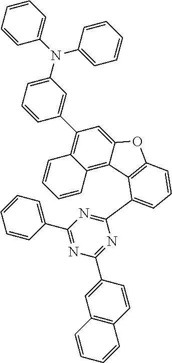 [SMILES: c1ccc(-c2nc(-c3ccc4ccccc4c3)nc(-c3cccc4oc5cc(-c6cccc(N(c7ccccc7)c7ccccc7)c6)c6ccccc6c5c34)n2)cc1] | 54% |
| 62 | 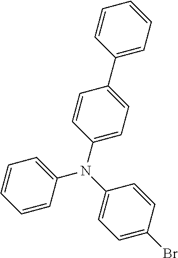 [SMILES: Brc1ccc(N(c2ccccc2)c2ccc(-c3ccccc3)cc2)cc1] | 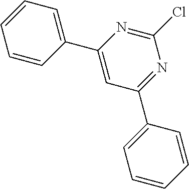 [SMILES: Clc1nc(-c2ccccc2)cc(-c2ccccc2)n1] | 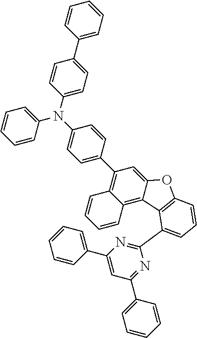 [SMILES: c1ccc(-c2ccc(N(c3ccccc3)c3ccc(-c4cc5oc6cccc(-c7nc(-c8ccccc8)cc(-c8ccccc8)n7)c6c5c5ccccc45)cc3)cc2)cc1] | 52% |
| 65 | 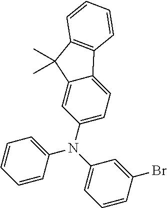 [SMILES: CC1(C)c2ccccc2-c2ccc(N(c3ccccc3)c3cccc(Br)c3)cc21] | | 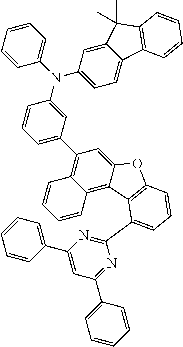 [SMILES: CC1(C)c2ccccc2-c2ccc(N(c3ccccc3)c3cccc(-c4cc5oc6cccc(-c7nc(-c8ccccc8)cc(-c8ccccc8)n7)c6c5c5ccccc45)c3)cc21] | 50% |
| 99 | 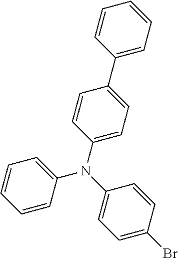 [SMILES: Brc1ccc(N(c2ccccc2)c2ccc(-c3ccccc3)cc2)cc1] | 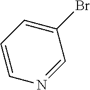 | 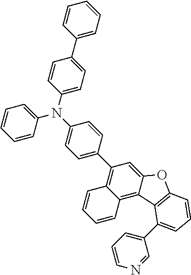 [SMILES: c1ccc(-c2ccc(N(c3ccccc3)c3ccc(-c4cc5oc6cccc(-c7cccnc7)c6c5c5ccccc45)cc3)cc2)cc1] | 43% |
| 101 | 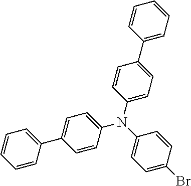 [SMILES: Brc1ccc(N(c2ccc(-c3ccccc3)cc2)c2ccc(-c3ccccc3)cc2)cc1] | 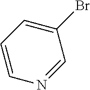 | 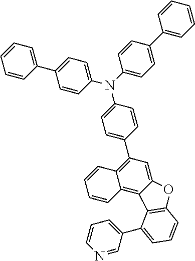 [SMILES: c1ccc(-c2ccc(N(c3ccc(-c4ccccc4)cc3)c3ccc(-c4cc5oc6cccc(-c7cccnc7)c6c5c5ccccc45)cc3)cc2)cc1] | 49% |
| 102 | 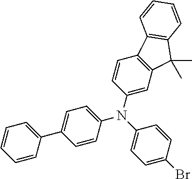 [SMILES: CC1(C)c2ccccc2-c2ccc(N(c3ccc(Br)cc3)c3ccc(-c4ccccc4)cc3)cc21] | 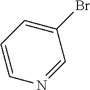 | 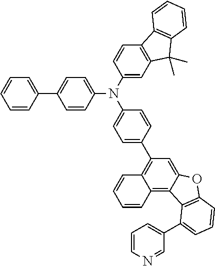 [SMILES: CC1(C)c2ccccc2-c2ccc(N(c3ccc(-c4ccccc4)cc3)c3ccc(-c4cc5oc6cccc(-c7cccnc7)c6c5c5ccccc45)cc3)cc21] | 46% |
| 117 | 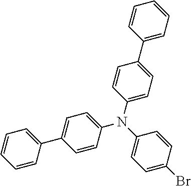 [SMILES: Brc1ccc(N(c2ccc(-c3ccccc3)cc2)c2ccc(-c3ccccc3)cc2)cc1] | 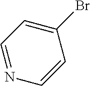 | 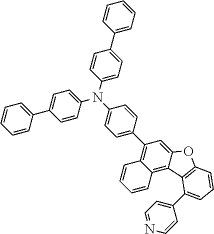 [SMILES: c1ccc(-c2ccc(N(c3ccc(-c4ccccc4)cc3)c3ccc(-c4cc5oc6cccc(-c7ccncc7)c6c5c5ccccc45)cc3)cc2)cc1] | 47% |
| 118 | 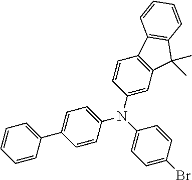 [SMILES: CC1(C)c2ccccc2-c2ccc(N(c3ccc(Br)cc3)c3ccc(-c4ccccc4)cc3)cc21] | 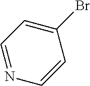 | 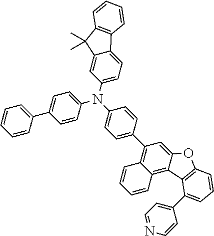 [SMILES: CC1(C)c2ccccc2-c2ccc(N(c3ccc(-c4ccccc4)cc3)c3ccc(-c4cc5oc6cccc(-c7ccncc7)c6c5c5ccccc45)cc3)cc21] | 48% |
| 120 | 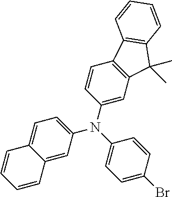 [SMILES: CC1(C)c2ccccc2-c2ccc(N(c3ccc(Br)cc3)c3ccc4ccccc4c3)cc21] | 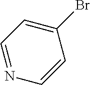 | 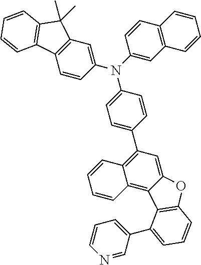 [SMILES: CC1(C)c2ccccc2-c2ccc(N(c3ccc(-c4cc5oc6cccc(-c7cccnc7)c6c5c5ccccc45)cc3)c3ccc4ccccc4c3)cc21] | 50% |
| 121 | 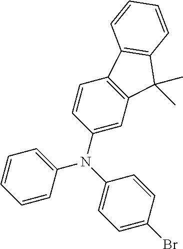 [SMILES: CC1(C)c2ccccc2-c2ccc(N(c3ccccc3)c3ccc(Br)cc3)cc21] | 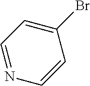 | 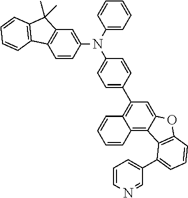 [SMILES: CC1(C)c2ccccc2-c2ccc(N(c3ccccc3)c3ccc(-c4cc5oc6cccc(-c7cccnc7)c6c5c5ccccc45)cc3)cc21] | 54% |
| 141 | 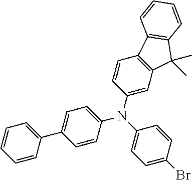 [SMILES: CC1(C)c2ccccc2-c2ccc(N(c3ccc(Br)cc3)c3ccc(-c4ccccc4)cc3)cc21] | 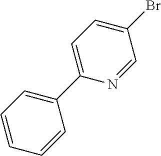 [SMILES: Brc1ccc(-c2ccccc2)nc1] | 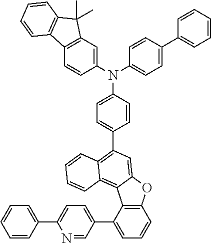 [SMILES: CC1(C)c2ccccc2-c2ccc(N(c3ccc(-c4ccccc4)cc3)c3ccc(-c4cc5oc6cccc(-c7ccc(-c8ccccc8)nc7)c6c5c5ccccc45)cc3)cc21] | 51% |
| 217 | 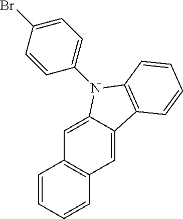 [SMILES: Brc1ccc(-n2c3ccccc3c3cc4ccccc4cc32)cc1] | 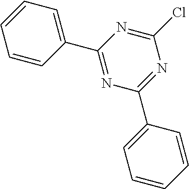 [SMILES: Clc1nc(-c2ccccc2)nc(-c2ccccc2)n1] | 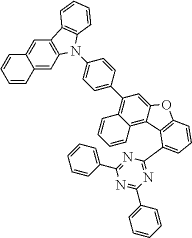 [SMILES: c1ccc(-c2nc(-c3ccccc3)nc(-c3cccc4oc5cc(-c6ccc(-n7c8ccccc8c8cc9ccccc9cc87)cc6)c6ccccc6c5c34)n2)cc1] | 37% |
| 218 | 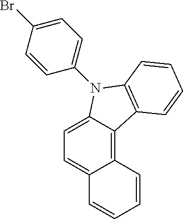 [SMILES: Brc1ccc(-n2c3ccccc3c3c4ccccc4ccc32)cc1] | | 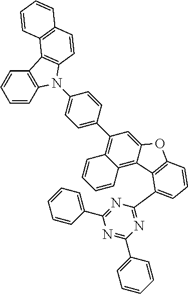 [SMILES: c1ccc(-c2nc(-c3ccccc3)nc(-c3cccc4oc5cc(-c6ccc(-n7c8ccccc8c8c9ccccc9ccc87)cc6)c6ccccc6c5c34)n2)cc1] | 42% |
| 252 | 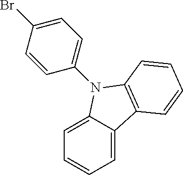 [SMILES: Brc1ccc(-n2c3ccccc3c3ccccc32)cc1] | | 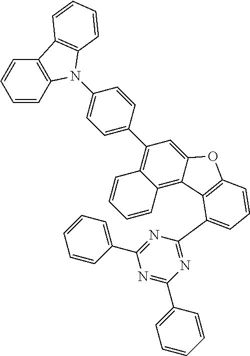 [SMILES: c1ccc(-c2nc(-c3ccccc3)nc(-c3cccc4oc5cc(-c6ccc(-n7c8ccccc8c8ccccc87)cc6)c6ccccc6c5c34)n2)cc1] | 46% |
| 255 | 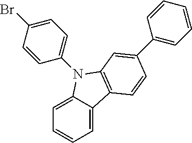 [SMILES: Brc1ccc(-n2c3ccccc3c3ccc(-c4ccccc4)cc32)cc1] | | 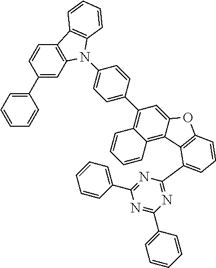 [SMILES: c1ccc(-c2ccc3c4ccccc4n(-c4ccc(-c5cc6oc7cccc(-c8nc(-c9ccccc9)nc(-c9ccccc9)n8)c7c6c6ccccc56)cc4)c3c2)cc1] | 49% |

### <Preparation Example 3> Preparation of Compound 145

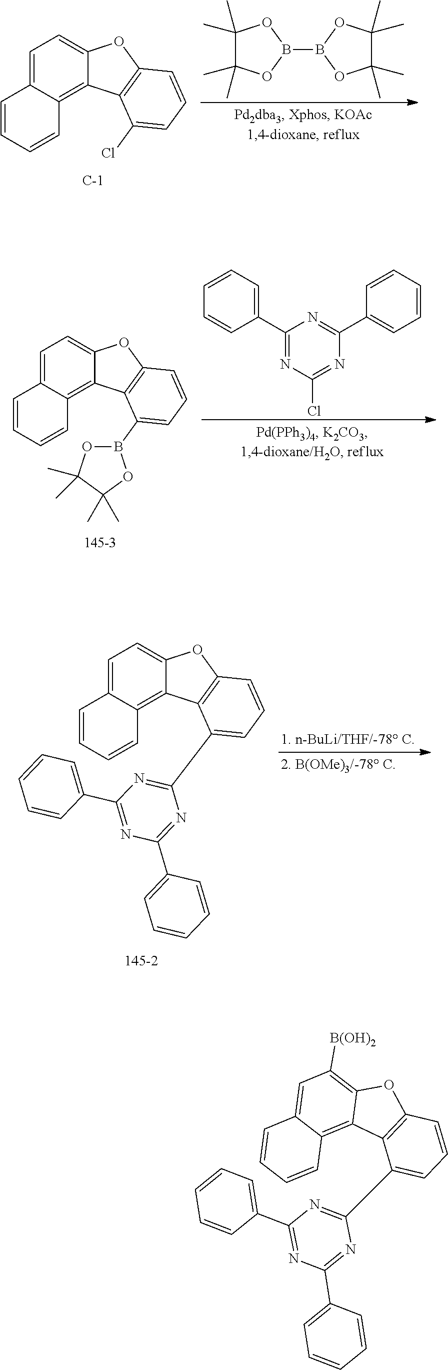

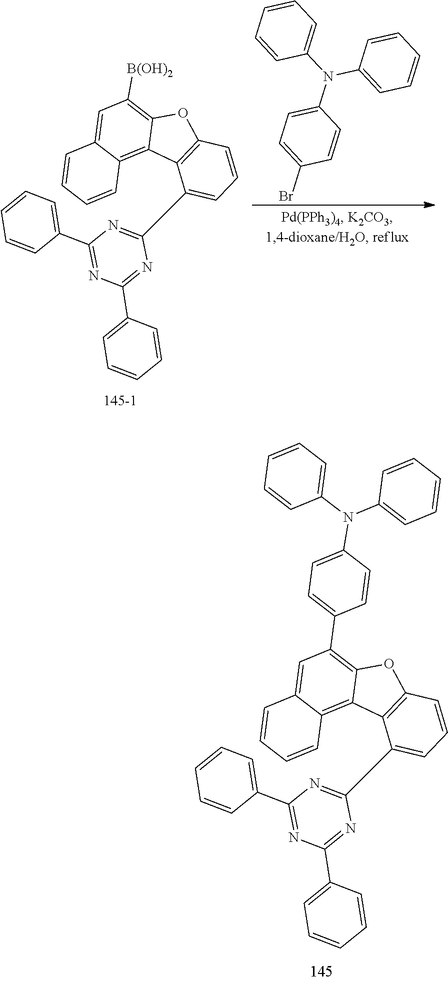 [SMILES: OB(O)c1cc2ccccc2c2c1oc1cccc(-c3nc(-c4ccccc4)nc(-c4ccccc4)n3)c12 ; Brc1ccc(N(c2ccccc2)c2ccccc2)cc1 ; c1ccc(-c2nc(-c3ccccc3)nc(-c3cccc4oc5c(-c6ccc(N(c7ccccc7)c7ccccc7)cc6)cc6ccccc6c5c34)n2)cc1]

### 1) Preparation of Compound 145-3

After dissolving Compound C-1 (10 g, 39.57 mmol), bis(pinacolato)diboron (4,4,4′,4′,5,5,5′,5′-octamethyl-2,2′-bi-1,3,2-dioxaborolane) (12.06 g, 47.49 mmol), Pd₂(dba)₃ (1.81 g, 1.98 mmol), Xphos (1.89 g, 3.96 mmol) and KOAc (7.77 g, 79.15 mmol) in 1,4-dioxane (100 mL), the mixture was refluxed for 12 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:2) to obtain target Compound 145-3 (8.58 g, 63%).

### 2) Preparation of Compound 145-2

After dissolving Compound 145-3 (8.58 g, 24.93 mmol), 2-chloro-4,6-diphenyl-1,3,5-triazine (7.34 g, 27.42 mmol), Pd(pph₃)₄ (1.44 g, 1.25 mmol) and K₂CO₃ (6.89 g, 49.85 mmol) in 1,4-dioxane/H₂O (100 mL/20 mL), the mixture was refluxed for 3 hours. After the reaction was completed, produced solids were filtered, washed with distilled water, and dried. The dried solids were boiled and dissolved in DCB, purified by silica, and the solvent was removed using a rotary evaporator. The result was recrystallized with acetone to obtain target Compound 145-2 (9.19 g, 82%).

### 3) Preparation of Compound 145-1

After dissolving Compound 145-2 (9.19 g, 20.44 mmol) in THF (120 ml) under nitrogen substitution, the temperature was lowered to −78° C. After dropping 2.5 M n-BuLi (8.59 ml, 21.47 mmol) thereto, the temperature was raised to room temperature, and the result was reacted for 1 hour. The temperature was lowered again to −78° C., and after dropping B(OMe)₃ (2.74 mL, 24.53 mmol, d: 0.932 g/ml) thereto, the temperature was raised to room temperature, and the result was reacted for 1 hour. After the reaction was completed, the reaction was terminated using MeOH, and after purifying the result by silica, the solvent was removed using a rotary evaporator. The result was recrystallized with EA and Hex to obtain target Compound 145-1 (5.24 g, 52%).

### 4) Preparation of Compound 145

After dissolving Compound 145-1 (5.24 g, 10.62 mmol), 4-bromo-N,N-diphenylaniline (3.79 g, 11.68 mmol), Pd(pph₃)₄ (0.61 g, 0.53 mmol) and K₂CO₃ (2.94 g, 21.24 mmol) in 1,4-dioxane/H₂O (50 mL/10 mL), the mixture was refluxed for 3 hours. After the reaction was completed, produced solids were filtered, washed with distilled water, and dried. The dried solids were boiled and dissolved in DCB, purified by silica, and the solvent was removed using a rotary evaporator. The result was recrystallized with acetone to obtain target Compound 145 (2.58 g, 35%).

Target compounds were synthesized in the same manner as in Preparation Example 3 except that Intermediate A-3 of the following Table 3 was used instead of 2-chloro-4,6-diphenyl-1,3,5-triazine, and Intermediate B-3 of the following Table 3 was used instead of 4-bromo-N,N-diphenylaniline.

TABLE 3

| Compound | Intermediate | Intermediate | | |
|---|---|---|---|---|
| No. | A-3 | B-3 | Target Compound | Yield |
| 148 | 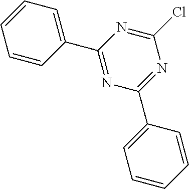 [SMILES: Clc1nc(-c2ccccc2)nc(-c2ccccc2)n1] | 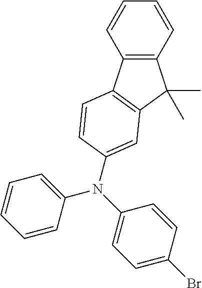 [SMILES: CC1(C)c2ccccc2-c2ccc(N(c3ccccc3)c3ccc(Br)cc3)cc21] | 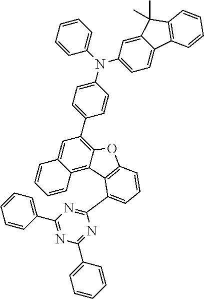 [SMILES: CC1(C)c2ccccc2-c2ccc(N(c3ccccc3)c3ccc(-c4cc5ccccc5c5c4oc4cccc(-c6nc(-c7ccccc7)nc(-c7ccccc7)n6)c45)cc3)cc21] | 40% |
| 151 | 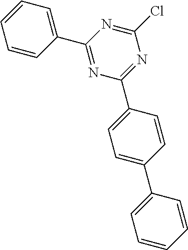 [SMILES: Clc1nc(-c2ccccc2)nc(-c2ccc(-c3ccccc3)cc2)n1] | 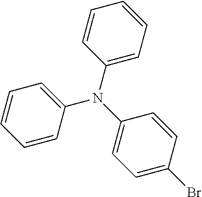 [SMILES: Brc1ccc(N(c2ccccc2)c2ccccc2)cc1] | 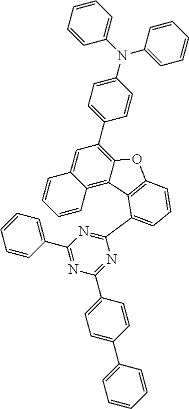 [SMILES: c1ccc(-c2ccc(-c3nc(-c4ccccc4)nc(-c4cccc5oc6c(-c7ccc(N(c8ccccc8)c8ccccc8)cc7)cc7ccccc7c6c45)n3)cc2)cc1] | 23% |
| 160 | 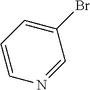 | 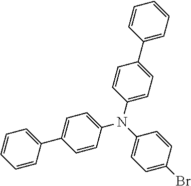 [SMILES: Brc1ccc(N(c2ccc(-c3ccccc3)cc2)c2ccc(-c3ccccc3)cc2)cc1] | 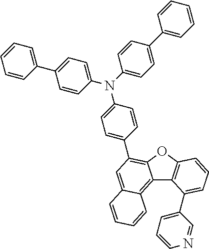 [SMILES: c1ccc(-c2ccc(N(c3ccc(-c4ccccc4)cc3)c3ccc(-c4cc5ccccc5c5c4oc4cccc(-c6cccnc6)c45)cc3)cc2)cc1] | 26% |
| 161 | 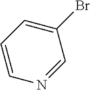 | 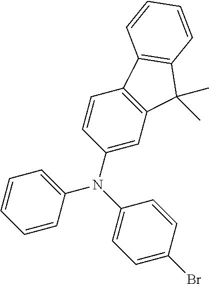 [SMILES: CC1(C)c2ccccc2-c2ccc(N(c3ccccc3)c3ccc(Br)cc3)cc21] | 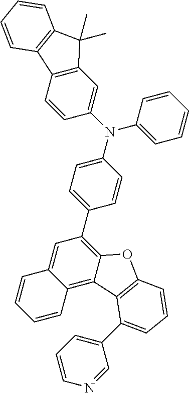 [SMILES: CC1(C)c2ccccc2-c2ccc(N(c3ccccc3)c3ccc(-c4cc5ccccc5c5c4oc4cccc(-c6cccnc6)c45)cc3)cc21] | 30% |
| 170 | 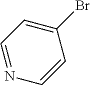 | 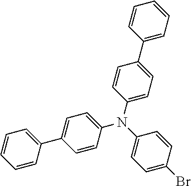 [SMILES: Brc1ccc(N(c2ccc(-c3ccccc3)cc2)c2ccc(-c3ccccc3)cc2)cc1] | 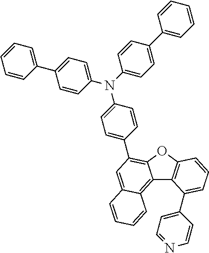 [SMILES: c1ccc(-c2ccc(N(c3ccc(-c4ccccc4)cc3)c3ccc(-c4cc5ccccc5c5c4oc4cccc(-c6ccncc6)c45)cc3)cc2)cc1] | 34% |
| 171 | 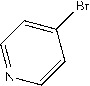 | 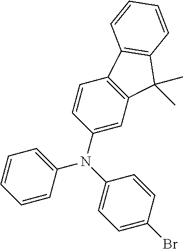 [SMILES: CC1(C)c2ccccc2-c2ccc(N(c3ccccc3)c3ccc(Br)cc3)cc21] | 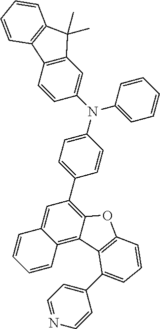 [SMILES: CC1(C)c2ccccc2-c2ccc(N(c3ccccc3)c3ccc(-c4cc5ccccc5c5c4oc4cccc(-c6ccncc6)c45)cc3)cc21] | 34% |
| 176 | 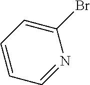 | 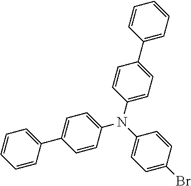 [SMILES: Brc1ccc(N(c2ccc(-c3ccccc3)cc2)c2ccc(-c3ccccc3)cc2)cc1] | 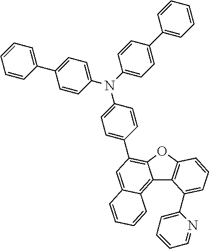 [SMILES: c1ccc(-c2ccc(N(c3ccc(-c4ccccc4)cc3)c3ccc(-c4cc5ccccc5c5c4oc4cccc(-c6ccccn6)c45)cc3)cc2)cc1] | 35% |
| 179 | 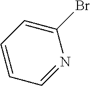 | 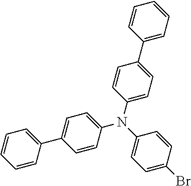 [SMILES: Brc1ccc(N(c2ccc(-c3ccccc3)cc2)c2ccc(-c3ccccc3)cc2)cc1] | 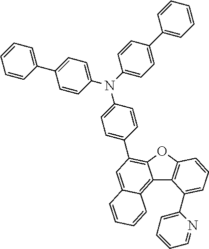 [SMILES: c1ccc(-c2ccc(N(c3ccc(-c4ccccc4)cc3)c3ccc(-c4cc5ccccc5c5c4oc4cccc(-c6ccccn6)c45)cc3)cc2)cc1] | 28% |
| 180 | 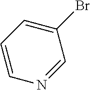 | 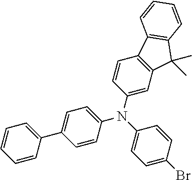 [SMILES: CC1(C)c2ccccc2-c2ccc(N(c3ccc(Br)cc3)c3ccc(-c4ccccc4)cc3)cc21] | 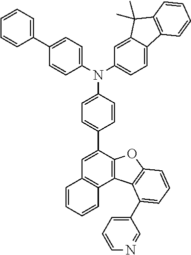 [SMILES: CC1(C)c2ccccc2-c2ccc(N(c3ccc(-c4ccccc4)cc3)c3ccc(-c4cc5ccccc5c5c4oc4cccc(-c6cccnc6)c45)cc3)cc21] | 26% |
| 183 | 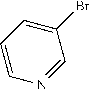 | 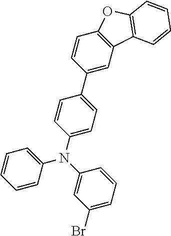 [SMILES: Brc1cccc(N(c2ccccc2)c2ccc(-c3ccc4oc5ccccc5c4c3)cc2)c1] | 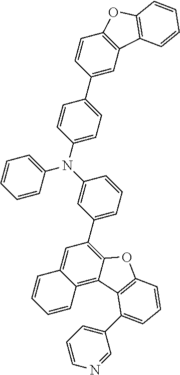 [SMILES: c1ccc(N(c2ccc(-c3ccc4oc5ccccc5c4c3)cc2)c2cccc(-c3cc4ccccc4c4c3oc3cccc(-c5cccnc5)c34)c2)cc1] | 29% |
| 191 | 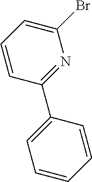 [SMILES: Brc1cccc(-c2ccccc2)n1] | 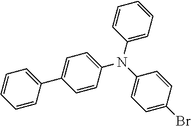 [SMILES: Brc1ccc(N(c2ccccc2)c2ccc(-c3ccccc3)cc2)cc1] | 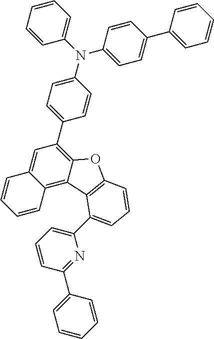 [SMILES: c1ccc(-c2ccc(N(c3ccccc3)c3ccc(-c4cc5ccccc5c5c4oc4cccc(-c6cccc(-c7ccccc7)n6)c45)cc3)cc2)cc1] | 16% |
| 194 | 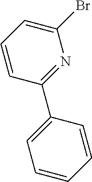 [SMILES: Brc1cccc(-c2ccccc2)n1] | 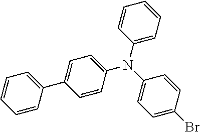 [SMILES: Brc1ccc(N(c2ccccc2)c2ccc(-c3ccccc3)cc2)cc1] | 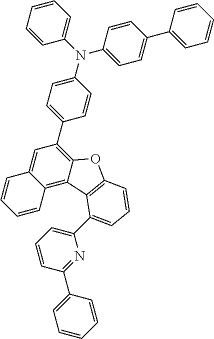 [SMILES: c1ccc(-c2ccc(N(c3ccccc3)c3ccc(-c4cc5ccccc5c5c4oc4cccc(-c6cccc(-c7ccccc7)n6)c45)cc3)cc2)cc1] | 20% |

Compounds described in the present specification were prepared in the same manner as in the preparation examples, and synthesis identification results for the prepared compounds are shown in the following Table 4 and Table 5. The following Table 4 shows measurement values of ¹H NMR (CDCl₃, 200 Mz), and the following Table 5 shows measurement values of FD-mass spectrometry (FD-MS: field desorption mass spectrometry).

TABLE 4

| Compound | ¹H NMR (CDCl₃, 200 Mz) |
|---|---|
| 1 | δ = 8.97(d, 1H), 8.36(d, 4H), 8.28(d, |
| | 1H), 8.03(s, 1H), 7.82(d, 1H), |
| | 7.69(d, 1H), 7.59~7.50(m, 9H), |
| | 7.24(d, 4H), 7.08-7.00(m, 6H) |
| 2 | δ = 8.97(d, 1H), 8.36(d, 3H), 8.28(d, |
| | 1H), 8.03(s, 1H), 7.82~7.69(m, |
| | 4H), 7.59~7.40(m, 13H), 7.24(d, |
| | 2H), 7.11-7.00(m, 4H) |
| 3 | δ = 8.97(d, 1H), 8.36(d, 4H), 8.28(d, |
| | 1H), 8.03(s, 1H), 7.82~7.69(m, |
| | 4H), 7.59~7.37(m, 16H), 7.24 |
| | (d, 2H), 7.08-7.00(m, 3H) |
| 5 | δ = 8.97(d, 1H), 8.36(d, 4H), 8.28(d, 1H), |
| | 8.03(s, 1H), 7.82~7.69(m, 6H), 7.59~7.37(m, 23H) |
| 8 | δ = 8.97(d, 1H), 8.36(d, 4H), 8.28 |
| | (d, 1H), 8.03(s, 1H), 7.90~7.82(m, |
| | 3H), 7.69(s, 1H), 7.59~7.50(m, 10H), |
| | 7.38~7.16(m, 6H), 7.08-7.00(m, 3H) |
| 13 | δ = 8.97(d, 2H), 8.36(d, 4H), 7.82 |
| | (d, 1H), 7.69(d, 1H), 7.57~7.50(m, |
| | 11H), 7.37~7.24(m, 7H), 7.08~7.00(m, 6H) |
| 14 | δ = 8.97(d, 2H), 8.36(d, 4H), 7.82~7.69(m, 4H), |
| | 7.57~7.24(m, 20H), 7.11~7.00(m, 4H) |
| 15 | δ = 8.97(d, 2H), 8.36(d, 4H), 7.82~7.69 |
| | (m, 4H), 7.57~7.24(m, 23H), 7.08~7.00(m, 3H) |
| 19 | δ = 8.97(d, 2H), 8.36(d, 4H), |
| | 7.90~7.82(m, 3H), 7.69(d, 1H), |
| | 7.57~~7.50(m, 12H), 7.38~7.16 |
| | (m, 9H), 7.08~7.00(m, 3H) |
| 22 | δ = 8.97(d, 2H), 8.36(d, 4H), 8.03~7.98 |
| | (m, 3H), 7.82~7.80(d, 2H), |
| | 7.69(d, 1H), 7.57~7.50(m, 11H), 7.39~ |
| | 7.17(m, 8H), 7.08~7.00(m, 3H), 6.91(d, 1H) |
| 24 | δ = 8.97(d, 2H), 8.36(d, 4H), |
| | 7.90~7.82(m, 3H), 7.69(d, 1H), |
| | 7.59~7.50(m, 11H), 7.38~7.17 |
| | (m, 10H), 7.08~7.00(m, 3H) |
| 26 | δ = 8.97(d, 1H), 8.36(d, 2H), |
| | 8.09~7.96(m, 7H), 7.82(d, 1H), |
| | 7.69~7.50(m, 10H), 7.38(d, 1H), |
| | 7.25~7.24(m, 6H), 7.08~7.00(m, 6H) |
| 28 | δ = 8.97(d, 1H), 8.36(d, 2H), 8.09~ |
| | 7.96(m, 7H), 7.82(d, 1H), |
| | 7.69~7.50(m, 10H), 7.38(d, 1H), |
| | 7.25~7.24(m, 6H), 7.08~7.00(m, 6H) |
| 32 | δ = 9.09(s, 1H), 8.97(d, 1H), 8.49 |
| | (d, 1H), 8.36(d, 2H), 8.16~8.00(m, |
| | 5H), 7.78~7.42(m, 16H), 7.24 |
| | (t, 2H), 7.11~7.00(m, 4H) |
| 37 | δ = 9.09(s, 1H), 8.97(d, 1H), 8.49 |
| | (d, 1H), 8.36(d, 2H), 8.16~8.00(m, |
| | 5H), 7.82~7.41(m, 19H), 7.24 |
| | (t, 2H), 7.08~7.00(m, 2H) |
| 39 | δ = 8.97(d, 1H), 8.36(d, 2H), 8.28~8.03 |
| | (m, 2H), 7.90~7.89(m, 2H), |
| | 7.82~7.75(m, 5H), 7.57~7.41 |
| | (m, 18H), 7.08~7.00(m, 3H) |
| 44 | δ = 9.09(s, 1H), 8.97(d, 1H), 8.49(d, |
| | 1H), 8.36(d, 2H), 8.16~7.98(m, |
| | 6H), 7.82(d, 1H), 7.69~7.50(m, 11H), |
| | 7.39~7.24(m, 5H), 7.08~6.97(m, 4H) |
| 48 | δ = 9.09(s, 1H), 8.97(d, 2H), 8.49 |
| | (d, 1H), 8.36(d, 2H), 8.18~8.00(m, |
| | 3H), 7.82(d, 1H), 7.69~7.50(m, 11H), |
| | 7.37~7.24(m, 7H), 7.08~7.00(m, 6H) |
| 51 | δ = 9.09(3, 1H), 8.97(d, 2H), 8.49 |
| | (d, 1H), 8.36(d, 2H), 8.16~8.00(m, |
| | 3H), 7.82(d, 1H), 7.69~7.50(m, |
| | 10H), 7.31~7.00(m, 14H) |
| 57 | δ = 8.97(d, 1H), 8.35(d, 2H), 8.23 |
| | (d, 1H), 8.17(d, 1H), 8.03(s, 1H), |
| | 7.94(d, 2H), 7.82~7.69(m, 6H), |
| | 7.57~7.37(m, 23H) |
| 60 | δ = 8.97(d, 1H), 8.23(s, 1H), |
| | 8.17(d, 1H), 8.03(s, 1H), 7.94~7.82(m, |
| | 7H), 7.69(d, 1H), 7.57~7.49(m, 10H), |
| | 7.38~7.24(m, 6H), 7.08~7.00(m, 3H) |
| 62 | δ = 8.97(d, 2H), 8.23(s, |
| | 1H), 7.94(d, 4H), 7.82~7.69 |
| | (m, 4H), 7.57~7.37(m, 23H), 7.08~7.00(m, 3H) |
| 65 | δ = 8.97(d, 2H), 8.23(s, 1H), |
| | 7.94~7.82(m, 7H), 7.69(d, 1H), |
| | 7.57~7.49(m, 11H), 7.38~7.24 |
| | (m, 10H), 7.08~7.00(m, 3H) |
| 70 | δ = 8.97(d, 1H), 8.23(s, 1H), 8.09~ |
| | 7.88(m, 3H), 7.94~7.89(m, 4H), |
| | 7.82~7.78(m, 2H), 7.69(d, 1H), |
| | 7.57~7.49(m, 7H), 7.38(t, 1H), |
| | 7.28~7.24(m, 5H), 7.08~7.00(m, 6H) |
| 72 | δ = 8.97(d, 1H), 8.23(s, 1H), |
| | 8.09~7.88(m, 3H), 7.94~7.78(m, 9H), |
| | 7.57~7.28(m, 18H), 7.08~7.00(m, 3H) |
| 79 | δ = 8.97(d, 2H), 8.36(d, 4H), 8.08~ |
| | 7.98(m, 5H), 7.82(d, 1H), 7.69(d, |
| | 1H), 7.57~7.50(m, 13H), 7.39~7.24 |
| | (m, 6H), 7.08~7.00(m, 3H) |
| 86 | δ = 9.24(s, 1H), 8.97(d, 1H), 8.70 |
| | (d, 1H), 8.42(d, 1H), 8.28(d, 1H), |
| | 8.03(s, 1H), 7.82~7.69(m, 4H), 7.59~ |
| | 7.37(m, 11H), 7.24(d, 2H), 7.08~7.00(m, 3H) |
| 88 | δ = 9.24(s, 1H), 8.97(d, 1H), |
| | 8.70(d, 1H), 8.42(d, 1H), 8.03(s, 1H), |
| | 7.82~7.69(m, 6H), 7.57~7.37(m, 18H) |
| 91 | δ = 9.24(3, 1H), 8.97(d, 1H), 8.70 |
| | (d, 1H), 8.42(d, 1H), 8.26(d, 1H), 8.03(s, |
| | 1H), 7.82~7.69(m, 6H), 7.57~7.37(m, 18H) |
| 92 | δ = 9.24(3, 1H), 8.97(d, 1H), 8.70(d, |
| | 1H), 8.42(d, 1H), 8.26(d, 1H), 8.03(s, 1H), |
| | 7.90~7.69(m, 6H), 7.57~7.28(m, 15H) |
| 95 | δ = 9.24(3, 1H), 8.97(d, 1H), |
| | 8.70(d, 1H), 8.42(d, 1H), 8.26(d, 1H), |
| | 8.03(s, 1H), 7.90~7.82(m, 3H), 7.69 |
| | (d, 1H), 7.59~7.50(m, 5H), |
| | 7.38~7.24(m, 6H), 7.08~7.00(m, 3H) |
| 96 | δ = 9.24(s, 1H), 8.97(d, 1H), 8.70 |
| | (d, 1H), 8.42(d, 1H), 8.26(d, 1H), |
| | 8.03(s, 1H), 7.90~7.82(m, 3H), 7.69 |
| | (d, 1H), 7.59~7.50(m, 5H), 7.38~7.00(m, 19H) |
| 99 | δ = 9.24(s, 1H), 8.97(d, 2H), 8.70(d, |
| | 1H), 8.42(d, 1H), 7.82~7.69(m, |
| | 4H), 7.59~7.24(m, 18H), 7.08~7.00(m, 3H) |
| 101 | δ = 9.24(s, 1H), 8.97(d, 2H), 8.70 |
| | (d, 1H), 8.42(d, 1H), 7.82~7.69(m, |
| | 6H), 7.59~7.37(m, 23H), 7.08~7.00(m, 3H) |
| 102 | δ = 9.24(s, 1H), 8.97(d, 2H), 8.70 |
| | (d, 1H), 8.42(d, 1H), 7.90~7.69(m, |
| | 6H), 7.59~7.28(m, 20H), 7.16(d, 1H) |
| 114 | δ = 8.97(d, 2H), 8.71(d, 2H), 8.08~8.00(m, 3H), |
| | 7.82~7.69(m, 6H), 7.59~7.37(m, 17H) |
| 116 | δ = 8.97(d, 2H), 8.71(d, 2H), 8.08~ |
| | 8.00(m, 3H), 7.90~7.69(m, 6H), |
| | 7.57~7.28(m, 14H), 7.16(d, 1H) |
| 117 | δ = 8.97(d, 2H), 8.71(d, 2H), 8.00(d, 2H), |
| | 7.82~7.69(m, 6H), 7.59~7.37(m, 22H) |
| 118 | δ = 8.97(d, 2H), 8.71(d, 2H), 8.00(d, 2H), |
| | 7.90~7.69(m, 6H), 7.59~7.28(m, 18H), 7.16(d, 1H) |
| 120 | δ = 9.24(d, 1H), 8.97(d, 2H), 8.70 |
| | (d, 1H), 8.42(d, 1H), 7.90~7.69(m, |
| | 6H), 7.59~7.28(m, 17H), 7.16~7.11(m, 2H) |
| 121 | δ = 9.24(d, 1H), 8.97(d, 2H), 8.70 |
| | (d, 1H), 8.42(d, 1H), 7.90~7.82(m, |
| | 3H), 7.69(d, 1H), 7.59~7.55(m, 7H), |
| | 7.38~7.24(m, 9H), 7.08~7.00(m, 3H), |
| 127 | δ = 8.97(d, 1H), 8.90(d, 1H), 8.33 |
| | (d, 2H), 8.28(d, 1H), 8.03~8.01(m, |
| | 2H), 7.90~7.69(m, 5H), 7.59~7.37 |
| | (m, 13H), 7.24(t, 2H), 7.08~7.00(m, 3H) |
| 129 | δ = 8.97(d, 1H), 8.90(d, 1H), 8.28~8.15 |
| | (m, 4H), 8.03(d, 2H), 7.81(d, 1H), |
| | 7.69~7.41(m, 13H), 7.24(t, 2H), 7.08~7.00(m, 3H) |
| 135 | δ = 8.97(d, 1H), 8.90(d, 1H), 8.33 |
| | (d, 2H), 8.26(d, 1H), 8.03~8.01(m, |
| | 2H), 7.90~7.82(m, 4H), 7.69(d, 1H), 7.59~7.49 |
| | (m, 7H), 7.38~7.24(m, 6H), 7.08~7.00(m, 3H) |
| 141 | δ = 9.34(d, 2H), 8.97(d, 2H), 8.43 |
| | (s, 2H), 7.95~7.85(m, 3H), 7.69(d, |
| | 1H), 7.59~7.24(m, 18H), 7.08~7.00(m, 3H) |
| 145 | δ = 8.36(d, 3H), 8.02~7.98(m, 2H), 7.82(d, 1H), |
| | 7.69(d, 1H), 7.59~7.50(m, 12H), 7.39~7.37 |
| | (m, 3H), 7.24(t, 3H), 7.08~7.00(m, 6H) |
| 148 | δ = 8.36(d, 4H), 7.90~7.82(m, 3H), |
| | 7.69(d, 1H), 7.59~7.50(m, 12H), |
| | 7.39~7.13(m, 9H), 7.08~7.00(m, 3H) |
| 151 | δ = 8.36(d, 2H), 7.96(d, 4H), 7.82~7.69(m, 4H), |
| | 7.59~7.37(m, 14H), |
| | 7.25(d, 2H), 7.24(t, 4H), 7.08~7.00(m, 6H) |
| 160 | δ = 9.24(d, 1H), 8.70(d, 1H), 8.02(d, 2H), 7.96 |
| | (d, 1H), 7.82~7.69(m, 6H), 7.59~7.37(m, 23H) |
| 161 | δ = 9.24(d, 1H), 8.70(d, 1H), |
| | 7.96(d, 1H), 7.90~7.82(m, 3H), 7.69(d, |
| | 1H), 7.59~7.55(m, 7H), 7.39~7.24 |
| | (m, 9H), 7.08~7.00(m, 3H) |
| 170 | δ = 8.71(d, 2H), 8.08~8.00(m, 4H), |
| | 7.82~7.69(m, 6H), 7.59~7.37(m, 22H) |
| 171 | δ = 8.71(d, 2H), 8.08~8.00(m, 4H), |
| | 7.90~7.82(m, 3H), 7.69(d, 1H), |
| | 7.59~7.55(m, 6H), 7.39~7.16 |
| | (m, 9H), 7.08~7.00(m, 3H) |
| 176 | δ = 8.37(d, 1H), 8.21(d, 1H), |
| | 8.02(d, 2H), 7.75~7.69(m, 5H), |
| | 7.59~7.37(m, 23H), 7.39(d, 1H), 6.90(t, 1H) |
| 179 | δ = 8.37(d, 1H), 8.21(d, 1H), |
| | 8.02(d, 2H), 7.75~7.69(m, 3H), |
| | 7.59~7.37(m, 16H), 7.24(d, 2H), |
| | 7.08~7.00(m, 3H), 6.90(t, 1H) |
| 180 | δ = 9.24(d, 1H), 8.70(d, 1H), 8.42 |
| | (d, 1H), 8.02(d, 2H), 7.90~7.69(m, |
| | 6H), 7.59~7.28(m, 20H), 7.16(d, 1H) |
| 183 | δ = 9.24(d, 1H), 8.70(d, 1H), 8.42 |
| | (d, 1H), 8.02(d, 2H), 7.88~7.82(m, |
| | 4H), 7.69(d, 1H), 7.59~7.55(m, 8H), |
| | 7.39~7.17(m, 10H), 7.08~7.00(m, 3H) |
| 191 | δ = 8.33(d, 2H), 8.21(d, 1H), |
| | 8.02~7.98(m, 2H), 7.75~7.69(m, 3H), |
| | 7.59~7.37(m, 18H), 7.24~7.18 |
| | (m, 3H), 7.08~7.00(m, 3H), 6.74(d, 2H) |
| 194 | δ = 8.90(d, 1H), 8.33(d, 2H), 8.02~ |
| | 7.98(m, 3H), 7.90~7.82(m, 4H), |
| | 7.69(d, 1H), 7.59~7.49(m, 9H), |
| | 7.39~7.24(m, 9H), 7.08~7.00(m, 3H) |
| 198 | δ = 8.97(d, 1H), 8.55(d, 1H), |
| | 8.36~8.28(m, 5H), 8.12(d, 1H), |
| | 8.11~7.98(m, 2H), 7.82~7.69 |
| | (m, 4H), 7.59~7.50(m, 11H), |
| | 7.40~7.35(d, 2H), 7.16(t, 1H) |
| 199 | δ = 8.97(d, 1H), 8.54(d, |
| | 2H), 8.36(d, 4H), 8.12(d, 1H), |
| | 7.99~7.74(m, 4H), 7.82(d, |
| | 1H), 7.69~7.50(m, 17H) |
| 207 | δ = 8.97(d, 1H), 8.55(d, 1H), 8.36~8.28 |
| | (m, 3H), 8.12(d, 2H), 7.96(d, |
| | 3H), 7.82~7.35(m, 19H), 7.25~7.16(d, 3H) |
| 217 | δ = 8.97(d, 2H), 8.55(d, 1H), |
| | 8.36~8.28(m, 5H), 8.11(d, 1H), |
| | 7.94~7.91(m, 5H), 7.82~7.69 |
| | (m, 4H), 7.59~7.50(d, 10H), |
| | 7.40~7.31(m, 3H), 7.16(t, 1H) |
| 218 | δ = 8.97(d, 2H), 8.55(d, 2H), 8.36 |
| | (d, 4H), 7.99~7.91(m, 5H), 7.82(d, |
| | 1H), 7.69~7.50(m, 14H), 7.35~ |
| | 7.31(m, 2H), 7.16(t, 1H) |
| 232 | δ = 9.24(d, 1H), 8.97(d, 1H), |
| | 8.70(d, 1H), 8.55(d, 1H), 8.42(d, 1H), |
| | 8.28(d, 1H), 8.12(d, 2H), 7.96 |
| | (d, 1H), 7.82~7.54(m, 10H), |
| | 7.40~7.35(m, 2H), 7.16(t, 1H) |
| 252 | δ = 8.97(d, 2H), 8.55(d, 1H), |
| | 8.36(d, 4H), 8.19(d, 1H), 7.94~7.91(m, |
| | 5H), 7.82(d, 1H), 7.69(d, 1H), |
| | 7.59~7.50(m, 11H), 7.35~ |
| | 7.31(m, 2H), 7.20~7.16(m, 2H) |
| 255 | δ = 8.97(d, 2H), 8.55(d, 1H), 8.36 |
| | (d, 4H), 8.31(d, 1H), 7.04~7.91(m, |
| | 6H), 7.82~7.69(m, 5H), 7.59~ |
| | 7.35(m, 14), 7.16(t, 1H) |

TABLE 5

| Compound | FD-Mass |
|---|---|
| 1 | m/z = 616.23(C43H28N4O, 616.72) |
| 2 | m/z = 666.24(C47H30N4O, 666.78) |
| 3 | m/z = 692.26(C49H32N4O, 692.82) |
| 4 | m/z = 716.26(C51H32N4O, 716.84) |
| 5 | m/z = 768.29(C55H36N4O, 768.92) |
| 6 | m/z = 666.24(C47H30N4O, 666.78) |
| 7 | m/z = 742.27(C53H34N4O, 742.88) |
| 8 | m/z = 732.29(C52H36N4O, 732.89) |
| 9 | m/z = 706.24(C49H30N4O2, 706. 81) |
| 10 | m/z = 722.21(C49H30N4OS,722.87) |
| 11 | m/z = 756.25(C53H32N4O2, 756.87) |
| 12 | m/z = 772.23(C53H32N4OS,772.93) |
| 13 | m/z = 692.26(C49H32N4O, 692.82) |
| 14 | m/z = 742.27(C53H34N4O,742.88) |
| 15 | m/z = 768.29(C55H36N4O, 768.92) |
| 16 | m/z = 768.29(C55H36N4O,768.92) |
| 17 | m/z = 782.27(C55H34N4O2, 782.90) |
| 18 | m/z = 798.25(C55H34N4OS, 798.96) |
| 19 | m/z = 808.32(C58H40N4O, 808.99) |
| 20 | m/z = 692.26(C49H32N4O, 692.82) |
| 21 | m/z = 742.27(C53H34N4O, 742.88) |
| 22 | m/z = 782.27(C55H34N4O2, 782.90) |
| 23 | m/z = 798.25(C55H34N4OS, 798.96) |
| 24 | m/z = 808.32(C58H40N4O, 808.99) |
| 25 | m/z = 666.24(C47H30N4O, 666.78) |
| 26 | m/z = 742.27(C53H34N4O, 742.88) |
| 27 | m/z = 716.26(C51H32N4O, 716.84) |
| 28 | m/z = 692.26(C49H32N4O, 692.82) |
| 29 | m/z = 706.24(C49H30N4O2, 706.81) |
| 30 | m/z = 722.21(C49H30N4OS, 722.87) |
| 31 | m/z = 732.29(C52H36N4O, 732.89) |
| 32 | m/z = 716.26(C51H32N4O, 716.84) |
| 33 | m/z = 792.29(C57H36N4O, 792.94) |
| 34 | m/z = 742.27(C53H34N4O, 742.88) |
| 35 | m/z = 782.30(C56H38N4O, 782.95) |
| 36 | m/z = 792.29(C57H36N4O, 792.94) |
| 37 | m/z = 742.27(C53H34N4O, 742.88) |
| 38 | m/z = 768.29(C55H36N4O, 768.92) |
| 39 | m/z = 808.32(C58H40N4O, 808.99) |
| 40 | m/z = 742.27(C53H34N4O, 742.88) |
| 41 | m/z = 782.27(C55H34N4O2, 782.90) |
| 42 | m/z = 808.32(C58H40N4O, 808.99) |
| 43 | m/z = 792.29(C57H36N4O, 792.94) |
| 44 | m/z = 756.25(C53H32N4O2, 756.87) |
| 45 | m/z = 782.30(C56H38N4O, 782.95) |
| 46 | m/z = 772.23(C53H32N4OS, 772.93) |
| 47 | m/z = 782.27(C55H34N4O2, 782.90) |
| 48 | m/z = 742.27(C53H34N4O, 742.88) |
| 49 | m/z = 768.29(C55H36N4O, 768.92) |
| 50 | m/z = 782.27(C55H34N4O2, 782.90) |
| 51 | m/z = 742.27(C53H34N4O, 742.88) |
| 52 | m/z = 798.25(C55H34N4OS, 798.96) |
| 53 | m/z = 615.23(C44H29N3O, 615.74) |
| 54 | m/z = 665.25(C48H31N3O, 665.80) |
| 55 | m/z = 691.26(C50H33N3O, 691.83) |
| 56 | m/z = 715.26(C52H33N3O, 715.86) |
| 57 | m/z = 767.29(C56H37N3O, 767.93) |
| 58 | m/z = 665.25(C48H31N3O, 665.80) |
| 59 | m/z = 741.28(C54H35N3O, 741.89) |
| 60 | m/z = 731.29(C53H37N3O, 731.90) |
| 61 | m/z = 691.26(C50H33N3O, 691.83) |
| 62 | m/z = 767.29(C56H37N3O, 767.93) |
| 63 | m/z = 781.27(C56H35N3O2, 781.91) |
| 64 | m/z = 741.28(C54H35N3O, 741.89) |
| 65 | m/z = 807.32(C59H41N3O, 808.00) |
| 66 | m/z = 797.25(C56H35N3OS, 797.98) |
| 67 | m/z = 665.25(C48H31N3O, 665.80) |
| 68 | m/z = 741.28(C54H35N3O, 741.89) |
| 69 | m/z = 741.28(C54H35N3O, 741.89) |
| 70 | m/z = 731.29(C53H37N3O, 731.90) |
| 71 | m/z = 741.28(C54H35N3O, 741.89) |
| 72 | m/z = 807.32(C59H41N3O, 808.00) |
| 73 | m/z = 781.31(C57H39N3O, 781.96) |
| 74 | m/z = 755.26(C54H33N3O2, 755.88) |
| 75 | m/z = 781.31(C57H39N3O, 781.96) |
| 76 | m/z = 781.27(C56H35N3O2, 781.91) |
| 77 | m/z = 797.25(C56H35N3OS, 797.98) |
| 78 | m/z = 797.25(C56H35N3OS, 797.98) |
| 79 | m/z = 782.27(C55H34N4O2, 782.90) |
| 80 | m/z = 782.27(C55H34N4O2, 782.90) |
| 81 | m/z = 798.25(C55H34N4OS, 798.96) |
| 82 | m/z = 797.25(C56H35N3OS, 797.98) |
| 83 | m/z = 798.25(C55H34N4OS, 798.96) |
| 84 | m/z = 462.17(C33H22N2O, 462.55) |
| 85 | m/z = 512.19(C37H24N2O, 512.61) |
| 86 | m/z = 538.20(C39H26N2O, 538.65) |
| 87 | m/z = 562.20(C41H26N2O, 562.67) |
| 88 | m/z = 614.24(C45H30N2O, 614.75) |
| 89 | m/z = 512.19(C37H24N2O, 512.61) |
| 90 | m/z = 588.22(C43H28N2O, 588.71) |
| 91 | m/z = 628.25(C46H32N2O, 628.77) |
| 92 | m/z = 654.27(C48H34N2O, 654.81) |
| 93 | m/z = 618.18(C43H26N2OS, 618.75) |
| 94 | m/z = 628.22(C45H28N2O2, 628.73) |
| 95 | m/z = 578.24(C42H30N2O, 578.72) |
| 96 | m/z = 702.27(C52H34N2O, 702.86) |
| 97 | m/z = 538.20(C39H26N2O, 538.65) |
| 98 | m/z = 588.22(C43H28N2O, 588.71) |
| 99 | m/z = 614.24(C45H30N2O, 614.75) |
| 100 | m/z = 638.24(C47H30N2O, 638.77) |
| 101 | m/z = 690.27(C51H34N2O, 690.85) |
| 102 | m/z = 730.30(C54H38N2O, 730.91) |
| 103 | m/z = 720.22(C51H32N2OS, 720.89) |
| 104 | m/z = 694.21(C49H30N2OS, 694.85) |
| 105 | m/z = 678.23(C49H30N2O2, 678.79) |
| 106 | m/z = 664.25(C49H32N2O, 664.81) |
| 107 | m/z = 664.25(C49H32N2O, 664.81) |
| 108 | m/z = 628.22(C45H28N2O2, 628.73) |
| 109 | m/z = 628.22(C45H28N2O2, 628.73) |
| 110 | m/z = 644.19(C45H28N2OS, 644.79) |
| 111 | m/z = 644.19(C45H28N2OS, 644.79) |
| 112 | m/z = 628.22(C45H28N2O2, 628.73) |
| 113 | m/z = 644.19(C45H28N2OS, 644.79) |
| 114 | m/z = 614.24(C45H30N2O, 614.75) |
| 115 | m/z = 538.20(C39H26N2O, 538.65) |
| 116 | m/z = 654.27(C48H34N2O, 654.81) |
| 117 | m/z = 690.27(C51H34N2O, 690.85) |
| 118 | m/z = 730.30(C54H38N2O, 730.91) |
| 119 | m/z = 588.22(C43H28N2O, 588.71) |
| 120 | m/z = 704.28(C52H36N2O, 704.87) |
| 121 | m/z = 654.27(C48H34N2O, 654.81) |
| 122 | m/z = 644.19(C45H28N2OS, 644.79) |
| 123 | m/z = 628.22(C45H28N2O2, 628.73) |
| 124 | m/z = 654.27(C48H34N2O, 654.81) |
| 125 | m/z = 538.20(C39H26N2O, 538.65) |
| 126 | m/z = 588.22(C43H28N2O, 588.71) |
| 127 | m/z = 614.24(C45H30N2O, 614.75) |
| 128 | m/z = 740.28(C55H36N2O, 740.91) |
| 129 | m/z = 588.22(C43H28N2O, 588.71) |
| 130 | m/z = 704.28(C52H36N2O, 704.87) |
| 131 | m/z = 730.30(C54H38N2O, 730.91) |
| 132 | m/z = 644.19(C45H28N2OS, 644.79) |
| 133 | m/z = 628.22(C45H28N2O2, 628.73) |
| 134 | m/z = 778.30(C58H38N2O, 778.95) |
| 135 | m/z = 654.27(C48H34N2O, 654.81) |
| 136 | m/z = 664.25(C49H32N2O, 664.81) |
| 137 | m/z = 664.25(C49H32N2O, 664.81) |
| 138 | m/z = 690.27(C51H34N2O, 690.85) |
| 139 | m/z = 766.30(C57H38N2O, 766.94) |
| 140 | m/z = 664.25(C49H32N2O, 664.81) |
| 141 | m/z = 806.33(C60H42N2O, 807.01) |
| 142 | m/z = 720.22(C51H32N2OS, 720.89) |
| 143 | m/z = 704.25(C51H32N2O2, 704.83) |
| 144 | m/z = 730.30(C54H38N2O, 730.91) |
| 145 | m/z = 692.26(C49H32N4O, 692.82) |
| 146 | m/z = 692.26(C49H32N4O, 692.82) |
| 147 | m/z = 768.29(C55H36N4O, 768.92) |
| 148 | m/z = 808.32(C58H40N4O, 808.99) |
| 149 | m/z = 808.32(C58H40N4O, 808.99) |
| 150 | m/z = 742.27(C53H34N4O, 742.88) |
| 151 | m/z = 768.29(C55H36N4O, 768.92) |
| 152 | m/z = 742.27(C53H34N4O, 742.88) |
| 153 | m/z = 808.32(C58H40N4O, 808.99) |
| 154 | m/z = 691.26(C50H33N3O, 691.83) |
| 155 | m/z = 781.27(C56H35N3O2, 781.91) |
| 156 | m/z = 741.28(C54H35N3O, 741.89) |
| 157 | m/z = 741.28(C54H35N3O, 741.89) |
| 158 | m/z = 538.20(C39H26N2O, 538.65) |
| 159 | m/z = 588.22(C43H28N2O, 588.71) |
| 160 | m/z = 690.27(C51H34N2O, 690.85) |
| 161 | m/z = 654.27(C48H34N2O, 654.81) |
| 162 | m/z = 628.22(C45H28N2O2, 628.73) |
| 163 | m/z = 704.25(C51H32N2O2, 704.83) |
| 164 | m/z = 770.33(C57H42N2O, 770.98) |
| 165 | m/z = 654.27(C48H34N2O, 654.81) |
| 166 | m/z = 720.22(C51H32N2OS, 720.89) |
| 167 | m/z = 704.25(C51H32N2O2, 704.83) |
| 168 | m/z = 538.20(C39H26N2O, 538.65) |
| 169 | m/z = 588.22(C43H28N2O, 588.71) |
| 170 | m/z = 690.27(C51H34N2O, 690.85) |
| 171 | m/z = 654.27(C48H34N2O, 654.81) |
| 172 | m/z = 628.22(C45H28N2O2, 628.73) |
| 173 | m/z = 614.24(C45H30N2O, 614.75) |
| 174 | m/z = 538.20(C39H26N2O, 538.65) |
| 175 | m/z = 588.22(C43H28N2O, 588.71) |
| 176 | m/z = 690.27(C51H34N2O, 690.85) |
| 177 | m/z = 654.27(C48H34N2O, 654.81) |
| 178 | m/z = 628.22(C45H28N2O2, 628.73) |
| 179 | m/z = 614.24(C45H30N2O, 614.75) |
| 180 | m/z = 730.30(C54H38N2O, 730.91) |
| 181 | m/z = 654.27(C48H34N2O, 654.81) |
| 182 | m/z = 654.27(C48H34N2O, 654.81) |
| 183 | m/z = 704.25(C51H32N2O2, 704.83) |
| 184 | m/z = 704.25(C51H32N2O2, 704.83) |
| 185 | m/z = 720.22(C51H32N2OS, 720.89) |
| 186 | m/z = 704.25(C51H32N2O2, 704.83) |
| 187 | m/z = 720.20(C51H32N2OS, 720.89) |
| 188 | m/z = 720.22(C51H32N2OS, 720.89) |
| 189 | m/z = 664.25(C49H32N2O, 664.81) |
| 190 | m/z = 664.25(C49H32N2O, 664.81) |
| 191 | m/z = 690.27(C51H34N2O, 690.85) |
| 192 | m/z = 766.30(C57H38N2O, 766.94) |
| 193 | m/z = 780.31(C58H40N2O, 780.97) |
| 194 | m/z = 730.30(C54H38N2O, 730.91) |
| 195 | m/z = 780.29(C57H36N2O2, 780.93) |
| 196 | m/z = 704.25(C51H32N2O2, 704.83) |
| 197 | m/z = 720.22(C51H32N2OS, 720.89) |
| 198 | m/z = 664.23(C47H28N4O, 664.77) |
| 199 | m/z = 714.24(C51H30N4O, 714.83) |
| 200 | m/z = 779.27(C55H33N5O, 779.90) |
| 201 | m/z = 746.21(C51H30N4OS, 746.89) |
| 202 | m/z = 654.21(C45H26N4O2, 654.73) |
| 203 | m/z = 714.24(C51H30N4O, 714.83) |
| 204 | m/z = 714.24(C51H30N4O, 714.83) |
| 205 | m/z = 720.20(C49H28N4OS, 720.85) |
| 206 | m/z = 704.22(C49H28N4O2, 704.79) |
| 207 | m/z = 740.26(C53H32N4O, 740.87) |
| 208 | m/z = 740.26(C53H32N4O, 740.87) |
| 209 | m/z = 663.23(C48H29N3O, 663.78) |
| 210 | m/z = 663.23(C48H29N3O, 663.78) |
| 211 | m/z = 713.25(C52H31N3O, 713.84) |
| 212 | m/z = 713.25(C52H31N3O, 713.84) |
| 213 | m/z = 663.23(C48H29N3O, 663.78) |
| 214 | m/z = 713.25(C52H31N3O, 713.84) |
| 215 | m/z = 713.25(C52H31N3O, 713.84) |
| 216 | m/z = 740.26(C53H32N4O, 740.87) |
| 217 | m/z = 740.26(C53H32N4O, 740.87) |
| 218 | m/z = 740.26(C53H32N4O, 740.87) |
| 219 | m/z = 740.26(C53H32N4O, 740.87) |
| 220 | m/z = 790.27(C57H34N4O, 790.93) |
| 221 | m/z = 790.27(C57H34N4O, 790.93) |
| 222 | m/z = 746.21(C51H30N4OS, 746.89) |
| 223 | m/z = 746.21(C51H30N4OS, 746.89) |
| 224 | m/z = 730.24(C51H30N4O2, 730.83) |
| 225 | m/z = 730.24(C51H30N4O2, 730.83) |
| 226 | m/z = 739.26(C54H33N3O, 739.88) |
| 227 | m/z = 739.26(C54H33N3O, 739.88) |
| 228 | m/z = 739.26(C54H33N3O, 739.88) |
| 229 | m/z = 739.26(C54H33N3O, 739.88) |
| 230 | m/z = 789.28(C58H35N3O, 789.94) |
| 231 | m/z = 789.28(C58H35N3O, 789.94) |
| 232 | m/z = 510.17(C37H22N2O, 510.60) |
| 233 | m/z = 510.17(C37H22N2O, 510.60) |
| 234 | m/z = 516.13(C35H20N2OS, 516.62) |
| 235 | m/z = 500.15(C35H20N2O2, 500.56) |
| 236 | m/z = 592.16(C41H24N2OS, 592.72) |
| 237 | m/z = 550.17(C39H22N2O2, 550.62) |
| 238 | m/z = 586.20(C43H26N2O, 586.69) |
| 239 | m/z = 586.20(C43H26N2O, 586.69) |
| 240 | m/z = 510.17(C37H22N2O, 510.60) |
| 241 | m/z = 510.17(C37H22N2O, 510.60) |
| 242 | m/z = 550.17(C39H22N2O2, 550.62) |
| 243 | m/z = 586.20(C43H26N2O, 586.69) |
| 244 | m/z = 586.20(C43H26N2O, 586.69) |
| 245 | m/z = 586.20(C43H26N2O, 586.69) |
| 246 | m/z = 576.22(C42H28N2O, 576.70) |
| 247 | m/z = 586.20(C43H26N2O, 586.69) |
| 248 | m/z = 586.20(C43H26N2O, 586.69) |
| 249 | m/z = 586.20(C43H26N2O, 586.69) |
| 250 | m/z = 636.22(C47H28N2O, 636.75) |
| 251 | m/z = 636.22(C47H28N2O, 636.75) |
| 252 | m/z = 690.24(C49H30N4O, 690.81) |
| 253 | m/z = 766.27(C55H34N4O, 766.90) |
| 254 | m/z = 766.27(C55H34N4O, 766.90) |
| 255 | m/z = 766.27(C55H34N4O, 766.90) |
| 256 | m/z = 690.24(C49H30N4O, 690.81) |

### Experimental Example

### Experimental Example 1

1) Manufacture of Organic Light Emitting Device (Rod Host)

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 500 Å using the following Compound A as a host and (piq)₂(Ir) (acac) as a red phosphorescent dopant by doping the (piq)₂(Ir) (acac) to the host in 3 wt %. After that, BCP was deposited to 60 Å as a hole blocking layer, and Alq₃ was deposited to 200 Å thereon as an electron transfer layer.

Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device (Comparative Example 1) was manufactured.

Meanwhile, all the organic compounds required to manufacture the OLED were vacuum sublimation purified under 10⁻⁸ torr to 10⁻⁶ torr for each material to be used in the OLED manufacture.

Organic light emitting devices of Comparative Examples 2 to 4 and Examples 1 to 65 were further manufactured in the same manner as in the process for manufacturing an organic light emitting device of Experimental Example 1 except that compounds listed in the following Table 6 were used instead of Compound A used as the host of the light emitting layer.

Specifically, the compounds used as the host of the light emitting layer in Examples 1 to 65 and Comparative Examples 1 to 4 are as shown in the following Table 6.

Herein, Compounds A to D of Comparative Examples 1 to 4 in the following Table 6 are as follows.

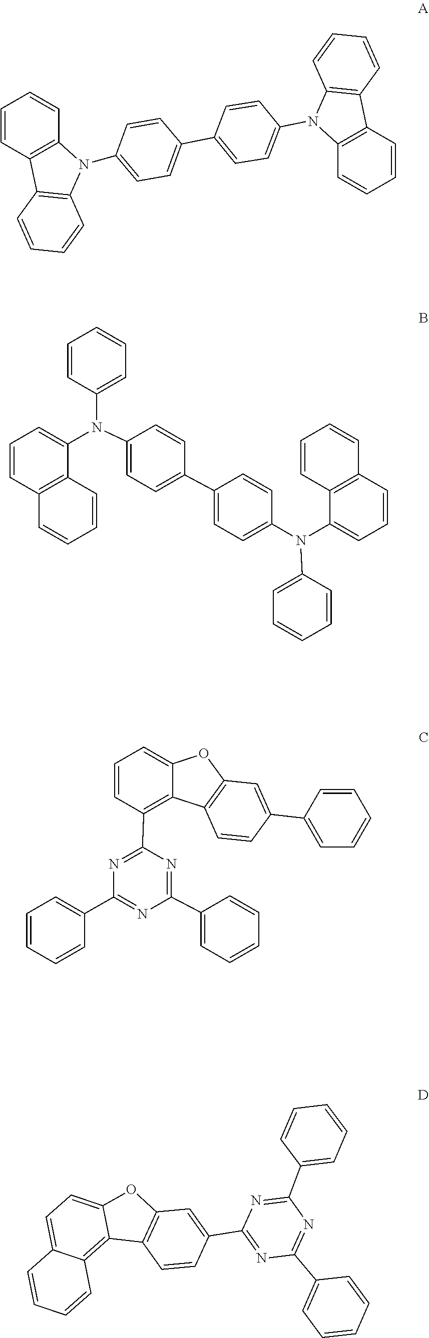 [SMILES: c1ccc2c(c1)c1ccccc1n2-c1ccc(-c2ccc(-n3c4ccccc4c4ccccc43)cc2)cc1 ; c1ccc(N(c2ccc(-c3ccc(N(c4ccccc4)c4cccc5ccccc45)cc3)cc2)c2cccc3ccccc23)cc1 ; c1ccc(-c2ccc3c(c2)oc2cccc(-c4nc(-c5ccccc5)nc(-c5ccccc5)n4)c23)cc1 ; c1ccc(-c2nc(-c3ccccc3)nc(-c3ccc4c(c3)oc3ccc5ccccc5c34)n2)cc1]

2) Driving Voltage and Light Emission Efficiency of Organic Light Emitting Device

For each of the organic light emitting devices of Examples 1 to 65 and Comparative Examples 1 to 4 manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc.

Measured properties of the organic light emitting devices of the present disclosure are as shown in the following Table 6.

TABLE 6

| | | Driving | | Color | Life- |
|---|---|---|---|---|---|
| | | Voltage | Efficiency | Coordinate | time |
| | Compound | (V) | (cd/A) | (x, y) | (T₉₀) |
| Comparative | A | 5.67 | 5.2 | (0.559, 0.350) | 11 |
| Example 1 | | | | | |
| Comparative | B | 6.77 | 4.8 | (0.660, 0.321) | 8 |
| Example 2 | | | | | |
| Comparative | C | 5.87 | 6.7 | (0.555, 0.335) | 25 |
| Example 3 | | | | | |
| Comparative | D | 4.97 | 7.1 | (0.660, 0.351) | 30 |
| Example 4 | | | | | |
| Example 1 | 1 | 3.56 | 15.4 | (0.659, 0.352) | 50 |
| Example 2 | 2 | 3.46 | 15.4 | (0.660, 0.352) | 25 |
| Example 3 | 3 | 3.53 | 14.7 | (0.661, 0.350) | 68 |
| Example 4 | 5 | 3.26 | 18.7 | (0.659, 0.349) | 76 |
| Example 5 | 8 | 3.79 | 13.5 | (0.668, 0.351) | 55 |
| Example 6 | 13 | 3.52 | 16.2 | (0.660, 0.350) | 67 |
| Example 7 | 14 | 3.49 | 16.8 | (0.661, 0.349) | 45 |
| Example 8 | 15 | 3.48 | 14.8 | (0.660, 0.348) | 70 |
| Example 9 | 19 | 4.06 | 12.5 | (0.659, 0.350) | 48 |
| Example 10 | 22 | 4.03 | 13.5 | (0.658, 0.351) | 59 |
| Example 11 | 24 | 4.10 | 10.9 | (0.660, 0.351) | 60 |
| Example 12 | 26 | 3.42 | 15.1 | (0.661, 0.350) | 66 |
| Example 13 | 28 | 3.77 | 16.5 | (0.660, 0.351) | 47 |
| Example 14 | 32 | 3.78 | 16.5 | (0.659, 0.349) | 69 |
| Example 15 | 37 | 3.60 | 15.5 | (0.658, 0.348) | 35 |
| Example 16 | 39 | 4.03 | 11.8 | (0.660, 0.351) | 42 |
| Example 17 | 44 | 3.51 | 17.5 | (0.661, 0.350) | 36 |
| Example 18 | 48 | 3.66 | 16.9 | (0.660, 0.350) | 55 |
| Example 19 | 51 | 4.03 | 14.9 | (0.658, 0.351) | 49 |
| Example 20 | 57 | 3.40 | 17.5 | (0.662, 0.349) | 68 |
| Example 21 | 60 | 3.59 | 14.9 | (0.657, 0.351) | 45 |
| Example 22 | 62 | 3.68 | 14.5 | (0.660, 0.351) | 55 |
| Example 23 | 65 | 3.55 | 15.1 | (0.660, 0.348) | 61 |
| Example 24 | 70 | 3.88 | 12.5 | (0.661, 0.352) | 49 |
| Example 25 | 72 | 3.71 | 13.4 | (0.658, 0.351) | 40 |
| Example 26 | 79 | 3.46 | 14.5 | (0.659, 0.348) | 38 |
| Example 27 | 86 | 3.55 | 15.5 | (0.661, 0.350) | 32 |
| Example 28 | 88 | 3.67 | 16.6 | (0.660, 0.350) | 42 |
| Example 29 | 91 | 3.51 | 15.1 | (0.662, 0.351) | 56 |
| Example 30 | 92 | 3.50 | 15.5 | (0.661, 0.352) | 55 |
| Example 31 | 95 | 4.02 | 12.5 | (0.661, 0.353) | 43 |
| Example 32 | 96 | 3.98 | 12.4 | (0.657, 0.350) | 49 |
| Example 33 | 99 | 3.46 | 14.9 | (0.657, 0.349) | 53 |
| Example 34 | 101 | 3.39 | 18.6 | (0.658, 0.349) | 54 |
| Example 35 | 102 | 4.00 | 12.5 | (0.661, 0.351) | 50 |
| Example 36 | 114 | 3.55 | 15.5 | (0.662, 0.350) | 58 |
| Example 37 | 116 | 3.46 | 16.1 | (0.660, 0.350) | 49 |
| Example 38 | 117 | 3.35 | 18.2 | (0.659, 0.350) | 75 |
| Example 39 | 118 | 4.01 | 12.5 | (0.661, 0.349) | 38 |
| Example 40 | 120 | 4.03 | 11.5 | (0.659, 0.350) | 62 |
| Example 41 | 121 | 3.89 | 12.6 | (0.662, 0.348) | 65 |
| Example 42 | 127 | 3.67 | 13.0 | (0.659, 0.350) | 64 |
| Example 43 | 129 | 3.88 | 15.5 | (0.662, 0.352) | 52 |
| Example 44 | 135 | 3.78 | 15.5 | (0.660, 0.351) | 53 |
| Example 45 | 141 | 3.89 | 16.6 | (0.659, 0.352) | 25 |
| Example 46 | 145 | 4.02 | 12.8 | (0.662, 0.350) | 55 |
| Example 47 | 148 | 4.07 | 12.9 | (0.661, 0.351) | 25 |
| Example 48 | 151 | 3.87 | 11.2 | (0.662, 0.350) | 56 |
| Example 49 | 160 | 3.45 | 17.8 | (0.660, 0.352) | 60 |
| Example 50 | 161 | 3.77 | 12.5 | (0.661, 0.349) | 26 |
| Example 51 | 170 | 3.40 | 16.4 | (0.660, 0.348) | 38 |
| Example 52 | 171 | 3.85 | 14.1 | (0.660, 0.351) | 44 |
| Example 53 | 176 | 3.50 | 12.7 | (0.657, 0.353) | 60 |
| Example 54 | 179 | 3.42 | 13.4 | (0.659, 0.350) | 55 |
| Example 55 | 180 | 3.56 | 12.7 | (0.661, 0.352) | 45 |
| Example 56 | 183 | 4.00 | 11.0 | (0.658, 0.353) | 41 |
| Example 56 | 191 | 3.59 | 15.5 | (0.659, 0.351) | 53 |
| Example 57 | 194 | 3.61 | 16.4 | (0.659, 0.350) | 36 |
| Example 58 | 198 | 3.89 | 18.1 | (0.662, 0.351) | 61 |
| Example 59 | 199 | 4.10 | 11.4 | (0.661, 0.350) | 42 |
| Example 60 | 207 | 4.15 | 12.1 | (0.661, 0.352) | 68 |
| Example 61 | 217 | 4.10 | 12.0 | (0.660, 0.348) | 32 |
| Example 62 | 218 | 4.09 | 11.6 | (0.660, 0.350) | 33 |
| Example 63 | 232 | 4.31 | 10.1 | (0.662, 0.349) | 45 |
| Example 64 | 252 | 4.20 | 11.0 | (0.661, 0.351) | 35 |
| Example 65 | 255 | 4.03 | 12.7 | (0.658, 0.350) | 25 |

From Experimental Example 1, it was identified that driving voltage and efficiency were improved when using the heterocyclic compound of Chemical Formula 1 as the host of the organic material layer, particularly the light emitting layer, of the organic light emitting device. Specifically, it was identified that, compared to Comparative Examples 1 to 4, Examples 1 to 65 using the heterocyclic compound of Chemical Formula 1 had an increased resonance effect as the benzene ring is extended in the central structure, and were thereby suitable as a red host. In addition, by having both a donor with a favorable hole transfer ability and an acceptor with a favorable electron transfer ability in one molecule and by fixing a substituent at a No. 11 position of the naphthobenzofuran, Examples 1 to 65 had a steric placement, and spatially separated HOMO (Highest Occupied Molecular Orbital) and LUMO (Lowest Unoccupied Molecular Orbital) allowing strong charge transfer, and high efficiency was expected when used as an organic material in the organic light emitting device.

In addition, when comparing Example 1 to Example 57 and Example 58 to Example 65, it was identified that, in Chemical Formula 1, a relatively low driving voltage was obtained when the hole unit corresponding to R₁ is an amine group compared to when the hole unit corresponding to R₁ is a carbazole group. The reason is considered to be due to the fact that hole mobility is relatively faster when the hole unit corresponding to R₁ is an amine group compared to when the hole unit corresponding to R₁ is a carbazole group, which enables light emission at a low voltage.

### Experimental Example 2

1) Manufacture of Organic Light Emitting Device (Red Host)

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 500 Å using a method of depositing heterocyclic compound 5 of the present disclosure as a first host and Compound 1-1 as a second host in one source of supply, and using (piq)₂(Ir) (acac) as a red phosphorescent dopant by doping the (piq)₂(Ir) (acac) to the host in 3 wt %. After that, BCP was deposited to 60 Å as a hole blocking layer, and Alq₃ was deposited to 200 Å thereon as an electron transfer layer.

Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device (Example 66) was manufactured.

Meanwhile, all the organic compounds required to manufacture the OLED were vacuum sublimation purified under 10⁻⁸ torr to 10⁻⁶ torr for each material to be used in the OLED manufacture.

Organic light emitting devices of Examples 66 to 113 were further manufactured in the same manner as in the process for manufacturing an organic light emitting device of Experimental Example 2 except that compounds listed in the following Table 7 were used instead of Compound 1-1 used as the second host of the light emitting layer.

Specifically, the compounds used as the first host and the second host of the light emitting layer in Examples 66 to 113 are as shown in the following Table 7.

2) Driving Voltage and Light Emission Efficiency of Organic Light Emitting Device

For each of the organic light emitting devices of Examples 66 to 113 manufactured as above, electroluminescent (EL) properties were measured using M7000 manufactured by McScience Inc., and with the measurement results, T₉₅ was measured when standard luminance was 6,000 cd/m² through a lifetime measurement system (M6000) manufactured by McScience Inc.

Measured properties of the organic light emitting devices of the present disclosure are as shown in the following Table 7.

Herein, in the following Table 7, Compounds 1-1 to 1-14 are as follows. The following Compounds 1-1 to 1-14 are p-host (p-type host) compounds having an excellent hole transfer ability.

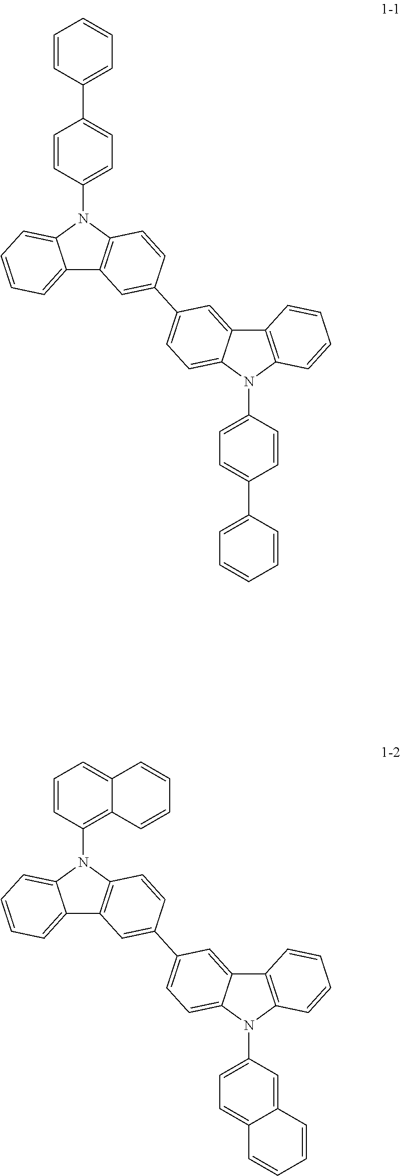 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

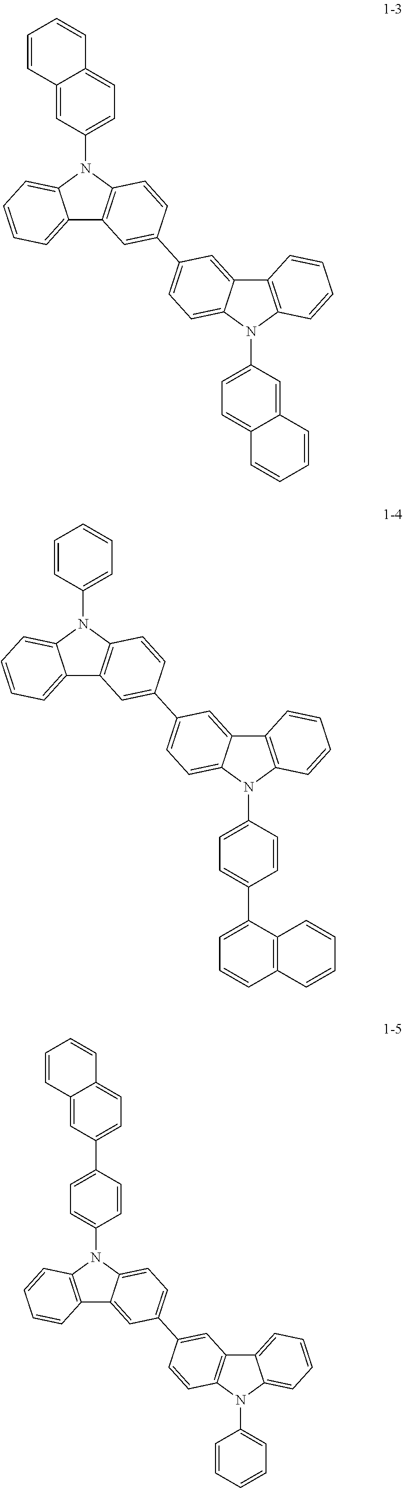 [SMILES: c1ccc2cc(-n3c4ccccc4c4cc(-c5ccc6c(c5)c5ccccc5n6-c5ccc6ccccc6c5)ccc43)ccc2c1 ; c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

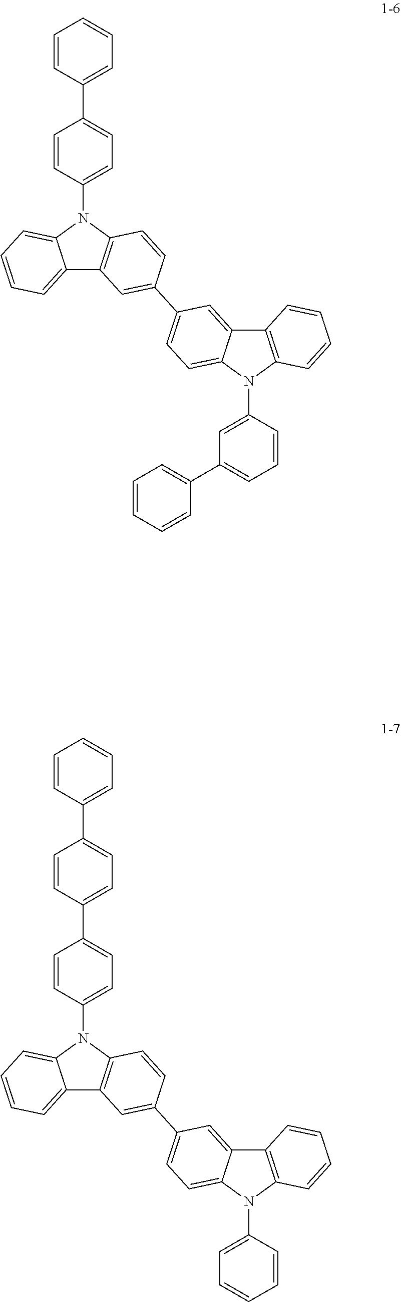 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

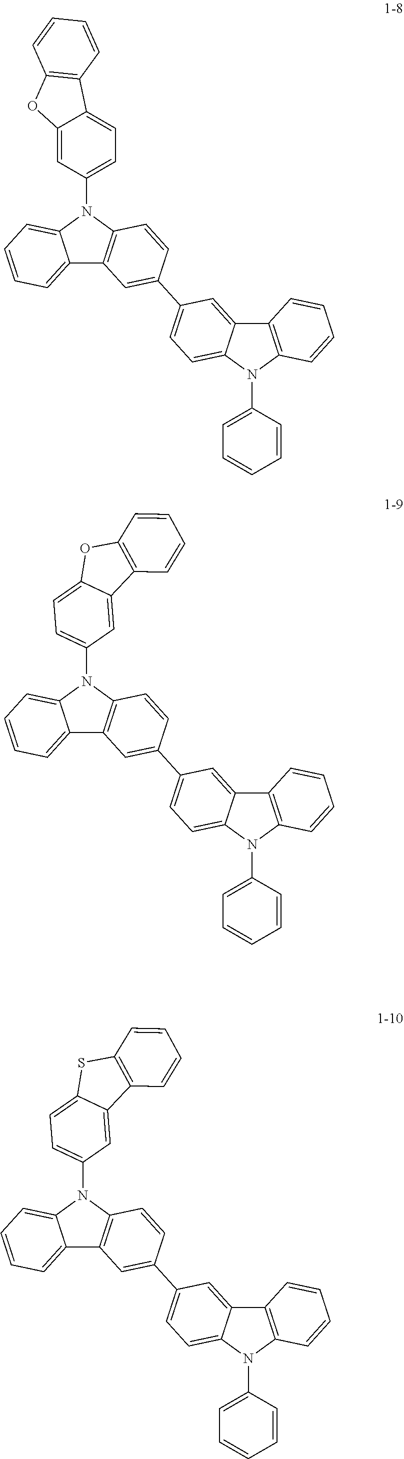 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

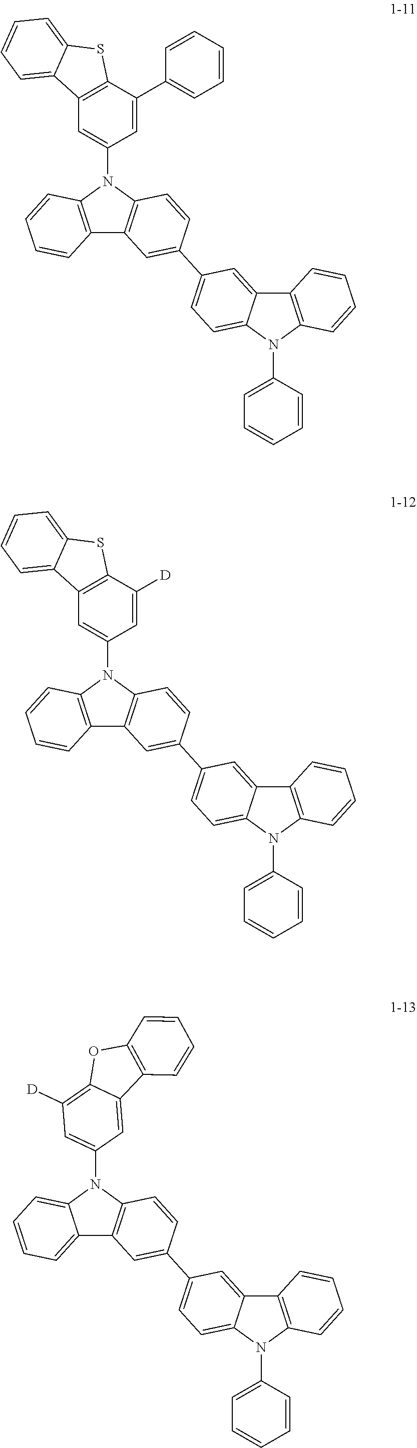 [SMILES: c1ccc(-c2cc(-n3c4ccccc4c4cc(-c5ccc6c(c5)c5ccccc5n6-c5ccccc5)ccc43)cc3c2sc2ccccc23)cc1 ; [2H]c1cc(-n2c3ccccc3c3cc(-c4ccc5c(c4)c4ccccc4n5-c4ccccc4)ccc32)cc2c1sc1ccccc12 ; [2H]c1cc(-n2c3ccccc3c3cc(-c4ccc5c(c4)c4ccccc4n5-c4ccccc4)ccc32)cc2c1oc1ccccc12]

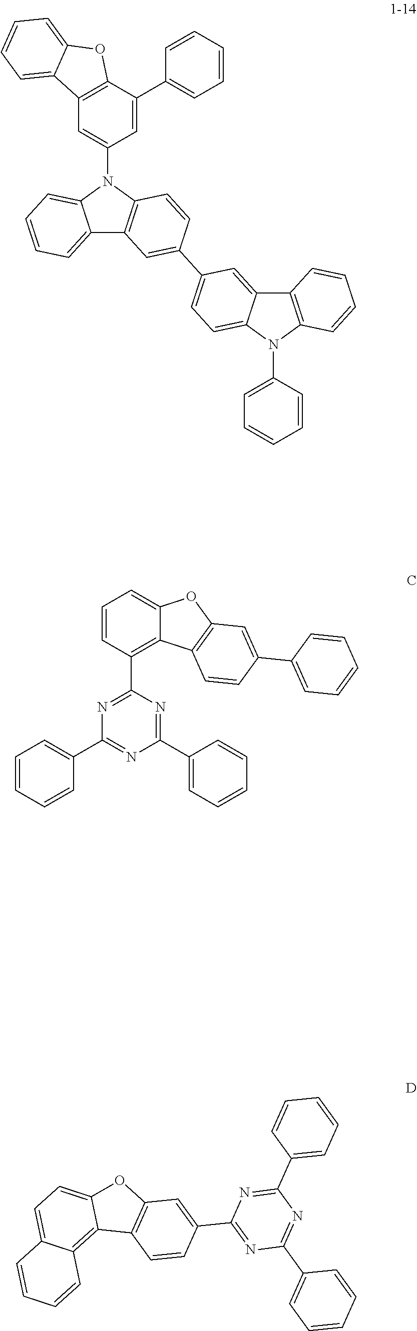 [SMILES: c1ccc(-c2cc(-n3c4ccccc4c4cc(-c5ccc6c(c5)c5ccccc5n6-c5ccccc5)ccc43)cc3c2oc2ccccc23)cc1 ; c1ccc(-c2ccc3c(c2)oc2cccc(-c4nc(-c5ccccc5)nc(-c5ccccc5)n4)c23)cc1 ; c1ccc(-c2nc(-c3ccccc3)nc(-c3ccc4c(c3)oc3ccc5ccccc5c34)n2)cc1]

TABLE 7

| | First | Second | Driving | Efficiency | Lifetime |
|---|---|---|---|---|---|
| | Host | Host | Voltage (V) | (cd/A) | (T₉₅) |
| Comparative | C | 1-4 | 4.87 | 11.7 | 60 |
| Example 5 | | | | | |
| Comparative | D | 1-12 | 3.97 | 13.1 | 65 |
| Example 6 | | | | | |
| Example 66 | 5 | 1-1 | 3.26 | 25.6 | 150 |
| Example 67 | | 1-2 | 3.25 | 23.4 | 125 |
| Example 68 | | 1-3 | 3.21 | 22.2 | 110 |
| Example 69 | | 1-4 | 3.22 | 24.1 | 151 |
| Example 70 | | 1-5 | 3.45 | 16.1 | 62 |
| Example 71 | | 1-6 | 3.44 | 15.9 | 65 |
| Example 72 | | 1-7 | 3.42 | 15.4 | 50 |
| Example 73 | | 1-8 | 3.45 | 16.6 | 70 |
| Example 74 | | 1-9 | 3.46 | 15.5 | 72 |
| Example 75 | | 1-10 | 3.44 | 15.7 | 68 |
| Example 76 | | 1-11 | 3.42 | 15.8 | 50 |
| Example 77 | | 1-12 | 3.40 | 15.9 | 72 |
| Example 88 | | 1-13 | 3.42 | 16.0 | 75 |
| Example 89 | | 1-14 | 3.39 | 15.5 | 66 |
| Example 90 | 15 | 1-1 | 3.44 | 20.8 | 87 |
| Example 91 | | 1-4 | 3.43 | 18.6 | 126 |
| Example 92 | | 1-8 | 3.45 | 15.3 | 77 |
| Example 93 | | 1-11 | 3.47 | 14.4 | 71 |
| Example 94 | 22 | 1-2 | 4.01 | 18.5 | 97 |
| Example 95 | | 1-5 | 4.00 | 22.3 | 80 |
| Example 96 | | 1-9 | 3.98 | 20.4 | 78 |
| Example 97 | 32 | 1-3 | 3.67 | 22.5 | 96 |
| Example 98 | | 1-6 | 3.67 | 22.3 | 100 |
| Example 99 | | 1-9 | 3.54 | 20.4 | 94 |
| Example 100 | | 1-11 | 3.55 | 17.6 | 70 |
| Example 101 | 48 | 1-7 | 3.54 | 17.8 | 63 |
| Example 102 | | 1-11 | 3.55 | 18.2 | 56 |
| Example 103 | | 1-13 | 3.58 | 17.0 | 60 |
| Example 104 | | 1-14 | 3.71 | 16.9 | 61 |
| Example 105 | 92 | 1-5 | 3.44 | 18.4 | 80 |
| Example 106 | | 1-8 | 3.49 | 16.5 | 77 |
| Example 107 | | 1-10 | 3.51 | 15.5 | 60 |
| Example 108 | 160 | 1-7 | 3.44 | 17.8 | 66 |
| Example 109 | | 1-12 | 3.43 | 18.3 | 74 |
| Example 110 | | 1-14 | 3.42 | 17.4 | 71 |
| Example 111 | 198 | 1-4 | 3.64 | 22.1 | 99 |
| Example 112 | | 1-6 | 3.80 | 19.0 | 85 |
| Example 113 | | 1-8 | 3.75 | 18.7 | 63 |

From Experimental Example 2, it was identified that driving voltage and efficiency were able to be improved when using the heterocyclic compound of the present disclosure as the first host of the organic material layer, particularly the light emitting layer, of the organic light emitting device, and using a specific compound having an excellent hole transfer ability as the second host. Specifically, it was identified that, when using Compounds 1-1 to 1-14 having a hole transfer ability with the heterocyclic compound of the present disclosure, a phenomenon of performance degradation caused by accumulation of electrons generated from the heterocyclic compound of the present disclosure was reduced, which improved a lifetime problem.

### Experimental Example 3

1) Manufacture of Organic Light Emitting Device (Red Host)

A glass substrate on which indium tin oxide (ITO) was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. The light emitting layer was deposited to 500 Å using a method of depositing heterocyclic compound 14 of the present disclosure as a first host and Compound 2-1 as a second host in one source of supply, and using (piq)₂(Ir) (acac) as a red phosphorescent dopant by doping the (piq)₂(Ir) (acac) to the host in 3 wt %. After that, BCP was deposited to 60 Å as a hole blocking layer, and Alq₃ was deposited to 200 Å thereon as an electron transfer layer.

Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device (Example 114) was manufactured.

Meanwhile, all the organic compounds required to manufacture the OLED were vacuum sublimation purified under 10⁻⁸ torr to 10⁻⁶ torr for each material to be used in the OLED manufacture.

Organic light emitting devices of Examples 114 to 153 were further manufactured in the same manner as in the process for manufacturing an organic light emitting device of Experimental Example 3 except that compounds listed in the following Table 8 were used instead of Compound 14 used as the first host of the light emitting layer and Compound 2-1.

Specifically, the compounds used as the first host and the second host of the light emitting layer in Examples 114 to 153 are as shown in the following Table 8.

2) Driving Voltage and Light Emission Efficiency of Organic Light Emitting Device

For each of the organic light emitting devices of Examples 114 to 153 manufactured as above, electroluminescent (EL) properties were measured using M7000 manufactured by McScience Inc., and with the measurement results, T₉₅ was measured when standard luminance was 6,000 cd/m² through a lifetime measurement system (M6000) manufactured by McScience Inc.

Measured properties of the organic light emitting devices of the present disclosure are as shown in the following Table 8.

Herein, in the following Table 8, Compounds 2-1 to 2-4 are as follows. The following Compounds 2-1 to 2-4 are n-host (n-type host) compounds having an excellent electron transfer ability.

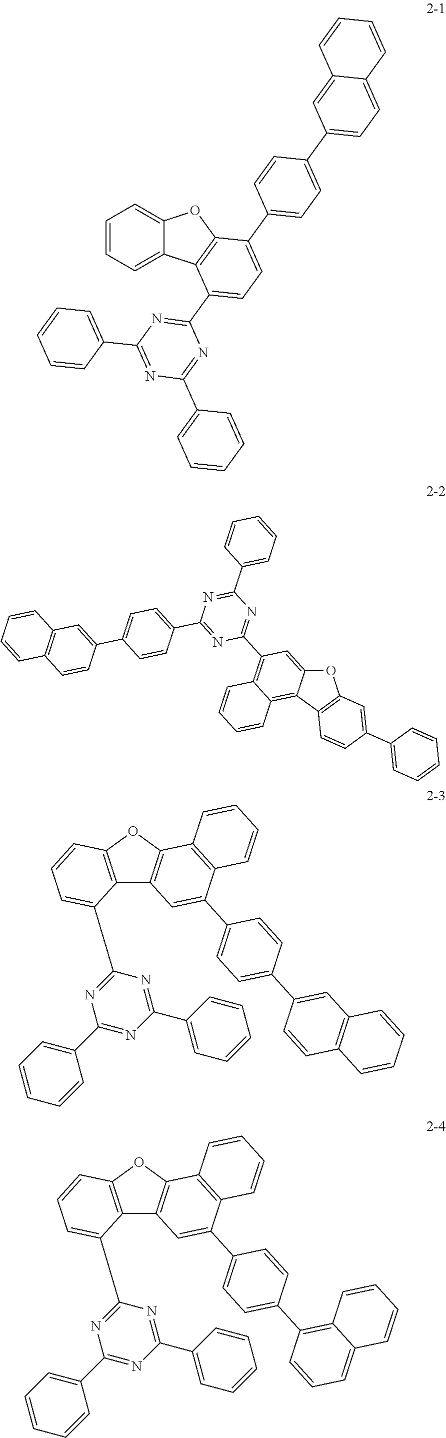 [SMILES: c1ccc(-c2nc(-c3ccccc3)nc(-c3ccc(-c4ccc(-c5ccc6ccccc6c5)cc4)c4oc5ccccc5c34)n2)cc1 ; c1ccc(-c2ccc3c(c2)oc2cc(-c4nc(-c5ccccc5)nc(-c5ccc(-c6ccc7ccccc7c6)cc5)n4)c4ccccc4c23)cc1 ; c1ccc(-c2nc(-c3ccccc3)nc(-c3cccc4oc5c6ccccc6c(-c6ccc(-c7ccc8ccccc8c7)cc6)cc5c34)n2)cc1 ; c1ccc(-c2nc(-c3ccccc3)nc(-c3cccc4oc5c6ccccc6c(-c6ccc(-c7cccc8ccccc78)cc6)cc5c34)n2)cc1]

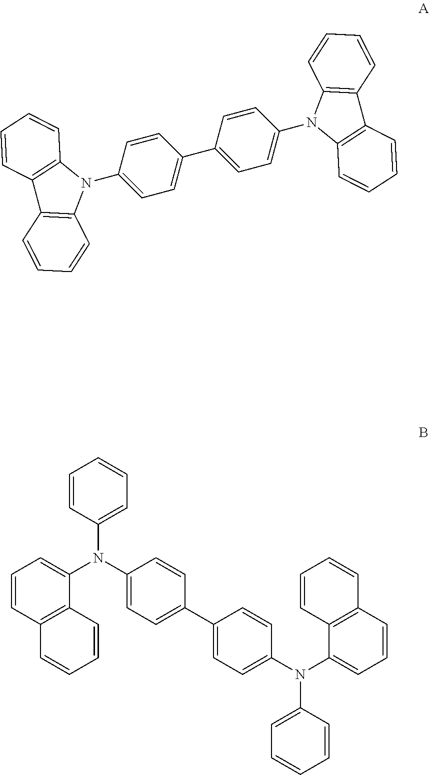 [SMILES: c1ccc2c(c1)c1ccccc1n2-c1ccc(-c2ccc(-n3c4ccccc4c4ccccc43)cc2)cc1 ; c1ccc(N(c2ccc(-c3ccc(N(c4ccccc4)c4cccc5ccccc45)cc3)cc2)c2cccc3ccccc23)cc1]

TABLE 8

| | First | Second | Driving | Efficiency | Lifetime |
|---|---|---|---|---|---|
| | Host | Host | Voltage (V) | (cd/A) | (T₉₅) |
| Comparative | A | 2-1 | 5.21 | 7.2 | 40 |
| Example 7 | | | | | |
| Comparative | B | 2-2 | 5.56 | 10.9 | 80 |
| Example 8 | | | | | |
| Example 114 | 14 | 2-1 | 3.35 | 22.8 | 125 |
| Example 115 | | 2-2 | 3.33 | 26.8 | 145 |
| Example 116 | | 2-3 | 3.34 | 20.7 | 138 |
| Example 117 | | 2-4 | 3.35 | 21.4 | 140 |
| Example 118 | 57 | 2-1 | 3.41 | 24.5 | 135 |
| Example 119 | | 2-2 | 3.38 | 25.5 | 146 |
| Example 120 | | 2-3 | 3.39 | 23.5 | 125 |
| Example 121 | | 2-4 | 3.40 | 24.5 | 130 |
| Example 122 | 88 | 2-1 | 3.55 | 16.6 | 115 |
| Example 123 | | 2-2 | 3.54 | 21.1 | 137 |
| Example 124 | | 2-3 | 3.60 | 20.3 | 126 |
| Example 125 | | 2-4 | 3.58 | 19.1 | 126 |
| Example 126 | 135 | 2-1 | 3.56 | 20.5 | 115 |
| Example 127 | | 2-2 | 3.58 | 25.5 | 147 |
| Example 128 | | 2-3 | 3.53 | 23.5 | 126 |
| Example 129 | | 2-4 | 3.60 | 21.5 | 130 |
| Example 130 | 160 | 2-1 | 3.42 | 20.4 | 120 |
| Example 131 | | 2-2 | 3.43 | 24.6 | 126 |
| Example 132 | | 2-3 | 3.45 | 22.3 | 115 |
| Example 133 | | 2-4 | 3.44 | 21.8 | 120 |
| Example 134 | 180 | 2-1 | 3.51 | 21.7 | 45 |
| Example 135 | | 2-2 | 3.50 | 22.7 | 100 |
| Example 136 | | 2-3 | 3.45 | 24.7 | 99 |
| Example 137 | | 2-4 | 3.56 | 20.7 | 89 |
| Example 138 | 48 | 2-1 | 3.54 | 20.4 | 101 |
| Example 139 | | 2-2 | 3.52 | 22.6 | 103 |
| Example 140 | | 2-3 | 3.62 | 21.4 | 111 |
| Example 141 | | 2-4 | 3.49 | 19.8 | 98 |
| Example 142 | 15 | 2-1 | 3.40 | 19.7 | 100 |
| Example 143 | | 2-2 | 3.41 | 18.1 | 112 |
| Example 144 | | 2-3 | 3.38 | 17.2 | 109 |
| Example 145 | | 2-4 | 3.40 | 19.0 | 107 |
| Example 146 | 37 | 2-1 | 3.54 | 20.4 | 70 |
| Example 147 | | 2-2 | 3.57 | 18.3 | 77 |
| Example 148 | | 2-3 | 3.60 | 19.1 | 70 |
| Example 149 | | 2-4 | 3.53 | 20.0 | 69 |
| Example 150 | 101 | 2-1 | 3.37 | 22.8 | 106 |
| Example 151 | | 2-2 | 3.34 | 24.0..6 | 114 |
| Example 152 | | 2-3 | 3.34 | 23.4 | 110 |
| Example 153 | | 2-4 | 3.35 | 22.9 | 99 |

From Experimental Example 3, it was identified that a device lifetime was able to be improved when using the heterocyclic compound of the present disclosure as the first host of the organic material layer, particularly the light emitting layer, of the organic light emitting device, and using a specific compound having an excellent electron transfer ability as the second host. Specifically, it was identified that, when using Compounds 2-1 to 2-4 having an electron transfer ability with the heterocyclic compound of the present disclosure, a charge balance in the light emitting layer was maximized enhancing light emitting properties.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

1. A heterocyclic compound represented by the following Chemical Formula 1: 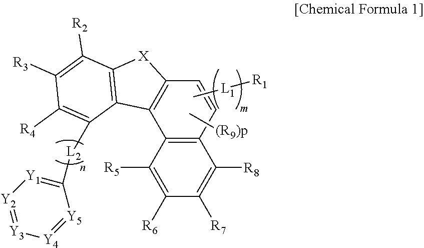
wherein, in Chemical Formula 1,
L₁ and L₂ are the same as or different from each other, and each independently a direct bond; a substituted or unsubstituted arylene group having 6 to 60 carbon atoms; or a substituted or unsubstituted heteroarylene group having 2 to 60 carbon atoms;
X is O; S; or NRa;
Y₁ to Y₅ are the same as or different from each other, and each independently N or CRb, at least one or more of Y₁ to Y₅ are N, and when there are two or more CRbs, Rbs are the same as or different from each other;
R₁ is represented by the following Chemical Formula A;
R₂ to R₉ are hydrogen; deuterium; a halogen group; a cyano group; a substituted or unsubstituted alkyl group; a substituted or unsubstituted cycloalkyl group; a substituted or unsubstituted aryl group having 6 to 40 carbon atoms; or a substituted or unsubstituted heteroaryl group having 2 to 40 carbon atoms;
Ra is a substituted or unsubstituted aryl group having 6 to 40 carbon atoms;
Rb is selected from the group consisting of hydrogen; deuterium; a substituted or unsubstituted aryl group having 6 to 60 carbon atoms; and a substituted or unsubstituted heteroaryl group having 2 to 60 carbon atoms, or two or more groups adjacent to each other bond to each other to form a substituted or unsubstituted aromatic hydrocarbon ring having 6 to 60 carbon atoms or a substituted or unsubstituted heteroring having 2 to 60 carbon atoms;
m and n are each independently an integer of 0 to 3, and when m and n are each 2 or greater, substituents in the parentheses are the same as or different from each other; and
p is 0 or 1, 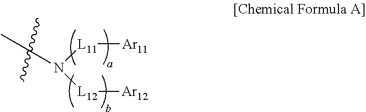
in Chemical Formula A,
L₁₁ and L₁₂ are the same as or different from each other, and each independently a direct bond; a substituted or unsubstituted arylene group having 6 to 40 carbon atoms; or a substituted or unsubstituted heteroarylene group having 2 to 40 carbon atoms;
Ar₁₁ and Ar₁₂ are the same as or different from each other, and each independently selected from the group consisting of a substituted or unsubstituted aryl group having 6 to 40 carbon atoms; and a substituted or unsubstituted heteroaryl group having 2 to 40 carbon atoms, or Ar₁₁ and Ar₁₂ bond to each other to form a substituted or unsubstituted aromatic hydrocarbon ring having 6 to 60 carbon atoms or a substituted or unsubstituted heteroring having 2 to 60 carbon atoms;
a and b are each 0 or 1; and 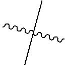
means a position bonding to L₁ of Chemical Formula 1.

2. The heterocyclic compound of claim 1, wherein the “substituted or unsubstituted” means being substituted with one or more substituents selected from the group consisting of a linear or branched alkyl group having 1 to 60 carbon atoms; a linear or branched alkenyl group having 2 to 60 carbon atoms; a linear or branched alkynyl group having 2 to 60 carbon atoms; a monocyclic or polycyclic cycloalkyl group having 3 to 60 carbon atoms; a monocyclic or polycyclic heterocycloalkyl group having 2 to 60 carbon atoms; a monocyclic or polycyclic aryl group having 6 to 60 carbon atoms; a monocyclic or polycyclic heteroaryl group having 2 to 60 carbon atoms; a silyl group; a phosphine oxide group; and an amine group, or being unsubstituted, or being substituted with a substituent linking two or more substituents selected from among the substituents illustrated above, or being unsubstituted.

3. The heterocyclic compound of claim 1, wherein Chemical Formula 1 is represented by the following Chemical Formula 2 or 3: 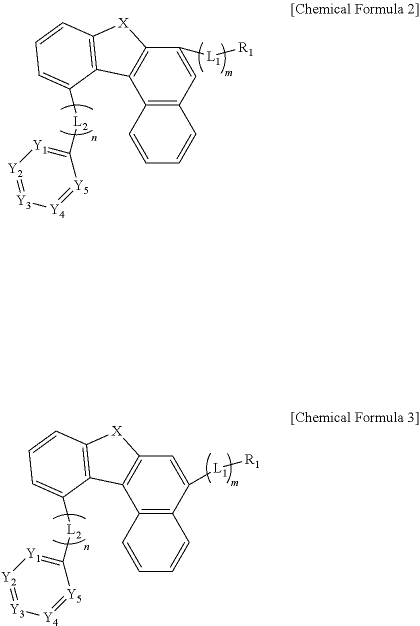
in Chemical Formulae 2 and 3,
each substituent has the same definition as in Chemical Formula 1.

4. The heterocyclic compound of claim 3, wherein Chemical Formula 2 is represented by any one of the following Chemical Formulae 2-1 to 2-6: 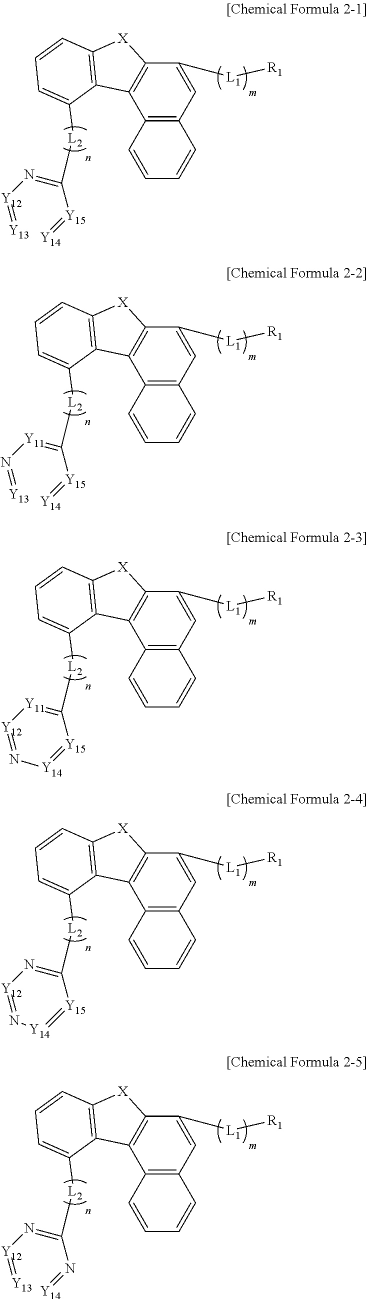 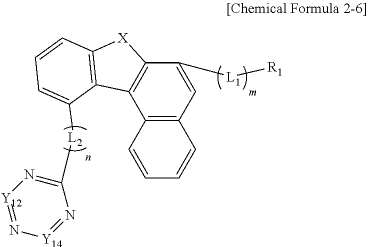
in Chemical Formulae 2-1 to 2-6,
L₁, L₂, X, R₁, m and n have the same definitions as in Chemical Formula 1; and
Y₁₁ to Y₁₅ are CRb, and Rb has the same definition as in Chemical Formula 1.

5. The heterocyclic compound of claim 3, wherein Chemical Formula 3 is represented by any one of the following Chemical Formulae 3-1 to 3-6: 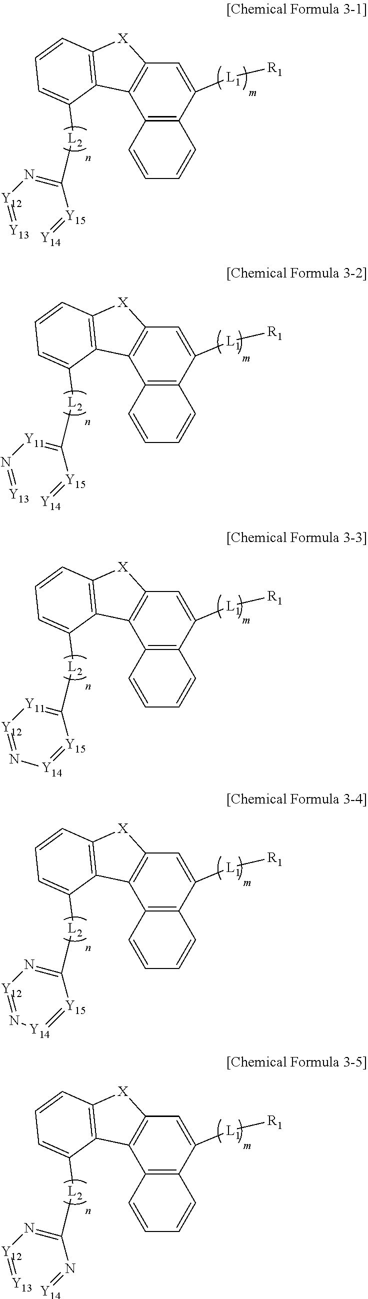 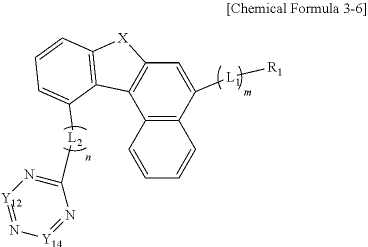
in Chemical Formulae 3-1 to 3-6,
L₁, L₂, X, R₁, m and n have the same definition as in Chemical Formula 1, Y₁₁ to Y₁₅ are CRb, and Rb has the same definition as in Chemical Formula 1.

6. The heterocyclic compound of claim 1, wherein Chemical Formula A is represented by any one of the following Chemical Formulae A-1 to A-5: 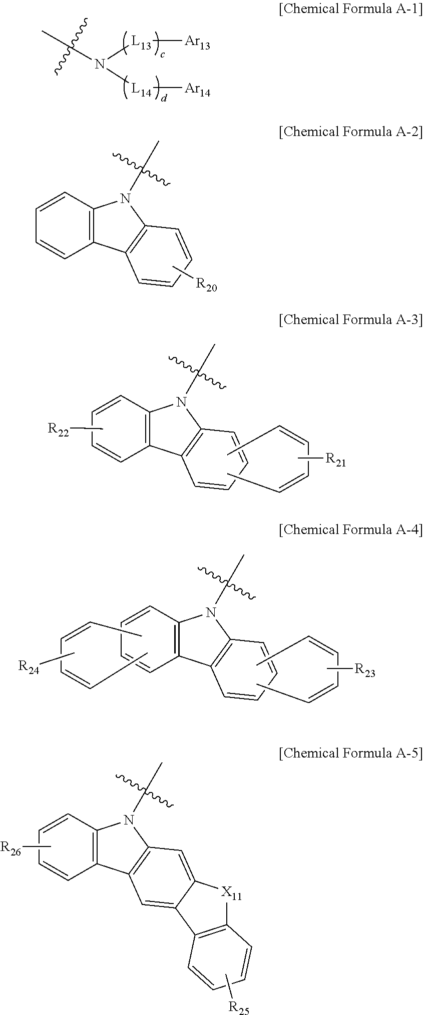
in Chemical Formulae A-1 to A-5,
L₁₃ and L₁₄ are the same as or different from each other, and each independently a direct bond; a substituted or unsubstituted arylene group having 6 to 40 carbon atoms; or a substituted or unsubstituted heteroarylene group having 2 to 40 carbon atoms;
Ar₁₃ and Ar₁₄ are the same as or different from each other, and each independently a substituted or unsubstituted aryl group having 6 to 40 carbon atoms; or a substituted or unsubstituted heteroaryl group having 2 to 40 carbon atoms;
R₂₀ to R₂₆, are each independently hydrogen; deuterium; a halogen group; a cyano group; a substituted or unsubstituted aryl group having 6 to 20 carbon atoms; or a substituted or unsubstituted heteroaryl group having 2 to 20 carbon atoms;
X₁₁ is O; S; or CRcRd, and Rc and Rd are the same as or different from each other and each independently a substituted or unsubstituted alkyl group having 1 to 10 carbon atoms;
c and d are each 0 or 1; and 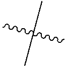
means a position bonding to L₁ of Chemical Formula 1.

7. The heterocyclic compound of claim 1, wherein Chemical Formula 1 is represented by any one of the following compounds:

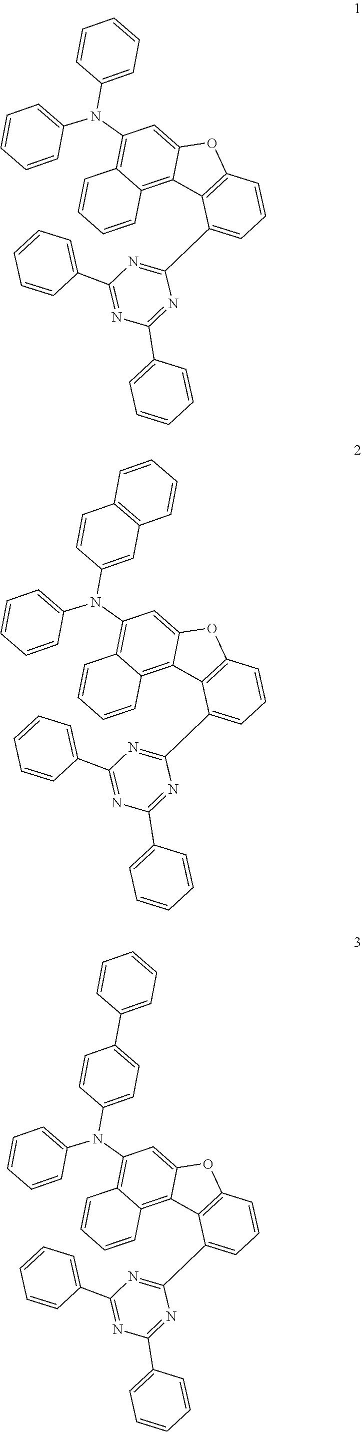

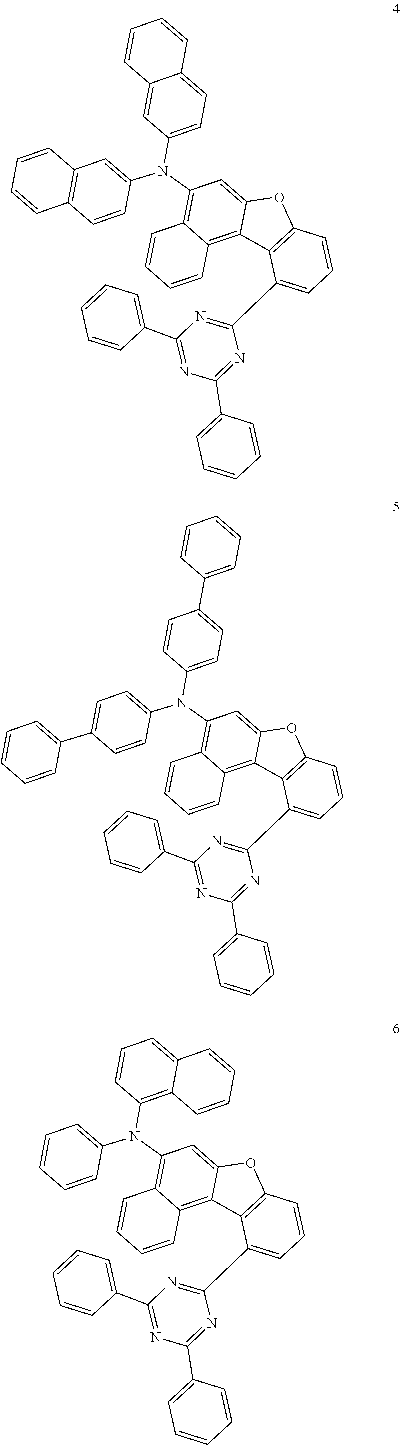

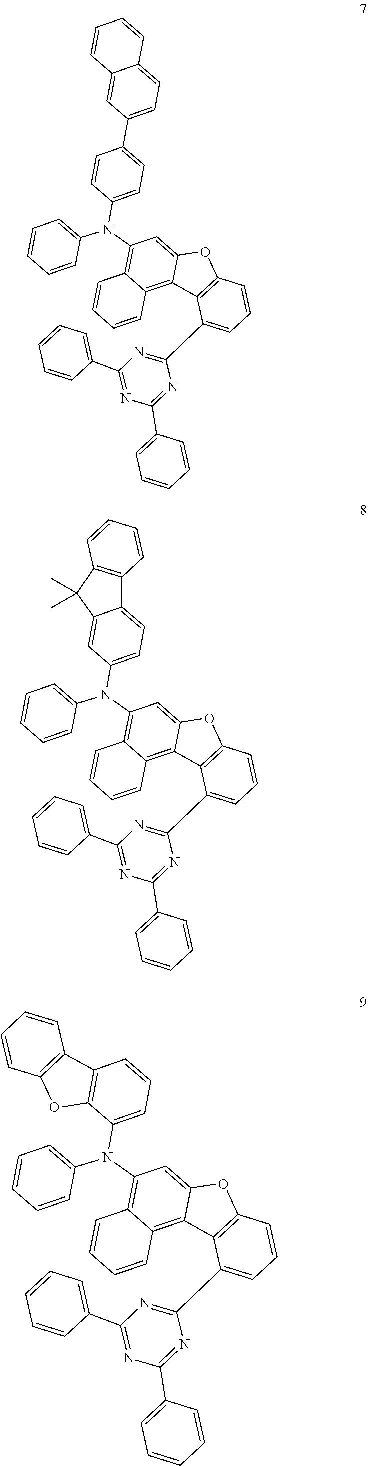 [SMILES: c1ccc(-c2nc(-c3ccccc3)nc(-c3cccc4oc5cc(N(c6ccccc6)c6ccc(-c7ccc8ccccc8c7)cc6)c6ccccc6c5c34)n2)cc1 ; CC1(C)c2ccccc2-c2ccc(N(c3ccccc3)c3cc4oc5cccc(-c6nc(-c7ccccc7)nc(-c7ccccc7)n6)c5c4c4ccccc34)cc21 ; c1ccc(-c2nc(-c3ccccc3)nc(-c3cccc4oc5cc(N(c6ccccc6)c6cccc7c6oc6ccccc67)c6ccccc6c5c34)n2)cc1]

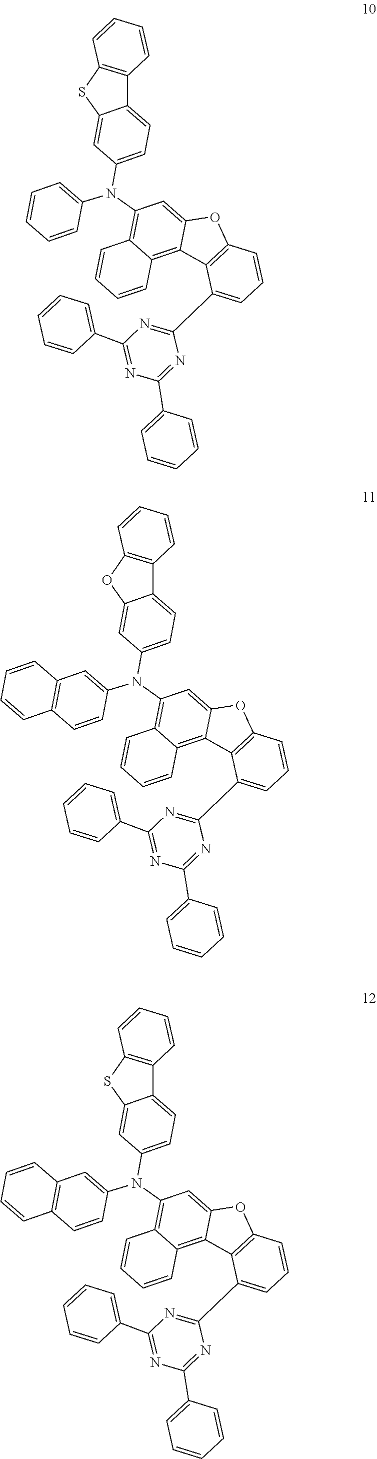 [SMILES: c1ccc(-c2nc(-c3ccccc3)nc(-c3cccc4oc5cc(N(c6ccccc6)c6ccc7c(c6)sc6ccccc67)c6ccccc6c5c34)n2)cc1 ; c1ccc(-c2nc(-c3ccccc3)nc(-c3cccc4oc5cc(N(c6ccc7ccccc7c6)c6ccc7c(c6)oc6ccccc67)c6ccccc6c5c34)n2)cc1 ; c1ccc(-c2nc(-c3ccccc3)nc(-c3cccc4oc5cc(N(c6ccc7ccccc7c6)c6ccc7c(c6)sc6ccccc67)c6ccccc6c5c34)n2)cc1]

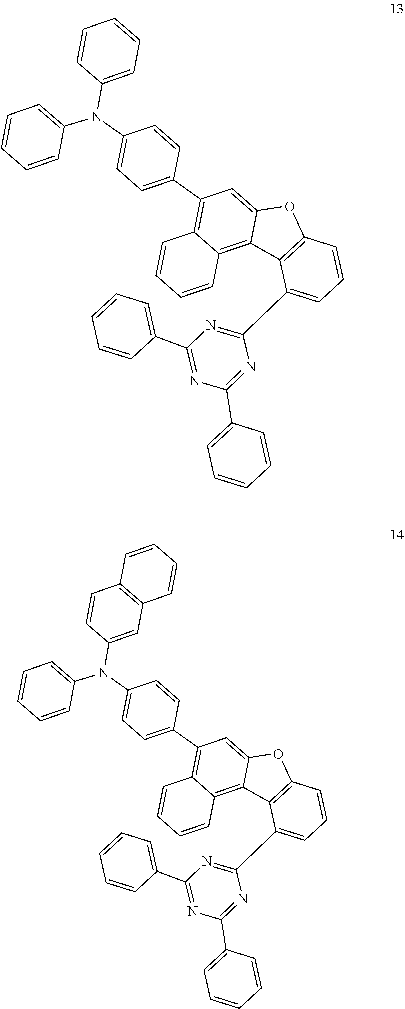 [SMILES: c1ccc(-c2nc(-c3ccccc3)nc(-c3cccc4oc5cc(-c6ccc(N(c7ccccc7)c7ccccc7)cc6)c6ccccc6c5c34)n2)cc1 ; c1ccc(-c2nc(-c3ccccc3)nc(-c3cccc4oc5cc(-c6ccc(N(c7ccccc7)c7ccc8ccccc8c7)cc6)c6ccccc6c5c34)n2)cc1]

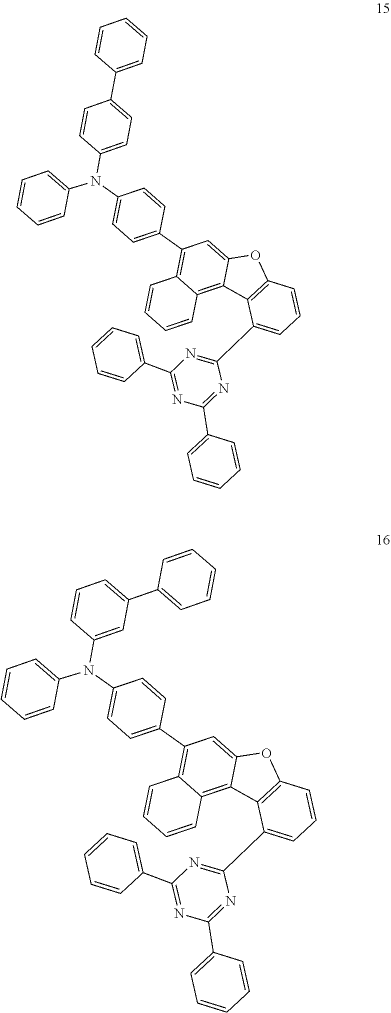 [SMILES: c1ccc(-c2ccc(N(c3ccccc3)c3ccc(-c4cc5oc6cccc(-c7nc(-c8ccccc8)nc(-c8ccccc8)n7)c6c5c5ccccc45)cc3)cc2)cc1 ; c1ccc(-c2cccc(N(c3ccccc3)c3ccc(-c4cc5oc6cccc(-c7nc(-c8ccccc8)nc(-c8ccccc8)n7)c6c5c5ccccc45)cc3)c2)cc1]

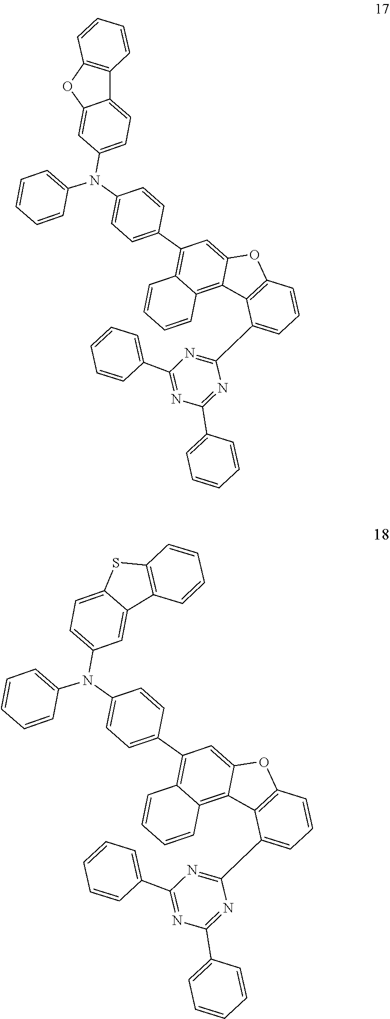 [SMILES: c1ccc(-c2nc(-c3ccccc3)nc(-c3cccc4oc5cc(-c6ccc(N(c7ccccc7)c7ccc8c(c7)oc7ccccc78)cc6)c6ccccc6c5c34)n2)cc1 ; c1ccc(-c2nc(-c3ccccc3)nc(-c3cccc4oc5cc(-c6ccc(N(c7ccccc7)c7ccc8sc9ccccc9c8c7)cc6)c6ccccc6c5c34)n2)cc1]

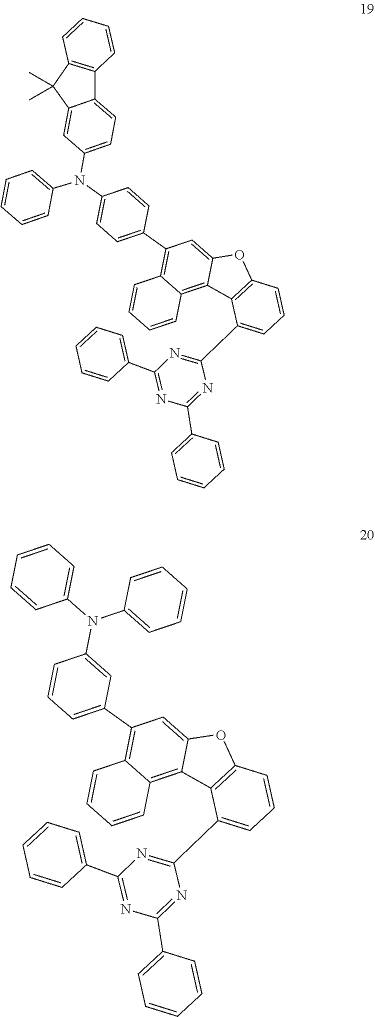 [SMILES: CC1(C)c2ccccc2-c2ccc(N(c3ccccc3)c3ccc(-c4cc5oc6cccc(-c7nc(-c8ccccc8)nc(-c8ccccc8)n7)c6c5c5ccccc45)cc3)cc21 ; c1ccc(-c2nc(-c3ccccc3)nc(-c3cccc4oc5cc(-c6cccc(N(c7ccccc7)c7ccccc7)c6)c6ccccc6c5c34)n2)cc1]

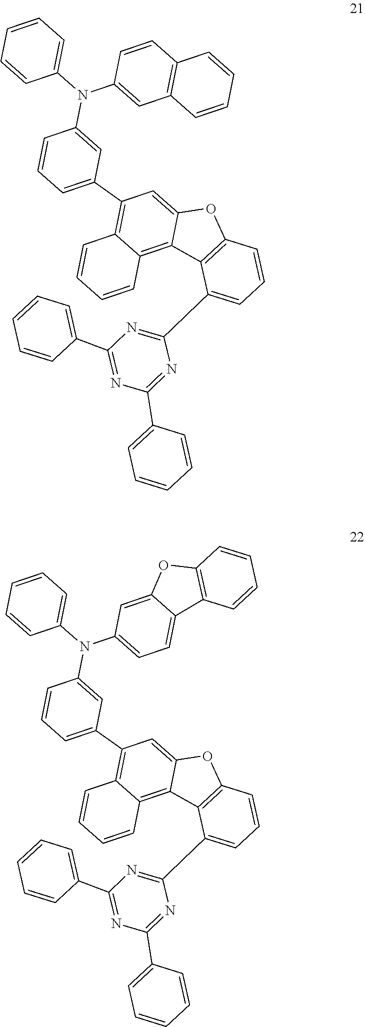 [SMILES: c1ccc(-c2nc(-c3ccccc3)nc(-c3cccc4oc5cc(-c6cccc(N(c7ccccc7)c7ccc8ccccc8c7)c6)c6ccccc6c5c34)n2)cc1 ; c1ccc(-c2nc(-c3ccccc3)nc(-c3cccc4oc5cc(-c6cccc(N(c7ccccc7)c7ccc8c(c7)oc7ccccc78)c6)c6ccccc6c5c34)n2)cc1]

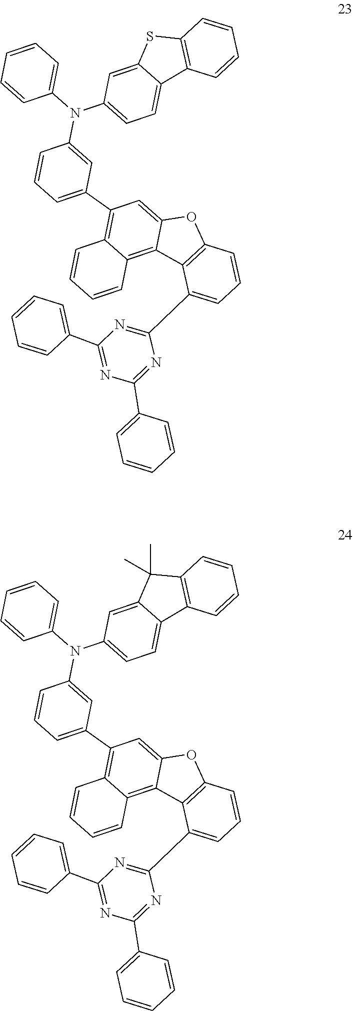 [SMILES: c1ccc(-c2nc(-c3ccccc3)nc(-c3cccc4oc5cc(-c6cccc(N(c7ccccc7)c7ccc8c(c7)sc7ccccc78)c6)c6ccccc6c5c34)n2)cc1 ; CC1(C)c2ccccc2-c2ccc(N(c3ccccc3)c3cccc(-c4cc5oc6cccc(-c7nc(-c8ccccc8)nc(-c8ccccc8)n7)c6c5c5ccccc45)c3)cc21]

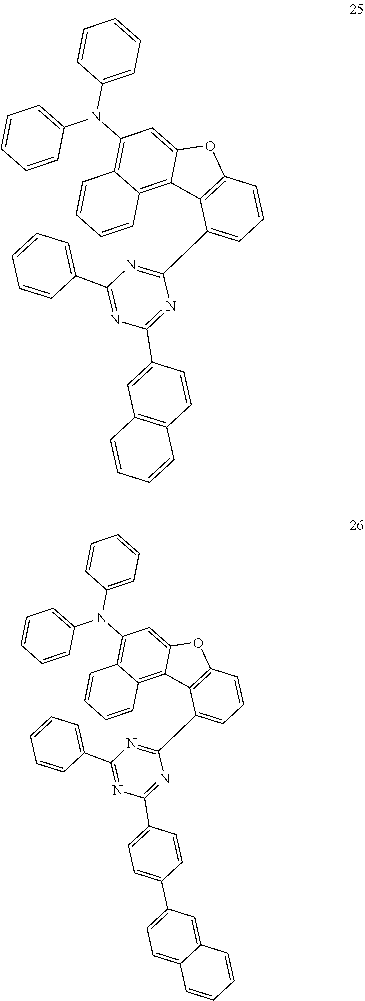 [SMILES: c1ccc(-c2nc(-c3ccc4ccccc4c3)nc(-c3cccc4oc5cc(N(c6ccccc6)c6ccccc6)c6ccccc6c5c34)n2)cc1 ; c1ccc(-c2nc(-c3ccc(-c4ccc5ccccc5c4)cc3)nc(-c3cccc4oc5cc(N(c6ccccc6)c6ccccc6)c6ccccc6c5c34)n2)cc1]

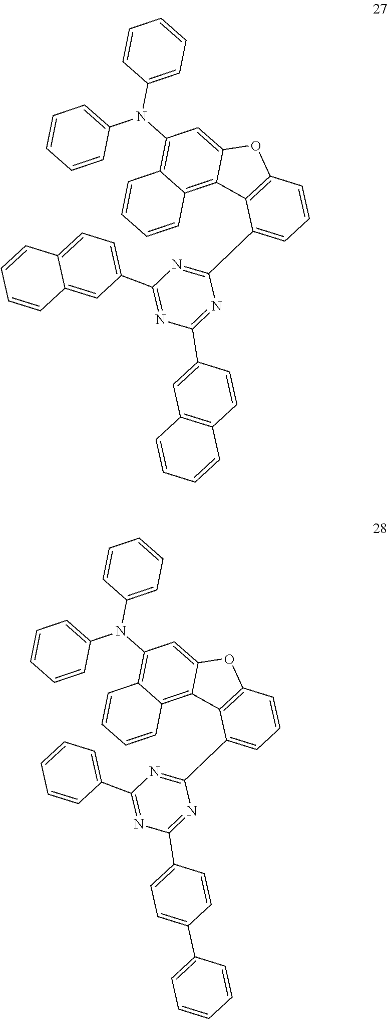 [SMILES: c1ccc(N(c2ccccc2)c2cc3oc4cccc(-c5nc(-c6ccc7ccccc7c6)nc(-c6ccc7ccccc7c6)n5)c4c3c3ccccc23)cc1 ; c1ccc(-c2ccc(-c3nc(-c4ccccc4)nc(-c4cccc5oc6cc(N(c7ccccc7)c7ccccc7)c7ccccc7c6c45)n3)cc2)cc1]

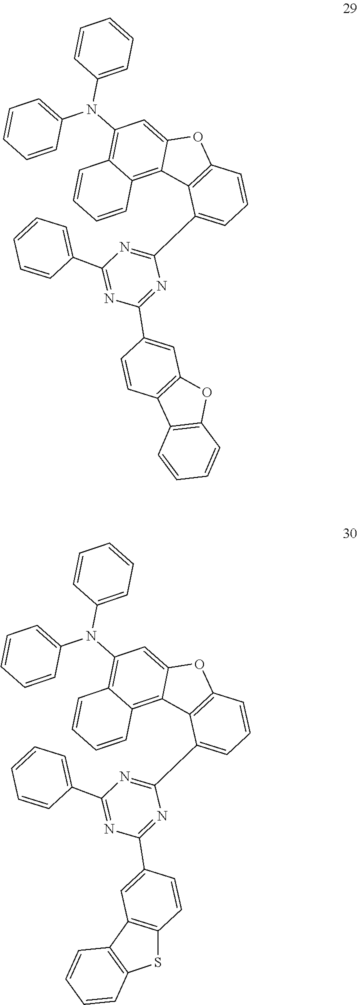 [SMILES: c1ccc(-c2nc(-c3ccc4c(c3)oc3ccccc34)nc(-c3cccc4oc5cc(N(c6ccccc6)c6ccccc6)c6ccccc6c5c34)n2)cc1 ; c1ccc(-c2nc(-c3ccc4sc5ccccc5c4c3)nc(-c3cccc4oc5cc(N(c6ccccc6)c6ccccc6)c6ccccc6c5c34)n2)cc1]

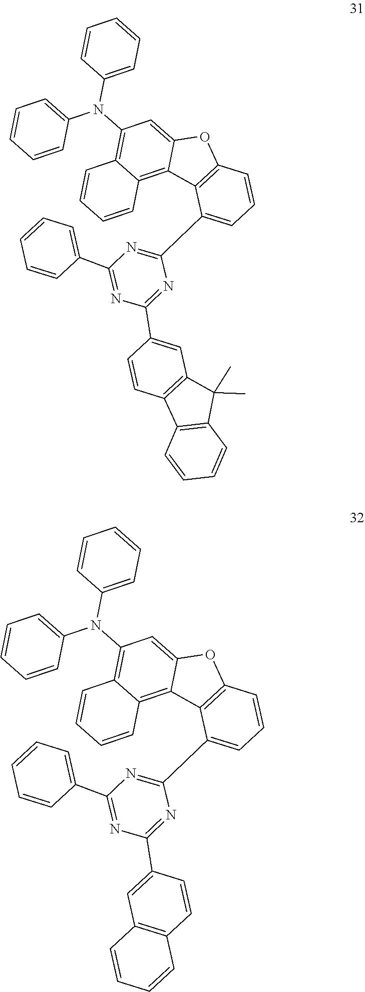 [SMILES: CC1(C)c2ccccc2-c2ccc(-c3nc(-c4ccccc4)nc(-c4cccc5oc6cc(N(c7ccccc7)c7ccccc7)c7ccccc7c6c45)n3)cc21 ; c1ccc(-c2nc(-c3ccc4ccccc4c3)nc(-c3cccc4oc5cc(N(c6ccccc6)c6ccccc6)c6ccccc6c5c34)n2)cc1]

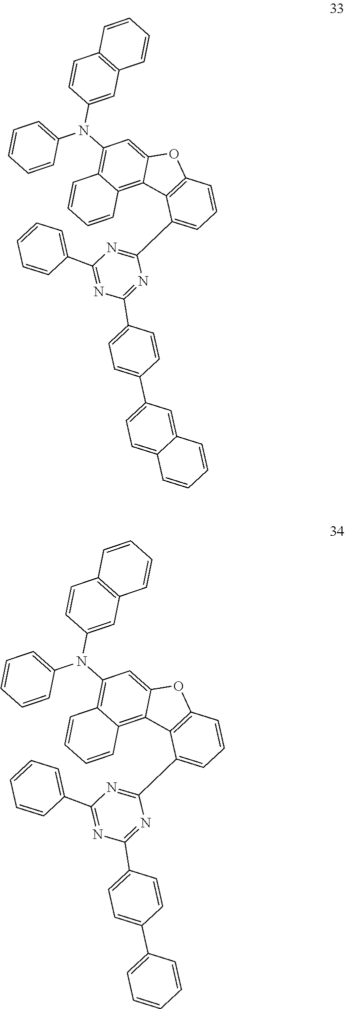 [SMILES: c1ccc(-c2nc(-c3ccc(-c4ccc5ccccc5c4)cc3)nc(-c3cccc4oc5cc(N(c6ccccc6)c6ccc7ccccc7c6)c6ccccc6c5c34)n2)cc1 ; c1ccc(-c2ccc(-c3nc(-c4ccccc4)nc(-c4cccc5oc6cc(N(c7ccccc7)c7ccc8ccccc8c7)c7ccccc7c6c45)n3)cc2)cc1]

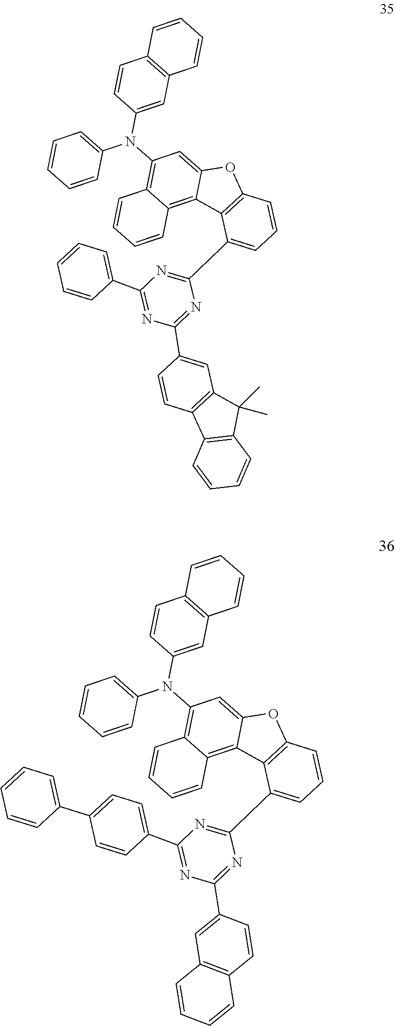 [SMILES: CC1(C)c2ccccc2-c2ccc(-c3nc(-c4ccccc4)nc(-c4cccc5oc6cc(N(c7ccccc7)c7ccc8ccccc8c7)c7ccccc7c6c45)n3)cc21 ; c1ccc(-c2ccc(-c3nc(-c4ccc5ccccc5c4)nc(-c4cccc5oc6cc(N(c7ccccc7)c7ccc8ccccc8c7)c7ccccc7c6c45)n3)cc2)cc1]

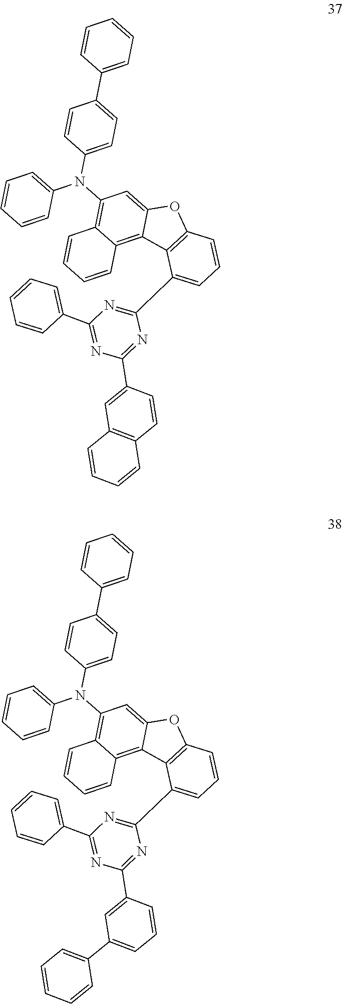 [SMILES: c1ccc(-c2ccc(N(c3ccccc3)c3cc4oc5cccc(-c6nc(-c7ccccc7)nc(-c7ccc8ccccc8c7)n6)c5c4c4ccccc34)cc2)cc1 ; c1ccc(-c2ccc(N(c3ccccc3)c3cc4oc5cccc(-c6nc(-c7ccccc7)nc(-c7cccc(-c8ccccc8)c7)n6)c5c4c4ccccc34)cc2)cc1]

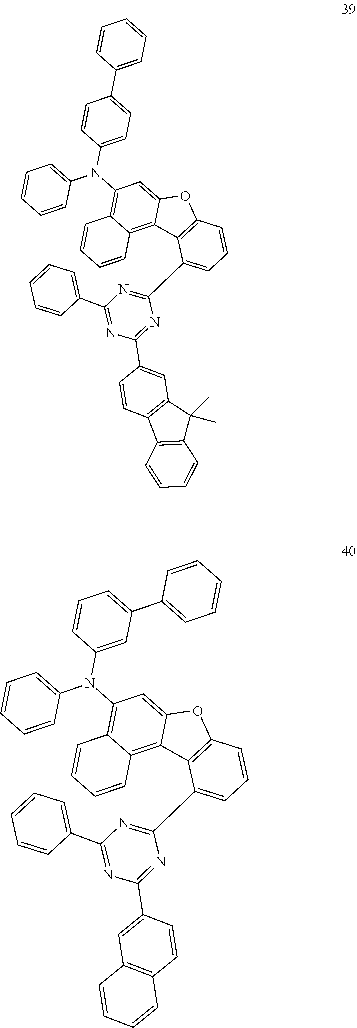 [SMILES: CC1(C)c2ccccc2-c2ccc(-c3nc(-c4ccccc4)nc(-c4cccc5oc6cc(N(c7ccccc7)c7ccc(-c8ccccc8)cc7)c7ccccc7c6c45)n3)cc21 ; c1ccc(-c2cccc(N(c3ccccc3)c3cc4oc5cccc(-c6nc(-c7ccccc7)nc(-c7ccc8ccccc8c7)n6)c5c4c4ccccc34)c2)cc1]

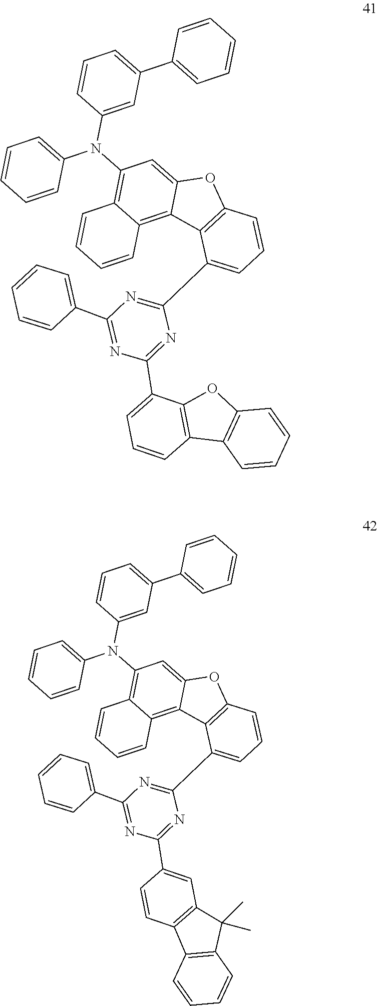 [SMILES: c1ccc(-c2cccc(N(c3ccccc3)c3cc4oc5cccc(-c6nc(-c7ccccc7)nc(-c7cccc8c7oc7ccccc78)n6)c5c4c4ccccc34)c2)cc1 ; CC1(C)c2ccccc2-c2ccc(-c3nc(-c4ccccc4)nc(-c4cccc5oc6cc(N(c7ccccc7)c7cccc(-c8ccccc8)c7)c7ccccc7c6c45)n3)cc21]

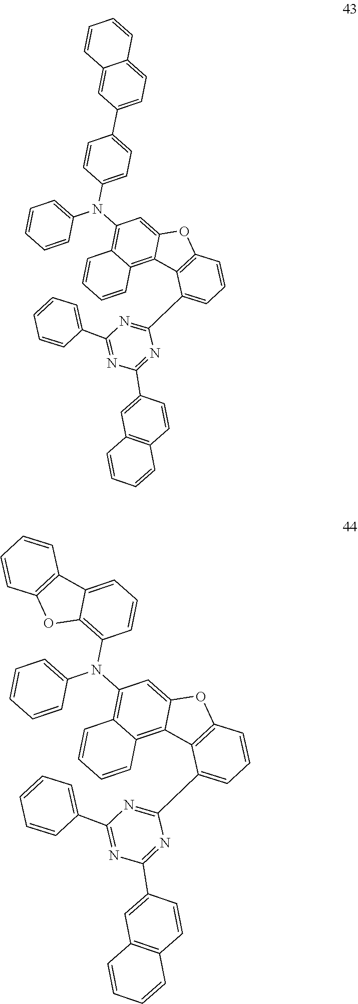 [SMILES: c1ccc(-c2nc(-c3ccc4ccccc4c3)nc(-c3cccc4oc5cc(N(c6ccccc6)c6ccc(-c7ccc8ccccc8c7)cc6)c6ccccc6c5c34)n2)cc1 ; c1ccc(-c2nc(-c3ccc4ccccc4c3)nc(-c3cccc4oc5cc(N(c6ccccc6)c6cccc7c6oc6ccccc67)c6ccccc6c5c34)n2)cc1]

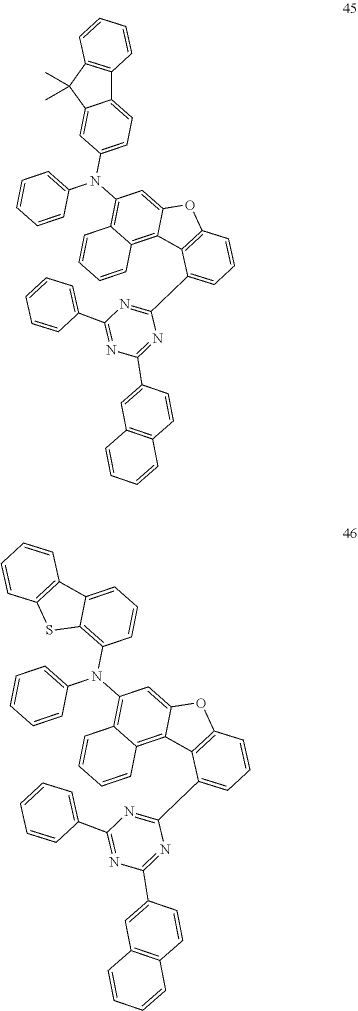 [SMILES: CC1(C)c2ccccc2-c2ccc(N(c3ccccc3)c3cc4oc5cccc(-c6nc(-c7ccccc7)nc(-c7ccc8ccccc8c7)n6)c5c4c4ccccc34)cc21 ; c1ccc(-c2nc(-c3ccc4ccccc4c3)nc(-c3cccc4oc5cc(N(c6ccccc6)c6cccc7c6sc6ccccc67)c6ccccc6c5c34)n2)cc1]

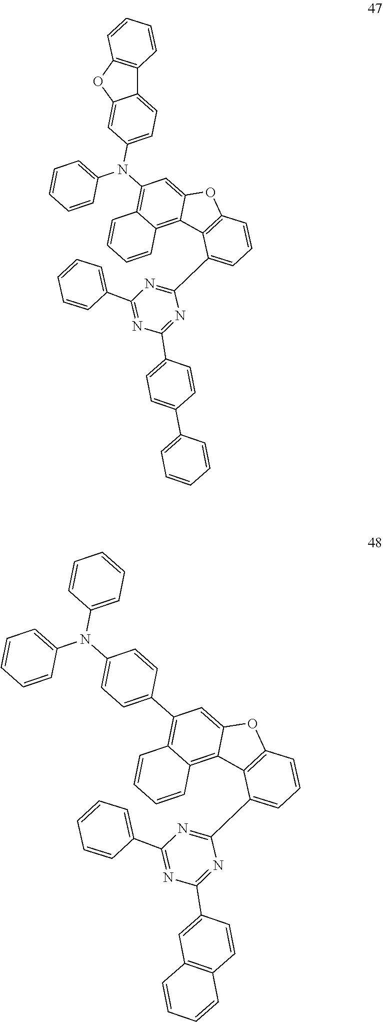 [SMILES: c1ccc(-c2ccc(-c3nc(-c4ccccc4)nc(-c4cccc5oc6cc(N(c7ccccc7)c7ccc8c(c7)oc7ccccc78)c7ccccc7c6c45)n3)cc2)cc1 ; c1ccc(-c2nc(-c3ccc4ccccc4c3)nc(-c3cccc4oc5cc(-c6ccc(N(c7ccccc7)c7ccccc7)cc6)c6ccccc6c5c34)n2)cc1]

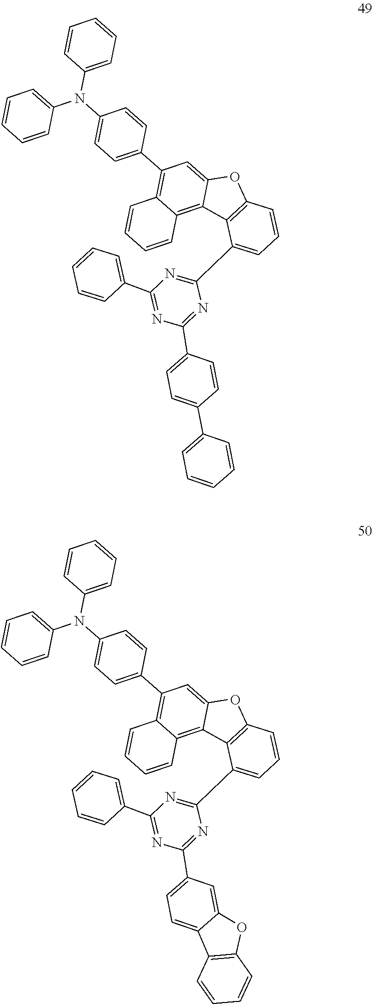 [SMILES: c1ccc(-c2ccc(-c3nc(-c4ccccc4)nc(-c4cccc5oc6cc(-c7ccc(N(c8ccccc8)c8ccccc8)cc7)c7ccccc7c6c45)n3)cc2)cc1 ; c1ccc(-c2nc(-c3ccc4c(c3)oc3ccccc34)nc(-c3cccc4oc5cc(-c6ccc(N(c7ccccc7)c7ccccc7)cc6)c6ccccc6c5c34)n2)cc1]

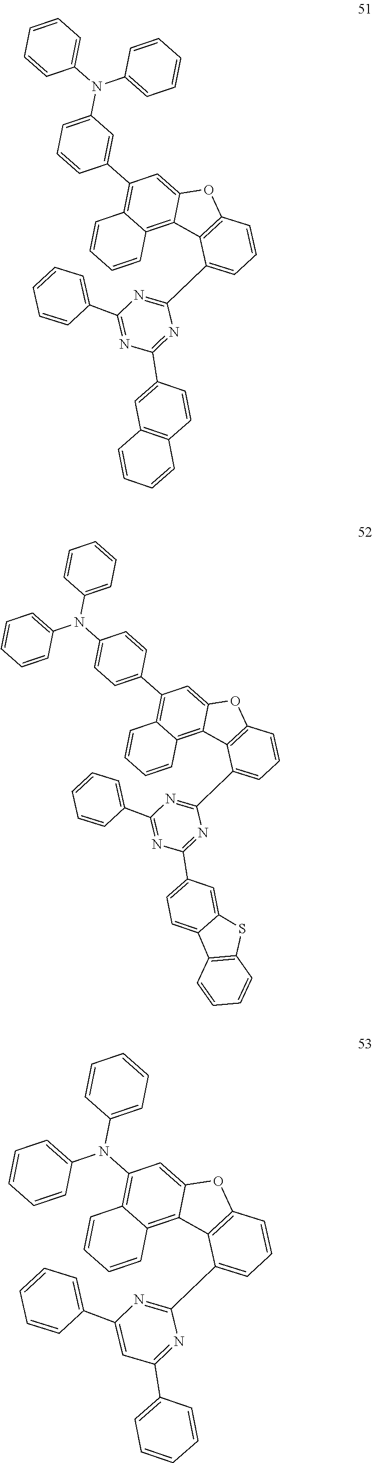 [SMILES: c1ccc(-c2nc(-c3ccc4ccccc4c3)nc(-c3cccc4oc5cc(-c6cccc(N(c7ccccc7)c7ccccc7)c6)c6ccccc6c5c34)n2)cc1 ; c1ccc(-c2nc(-c3ccc4c(c3)sc3ccccc34)nc(-c3cccc4oc5cc(-c6ccc(N(c7ccccc7)c7ccccc7)cc6)c6ccccc6c5c34)n2)cc1 ; c1ccc(-c2cc(-c3ccccc3)nc(-c3cccc4oc5cc(N(c6ccccc6)c6ccccc6)c6ccccc6c5c34)n2)cc1]

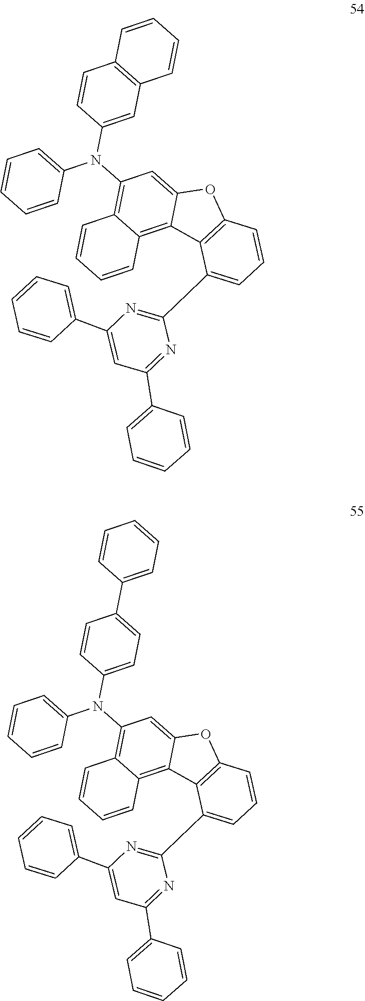 [SMILES: c1ccc(-c2cc(-c3ccccc3)nc(-c3cccc4oc5cc(N(c6ccccc6)c6ccc7ccccc7c6)c6ccccc6c5c34)n2)cc1 ; c1ccc(-c2ccc(N(c3ccccc3)c3cc4oc5cccc(-c6nc(-c7ccccc7)cc(-c7ccccc7)n6)c5c4c4ccccc34)cc2)cc1]

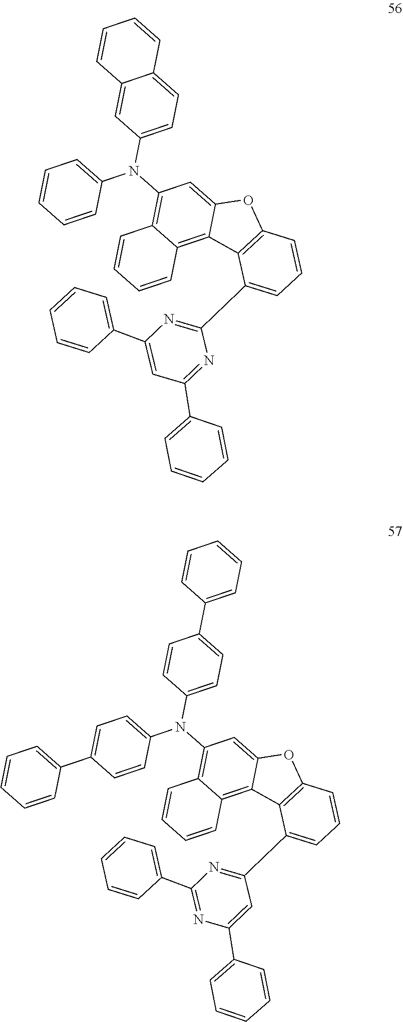 [SMILES: c1ccc(-c2cc(-c3ccccc3)nc(-c3cccc4oc5cc(N(c6ccccc6)c6ccc7ccccc7c6)c6ccccc6c5c34)n2)cc1 ; c1ccc(-c2ccc(N(c3ccc(-c4ccccc4)cc3)c3cc4oc5cccc(-c6cc(-c7ccccc7)nc(-c7ccccc7)n6)c5c4c4ccccc34)cc2)cc1]

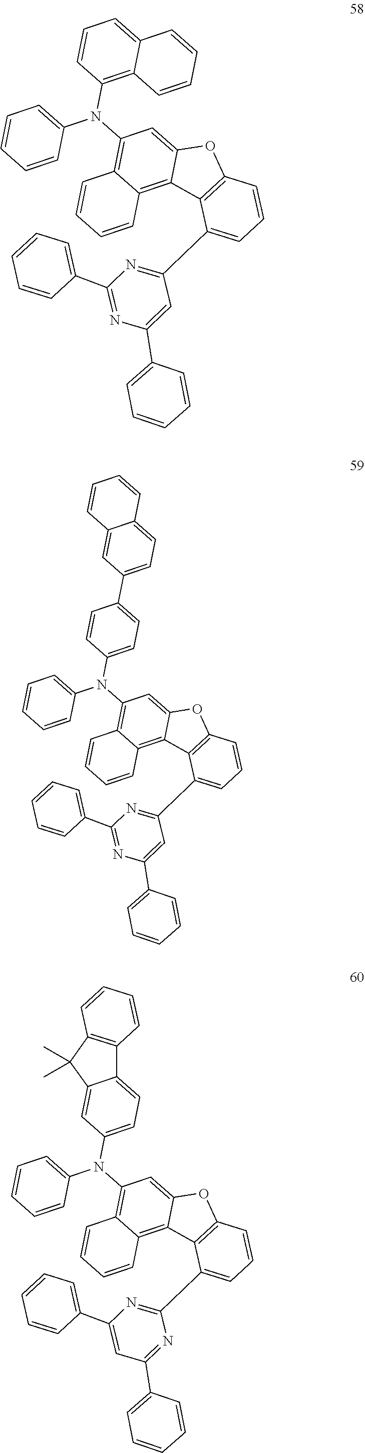 [SMILES: c1ccc(-c2cc(-c3cccc4oc5cc(N(c6ccccc6)c6cccc7ccccc67)c6ccccc6c5c34)nc(-c3ccccc3)n2)cc1 ; c1ccc(-c2cc(-c3cccc4oc5cc(N(c6ccccc6)c6ccc(-c7ccc8ccccc8c7)cc6)c6ccccc6c5c34)nc(-c3ccccc3)n2)cc1 ; CC1(C)c2ccccc2-c2ccc(N(c3ccccc3)c3cc4oc5cccc(-c6nc(-c7ccccc7)cc(-c7ccccc7)n6)c5c4c4ccccc34)cc21]

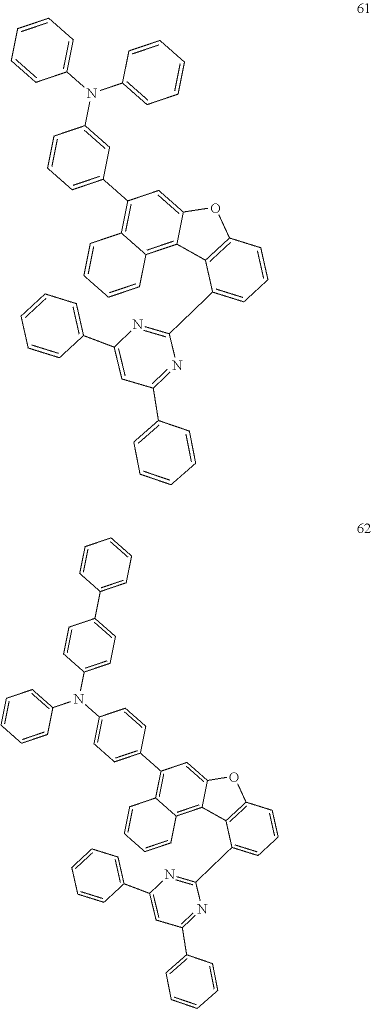 [SMILES: c1ccc(-c2cc(-c3ccccc3)nc(-c3cccc4oc5cc(-c6cccc(N(c7ccccc7)c7ccccc7)c6)c6ccccc6c5c34)n2)cc1 ; c1ccc(-c2ccc(N(c3ccccc3)c3ccc(-c4cc5oc6cccc(-c7nc(-c8ccccc8)cc(-c8ccccc8)n7)c6c5c5ccccc45)cc3)cc2)cc1]

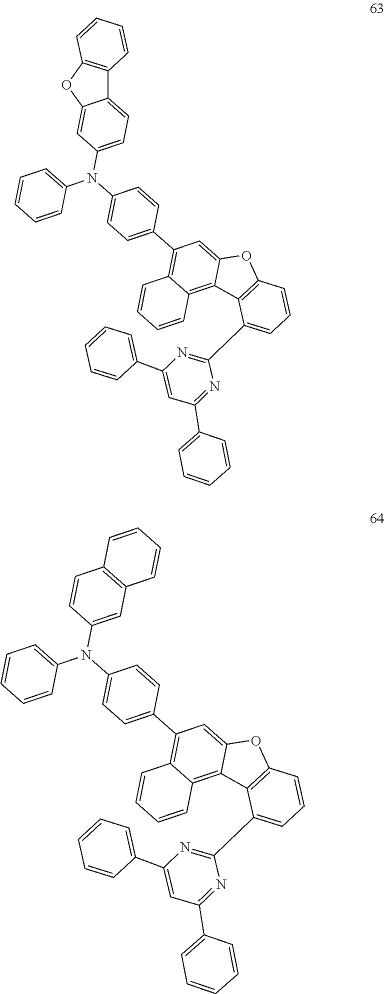 [SMILES: c1ccc(-c2cc(-c3ccccc3)nc(-c3cccc4oc5cc(-c6ccc(N(c7ccccc7)c7ccc8c(c7)oc7ccccc78)cc6)c6ccccc6c5c34)n2)cc1 ; c1ccc(-c2cc(-c3ccccc3)nc(-c3cccc4oc5cc(-c6ccc(N(c7ccccc7)c7ccc8ccccc8c7)cc6)c6ccccc6c5c34)n2)cc1]

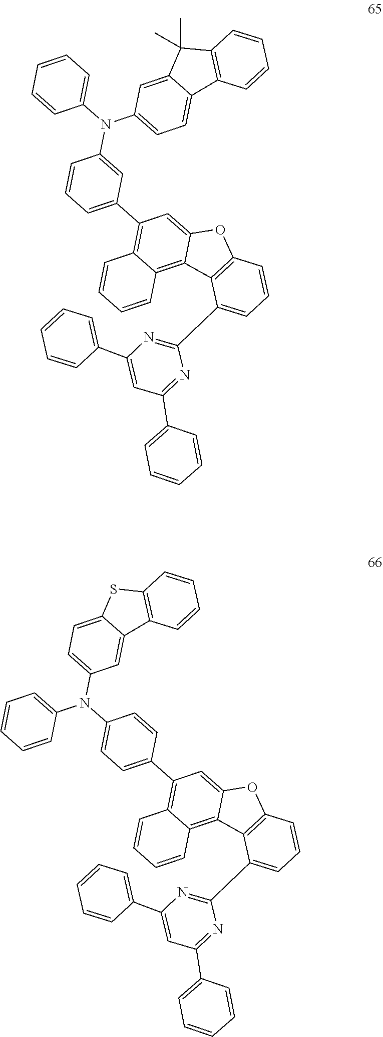 [SMILES: CC1(C)c2ccccc2-c2ccc(N(c3ccccc3)c3cccc(-c4cc5oc6cccc(-c7nc(-c8ccccc8)cc(-c8ccccc8)n7)c6c5c5ccccc45)c3)cc21 ; c1ccc(-c2cc(-c3ccccc3)nc(-c3cccc4oc5cc(-c6ccc(N(c7ccccc7)c7ccc8sc9ccccc9c8c7)cc6)c6ccccc6c5c34)n2)cc1]

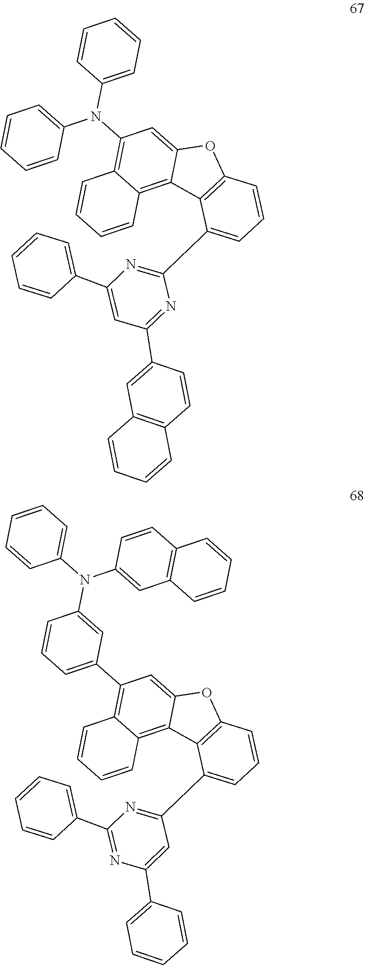 [SMILES: c1ccc(-c2cc(-c3ccc4ccccc4c3)nc(-c3cccc4oc5cc(N(c6ccccc6)c6ccccc6)c6ccccc6c5c34)n2)cc1 ; c1ccc(-c2cc(-c3cccc4oc5cc(-c6cccc(N(c7ccccc7)c7ccc8ccccc8c7)c6)c6ccccc6c5c34)nc(-c3ccccc3)n2)cc1]

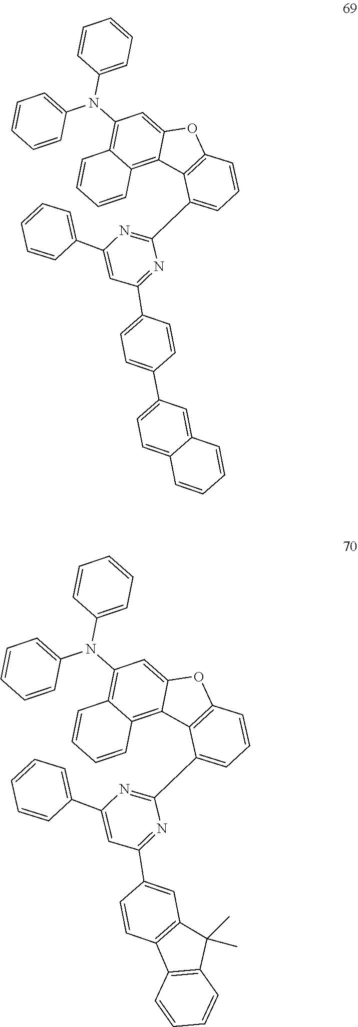 [SMILES: c1ccc(-c2cc(-c3ccc(-c4ccc5ccccc5c4)cc3)nc(-c3cccc4oc5cc(N(c6ccccc6)c6ccccc6)c6ccccc6c5c34)n2)cc1 ; CC1(C)c2ccccc2-c2ccc(-c3cc(-c4ccccc4)nc(-c4cccc5oc6cc(N(c7ccccc7)c7ccccc7)c7ccccc7c6c45)n3)cc21]

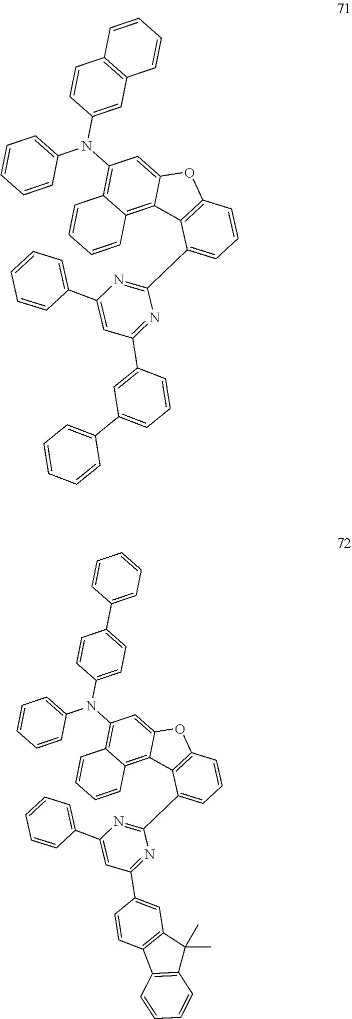 [SMILES: c1ccc(-c2cccc(-c3cc(-c4ccccc4)nc(-c4cccc5oc6cc(N(c7ccccc7)c7ccc8ccccc8c7)c7ccccc7c6c45)n3)c2)cc1 ; CC1(C)c2ccccc2-c2ccc(-c3cc(-c4ccccc4)nc(-c4cccc5oc6cc(N(c7ccccc7)c7ccc(-c8ccccc8)cc7)c7ccccc7c6c45)n3)cc21]

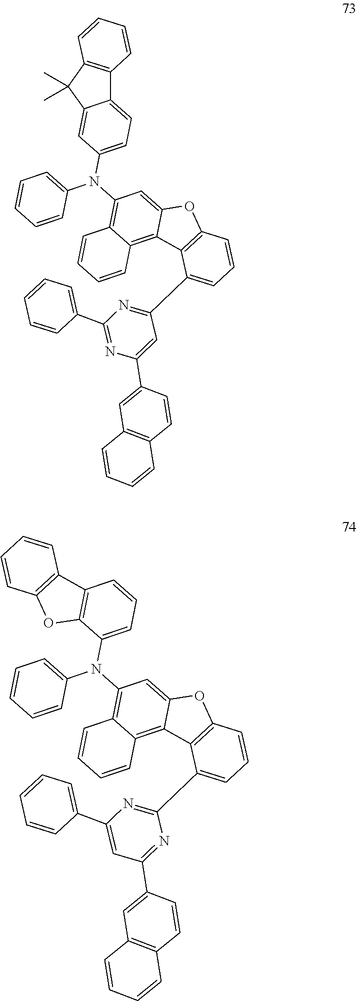 [SMILES: CC1(C)c2ccccc2-c2ccc(N(c3ccccc3)c3cc4oc5cccc(-c6cc(-c7ccc8ccccc8c7)nc(-c7ccccc7)n6)c5c4c4ccccc34)cc21 ; c1ccc(-c2cc(-c3ccc4ccccc4c3)nc(-c3cccc4oc5cc(N(c6ccccc6)c6cccc7c6oc6ccccc67)c6ccccc6c5c34)n2)cc1]

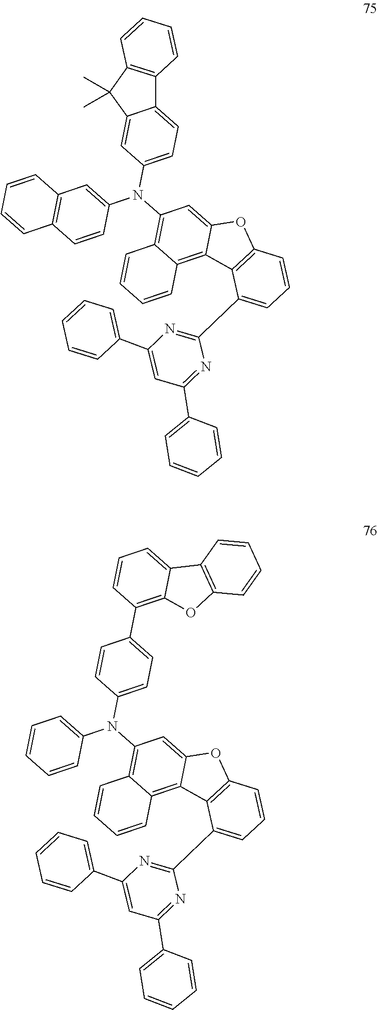 [SMILES: CC1(C)c2ccccc2-c2ccc(N(c3ccc4ccccc4c3)c3cc4oc5cccc(-c6nc(-c7ccccc7)cc(-c7ccccc7)n6)c5c4c4ccccc34)cc21 ; c1ccc(-c2cc(-c3ccccc3)nc(-c3cccc4oc5cc(N(c6ccccc6)c6ccc(-c7cccc8c7oc7ccccc78)cc6)c6ccccc6c5c34)n2)cc1]

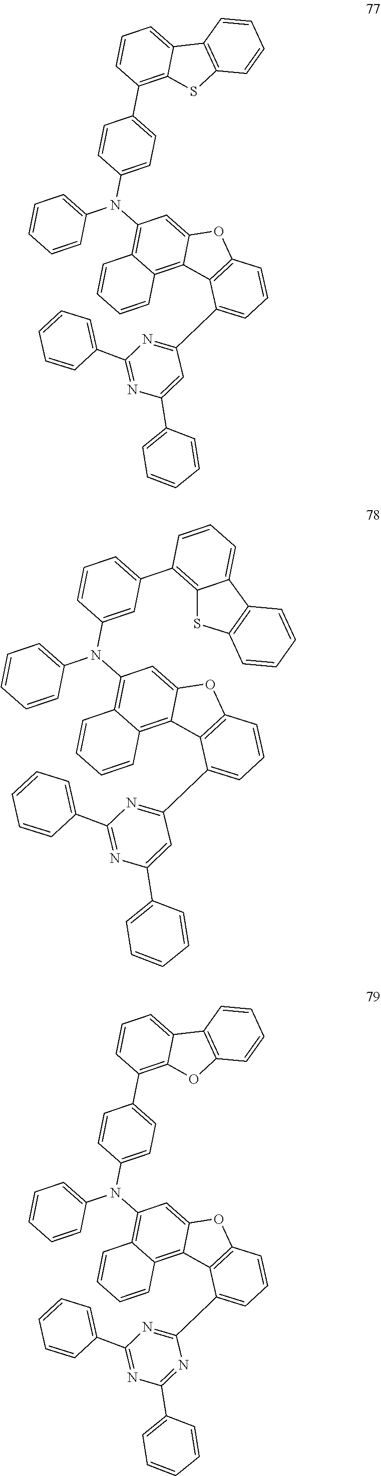 [SMILES: c1ccc(-c2cc(-c3cccc4oc5cc(N(c6ccccc6)c6ccc(-c7cccc8c7sc7ccccc78)cc6)c6ccccc6c5c34)nc(-c3ccccc3)n2)cc1 ; c1ccc(-c2cc(-c3cccc4oc5cc(N(c6ccccc6)c6cccc(-c7cccc8c7sc7ccccc78)c6)c6ccccc6c5c34)nc(-c3ccccc3)n2)cc1 ; c1ccc(-c2nc(-c3ccccc3)nc(-c3cccc4oc5cc(N(c6ccccc6)c6ccc(-c7cccc8c7oc7ccccc78)cc6)c6ccccc6c5c34)n2)cc1]

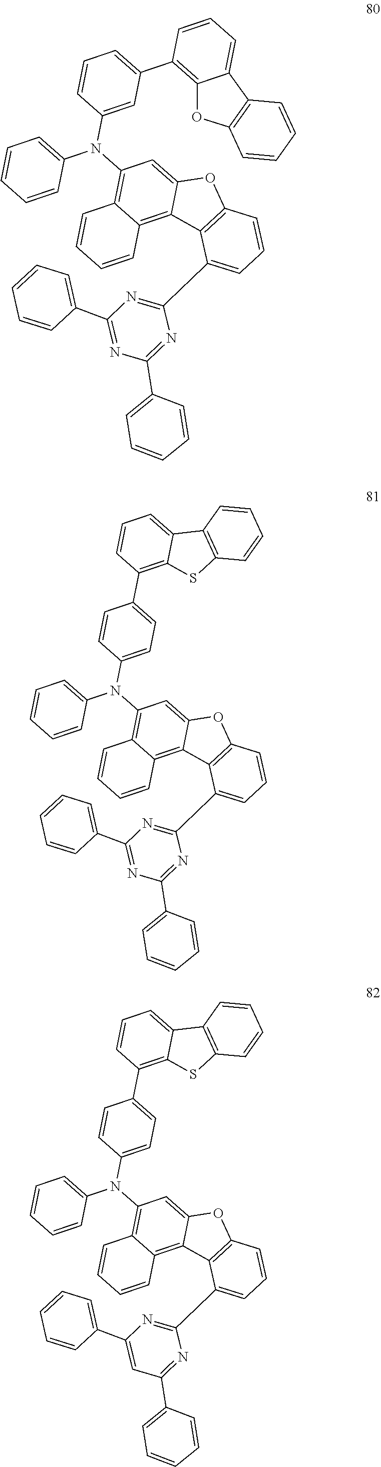 [SMILES: c1ccc(-c2nc(-c3ccccc3)nc(-c3cccc4oc5cc(N(c6ccccc6)c6cccc(-c7cccc8c7oc7ccccc78)c6)c6ccccc6c5c34)n2)cc1 ; c1ccc(-c2nc(-c3ccccc3)nc(-c3cccc4oc5cc(N(c6ccccc6)c6ccc(-c7cccc8c7sc7ccccc78)cc6)c6ccccc6c5c34)n2)cc1 ; c1ccc(-c2cc(-c3ccccc3)nc(-c3cccc4oc5cc(N(c6ccccc6)c6ccc(-c7cccc8c7sc7ccccc78)cc6)c6ccccc6c5c34)n2)cc1]

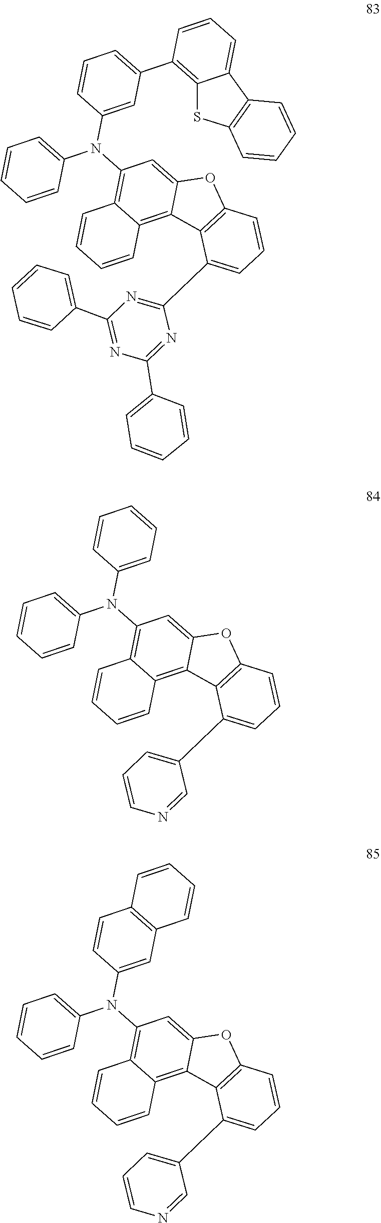 [SMILES: c1ccc(-c2nc(-c3ccccc3)nc(-c3cccc4oc5cc(N(c6ccccc6)c6cccc(-c7cccc8c7sc7ccccc78)c6)c6ccccc6c5c34)n2)cc1 ; c1ccc(N(c2ccccc2)c2cc3oc4cccc(-c5cccnc5)c4c3c3ccccc23)cc1 ; c1ccc(N(c2ccc3ccccc3c2)c2cc3oc4cccc(-c5cccnc5)c4c3c3ccccc23)cc1]

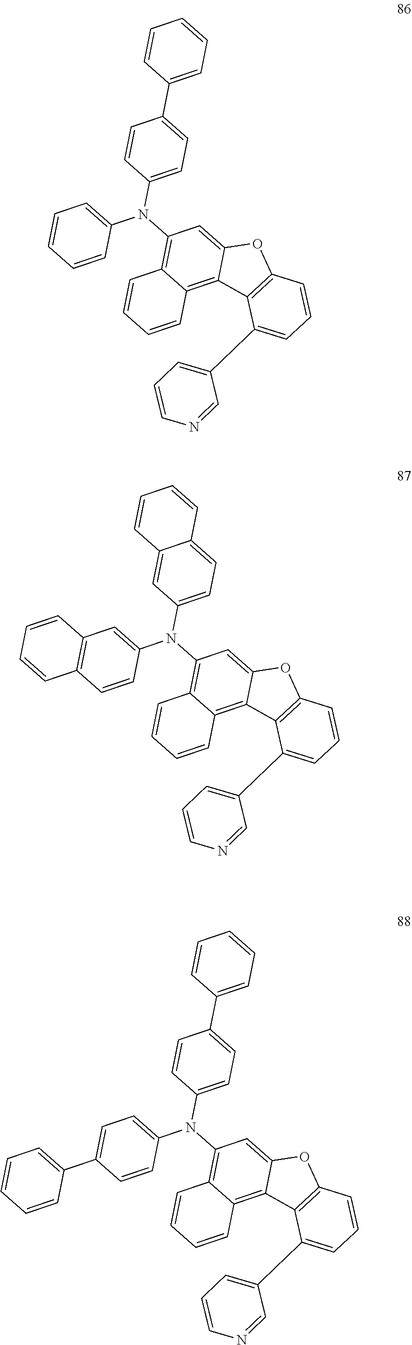 [SMILES: c1ccc(-c2ccc(N(c3ccccc3)c3cc4oc5cccc(-c6cccnc6)c5c4c4ccccc34)cc2)cc1 ; c1cncc(-c2cccc3oc4cc(N(c5ccc6ccccc6c5)c5ccc6ccccc6c5)c5ccccc5c4c23)c1 ; c1ccc(-c2ccc(N(c3ccc(-c4ccccc4)cc3)c3cc4oc5cccc(-c6cccnc6)c5c4c4ccccc34)cc2)cc1]

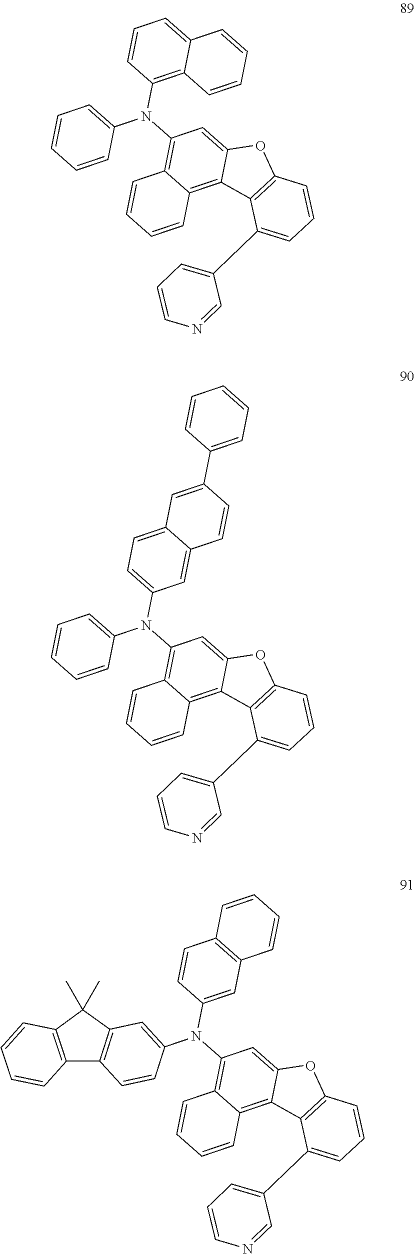 [SMILES: c1ccc(N(c2cccc3ccccc23)c2cc3oc4cccc(-c5cccnc5)c4c3c3ccccc23)cc1 ; c1ccc(-c2ccc3cc(N(c4ccccc4)c4cc5oc6cccc(-c7cccnc7)c6c5c5ccccc45)ccc3c2)cc1 ; CC1(C)c2ccccc2-c2ccc(N(c3ccc4ccccc4c3)c3cc4oc5cccc(-c6cccnc6)c5c4c4ccccc34)cc21]

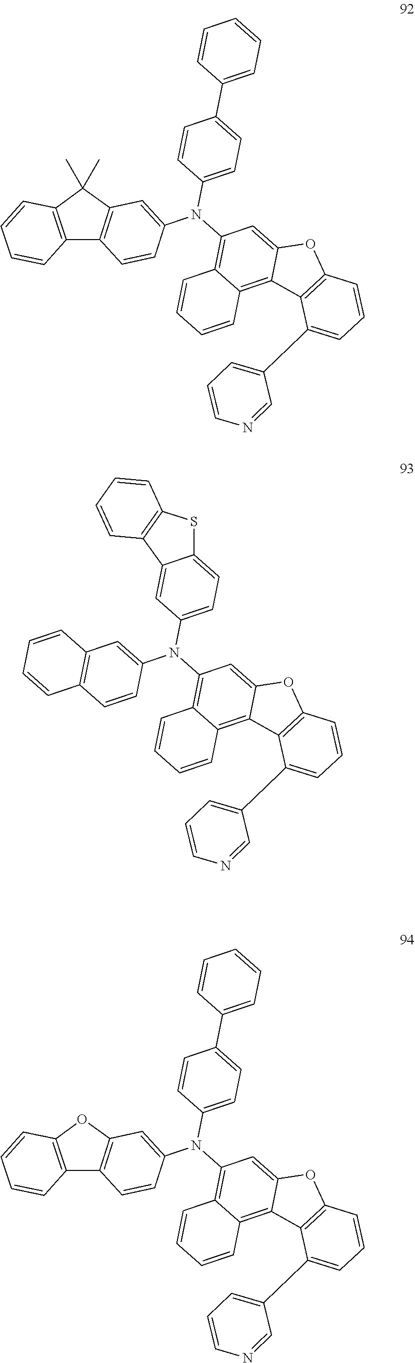 [SMILES: CC1(C)c2ccccc2-c2ccc(N(c3ccc(-c4ccccc4)cc3)c3cc4oc5cccc(-c6cccnc6)c5c4c4ccccc34)cc21 ; c1cncc(-c2cccc3oc4cc(N(c5ccc6ccccc6c5)c5ccc6sc7ccccc7c6c5)c5ccccc5c4c23)c1 ; c1ccc(-c2ccc(N(c3ccc4c(c3)oc3ccccc34)c3cc4oc5cccc(-c6cccnc6)c5c4c4ccccc34)cc2)cc1]

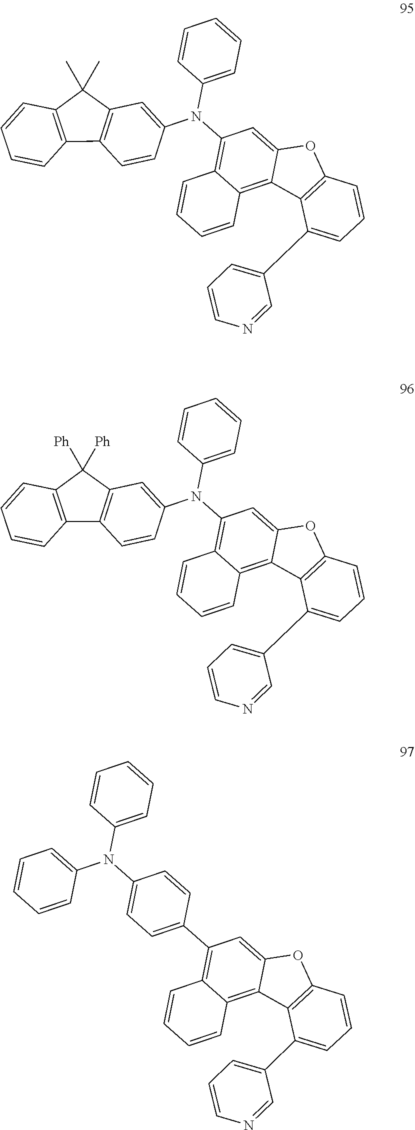 [SMILES: CC1(C)c2ccccc2-c2ccc(N(c3ccccc3)c3cc4oc5cccc(-c6cccnc6)c5c4c4ccccc34)cc21 ; c1ccc(N(c2ccc3c(c2)C(c2ccccc2)(c2ccccc2)c2ccccc2-3)c2cc3oc4cccc(-c5cccnc5)c4c3c3ccccc23)cc1 ; c1ccc(N(c2ccccc2)c2ccc(-c3cc4oc5cccc(-c6cccnc6)c5c4c4ccccc34)cc2)cc1]

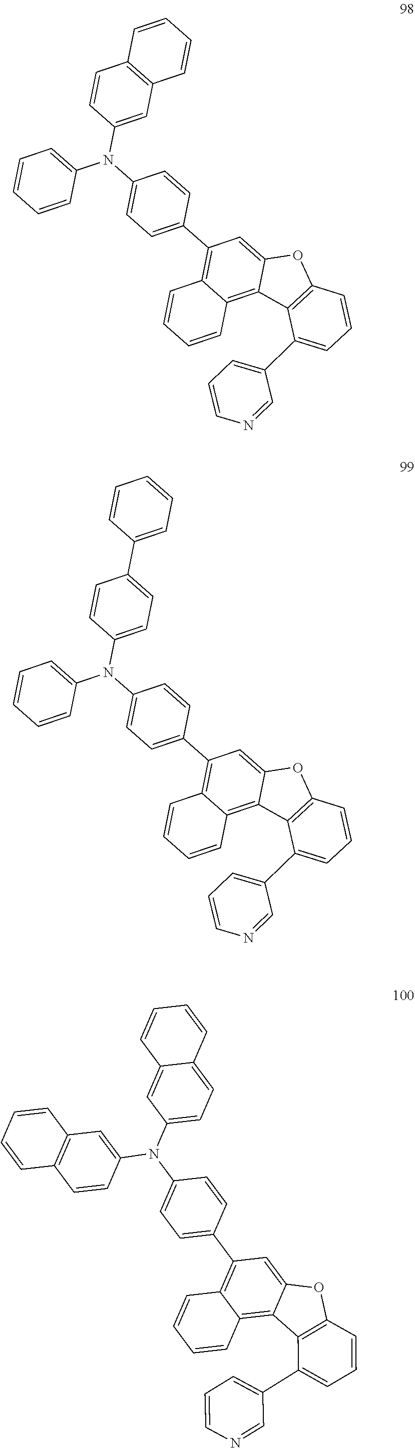 [SMILES: c1ccc(N(c2ccc(-c3cc4oc5cccc(-c6cccnc6)c5c4c4ccccc34)cc2)c2ccc3ccccc3c2)cc1 ; c1ccc(-c2ccc(N(c3ccccc3)c3ccc(-c4cc5oc6cccc(-c7cccnc7)c6c5c5ccccc45)cc3)cc2)cc1 ; c1cncc(-c2cccc3oc4cc(-c5ccc(N(c6ccc7ccccc7c6)c6ccc7ccccc7c6)cc5)c5ccccc5c4c23)c1]

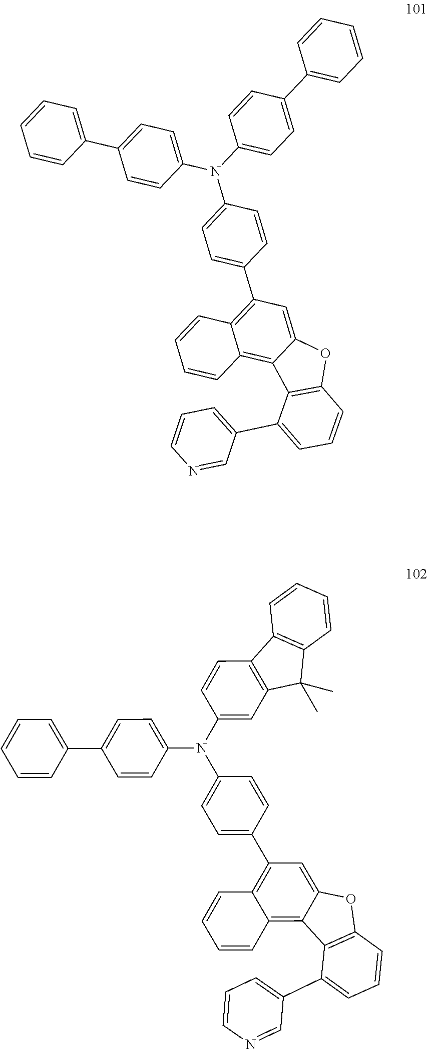 [SMILES: c1ccc(-c2ccc(N(c3ccc(-c4ccccc4)cc3)c3ccc(-c4cc5oc6cccc(-c7cccnc7)c6c5c5ccccc45)cc3)cc2)cc1 ; CC1(C)c2ccccc2-c2ccc(N(c3ccc(-c4ccccc4)cc3)c3ccc(-c4cc5oc6cccc(-c7cccnc7)c6c5c5ccccc45)cc3)cc21]

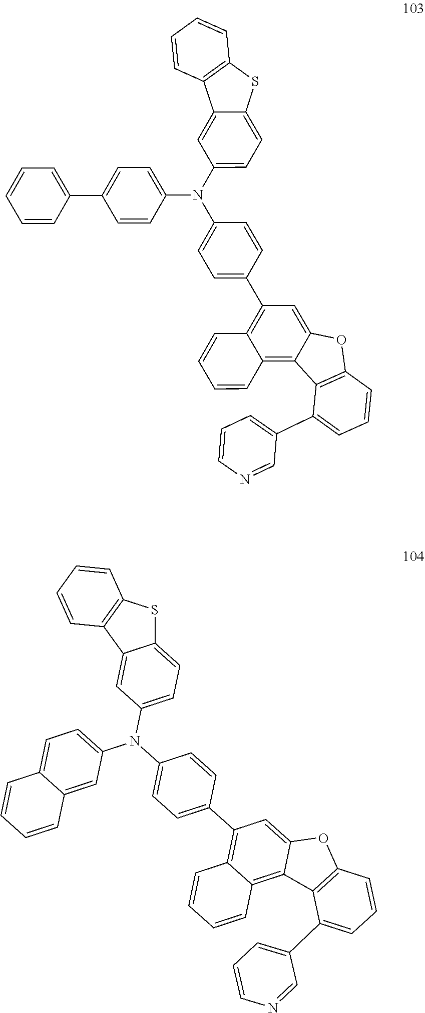 [SMILES: c1ccc(-c2ccc(N(c3ccc(-c4cc5oc6cccc(-c7cccnc7)c6c5c5ccccc45)cc3)c3ccc4sc5ccccc5c4c3)cc2)cc1 ; c1cncc(-c2cccc3oc4cc(-c5ccc(N(c6ccc7ccccc7c6)c6ccc7sc8ccccc8c7c6)cc5)c5ccccc5c4c23)c1]

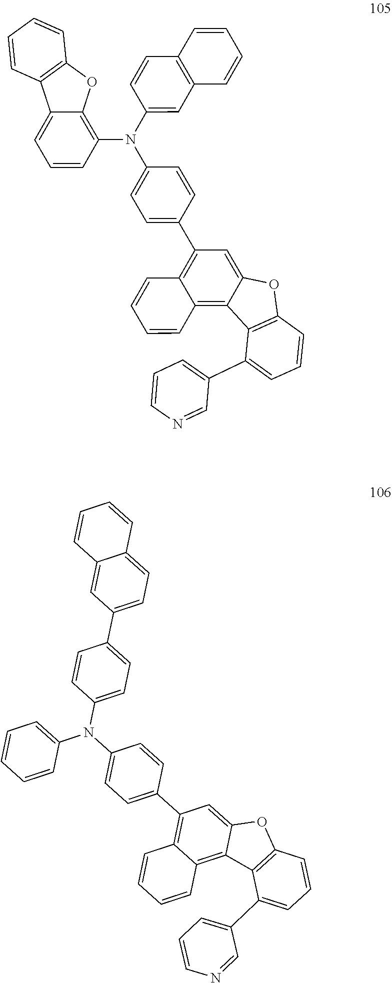 [SMILES: c1cncc(-c2cccc3oc4cc(-c5ccc(N(c6ccc7ccccc7c6)c6cccc7c6oc6ccccc67)cc5)c5ccccc5c4c23)c1 ; c1ccc(N(c2ccc(-c3ccc4ccccc4c3)cc2)c2ccc(-c3cc4oc5cccc(-c6cccnc6)c5c4c4ccccc34)cc2)cc1]

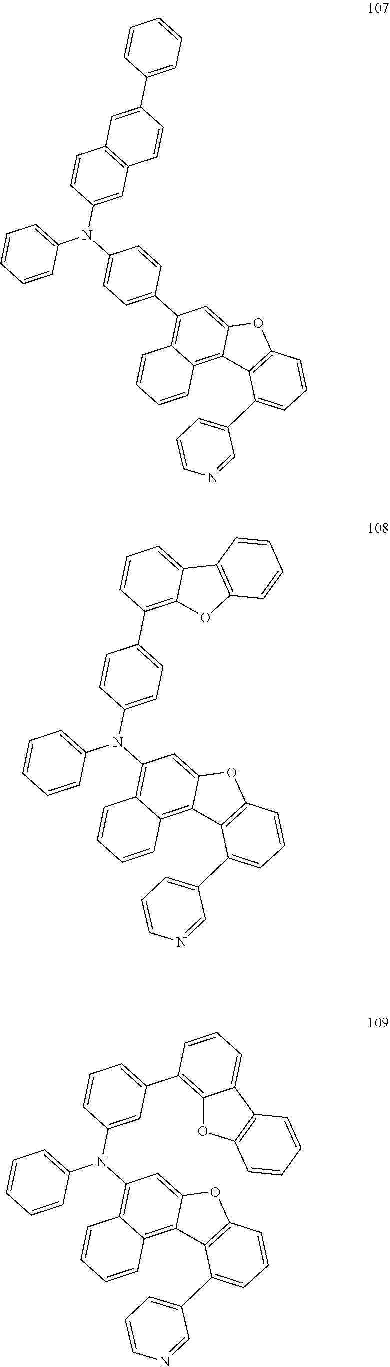 [SMILES: c1ccc(-c2ccc3cc(N(c4ccccc4)c4ccc(-c5cc6oc7cccc(-c8cccnc8)c7c6c6ccccc56)cc4)ccc3c2)cc1 ; c1ccc(N(c2ccc(-c3cccc4c3oc3ccccc34)cc2)c2cc3oc4cccc(-c5cccnc5)c4c3c3ccccc23)cc1 ; c1ccc(N(c2cccc(-c3cccc4c3oc3ccccc34)c2)c2cc3oc4cccc(-c5cccnc5)c4c3c3ccccc23)cc1]

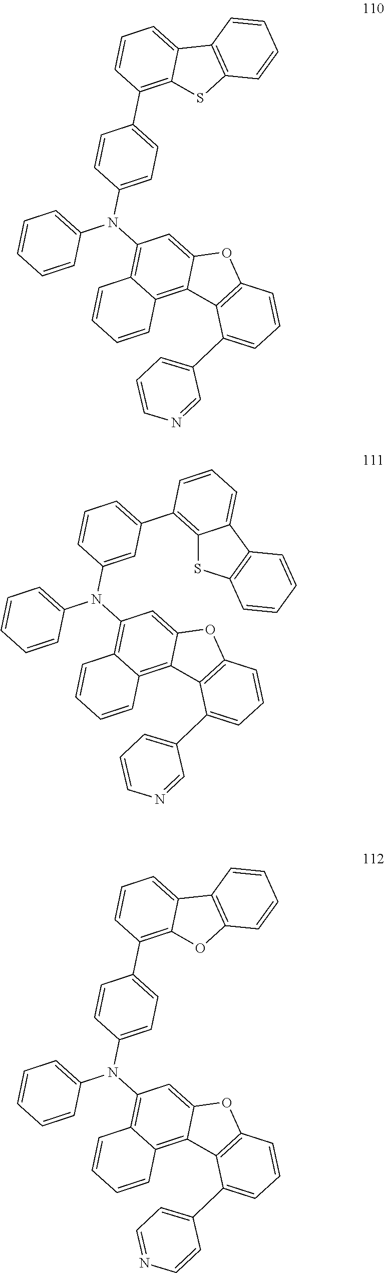 [SMILES: c1ccc(N(c2ccc(-c3cccc4c3sc3ccccc34)cc2)c2cc3oc4cccc(-c5cccnc5)c4c3c3ccccc23)cc1 ; c1ccc(N(c2cccc(-c3cccc4c3sc3ccccc34)c2)c2cc3oc4cccc(-c5cccnc5)c4c3c3ccccc23)cc1 ; c1ccc(N(c2ccc(-c3cccc4c3oc3ccccc34)cc2)c2cc3oc4cccc(-c5ccncc5)c4c3c3ccccc23)cc1]

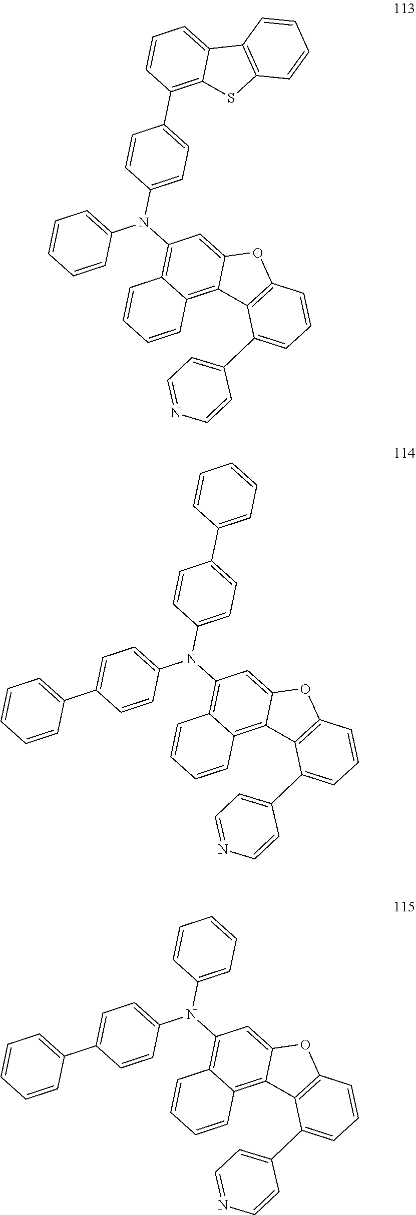 [SMILES: c1ccc(N(c2ccc(-c3cccc4c3sc3ccccc34)cc2)c2cc3oc4cccc(-c5ccncc5)c4c3c3ccccc23)cc1 ; c1ccc(-c2ccc(N(c3ccc(-c4ccccc4)cc3)c3cc4oc5cccc(-c6ccncc6)c5c4c4ccccc34)cc2)cc1 ; c1ccc(-c2ccc(N(c3ccccc3)c3cc4oc5cccc(-c6ccncc6)c5c4c4ccccc34)cc2)cc1]

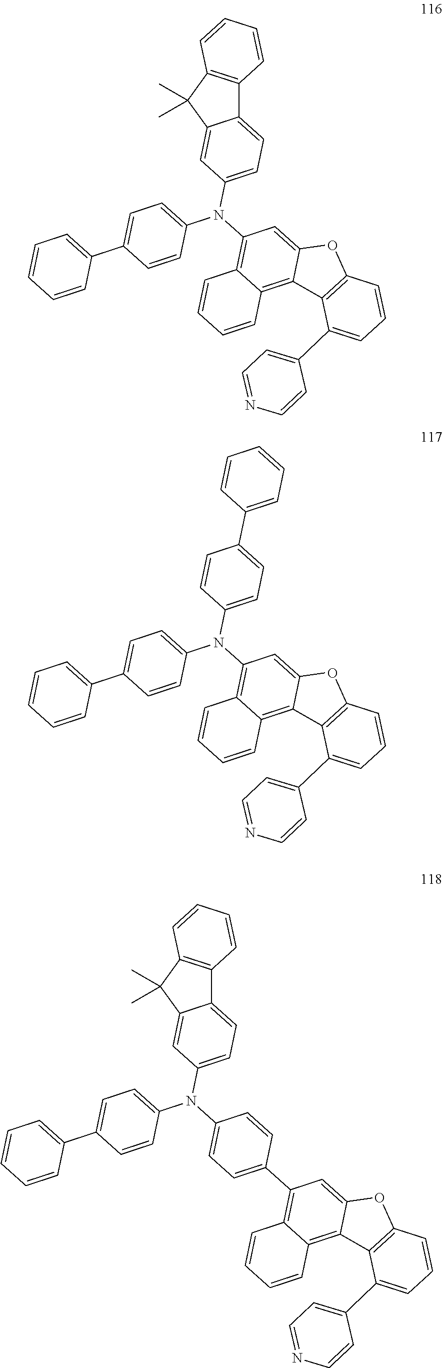

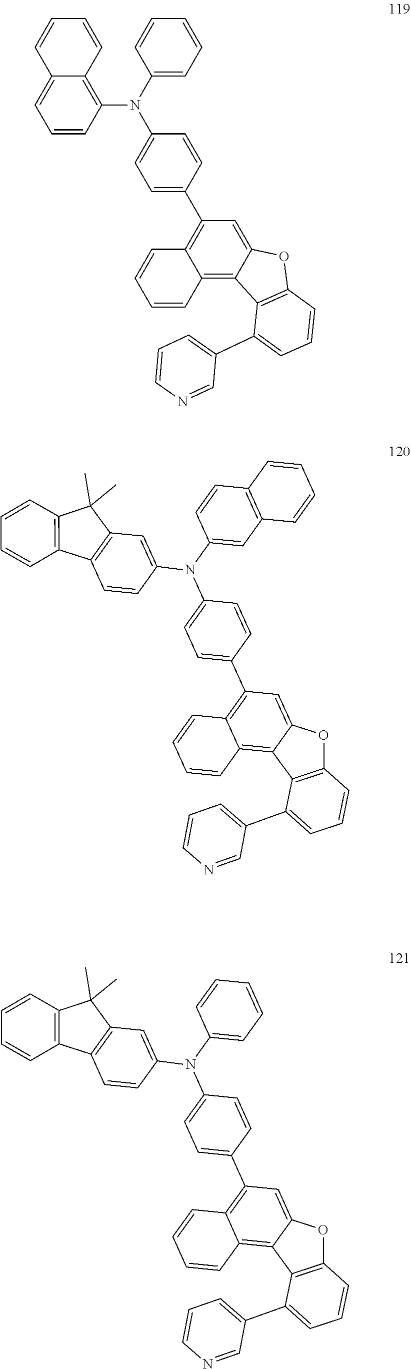 [SMILES: c1ccc(N(c2ccc(-c3cc4oc5cccc(-c6cccnc6)c5c4c4ccccc34)cc2)c2cccc3ccccc23)cc1 ; CC1(C)c2ccccc2-c2ccc(N(c3ccc(-c4cc5oc6cccc(-c7cccnc7)c6c5c5ccccc45)cc3)c3ccc4ccccc4c3)cc21 ; CC1(C)c2ccccc2-c2ccc(N(c3ccccc3)c3ccc(-c4cc5oc6cccc(-c7cccnc7)c6c5c5ccccc45)cc3)cc21]

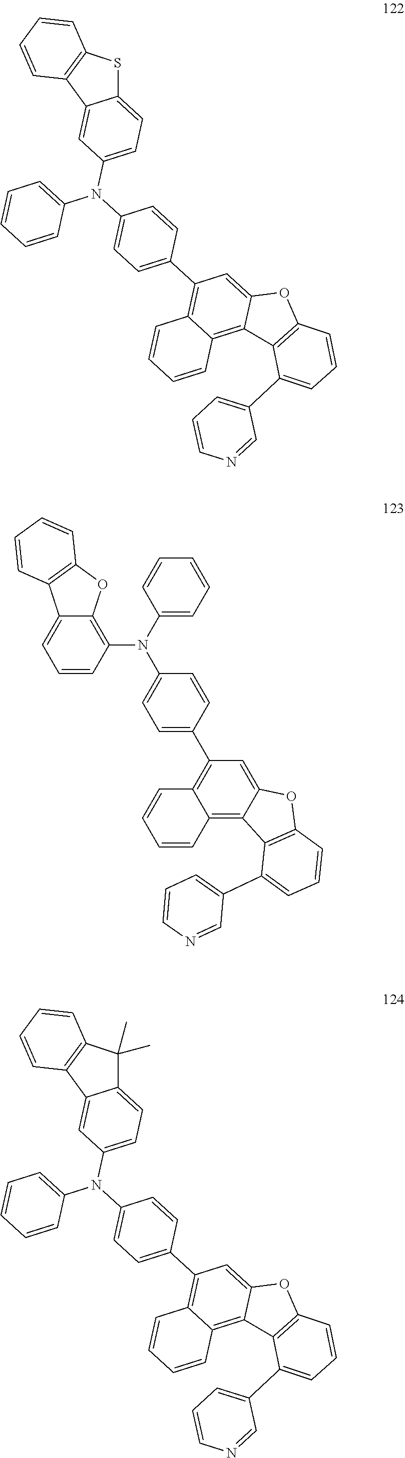 [SMILES: c1ccc(N(c2ccc(-c3cc4oc5cccc(-c6cccnc6)c5c4c4ccccc34)cc2)c2ccc3sc4ccccc4c3c2)cc1 ; c1ccc(N(c2ccc(-c3cc4oc5cccc(-c6cccnc6)c5c4c4ccccc34)cc2)c2cccc3c2oc2ccccc23)cc1 ; CC1(C)c2ccccc2-c2cc(N(c3ccccc3)c3ccc(-c4cc5oc6cccc(-c7cccnc7)c6c5c5ccccc45)cc3)ccc21]

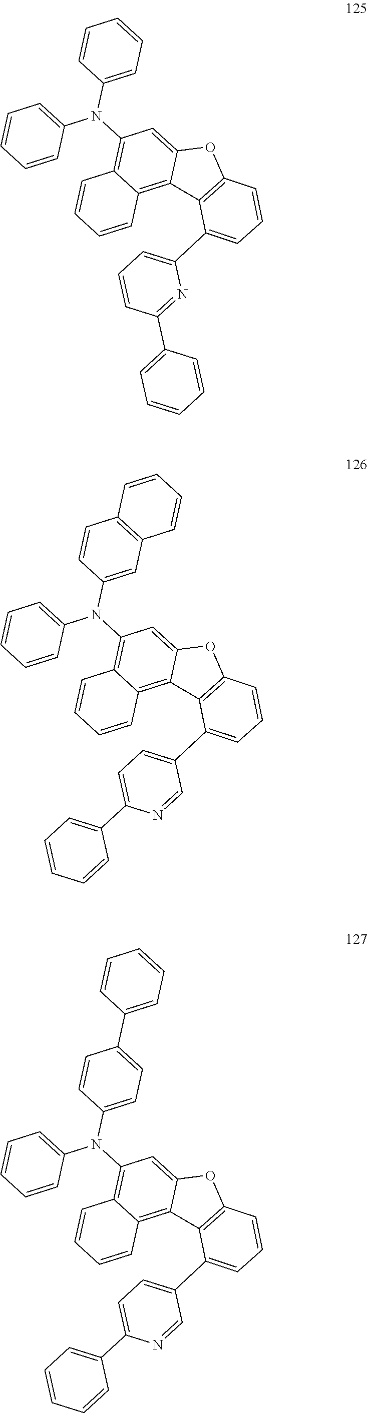 [SMILES: c1ccc(-c2cccc(-c3cccc4oc5cc(N(c6ccccc6)c6ccccc6)c6ccccc6c5c34)n2)cc1 ; c1ccc(-c2ccc(-c3cccc4oc5cc(N(c6ccccc6)c6ccc7ccccc7c6)c6ccccc6c5c34)cn2)cc1 ; c1ccc(-c2ccc(N(c3ccccc3)c3cc4oc5cccc(-c6ccc(-c7ccccc7)nc6)c5c4c4ccccc34)cc2)cc1]

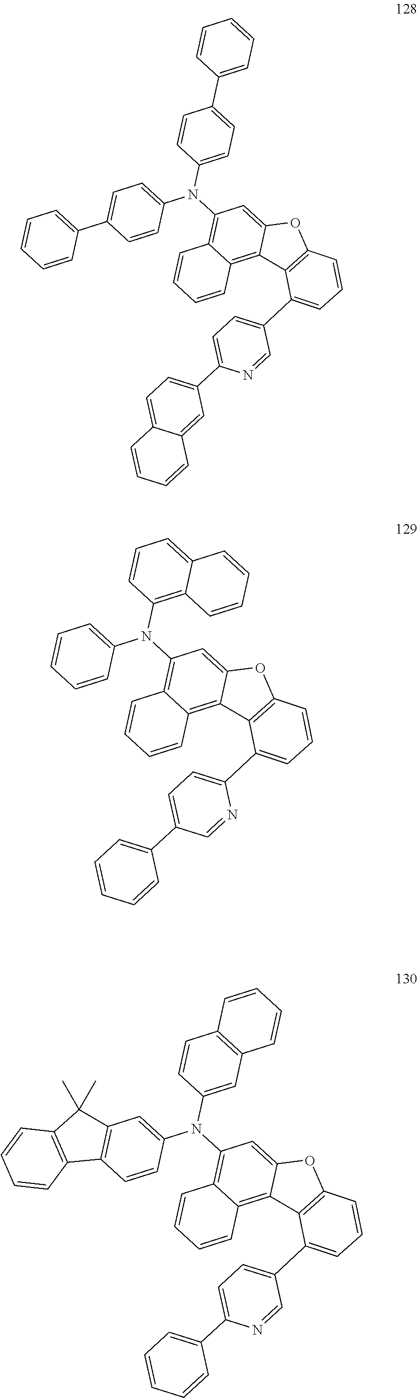 [SMILES: c1ccc(-c2ccc(N(c3ccc(-c4ccccc4)cc3)c3cc4oc5cccc(-c6ccc(-c7ccc8ccccc8c7)nc6)c5c4c4ccccc34)cc2)cc1 ; c1ccc(-c2ccc(-c3cccc4oc5cc(N(c6ccccc6)c6cccc7ccccc67)c6ccccc6c5c34)nc2)cc1 ; CC1(C)c2ccccc2-c2ccc(N(c3ccc4ccccc4c3)c3cc4oc5cccc(-c6ccc(-c7ccccc7)nc6)c5c4c4ccccc34)cc21]

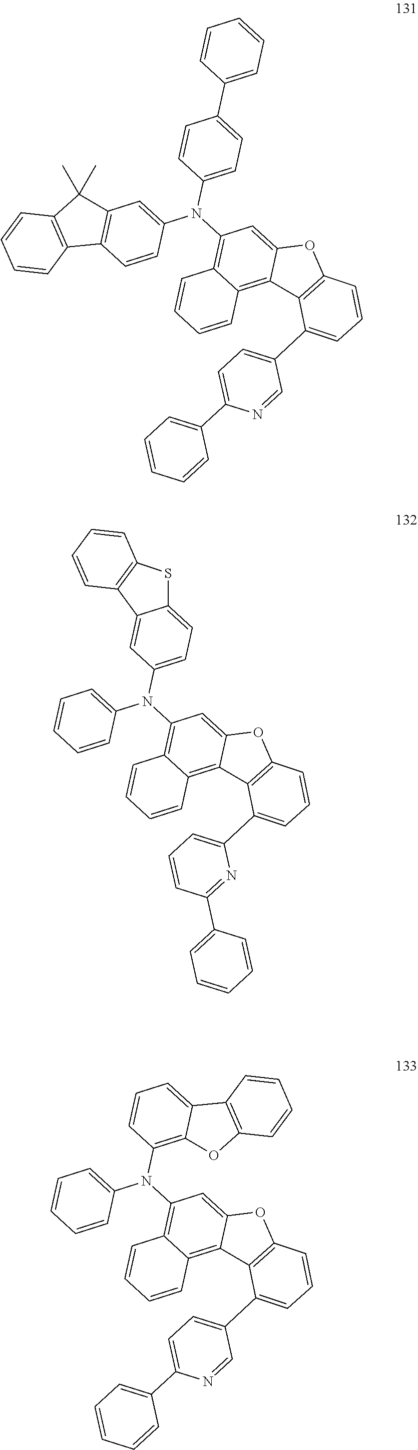 [SMILES: CC1(C)c2ccccc2-c2ccc(N(c3ccc(-c4ccccc4)cc3)c3cc4oc5cccc(-c6ccc(-c7ccccc7)nc6)c5c4c4ccccc34)cc21 ; c1ccc(-c2cccc(-c3cccc4oc5cc(N(c6ccccc6)c6ccc7sc8ccccc8c7c6)c6ccccc6c5c34)n2)cc1 ; c1ccc(-c2ccc(-c3cccc4oc5cc(N(c6ccccc6)c6cccc7c6oc6ccccc67)c6ccccc6c5c34)cn2)cc1]

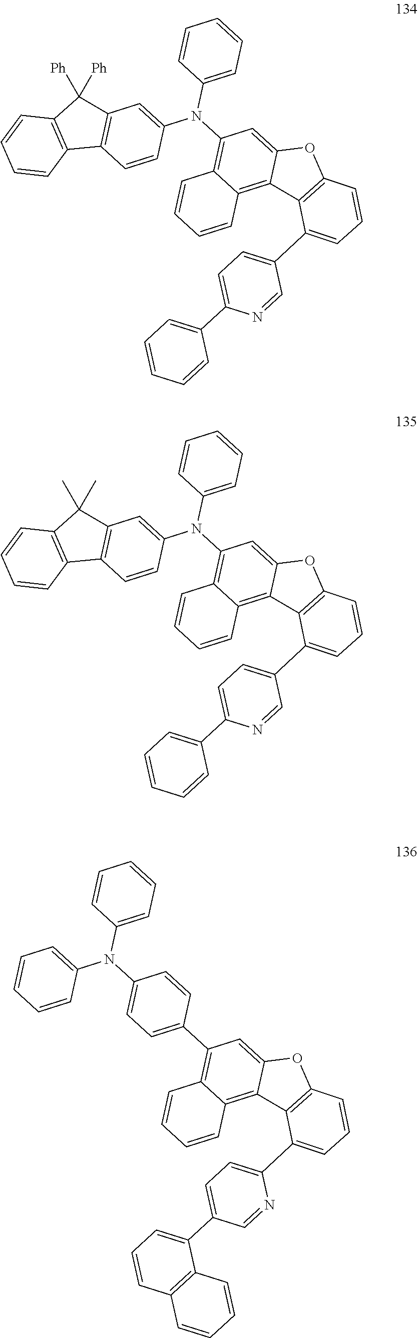 [SMILES: c1ccc(-c2ccc(-c3cccc4oc5cc(N(c6ccccc6)c6ccc7c(c6)C(c6ccccc6)(c6ccccc6)c6ccccc6-7)c6ccccc6c5c34)cn2)cc1 ; CC1(C)c2ccccc2-c2ccc(N(c3ccccc3)c3cc4oc5cccc(-c6ccc(-c7ccccc7)nc6)c5c4c4ccccc34)cc21 ; c1ccc(N(c2ccccc2)c2ccc(-c3cc4oc5cccc(-c6ccc(-c7cccc8ccccc78)cn6)c5c4c4ccccc34)cc2)cc1]

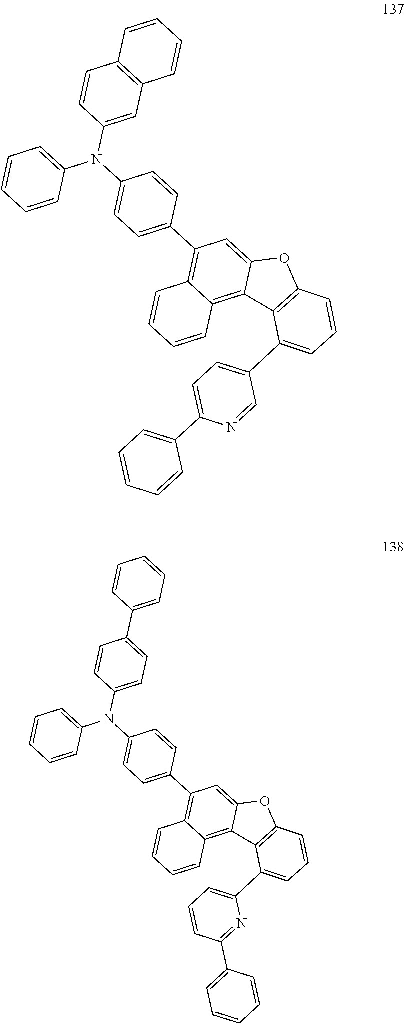 [SMILES: c1ccc(-c2ccc(-c3cccc4oc5cc(-c6ccc(N(c7ccccc7)c7ccc8ccccc8c7)cc6)c6ccccc6c5c34)cn2)cc1 ; c1ccc(-c2ccc(N(c3ccccc3)c3ccc(-c4cc5oc6cccc(-c7cccc(-c8ccccc8)n7)c6c5c5ccccc45)cc3)cc2)cc1]

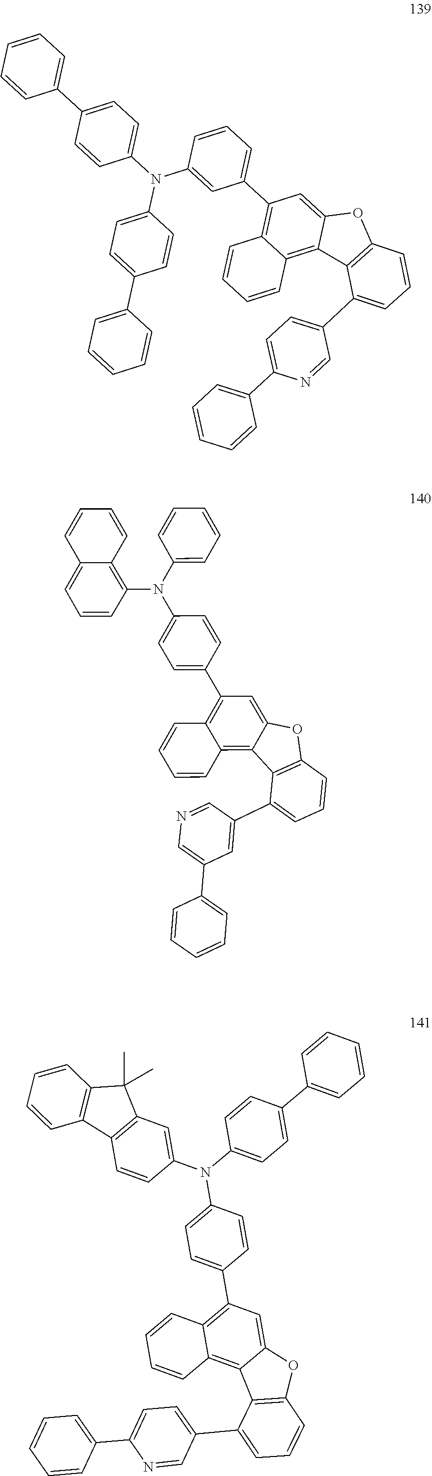 [SMILES: c1ccc(-c2ccc(N(c3ccc(-c4ccccc4)cc3)c3cccc(-c4cc5oc6cccc(-c7ccc(-c8ccccc8)nc7)c6c5c5ccccc45)c3)cc2)cc1 ; c1ccc(-c2cncc(-c3cccc4oc5cc(-c6ccc(N(c7ccccc7)c7cccc8ccccc78)cc6)c6ccccc6c5c34)c2)cc1 ; CC1(C)c2ccccc2-c2ccc(N(c3ccc(-c4ccccc4)cc3)c3ccc(-c4cc5oc6cccc(-c7ccc(-c8ccccc8)nc7)c6c5c5ccccc45)cc3)cc21]

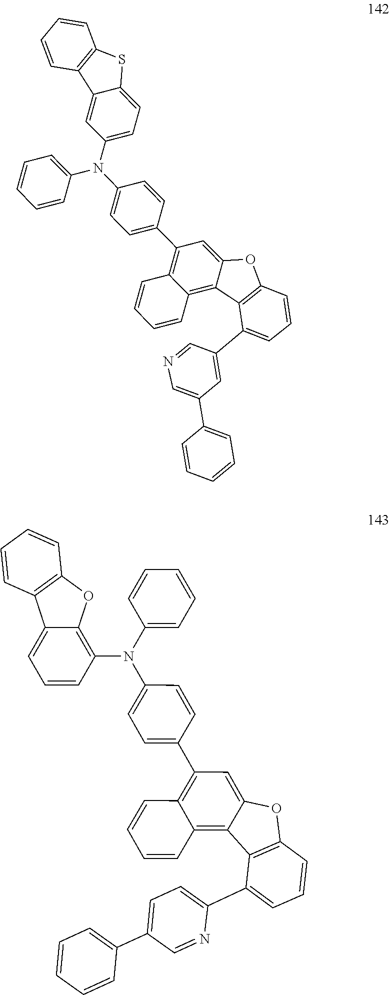 [SMILES: c1ccc(-c2cncc(-c3cccc4oc5cc(-c6ccc(N(c7ccccc7)c7ccc8sc9ccccc9c8c7)cc6)c6ccccc6c5c34)c2)cc1 ; c1ccc(-c2ccc(-c3cccc4oc5cc(-c6ccc(N(c7ccccc7)c7cccc8c7oc7ccccc78)cc6)c6ccccc6c5c34)nc2)cc1]

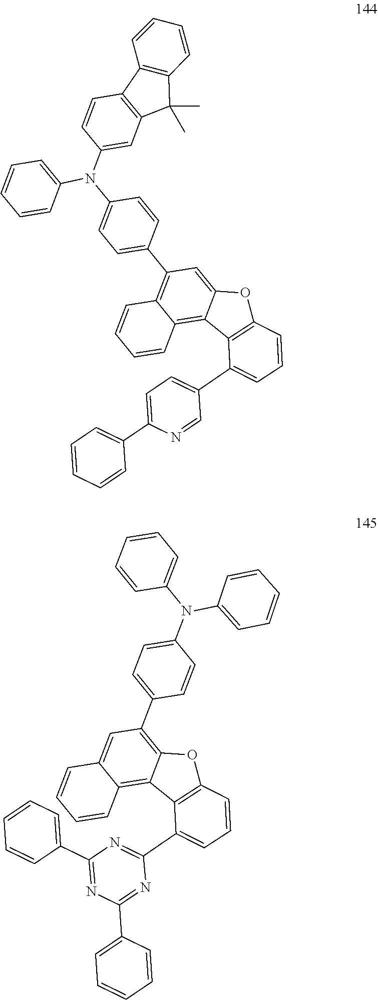 [SMILES: CC1(C)c2ccccc2-c2ccc(N(c3ccccc3)c3ccc(-c4cc5oc6cccc(-c7ccc(-c8ccccc8)nc7)c6c5c5ccccc45)cc3)cc21 ; c1ccc(-c2nc(-c3ccccc3)nc(-c3cccc4oc5c(-c6ccc(N(c7ccccc7)c7ccccc7)cc6)cc6ccccc6c5c34)n2)cc1]

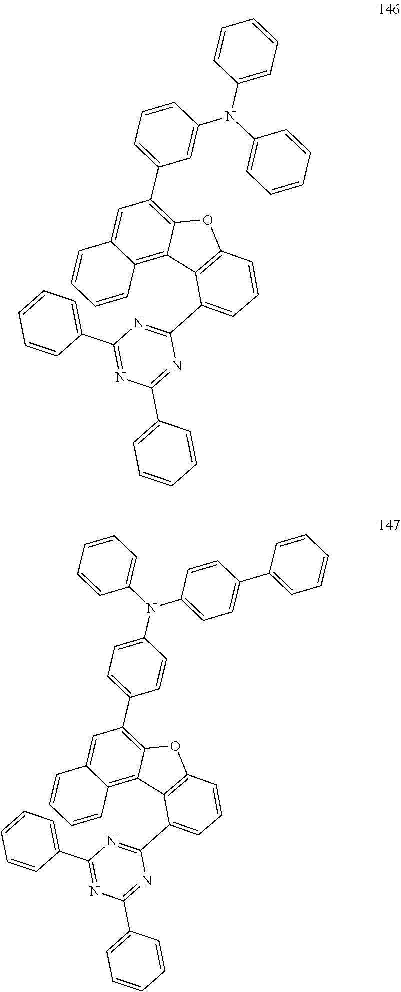 [SMILES: c1ccc(-c2nc(-c3ccccc3)nc(-c3cccc4oc5c(-c6cccc(N(c7ccccc7)c7ccccc7)c6)cc6ccccc6c5c34)n2)cc1 ; c1ccc(-c2ccc(N(c3ccccc3)c3ccc(-c4cc5ccccc5c5c4oc4cccc(-c6nc(-c7ccccc7)nc(-c7ccccc7)n6)c45)cc3)cc2)cc1]

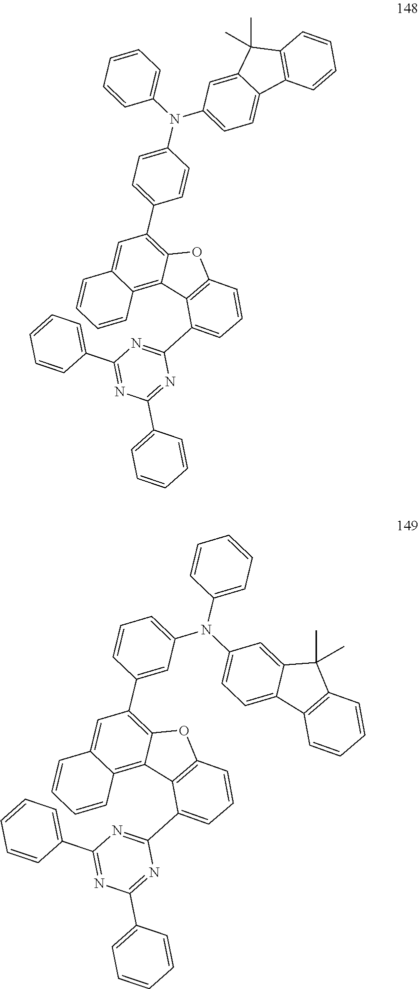 [SMILES: CC1(C)c2ccccc2-c2ccc(N(c3ccccc3)c3ccc(-c4cc5ccccc5c5c4oc4cccc(-c6nc(-c7ccccc7)nc(-c7ccccc7)n6)c45)cc3)cc21 ; CC1(C)c2ccccc2-c2ccc(N(c3ccccc3)c3cccc(-c4cc5ccccc5c5c4oc4cccc(-c6nc(-c7ccccc7)nc(-c7ccccc7)n6)c45)c3)cc21]

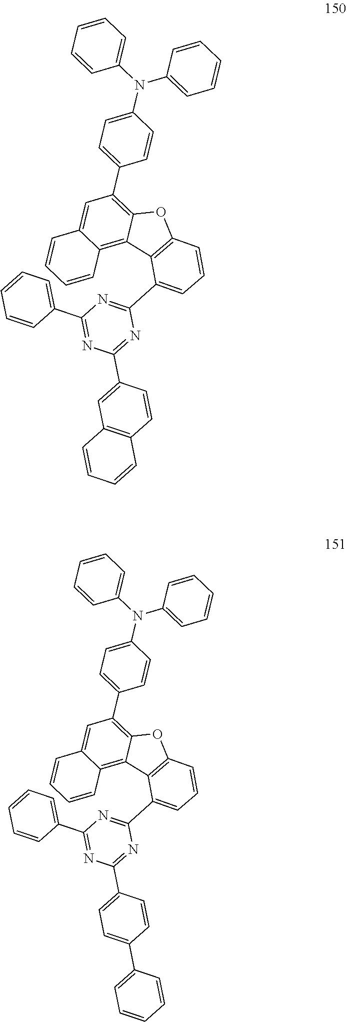 [SMILES: c1ccc(-c2nc(-c3ccc4ccccc4c3)nc(-c3cccc4oc5c(-c6ccc(N(c7ccccc7)c7ccccc7)cc6)cc6ccccc6c5c34)n2)cc1 ; c1ccc(-c2ccc(-c3nc(-c4ccccc4)nc(-c4cccc5oc6c(-c7ccc(N(c8ccccc8)c8ccccc8)cc7)cc7ccccc7c6c45)n3)cc2)cc1]

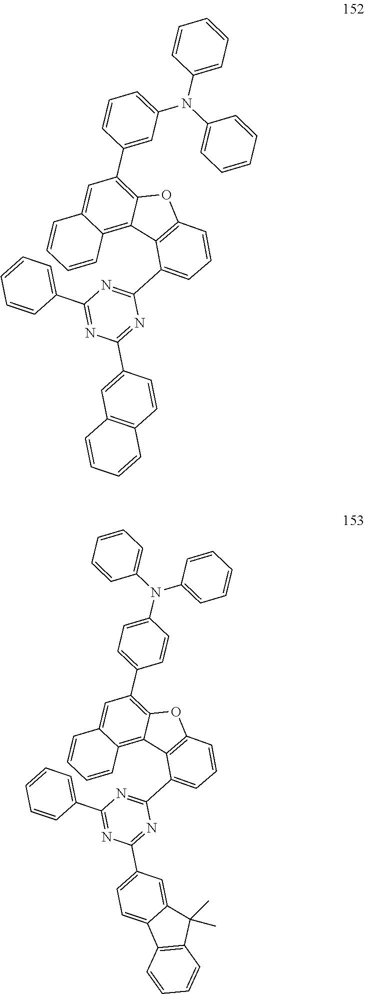 [SMILES: c1ccc(-c2nc(-c3ccc4ccccc4c3)nc(-c3cccc4oc5c(-c6cccc(N(c7ccccc7)c7ccccc7)c6)cc6ccccc6c5c34)n2)cc1 ; CC1(C)c2ccccc2-c2ccc(-c3nc(-c4ccccc4)nc(-c4cccc5oc6c(-c7ccc(N(c8ccccc8)c8ccccc8)cc7)cc7ccccc7c6c45)n3)cc21]

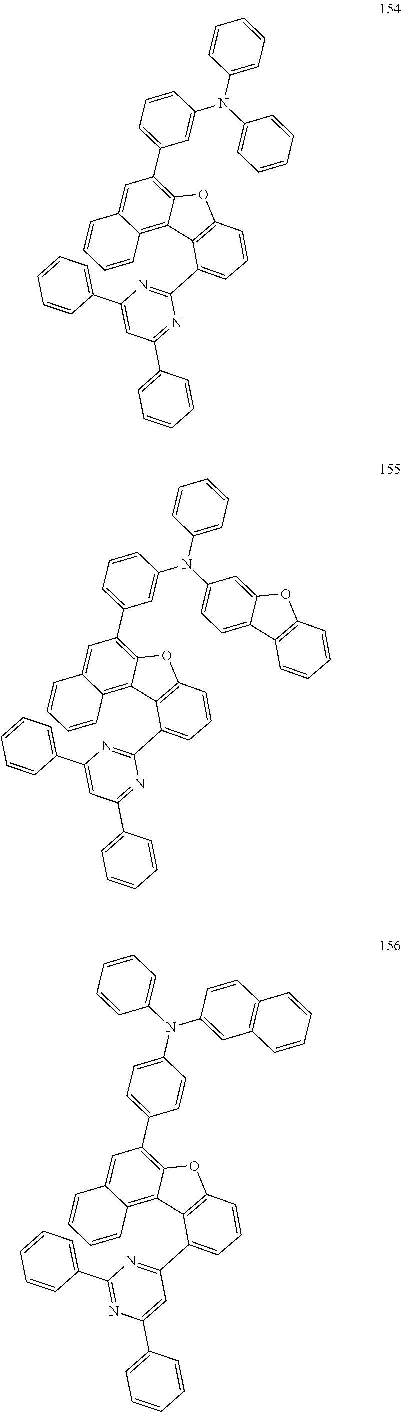 [SMILES: c1ccc(-c2cc(-c3ccccc3)nc(-c3cccc4oc5c(-c6cccc(N(c7ccccc7)c7ccccc7)c6)cc6ccccc6c5c34)n2)cc1 ; c1ccc(-c2cc(-c3ccccc3)nc(-c3cccc4oc5c(-c6cccc(N(c7ccccc7)c7ccc8c(c7)oc7ccccc78)c6)cc6ccccc6c5c34)n2)cc1 ; c1ccc(-c2cc(-c3cccc4oc5c(-c6ccc(N(c7ccccc7)c7ccc8ccccc8c7)cc6)cc6ccccc6c5c34)nc(-c3ccccc3)n2)cc1]

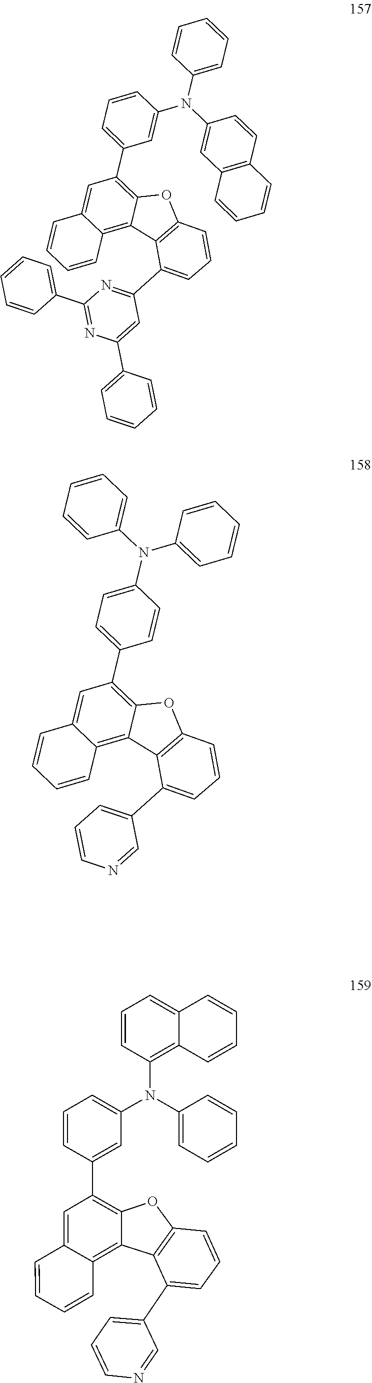 [SMILES: c1ccc(-c2cc(-c3cccc4oc5c(-c6cccc(N(c7ccccc7)c7ccc8ccccc8c7)c6)cc6ccccc6c5c34)nc(-c3ccccc3)n2)cc1 ; c1ccc(N(c2ccccc2)c2ccc(-c3cc4ccccc4c4c3oc3cccc(-c5cccnc5)c34)cc2)cc1 ; c1ccc(N(c2cccc(-c3cc4ccccc4c4c3oc3cccc(-c5cccnc5)c34)c2)c2cccc3ccccc23)cc1]

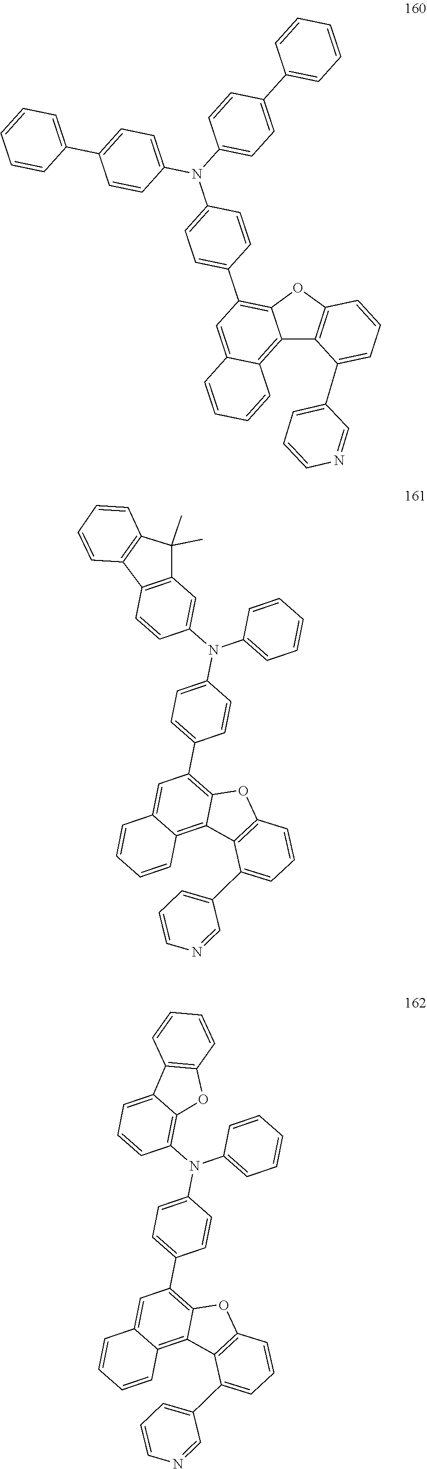 [SMILES: c1ccc(-c2ccc(N(c3ccc(-c4ccccc4)cc3)c3ccc(-c4cc5ccccc5c5c4oc4cccc(-c6cccnc6)c45)cc3)cc2)cc1 ; CC1(C)c2ccccc2-c2ccc(N(c3ccccc3)c3ccc(-c4cc5ccccc5c5c4oc4cccc(-c6cccnc6)c45)cc3)cc21 ; c1ccc(N(c2ccc(-c3cc4ccccc4c4c3oc3cccc(-c5cccnc5)c34)cc2)c2cccc3c2oc2ccccc23)cc1]

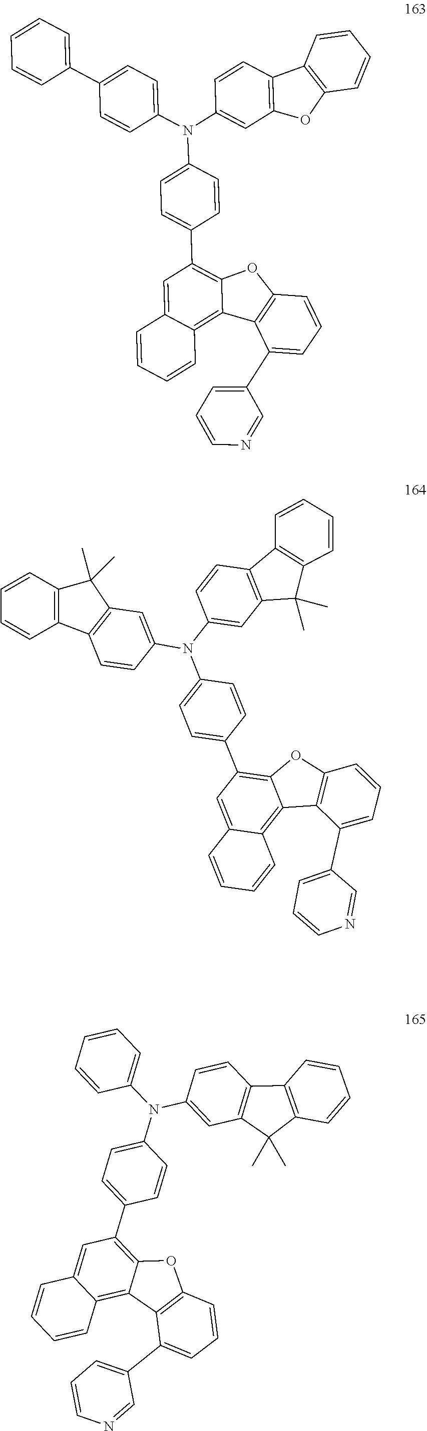 [SMILES: c1ccc(-c2ccc(N(c3ccc(-c4cc5ccccc5c5c4oc4cccc(-c6cccnc6)c45)cc3)c3ccc4c(c3)oc3ccccc34)cc2)cc1 ; CC1(C)c2ccccc2-c2ccc(N(c3ccc(-c4cc5ccccc5c5c4oc4cccc(-c6cccnc6)c45)cc3)c3ccc4c(c3)C(C)(C)c3ccccc3-4)cc21 ; CC1(C)c2ccccc2-c2ccc(N(c3ccccc3)c3ccc(-c4cc5ccccc5c5c4oc4cccc(-c6cccnc6)c45)cc3)cc21]

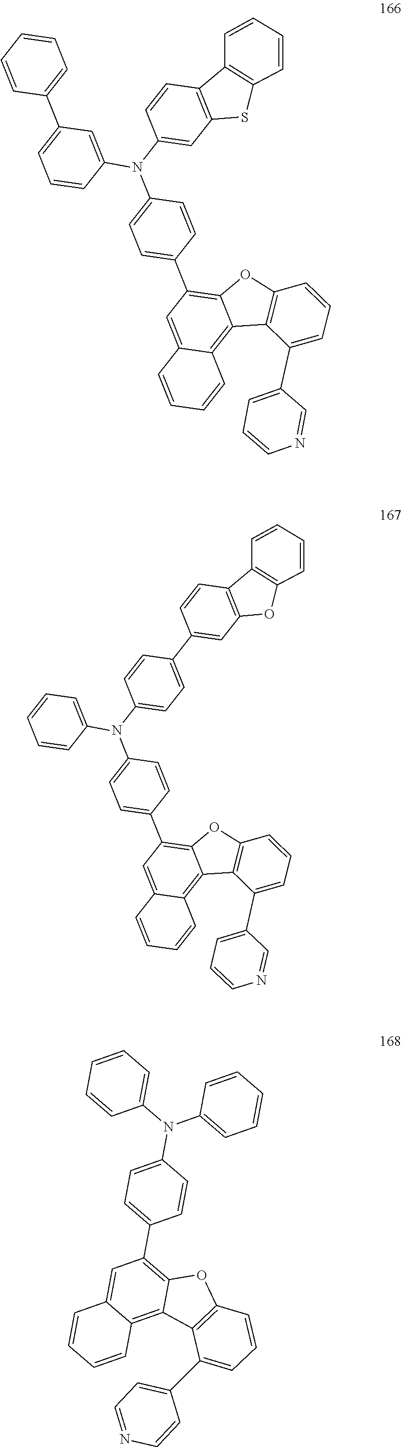 [SMILES: c1ccc(-c2cccc(N(c3ccc(-c4cc5ccccc5c5c4oc4cccc(-c6cccnc6)c45)cc3)c3ccc4c(c3)sc3ccccc34)c2)cc1 ; c1ccc(N(c2ccc(-c3ccc4c(c3)oc3ccccc34)cc2)c2ccc(-c3cc4ccccc4c4c3oc3cccc(-c5cccnc5)c34)cc2)cc1 ; c1ccc(N(c2ccccc2)c2ccc(-c3cc4ccccc4c4c3oc3cccc(-c5ccncc5)c34)cc2)cc1]

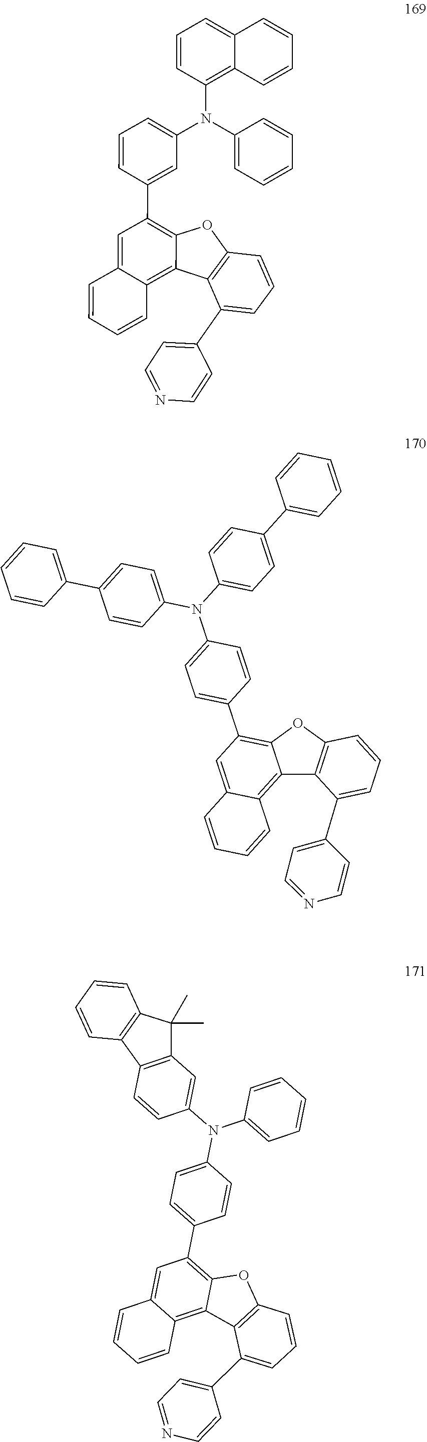 [SMILES: c1ccc(N(c2cccc(-c3cc4ccccc4c4c3oc3cccc(-c5ccncc5)c34)c2)c2cccc3ccccc23)cc1 ; c1ccc(-c2ccc(N(c3ccc(-c4ccccc4)cc3)c3ccc(-c4cc5ccccc5c5c4oc4cccc(-c6ccncc6)c45)cc3)cc2)cc1 ; CC1(C)c2ccccc2-c2ccc(N(c3ccccc3)c3ccc(-c4cc5ccccc5c5c4oc4cccc(-c6ccncc6)c45)cc3)cc21]

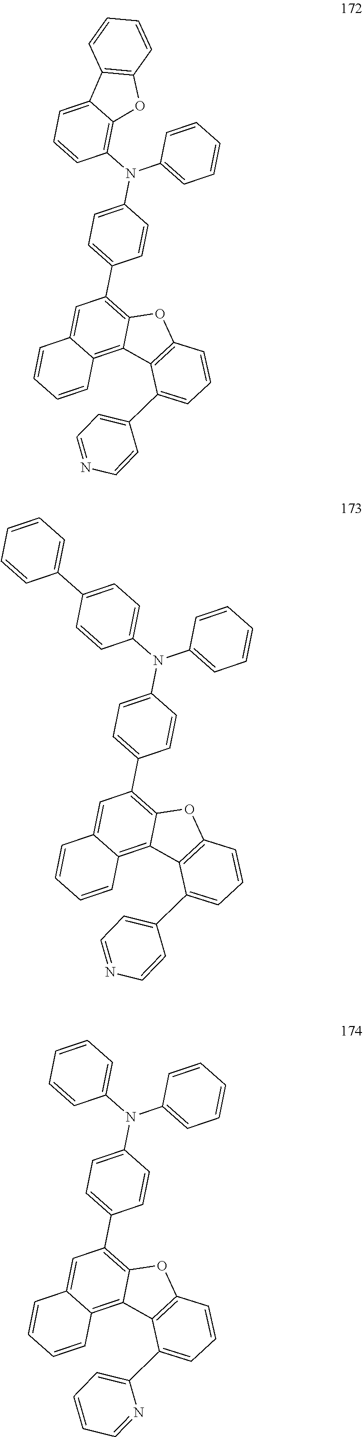 [SMILES: c1ccc(N(c2ccc(-c3cc4ccccc4c4c3oc3cccc(-c5ccncc5)c34)cc2)c2cccc3c2oc2ccccc23)cc1 ; c1ccc(-c2ccc(N(c3ccccc3)c3ccc(-c4cc5ccccc5c5c4oc4cccc(-c6ccncc6)c45)cc3)cc2)cc1 ; c1ccc(N(c2ccccc2)c2ccc(-c3cc4ccccc4c4c3oc3cccc(-c5ccccn5)c34)cc2)cc1]

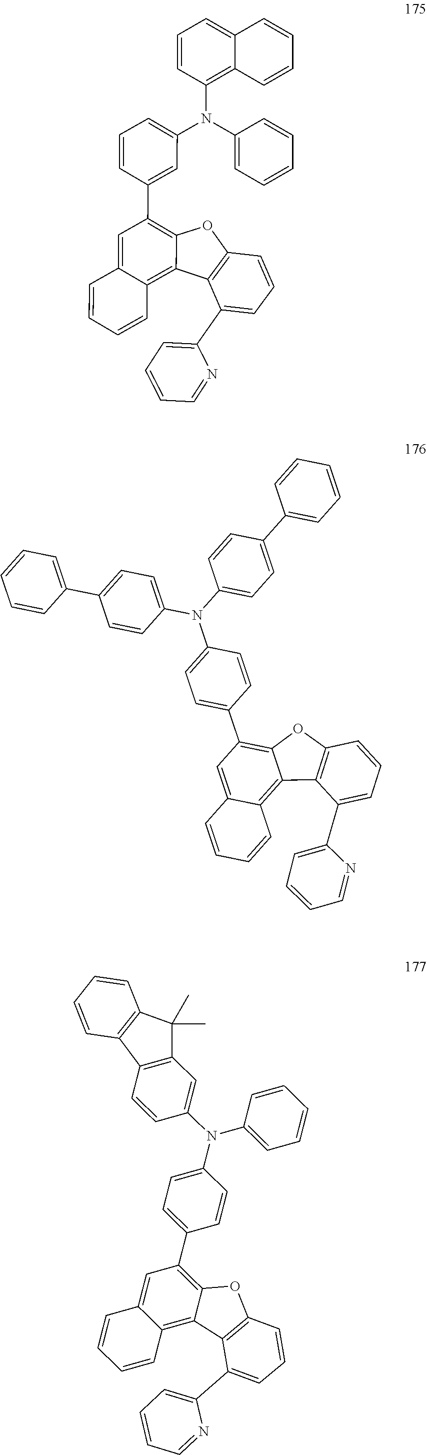 [SMILES: c1ccc(N(c2cccc(-c3cc4ccccc4c4c3oc3cccc(-c5ccccn5)c34)c2)c2cccc3ccccc23)cc1 ; c1ccc(-c2ccc(N(c3ccc(-c4ccccc4)cc3)c3ccc(-c4cc5ccccc5c5c4oc4cccc(-c6ccccn6)c45)cc3)cc2)cc1 ; CC1(C)c2ccccc2-c2ccc(N(c3ccccc3)c3ccc(-c4cc5ccccc5c5c4oc4cccc(-c6ccccn6)c45)cc3)cc21]

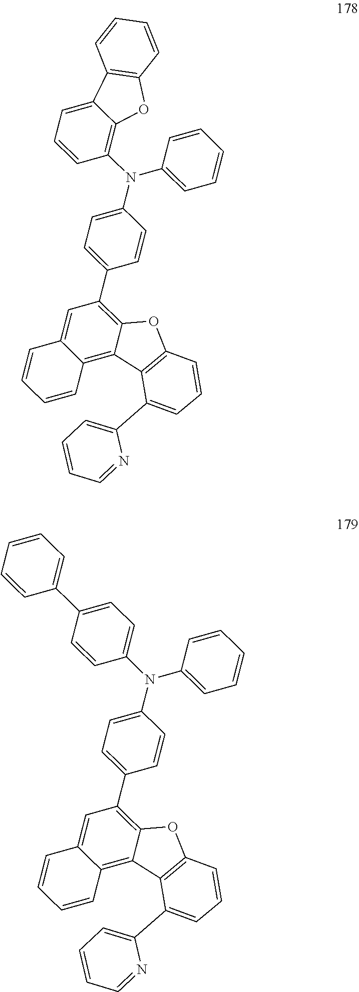 [SMILES: c1ccc(N(c2ccc(-c3cc4ccccc4c4c3oc3cccc(-c5ccccn5)c34)cc2)c2cccc3c2oc2ccccc23)cc1 ; c1ccc(-c2ccc(N(c3ccccc3)c3ccc(-c4cc5ccccc5c5c4oc4cccc(-c6ccccn6)c45)cc3)cc2)cc1]

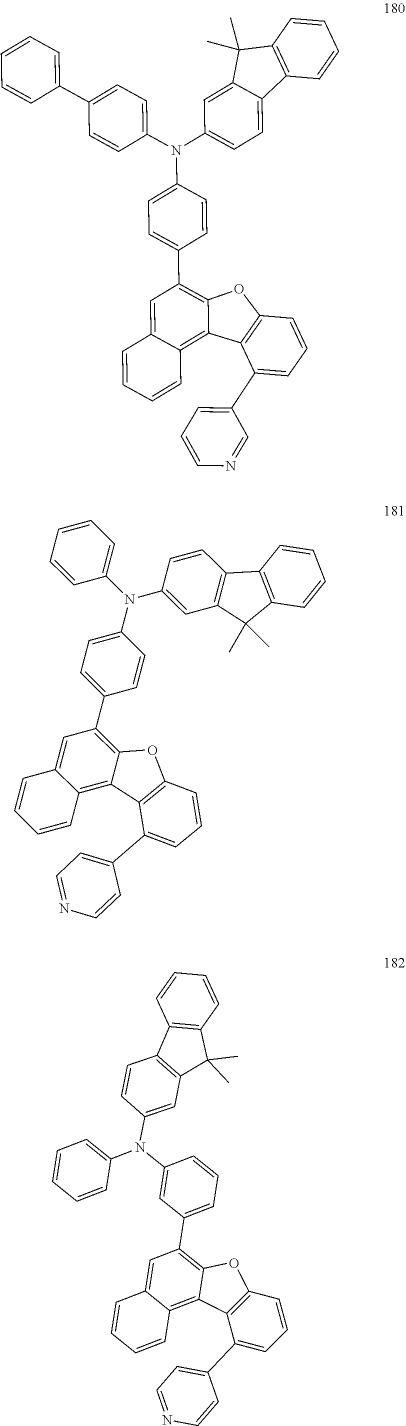 [SMILES: CC1(C)c2ccccc2-c2ccc(N(c3ccc(-c4ccccc4)cc3)c3ccc(-c4cc5ccccc5c5c4oc4cccc(-c6cccnc6)c45)cc3)cc21 ; CC1(C)c2ccccc2-c2ccc(N(c3ccccc3)c3ccc(-c4cc5ccccc5c5c4oc4cccc(-c6ccncc6)c45)cc3)cc21 ; CC1(C)c2ccccc2-c2ccc(N(c3ccccc3)c3cccc(-c4cc5ccccc5c5c4oc4cccc(-c6ccncc6)c45)c3)cc21]

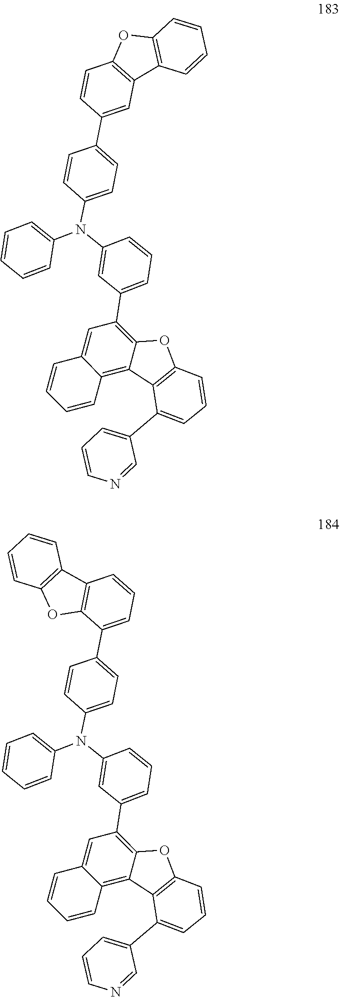 [SMILES: c1ccc(N(c2ccc(-c3ccc4oc5ccccc5c4c3)cc2)c2cccc(-c3cc4ccccc4c4c3oc3cccc(-c5cccnc5)c34)c2)cc1 ; c1ccc(N(c2ccc(-c3cccc4c3oc3ccccc34)cc2)c2cccc(-c3cc4ccccc4c4c3oc3cccc(-c5cccnc5)c34)c2)cc1]

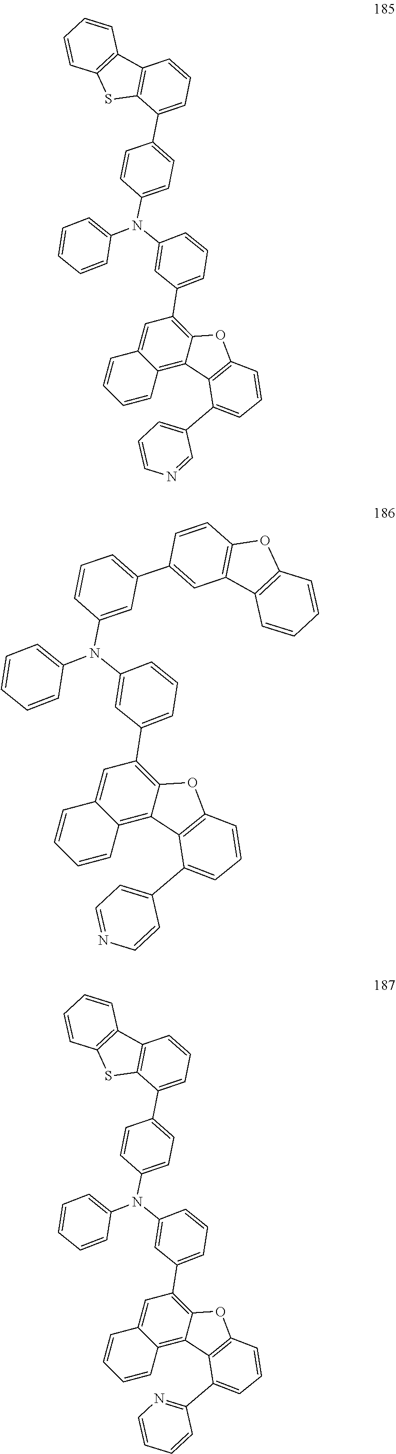 [SMILES: c1ccc(N(c2ccc(-c3cccc4c3sc3ccccc34)cc2)c2cccc(-c3cc4ccccc4c4c3oc3cccc(-c5cccnc5)c34)c2)cc1 ; c1ccc(N(c2cccc(-c3ccc4oc5ccccc5c4c3)c2)c2cccc(-c3cc4ccccc4c4c3oc3cccc(-c5ccncc5)c34)c2)cc1 ; c1ccc(N(c2ccc(-c3cccc4c3sc3ccccc34)cc2)c2cccc(-c3cc4ccccc4c4c3oc3cccc(-c5ccccn5)c34)c2)cc1]

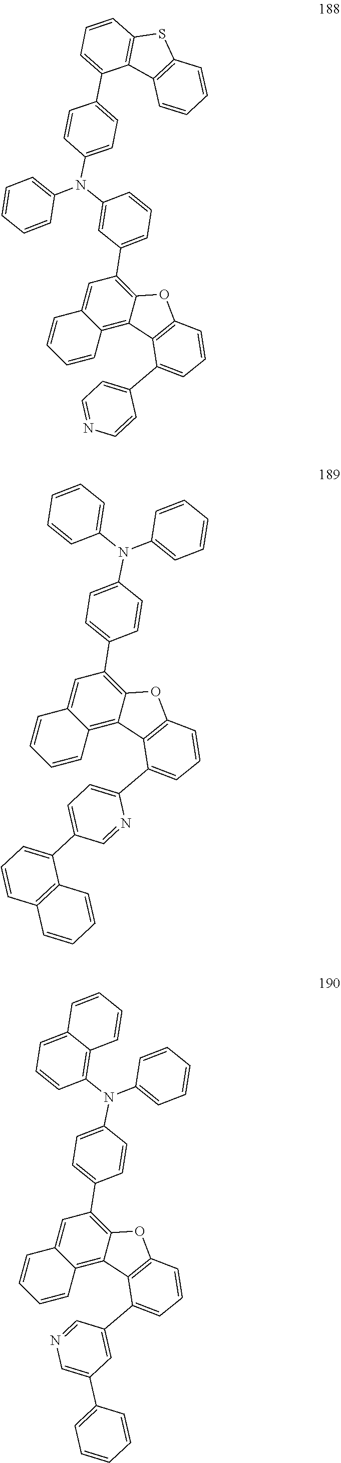 [SMILES: c1ccc(N(c2ccc(-c3cccc4sc5ccccc5c34)cc2)c2cccc(-c3cc4ccccc4c4c3oc3cccc(-c5ccncc5)c34)c2)cc1 ; c1ccc(N(c2ccccc2)c2ccc(-c3cc4ccccc4c4c3oc3cccc(-c5ccc(-c6cccc7ccccc67)cn5)c34)cc2)cc1 ; c1ccc(-c2cncc(-c3cccc4oc5c(-c6ccc(N(c7ccccc7)c7cccc8ccccc78)cc6)cc6ccccc6c5c34)c2)cc1]

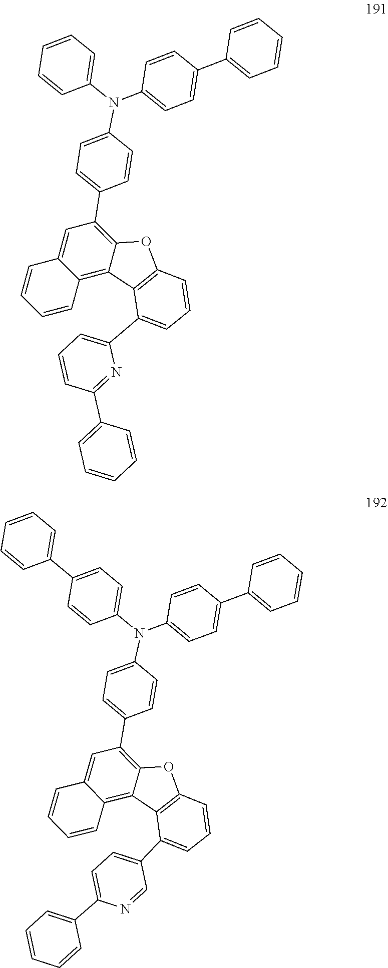 [SMILES: c1ccc(-c2ccc(N(c3ccccc3)c3ccc(-c4cc5ccccc5c5c4oc4cccc(-c6cccc(-c7ccccc7)n6)c45)cc3)cc2)cc1 ; c1ccc(-c2ccc(N(c3ccc(-c4ccccc4)cc3)c3ccc(-c4cc5ccccc5c5c4oc4cccc(-c6ccc(-c7ccccc7)nc6)c45)cc3)cc2)cc1]

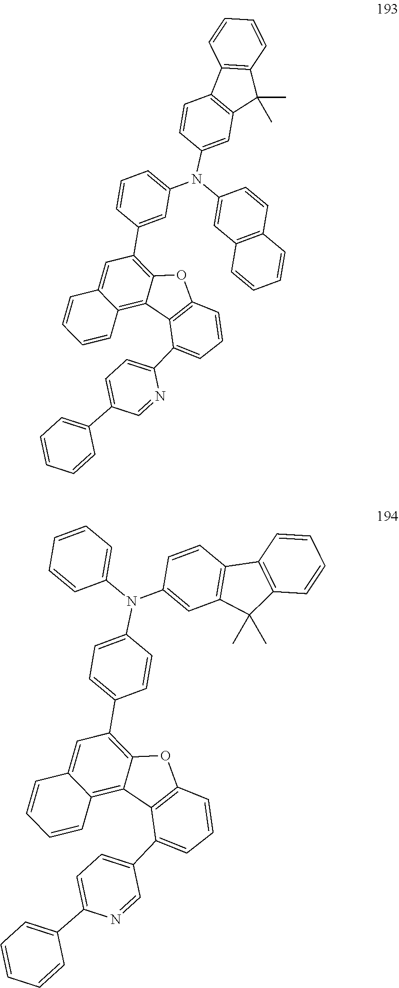 [SMILES: CC1(C)c2ccccc2-c2ccc(N(c3cccc(-c4cc5ccccc5c5c4oc4cccc(-c6ccc(-c7ccccc7)cn6)c45)c3)c3ccc4ccccc4c3)cc21 ; CC1(C)c2ccccc2-c2ccc(N(c3ccccc3)c3ccc(-c4cc5ccccc5c5c4oc4cccc(-c6ccc(-c7ccccc7)nc6)c45)cc3)cc21]

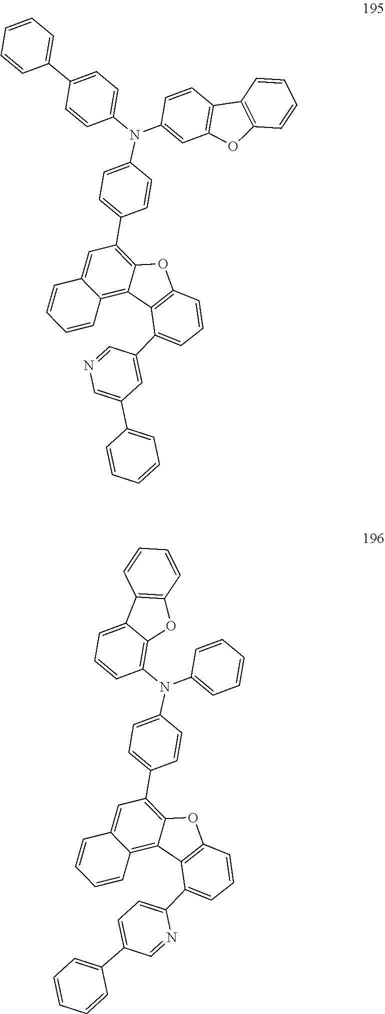 [SMILES: c1ccc(-c2ccc(N(c3ccc(-c4cc5ccccc5c5c4oc4cccc(-c6cncc(-c7ccccc7)c6)c45)cc3)c3ccc4c(c3)oc3ccccc34)cc2)cc1 ; c1ccc(-c2ccc(-c3cccc4oc5c(-c6ccc(N(c7ccccc7)c7cccc8c7oc7ccccc78)cc6)cc6ccccc6c5c34)nc2)cc1]

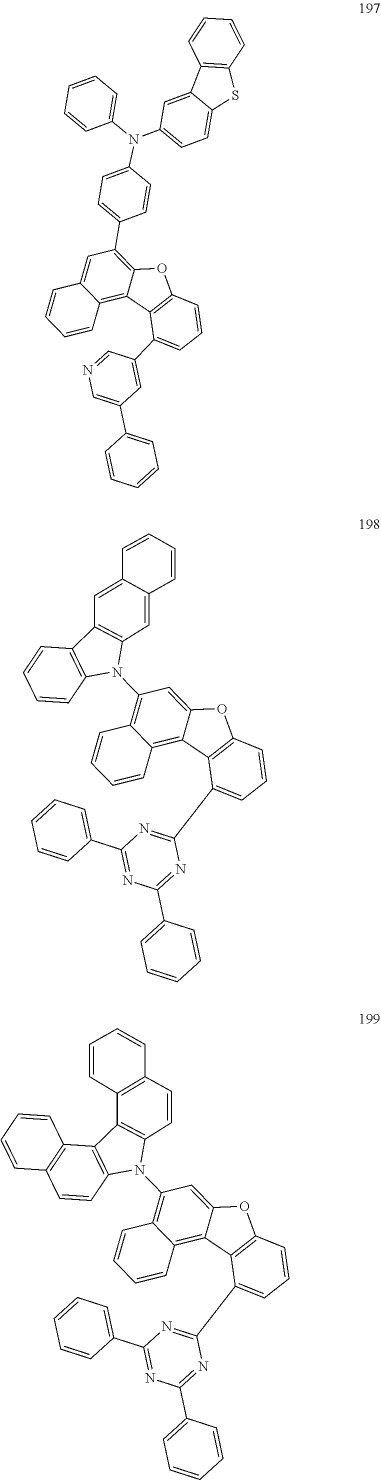 [SMILES: c1ccc(-c2cncc(-c3cccc4oc5c(-c6ccc(N(c7ccccc7)c7ccc8sc9ccccc9c8c7)cc6)cc6ccccc6c5c34)c2)cc1 ; c1ccc(-c2nc(-c3ccccc3)nc(-c3cccc4oc5cc(-n6c7ccccc7c7cc8ccccc8cc76)c6ccccc6c5c34)n2)cc1 ; c1ccc(-c2nc(-c3ccccc3)nc(-c3cccc4oc5cc(-n6c7ccc8ccccc8c7c7c8ccccc8ccc76)c6ccccc6c5c34)n2)cc1]

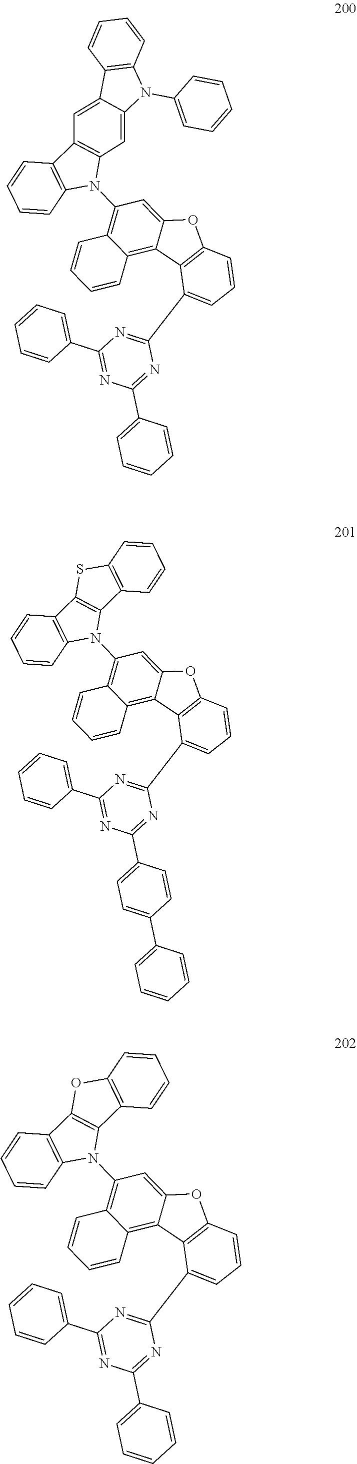 [SMILES: c1ccc(-c2nc(-c3ccccc3)nc(-c3cccc4oc5cc(-n6c7ccccc7c7cc8c9ccccc9n(-c9ccccc9)c8cc76)c6ccccc6c5c34)n2)cc1 ; c1ccc(-c2ccc(-c3nc(-c4ccccc4)nc(-c4cccc5oc6cc(-n7c8ccccc8c8sc9ccccc9c87)c7ccccc7c6c45)n3)cc2)cc1 ; c1ccc(-c2nc(-c3ccccc3)nc(-c3cccc4oc5cc(-n6c7ccccc7c7oc8ccccc8c76)c6ccccc6c5c34)n2)cc1]

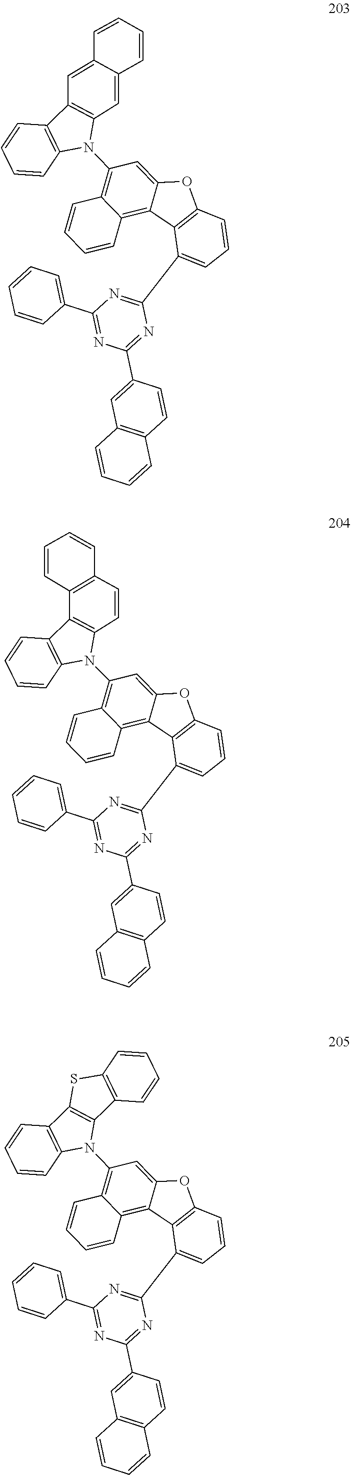 [SMILES: c1ccc(-c2nc(-c3ccc4ccccc4c3)nc(-c3cccc4oc5cc(-n6c7ccccc7c7cc8ccccc8cc76)c6ccccc6c5c34)n2)cc1 ; c1ccc(-c2nc(-c3ccc4ccccc4c3)nc(-c3cccc4oc5cc(-n6c7ccccc7c7c8ccccc8ccc76)c6ccccc6c5c34)n2)cc1 ; c1ccc(-c2nc(-c3ccc4ccccc4c3)nc(-c3cccc4oc5cc(-n6c7ccccc7c7sc8ccccc8c76)c6ccccc6c5c34)n2)cc1]

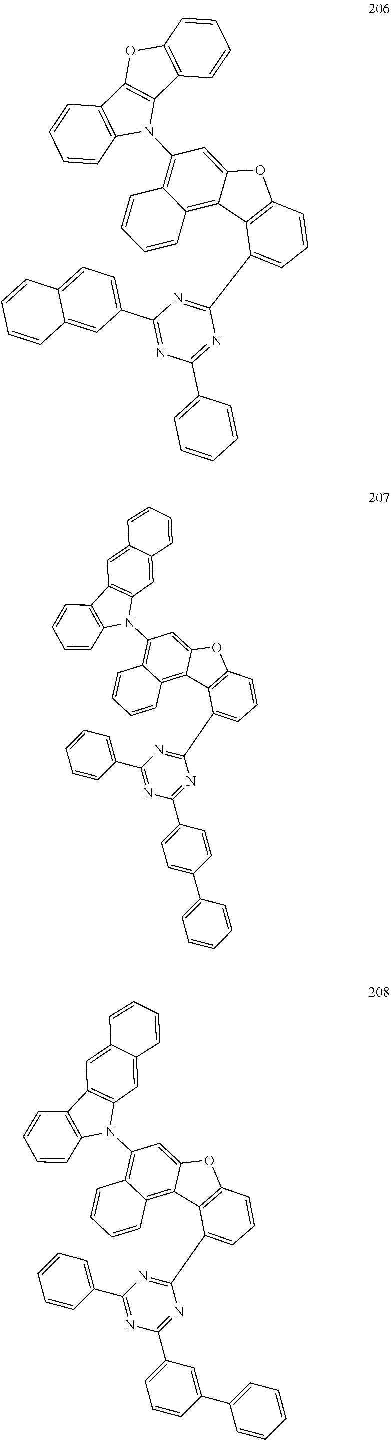 [SMILES: c1ccc(-c2nc(-c3ccc4ccccc4c3)nc(-c3cccc4oc5cc(-n6c7ccccc7c7oc8ccccc8c76)c6ccccc6c5c34)n2)cc1 ; c1ccc(-c2ccc(-c3nc(-c4ccccc4)nc(-c4cccc5oc6cc(-n7c8ccccc8c8cc9ccccc9cc87)c7ccccc7c6c45)n3)cc2)cc1 ; c1ccc(-c2cccc(-c3nc(-c4ccccc4)nc(-c4cccc5oc6cc(-n7c8ccccc8c8cc9ccccc9cc87)c7ccccc7c6c45)n3)c2)cc1]

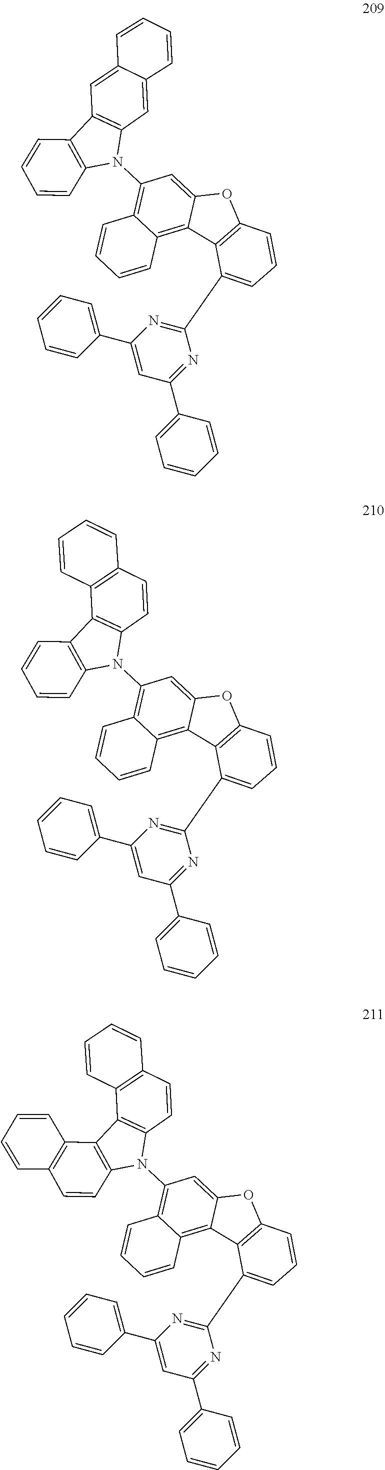 [SMILES: c1ccc(-c2cc(-c3ccccc3)nc(-c3cccc4oc5cc(-n6c7ccccc7c7cc8ccccc8cc76)c6ccccc6c5c34)n2)cc1 ; c1ccc(-c2cc(-c3ccccc3)nc(-c3cccc4oc5cc(-n6c7ccccc7c7c8ccccc8ccc76)c6ccccc6c5c34)n2)cc1 ; c1ccc(-c2cc(-c3ccccc3)nc(-c3cccc4oc5cc(-n6c7ccc8ccccc8c7c7c8ccccc8ccc76)c6ccccc6c5c34)n2)cc1]

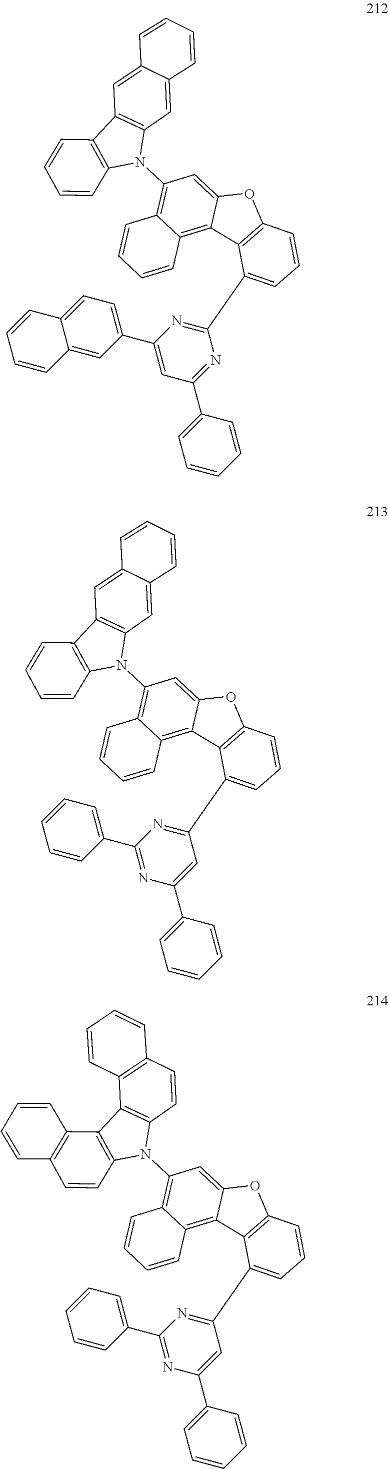 [SMILES: c1ccc(-c2cc(-c3ccc4ccccc4c3)nc(-c3cccc4oc5cc(-n6c7ccccc7c7cc8ccccc8cc76)c6ccccc6c5c34)n2)cc1 ; c1ccc(-c2cc(-c3cccc4oc5cc(-n6c7ccccc7c7cc8ccccc8cc76)c6ccccc6c5c34)nc(-c3ccccc3)n2)cc1 ; c1ccc(-c2cc(-c3cccc4oc5cc(-n6c7ccc8ccccc8c7c7c8ccccc8ccc76)c6ccccc6c5c34)nc(-c3ccccc3)n2)cc1]

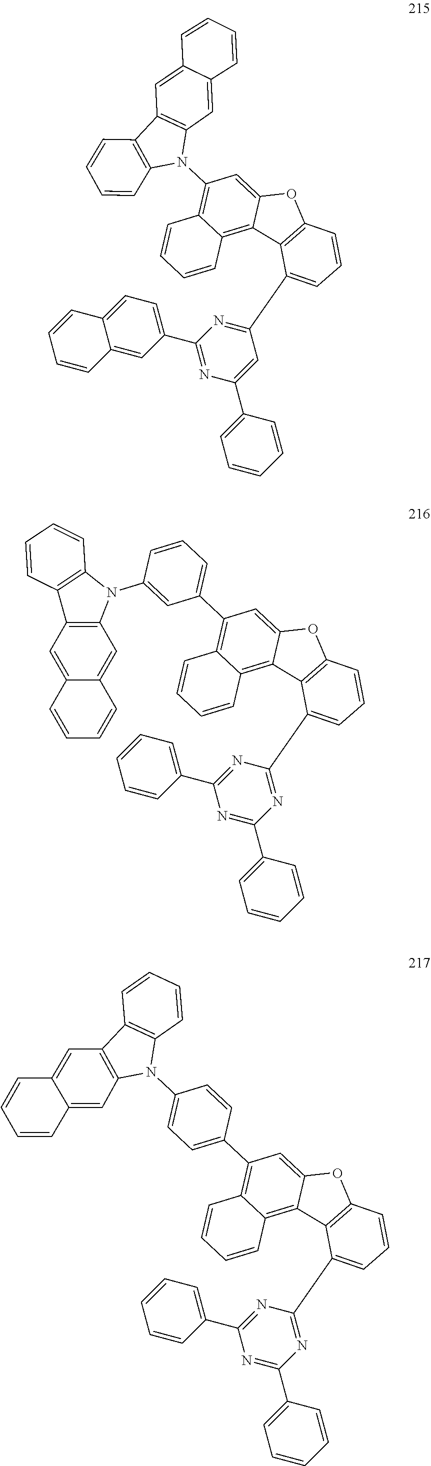 [SMILES: c1ccc(-c2cc(-c3cccc4oc5cc(-n6c7ccccc7c7cc8ccccc8cc76)c6ccccc6c5c34)nc(-c3ccc4ccccc4c3)n2)cc1 ; c1ccc(-c2nc(-c3ccccc3)nc(-c3cccc4oc5cc(-c6cccc(-n7c8ccccc8c8cc9ccccc9cc87)c6)c6ccccc6c5c34)n2)cc1 ; c1ccc(-c2nc(-c3ccccc3)nc(-c3cccc4oc5cc(-c6ccc(-n7c8ccccc8c8cc9ccccc9cc87)cc6)c6ccccc6c5c34)n2)cc1]

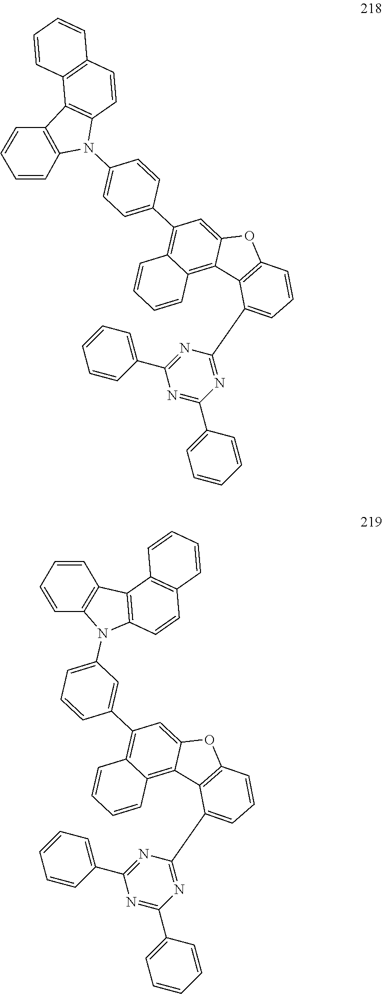 [SMILES: c1ccc(-c2nc(-c3ccccc3)nc(-c3cccc4oc5cc(-c6ccc(-n7c8ccccc8c8c9ccccc9ccc87)cc6)c6ccccc6c5c34)n2)cc1 ; c1ccc(-c2nc(-c3ccccc3)nc(-c3cccc4oc5cc(-c6cccc(-n7c8ccccc8c8c9ccccc9ccc87)c6)c6ccccc6c5c34)n2)cc1]

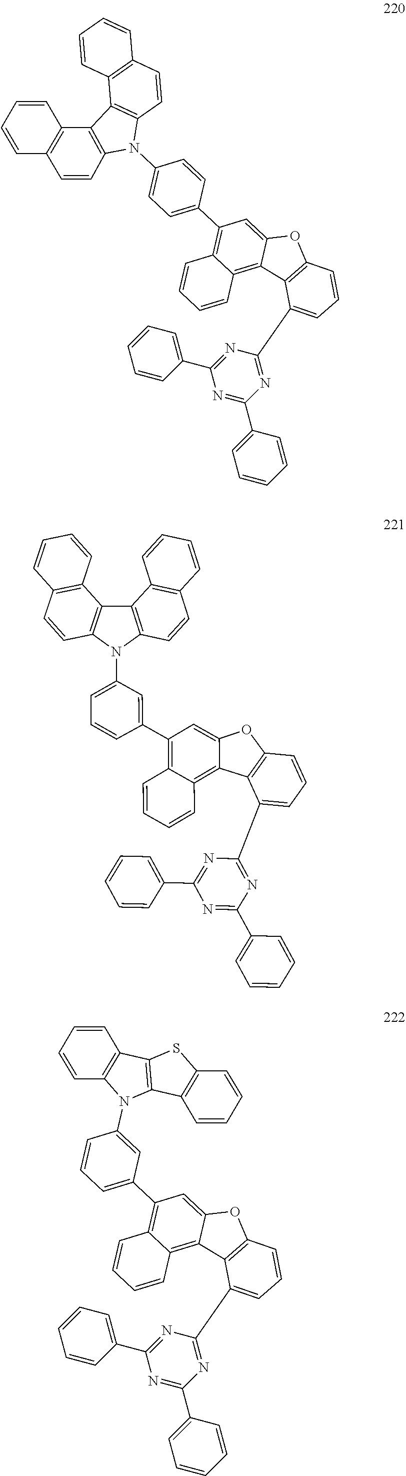 [SMILES: c1ccc(-c2nc(-c3ccccc3)nc(-c3cccc4oc5cc(-c6ccc(-n7c8ccc9ccccc9c8c8c9ccccc9ccc87)cc6)c6ccccc6c5c34)n2)cc1 ; c1ccc(-c2nc(-c3ccccc3)nc(-c3cccc4oc5cc(-c6cccc(-n7c8ccc9ccccc9c8c8c9ccccc9ccc87)c6)c6ccccc6c5c34)n2)cc1 ; c1ccc(-c2nc(-c3ccccc3)nc(-c3cccc4oc5cc(-c6cccc(-n7c8ccccc8c8sc9ccccc9c87)c6)c6ccccc6c5c34)n2)cc1]

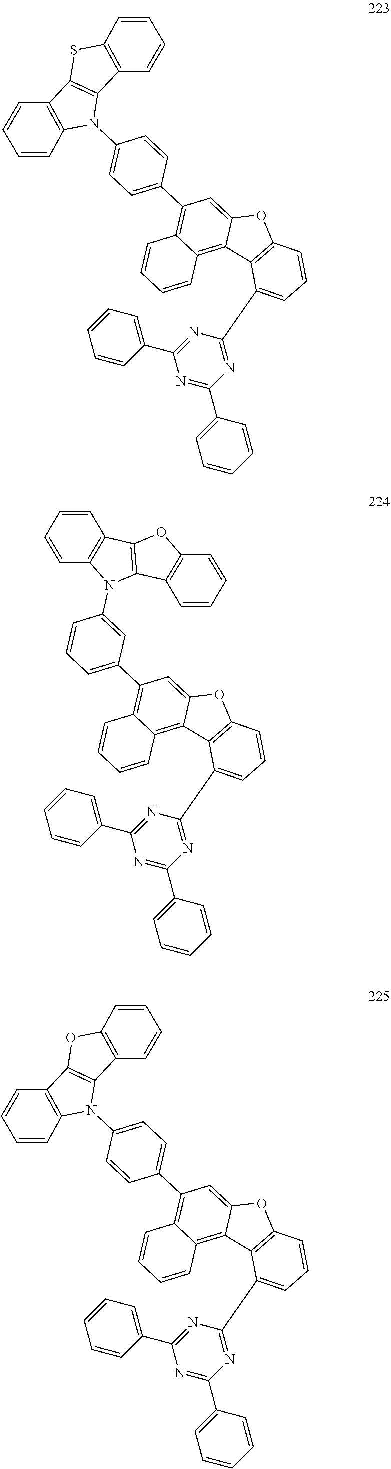 [SMILES: c1ccc(-c2nc(-c3ccccc3)nc(-c3cccc4oc5cc(-c6ccc(-n7c8ccccc8c8sc9ccccc9c87)cc6)c6ccccc6c5c34)n2)cc1 ; c1ccc(-c2nc(-c3ccccc3)nc(-c3cccc4oc5cc(-c6cccc(-n7c8ccccc8c8oc9ccccc9c87)c6)c6ccccc6c5c34)n2)cc1 ; c1ccc(-c2nc(-c3ccccc3)nc(-c3cccc4oc5cc(-c6ccc(-n7c8ccccc8c8oc9ccccc9c87)cc6)c6ccccc6c5c34)n2)cc1]

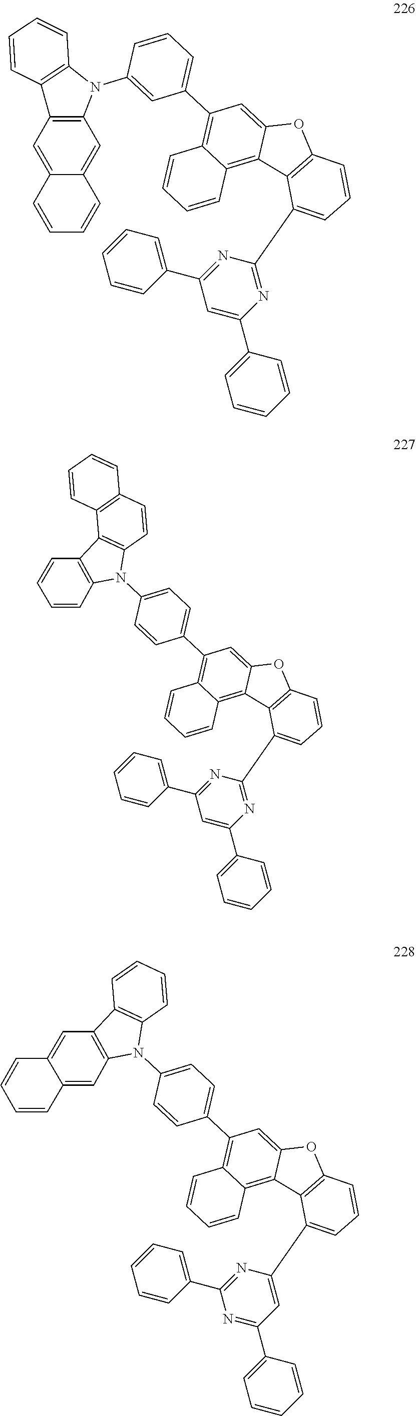 [SMILES: c1ccc(-c2cc(-c3ccccc3)nc(-c3cccc4oc5cc(-c6cccc(-n7c8ccccc8c8cc9ccccc9cc87)c6)c6ccccc6c5c34)n2)cc1 ; c1ccc(-c2cc(-c3ccccc3)nc(-c3cccc4oc5cc(-c6ccc(-n7c8ccccc8c8c9ccccc9ccc87)cc6)c6ccccc6c5c34)n2)cc1 ; c1ccc(-c2cc(-c3cccc4oc5cc(-c6ccc(-n7c8ccccc8c8cc9ccccc9cc87)cc6)c6ccccc6c5c34)nc(-c3ccccc3)n2)cc1]

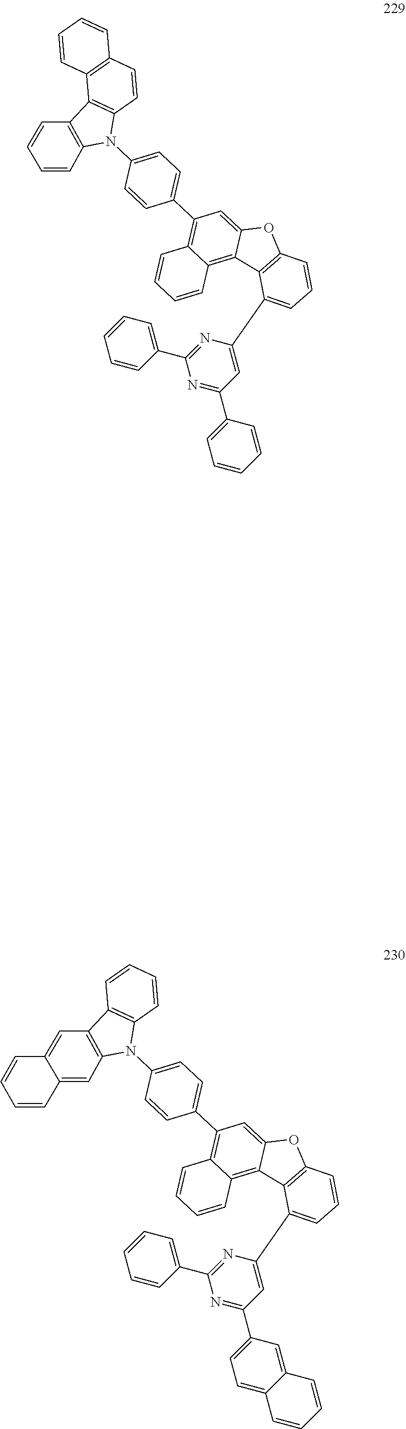 [SMILES: c1ccc(-c2cc(-c3cccc4oc5cc(-c6ccc(-n7c8ccccc8c8c9ccccc9ccc87)cc6)c6ccccc6c5c34)nc(-c3ccccc3)n2)cc1 ; c1ccc(-c2nc(-c3ccc4ccccc4c3)cc(-c3cccc4oc5cc(-c6ccc(-n7c8ccccc8c8cc9ccccc9cc87)cc6)c6ccccc6c5c34)n2)cc1]

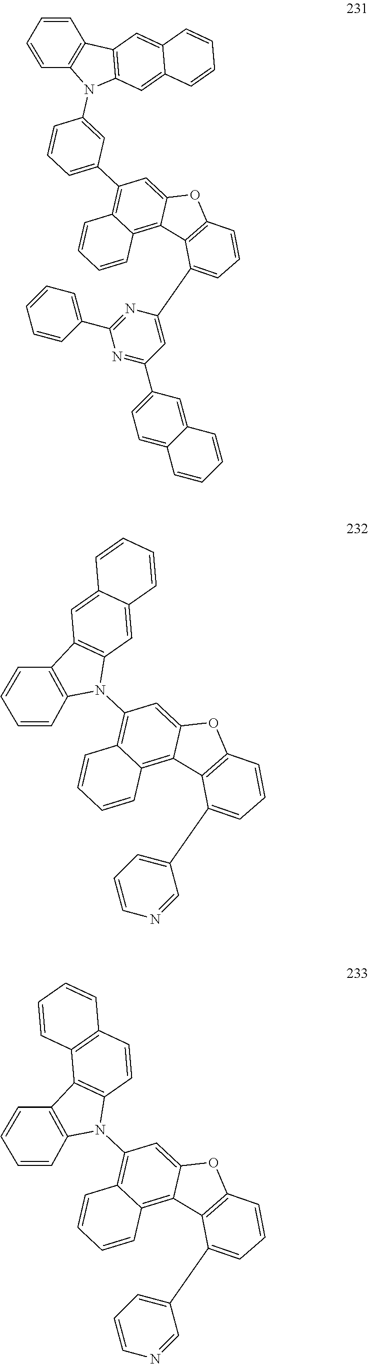 [SMILES: c1ccc(-c2nc(-c3ccc4ccccc4c3)cc(-c3cccc4oc5cc(-c6cccc(-n7c8ccccc8c8cc9ccccc9cc87)c6)c6ccccc6c5c34)n2)cc1 ; c1cncc(-c2cccc3oc4cc(-n5c6ccccc6c6cc7ccccc7cc65)c5ccccc5c4c23)c1 ; c1cncc(-c2cccc3oc4cc(-n5c6ccccc6c6c7ccccc7ccc65)c5ccccc5c4c23)c1]

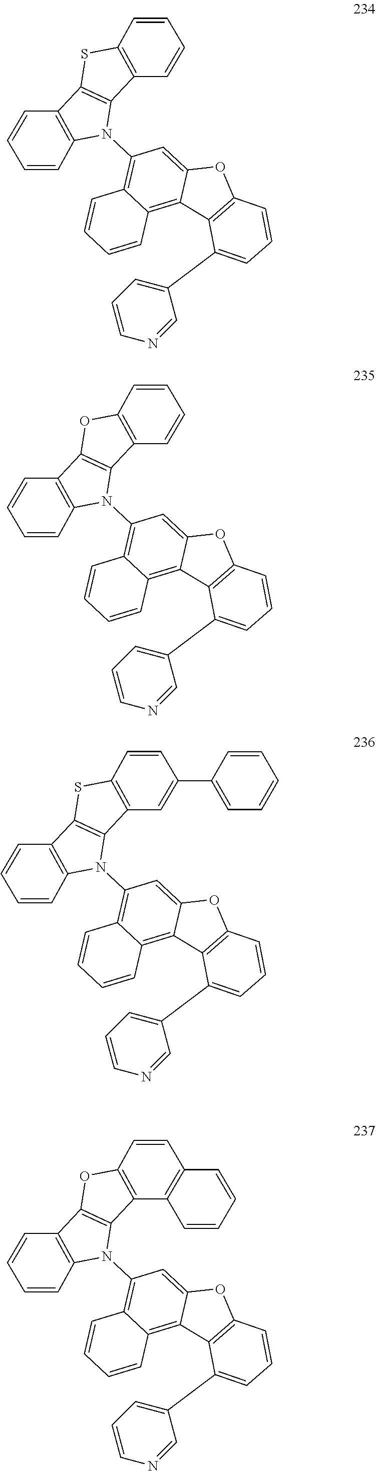

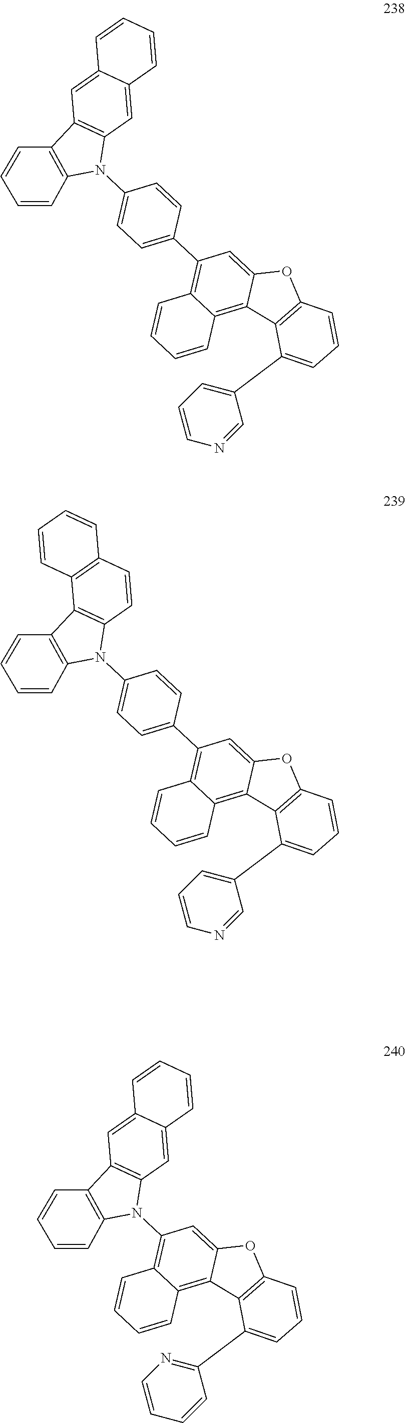 [SMILES: c1cncc(-c2cccc3oc4cc(-c5ccc(-n6c7ccccc7c7cc8ccccc8cc76)cc5)c5ccccc5c4c23)c1 ; c1cncc(-c2cccc3oc4cc(-c5ccc(-n6c7ccccc7c7c8ccccc8ccc76)cc5)c5ccccc5c4c23)c1 ; c1ccc(-c2cccc3oc4cc(-n5c6ccccc6c6cc7ccccc7cc65)c5ccccc5c4c23)nc1]

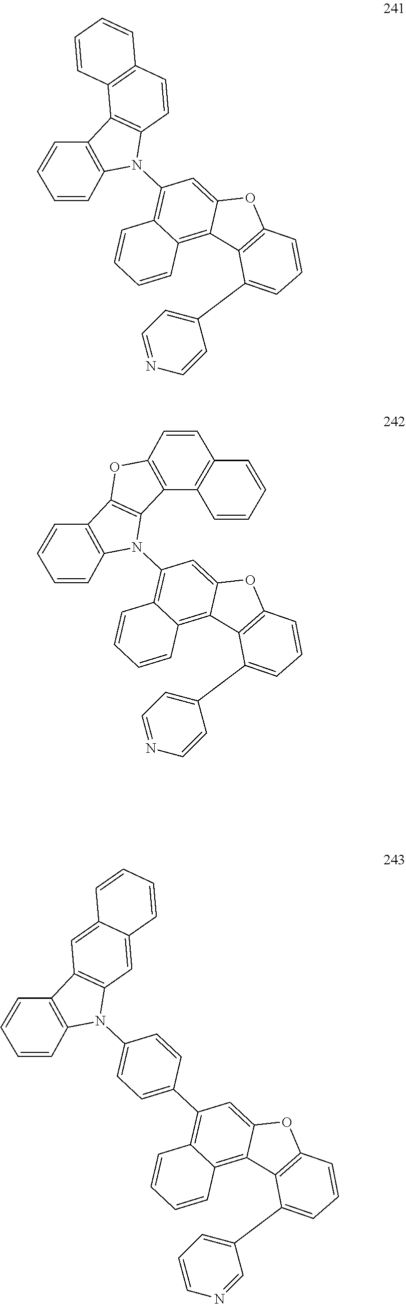 [SMILES: c1ccc2c(c1)ccc1c2c2ccccc2n1-c1cc2oc3cccc(-c4ccncc4)c3c2c2ccccc12 ; c1ccc2c(c1)ccc1oc3c4ccccc4n(-c4cc5oc6cccc(-c7ccncc7)c6c5c5ccccc45)c3c12 ; c1cncc(-c2cccc3oc4cc(-c5ccc(-n6c7ccccc7c7cc8ccccc8cc76)cc5)c5ccccc5c4c23)c1]

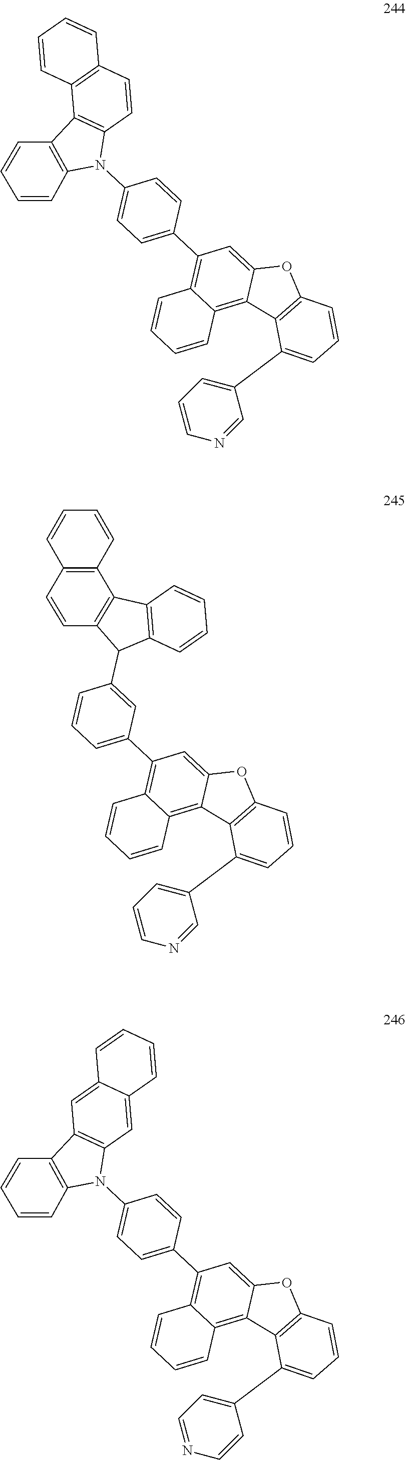 [SMILES: c1cncc(-c2cccc3oc4cc(-c5ccc(-n6c7ccccc7c7c8ccccc8ccc76)cc5)c5ccccc5c4c23)c1 ; c1cncc(-c2cccc3oc4cc(-c5cccc(C6c7ccccc7-c7c6ccc6ccccc76)c5)c5ccccc5c4c23)c1 ; c1ccc2cc3c(cc2c1)c1ccccc1n3-c1ccc(-c2cc3oc4cccc(-c5ccncc5)c4c3c3ccccc23)cc1]

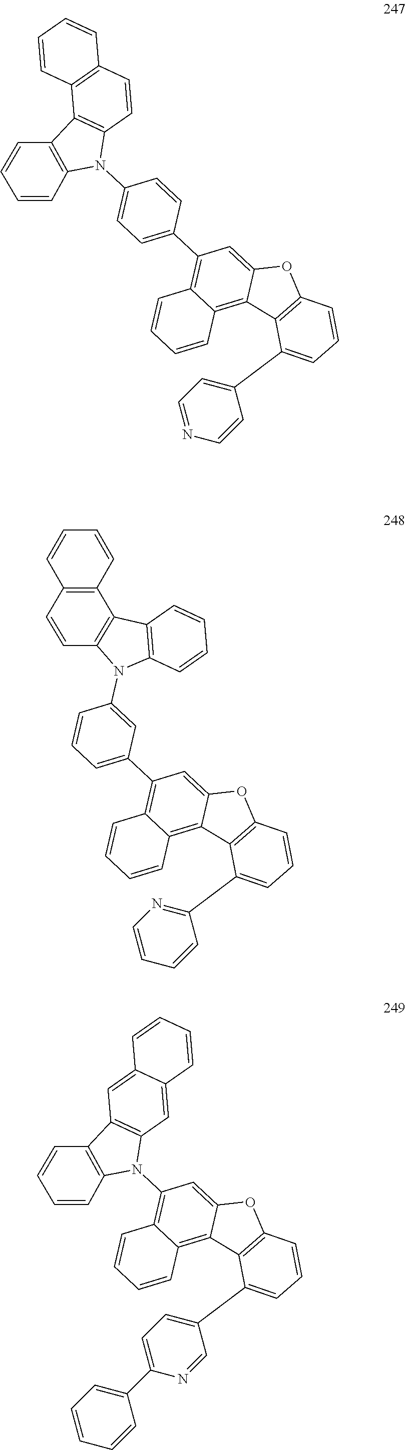 [SMILES: c1ccc2c(c1)ccc1c2c2ccccc2n1-c1ccc(-c2cc3oc4cccc(-c5ccncc5)c4c3c3ccccc23)cc1 ; c1ccc(-c2cccc3oc4cc(-c5cccc(-n6c7ccccc7c7c8ccccc8ccc76)c5)c5ccccc5c4c23)nc1 ; c1ccc(-c2ccc(-c3cccc4oc5cc(-n6c7ccccc7c7cc8ccccc8cc76)c6ccccc6c5c34)cn2)cc1]

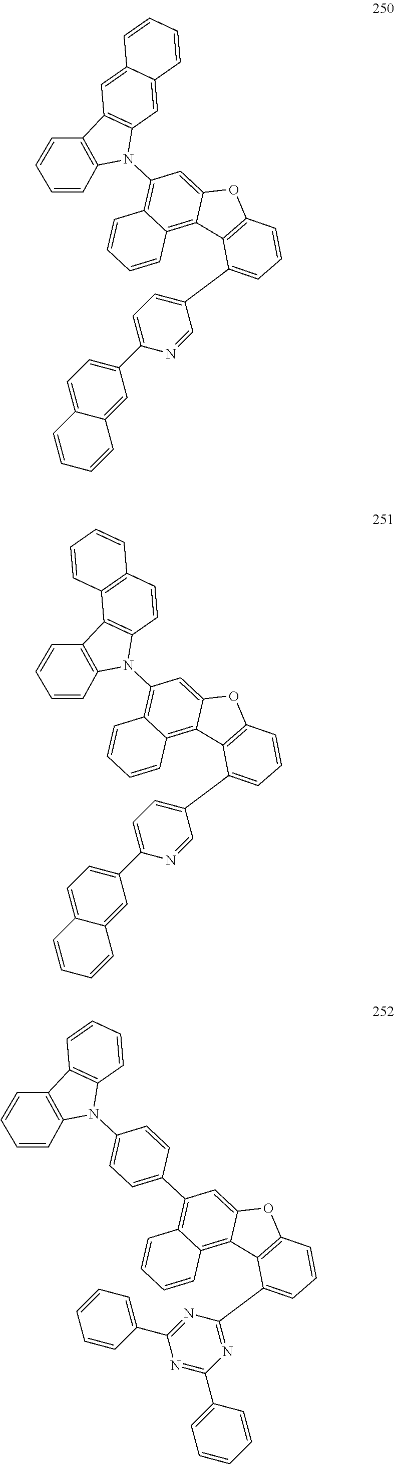

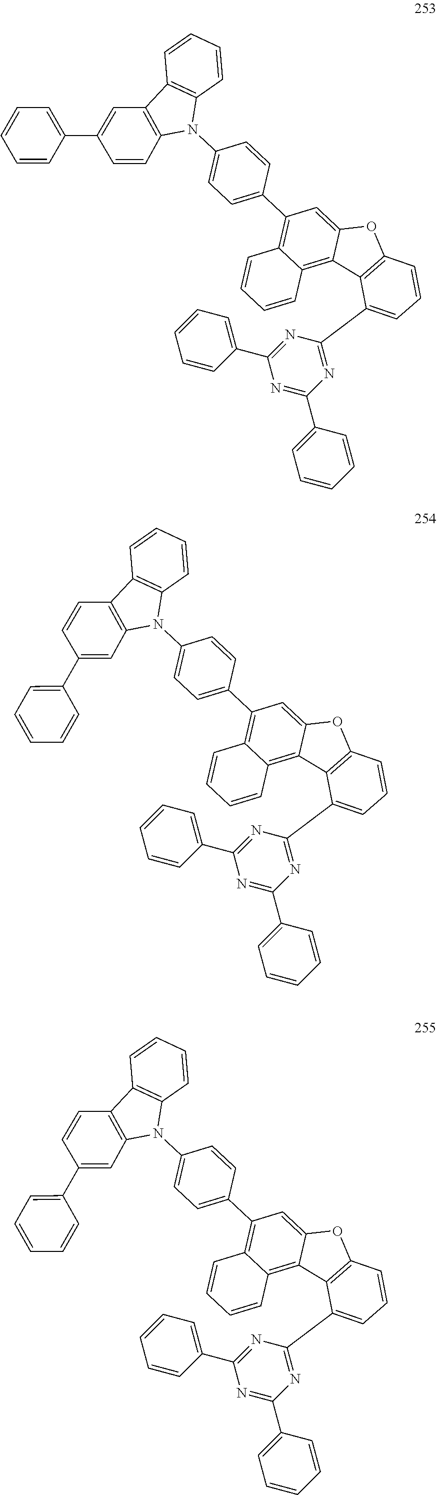 [SMILES: c1ccc(-c2ccc3c(c2)c2ccccc2n3-c2ccc(-c3cc4oc5cccc(-c6nc(-c7ccccc7)nc(-c7ccccc7)n6)c5c4c4ccccc34)cc2)cc1 ; c1ccc(-c2ccc3c4ccccc4n(-c4ccc(-c5cc6oc7cccc(-c8nc(-c9ccccc9)nc(-c9ccccc9)n8)c7c6c6ccccc56)cc4)c3c2)cc1 ; c1ccc(-c2ccc3c4ccccc4n(-c4ccc(-c5cc6oc7cccc(-c8nc(-c9ccccc9)nc(-c9ccccc9)n8)c7c6c6ccccc56)cc4)c3c2)cc1]

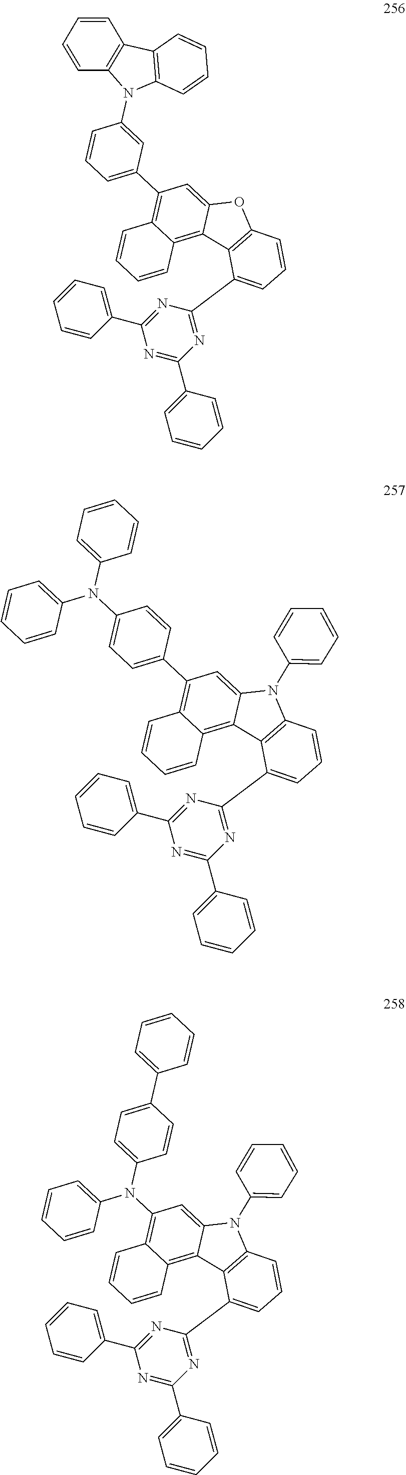 [SMILES: c1ccc(-c2nc(-c3ccccc3)nc(-c3cccc4oc5cc(-c6cccc(-n7c8ccccc8c8ccccc87)c6)c6ccccc6c5c34)n2)cc1 ; c1ccc(-c2nc(-c3ccccc3)nc(-c3cccc4c3c3c5ccccc5c(-c5ccc(N(c6ccccc6)c6ccccc6)cc5)cc3n4-c3ccccc3)n2)cc1 ; c1ccc(-c2ccc(N(c3ccccc3)c3cc4c(c5ccccc35)c3c(-c5nc(-c6ccccc6)nc(-c6ccccc6)n5)cccc3n4-c3ccccc3)cc2)cc1]

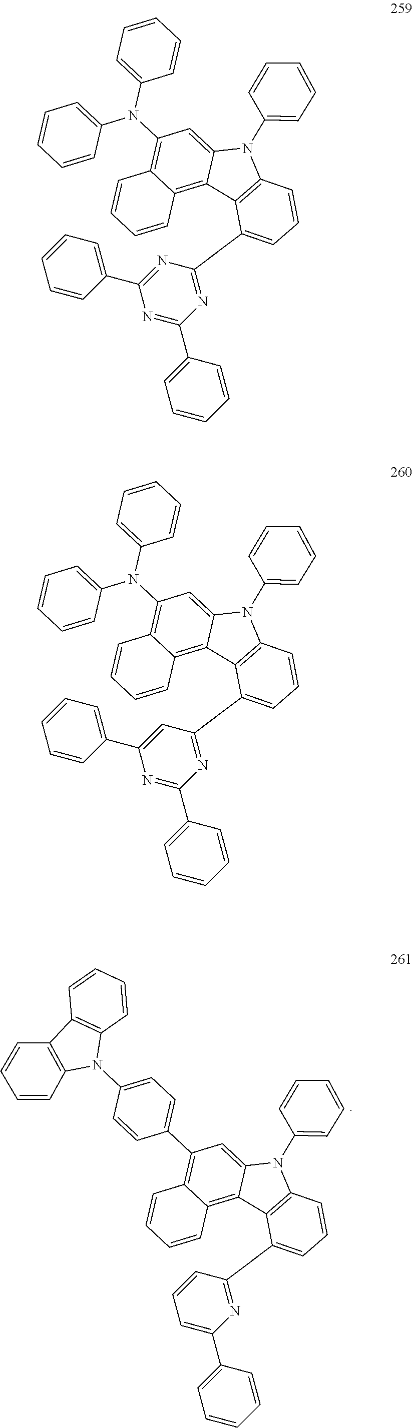 [SMILES: c1ccc(-c2nc(-c3ccccc3)nc(-c3cccc4c3c3c5ccccc5c(N(c5ccccc5)c5ccccc5)cc3n4-c3ccccc3)n2)cc1 ; c1ccc(-c2cc(-c3cccc4c3c3c5ccccc5c(N(c5ccccc5)c5ccccc5)cc3n4-c3ccccc3)nc(-c3ccccc3)n2)cc1 ; c1ccc(-c2cccc(-c3cccc4c3c3c5ccccc5c(-c5ccc(-n6c7ccccc7c7ccccc76)cc5)cc3n4-c3ccccc3)n2)cc1]

8. An organic light emitting device comprising:
a first electrode;
a second electrode; and
one or more organic material layers provided between the first electrode and the second electrode,
wherein one or more layers of the organic material layers comprise the heterocyclic compound of claim 1.

9. The organic light emitting device of claim 8, wherein the organic material layer comprises a light emitting layer, and the light emitting layer comprises the heterocyclic compound.

10. The organic light emitting device of claim 8, wherein the organic material layer comprises a light emitting layer, and the light emitting layer comprises the heterocyclic compound as a host material of a light emitting material.

11. The organic light emitting device of claim 10, wherein the light emitting layer comprises two or more host materials, and at least one of the host materials is the heterocyclic compound.

12. The organic light emitting device of claim 8, further comprising one, two or more layers selected from the group consisting of a light emitting layer, a hole injection layer, a hole transfer layer, an electron injection layer, an electron transfer layer, an electron blocking layer, a hole auxiliary layer and a hole blocking layer.

13. A composition for an organic material layer of an organic light emitting device, the composition comprising:
the heterocyclic compound represented by Chemical Formula 1 of claim 1; and
any one of the following heterocyclic compounds 1-1 to 1-14:

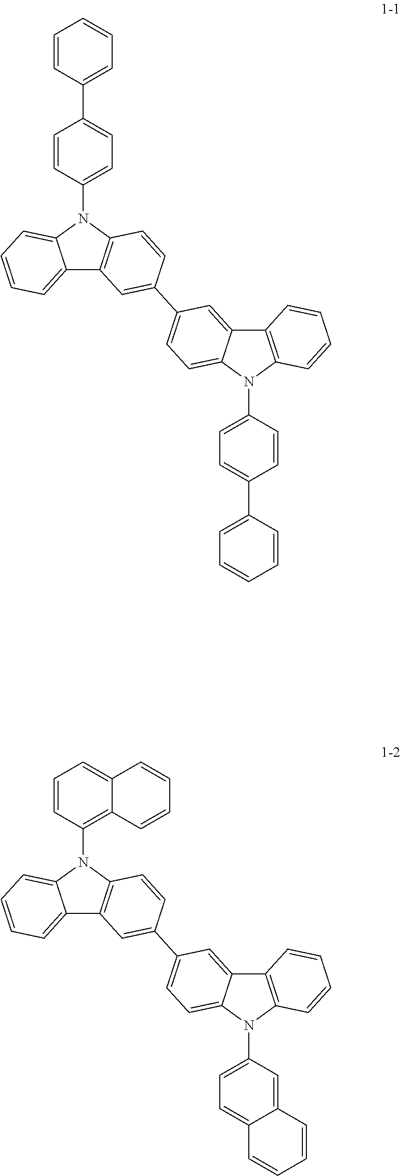 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

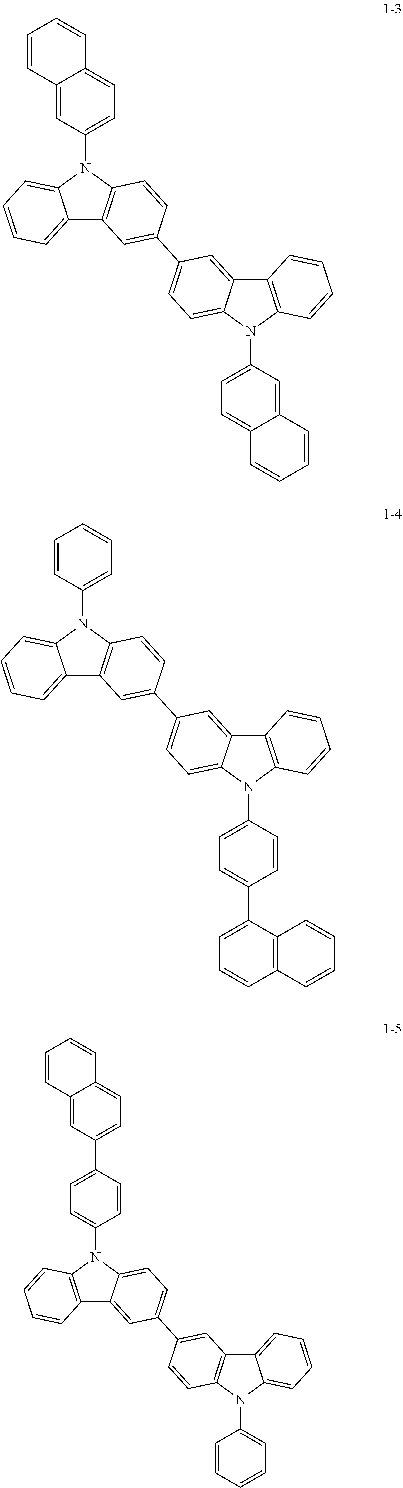 [SMILES: c1ccc2cc(-n3c4ccccc4c4cc(-c5ccc6c(c5)c5ccccc5n6-c5ccc6ccccc6c5)ccc43)ccc2c1 ; c1ccc(-n2c3ccccc3c3cc(-c4ccc5c(c4)c4ccccc4n5-c4ccc(-c5cccc6ccccc56)cc4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

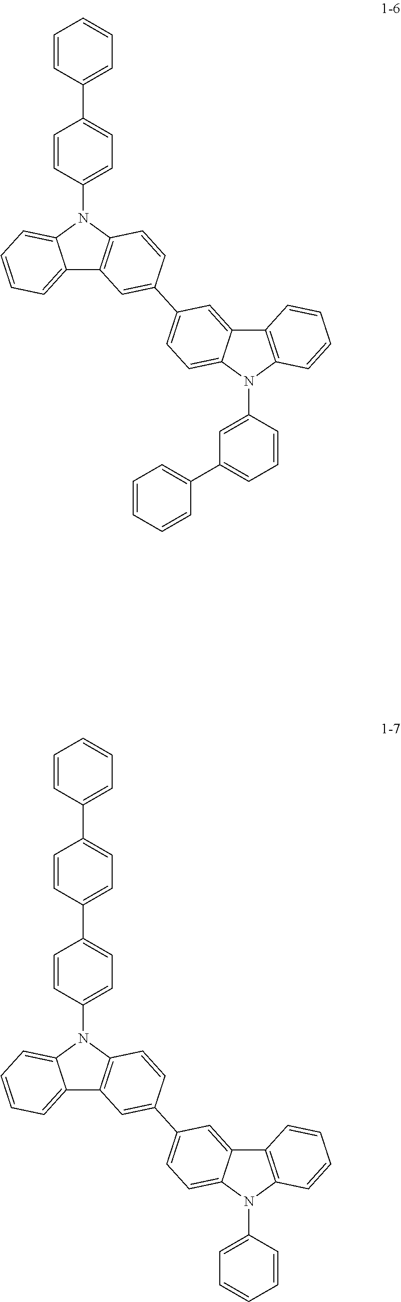 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

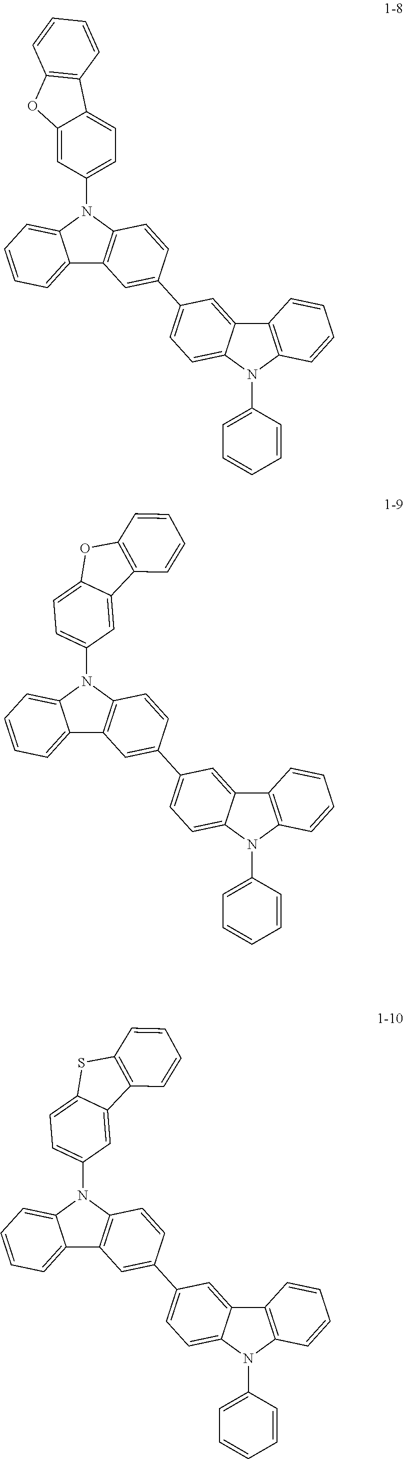 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc5sc6ccccc6c5c4)ccc32)cc1]

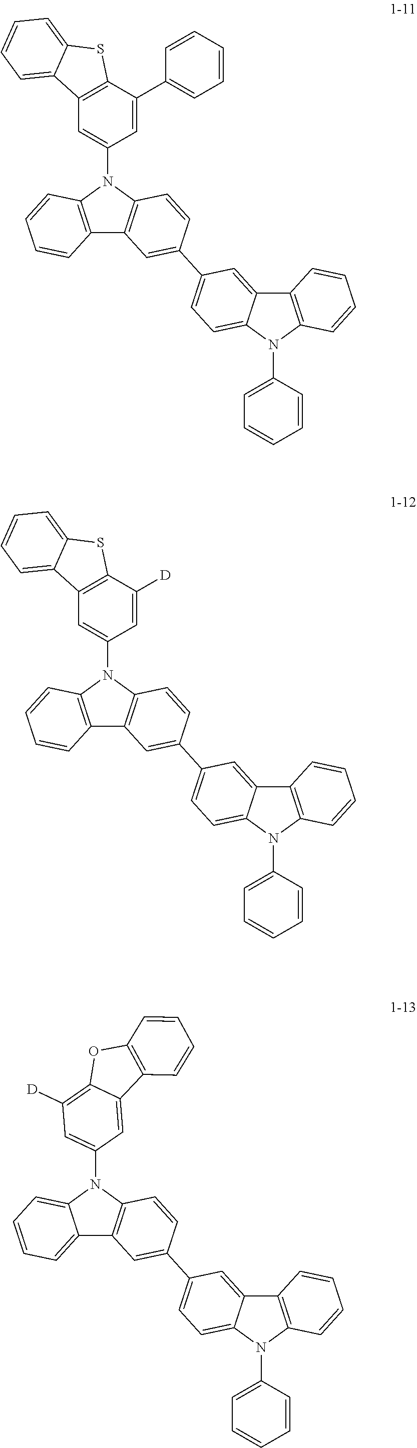 [SMILES: c1ccc(-c2cc(-n3c4ccccc4c4cc(-c5ccc6c(c5)c5ccccc5n6-c5ccccc5)ccc43)cc3c2sc2ccccc23)cc1 ; [2H]c1cc(-n2c3ccccc3c3cc(-c4ccc5c(c4)c4ccccc4n5-c4ccccc4)ccc32)cc2c1sc1ccccc12 ; [2H]c1cc(-n2c3ccccc3c3cc(-c4ccc5c(c4)c4ccccc4n5-c4ccccc4)ccc32)cc2c1oc1ccccc12]

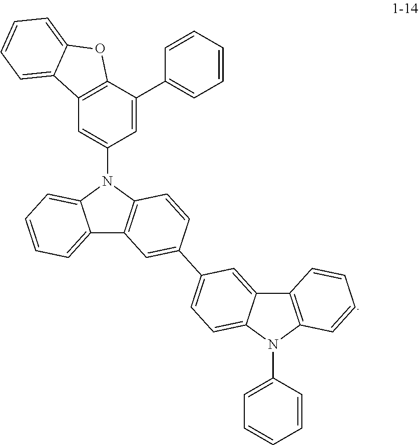 [SMILES: c1ccc(-c2cc(-n3c4ccccc4c4cc(-c5ccc6c(c5)c5ccccc5n6-c5ccccc5)ccc43)cc3c2oc2ccccc23)cc1]

14. The composition for an organic material layer of an organic light emitting device of claim 13, wherein, in the composition, the heterocyclic compound represented by Chemical Formula 1 and any one of the heterocyclic compounds 1-1 to 1-14 have a weight ratio of 1:10 to 10:1.
